(19) United States
(12) Patent Application Publication
(10) Pub. No.: US 2026/0193272 A1
(43) Pub. Date: Jul. 9, 2026

(54) SELENIUM CONTAINING HETEROCYCLE COMPOUNDS AND USE THEREOF

(71) Applicant: HELIOS HUAMING BIOPHARMA CO., LTD., Suzhou (CN)

(72) Inventors: Yanming WANG, Suzhou (CN); Zhengwei GUO, Suzhou (CN); Yanfei XIN, Suzhou (CN); Mingde XIA, Suzhou (CN)

(73) Assignee: HELIOS HUAMING BIOPHARMA CO., LTD., Suzhou (CN)

(21) Appl. No.: 19/132,870

(22) PCT Filed: Nov. 24, 2023
(86) PCT No.: PCT/CN2023/134167

§ 371 Date: May 27, 2025

(30) Foreign Application Priority Data
Nov. 24, 2022 (WO) PCT/CN2022/133855

(51) Int. Cl. C07D 517/04; A61K 31/4184; A61K 31/4439; A61K 31/444; A61K 31/454; A61K 31/4545; A61K 45/06; C07D 293/06

(52) U.S. Cl. CPC C07D 517/04; A61K 31/4184; A61K 31/4439; A61K 31/444; A61K 31/454; A61K 31/4545; A61K 45/06; C07D 293/06

(57) ABSTRACT

A series of selenium containing heterocycle compounds, its pharmaceutically acceptable salt, stereoisomer or solvate are provided. A pharmaceutical composition including the compound, and use of the compound as PAD inhibitors, especially PAD4 inhibitors, are also provided. The selenium containing heterocycle compounds exhibit greatly improved enzymatic PAD4 inhibition activities and/or cellular PAD4 inhibition activities and/or cellular citrullination inhibitory activity and/or cellular NET formation inhibitory activity over the oxygen or sulfur containing compounds.

### CROSS REFERENCE TO THE RELATED APPLICATIONS

This application is the national phase entry of International Application No. PCT/CN2023/134167, filed on Nov. 24, 2023, which is based upon and claims priority to International Application No. PCT/CN2022/133855, filed on Nov. 24, 2022, the entire contents of which are incorporated herein by reference.

### TECHNICAL FIELD

The present disclosure pertains to the biomedical field, and particularly relates to a series of selenium containing heterocycle compounds. The present disclosure also relates to a pharmaceutical composition comprising the compound, and the use of the compound as PAD inhibitors, especially PAD4 inhibitors.

### BACKGROUND

Post-translational modifications of histones have a major impact on the protein structure and function. Post-translational modifications of histones can enhance the functional diversity of proteins through proteolysis and protein degradation, such as phosphorylation, glycosylation, ubiquitination, nitrosylation, methylation, citrullination, acetylation and lipidation, and thereby affecting the occurrence and development of various diseases. Therefore, study on the cellular biology of post-translational modifications of histones is critical for disease diagnosis and prevention. Peptidyl arginine deiminases (PADs) can catalyze conversion of the positively charged arginine (Arginine, R) or monomethyl arginine in substrate proteins (such as transcription factors, histones, etc.) to electrically neutral citrulline (Citrulline, Cit) in the presence of Ca²⁺. The citrullinated protein often changes its original molecular conformation, resulting in changes in the biochemical activities of the protein, and participates in the occurrence and development of various disease processes.

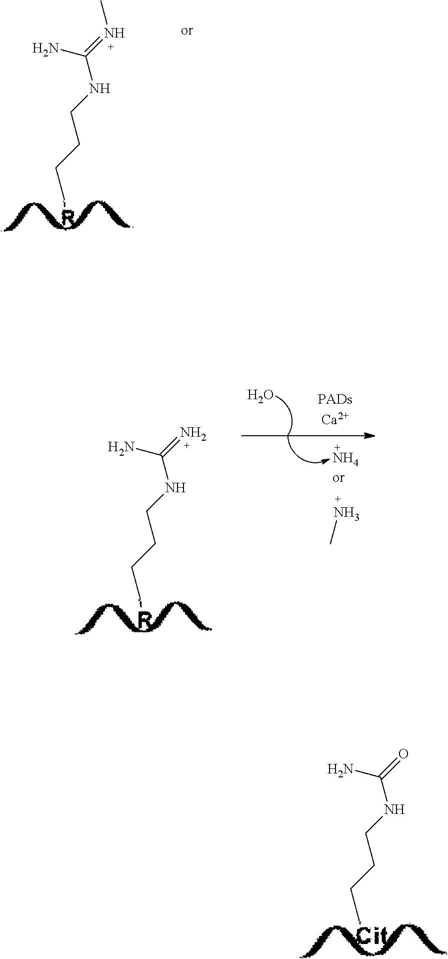

### PAD Catalyzes Citrullination of the Arginine Residue

The peptidyl arginine deiminase (PAD) family consists of five isoenzymes PAD1, PAD2, PAD3, PAD4 and PAD6. PAD family members are expressed in specific tissues, for example, PAD4 is mainly expressed in myeloid lineage cells such as neutrophils, monocytes and macrophages. PAD4 is the only PAD isozyme with a nuclear localization signal, and PAD4-mediated post-translational citrullination is involved in the expression and regulation of multiple regulators involved in transcriptional regulation, cell cycle, apoptosis, and formation of neutrophil extracellular traps (NETs). As such, PAD4 promotes the pathological process of various diseases such as autoimmune diseases, cardiovascular diseases, neurodegenerative diseases and tumors through citrullination.

PAD4 regulates the formation of NETs by catalyzing the citrullination of histone arginine. NETs are highly concentrated and reticulated DNA/protein complexes released extracellularly by neutrophils activated through different means, and in addition to depolymerized chromatin DNA, the reticulated structure also carries various proteins and enzymes such as citrullination histone H3 (CitH3), neutrophil elastase (NE), myeloperoxidase (MPO), etc. NETs are associated with the pathogenesis of autoimmune diseases such as systemic lupus erythematosus, rheumatoid arthritis, psoriasis, etc., as well as other non-infectious pathological processes such as coagulation disorders, thrombosis, diabetes, atherosclerosis, vasculitis, and cancer. In recent years, data from animal models and tumor patients have shown that PAD4 is highly expressed in various tumor tissues, and the formation of NETs mediated by PAD4 is involved in the progression, metastasis and recurrence of various tumors. In the tumor microenvironment, neutrophils recruited by tumor cells (tumor-associated neutrophils) and the formation of NETs are closely related. This relationship was found in both primary and metastasis tumors. NETs can capture circulating tumor cells and promote tumor metastasis. NETs can awaken dormant tumor cells and promote tumor recurrence and metastasis. The formation of NETs can be regulated through inhibiting the activity of PAD4 with small molecule PAD4 inhibitors, thereby playing a role in the treatment of NETs-related tumors and other related diseases. (Frontiers in immunology, 2020, 11, 1749; Acta Biochim Biophys Sin, 2017, 49(7), 567-572; Acc. Chem. Res. 2019, 52, 818-832; Int. J. Cancer. 2021, 148:267-276)

The PAD inhibitors are mainly divided into two categories: covalent irreversible inhibitors and non-covalent reversible inhibitors. The first type of irreversible inhibitors is mainly obtained through modification of the substrate arginine structure of PADs, which is represented by halogenamidine compounds. The mechanism of action is that the cysteine in the active center of PADs can undergo substitution reaction with the halogenamidine compounds to generate a covalent bond, and thus deactivating the PAD enzymes. Among them, Cl-amidine and YW3-56 are two representative examples. Although these inhibitors have achieved good efficacy in cell and animal studies, they have certain limitations, such as lack of selectivity among PADs, short in vivo half-life, relatively low activity, and lack of oral bioavailability, etc.

The second type of reversible PAD inhibitors is now being studied more widely. This type of inhibitors is mainly allosteric inhibitors for PAD4. This type of inhibitors not only has improved selectivity on PAD4, but also has greatly improved inhibitory activity on PAD4. The IC₅₀ of in vitro PAD4 inhibition has reached nM level. For example, the IC₅₀ of the PAD4 inhibitor I-2 published in WO2017/100601 reached 120 nM. The subsequent patents of BMS Company further expanded this type of molecules, and obtained a number of compounds with different structures. The compound with the highest enzymatic activity has even reached an IC₅₀ of 5 nM (BMS-390) according to their assay method reported. However, the cellular citrullination inhibition activity of these molecules was not improved much, and their activity of inhibiting the formation of NETs at the cellular level is still far from satisfactory.

There is an urgent need to develop novel PAD4 inhibitors to overcome the problems of current PAD4 inhibitors, including poor selectivity, low bioavailability and low potency. PAD4 as a potential multi-disease target will receive more and more attention. The present disclosure developed new PAD4 inhibitors with higher in vitro enzymatic activities, higher cellular activities and preferably better druggability.

### SUMMARY

In one aspect, the present disclosure provides a new series of selenium containing heterocycle compounds, its pharmaceutically acceptable salt, stereoisomer or solvate. For the first time, the present disclosure introduces the selenium atom into the backbone of the PAD4 inhibitor, and surprisingly finds that such selenium containing heterocycle compounds exhibit greatly improved enzymatic PAD4 inhibition activities and/or cellular PAD4 inhibition activities and/or cellular citrullination inhibitory activity and/or cellular NET formation inhibitory activity over the oxygen or sulfur containing compounds.

In preferred embodiments, the selenium containing heterocycle compounds of the present disclosure have enzymatic PAD4 inhibitory activity of three or more folds improved compared with the non-selenium containing compounds.

In preferred embodiments, the selenium containing heterocycle compounds of the present disclosure have cellular PAD4 inhibitory activity/cellular citrullination inhibitory activity/cellular NET formation inhibitory activity of four or more folds improved compared with the non-selenium containing compounds.

In preferred embodiments, the selenium containing heterocycle compounds of the present disclosure have cellular PAD4 catalyzed citrullination inhibitory EC₅₁ of below 1.5 μM, preferably below 1 μM, more preferably below 0.5 μM, and most preferably below 0.2 μM.

In preferred embodiments, the selenium containing heterocycle compounds of the present disclosure have improved selectivity on PAD4 over other PAD isoenzymes.

In another aspect, the present disclosure provides a method for treating PAD or NET mediated diseases, the method comprises administering to an individual a therapeutically effective amount of the selenium containing heterocycle compounds.

In another aspect, the present disclosure provides use of the selenium containing heterocycle compounds for preparing a medicament for treating PAD or NET mediated diseases in an individual.

In another aspect, the present disclosure provides a method of treating PAD or NET mediated diseases in an individual with the selenium containing heterocycle compounds.

In a preferred embodiment, the present disclosure provides compounds of the Formula (I), its pharmaceutically acceptable salt, stereoisomer or solvate,

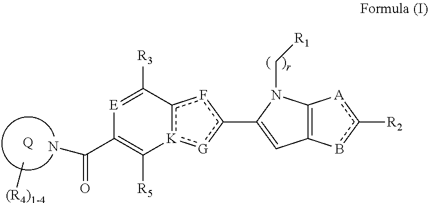

wherein,
one of A and B is Se, and the other one is N or CH;
E is N or CR₆;
K is C or N;
F and G are independently selected from the group consisting of N, NR₇, and CR₇; R₇ is selected from the group consisting of hydrogen, and C₁-C₈ alkyl;
Ring Q is 4- to 15-membered heterocyclyl optionally substituted with 1-4 R₄;
the dashed lines denote the optionally existing double bond, wherein the five-membered ring containing A and B is a heteroaryl ring, and the five-membered ring containing F, G, and H is a heteroaryl ring;
r, at each occurrence, is independently selected from 0, 1, 2, 3 and 4;
R₁ and R₂ are independently selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl and C₅-C₁₀ heteroaryl; which is optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy or C₁-C₈ haloalkyl;
R₃, R₅ and R₆ are independently selected from the group consisting of H, F, Cl, Br, C₁-C₄ alkyl, and C₁-C₄ alkoxy;
R₄ is selected from the group consisting of H, F, Cl, Br, —CN, —NH₂, —OH, —NO₂, C₁-C₄ alkyl, —(CH₂)ₚNRₐRₐ, —(CH₂)ₚOR_{b}, optionally two R₄ groups are taken together to form a carbocyclyl or heterocyclyl;
Rₐ and R_{b}, at each occurrence, is independently selected from H and C₁-C₄ alkyl;
p, at each occurrence, is independently selected from 0, 1 and 2.

In a preferred embodiment, the present disclosure provides compounds, its pharmaceutically acceptable salt, stereoisomer or solvate, the compounds have the Formula (IIa) or (IIb):

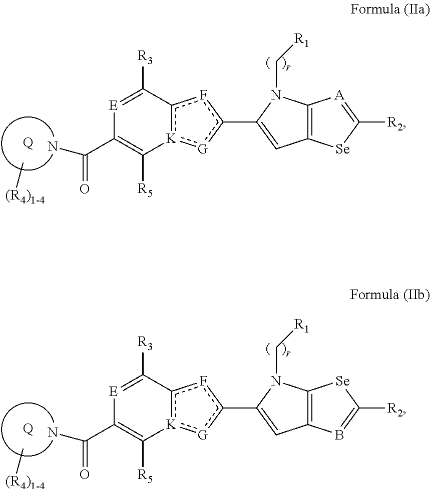

wherein when the compound has the Formula (IIa), A is N or CH; when the compound has the Formula (IIb), B is N or CH;
Ring Q is 4- to 12-membered heterocyclyl, preferably 4- to 7-membered monoheterocyclyl, 7- to 12-membered spiro heterocyclyl, or 6- to 10-membered bridged heterocyclyl; which is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, —(CH₂)ₚOR_{b};
E, F, G, K, R₁, R₂, R₃, R₄, R₅, R₆, R₇, Rₐ, R_{b}, R_{c}, r, and p have the meanings as defined above.

In a preferred embodiment, the present disclosure provides compounds, its pharmaceutically acceptable salt, stereoisomer or solvate, the compounds have the Formula (IIIa) or (IIb):

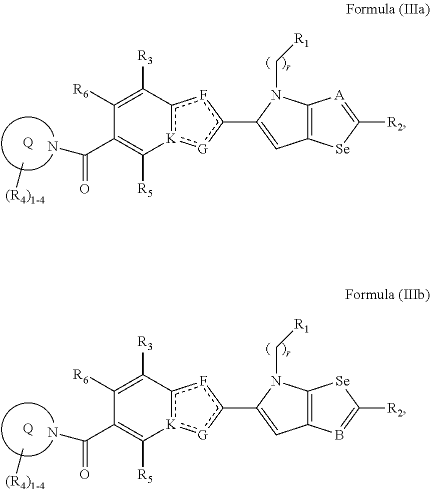

wherein when the compound has the Formula (IIIa), A is N or CH; when the compound has the Formula (IIIb), B is N or CH;
Ring Q is 4- to 12-membered heterocyclyl, preferably 4- to 7-membered monoheterocyclyl, 7- to 12-membered spiro heterocyclyl, or 6- to 10-membered bridged heterocyclyl; which is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, —(CH₂)ₚOR_{b};
E, F, G, K, R₁, R₂, R₃, R₄, R₅, R₆, R₇, Rₐ, R_{b}, R_{c}, r, and p have the meanings as defined above.

In a preferred embodiment, the present disclosure provides compounds of above formulae, its pharmaceutically acceptable salt, stereoisomer or solvate, wherein
B is Se;
A is N or CH;
the bicyclic ring containing E, F, G and K is selected from the group consisting of the following structures:

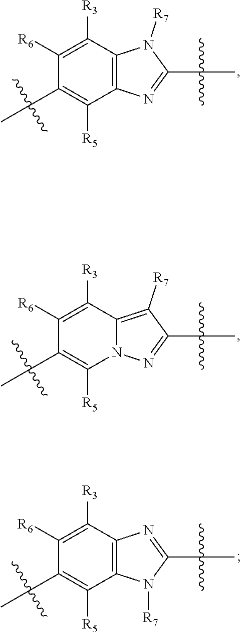

R₁, R₂, R₃, R₄, R₅, R₆, R₇, Rₐ, R_{b}, R_{c}, r, and p have the meanings as defined above.

In a preferred embodiment, the present disclosure provides compounds of the above formulae, its pharmaceutically acceptable salt, stereoisomer or solvate,
wherein Ring Q is selected from the group consisting of:

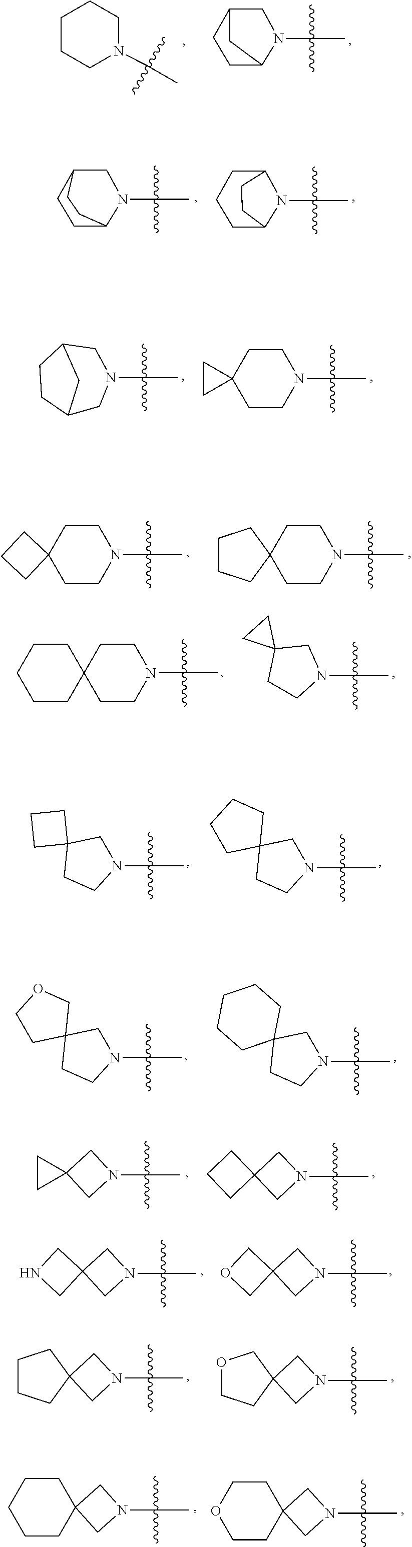

which is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, —(CH₂)ₚOR_{b}; Rₐ, R_{b}, and p have the meanings as defined above.

In a preferred embodiment, the present disclosure provides compounds, its pharmaceutically acceptable salt, stereoisomer or solvate, the compound has the Formula (IVa) or (IVb):

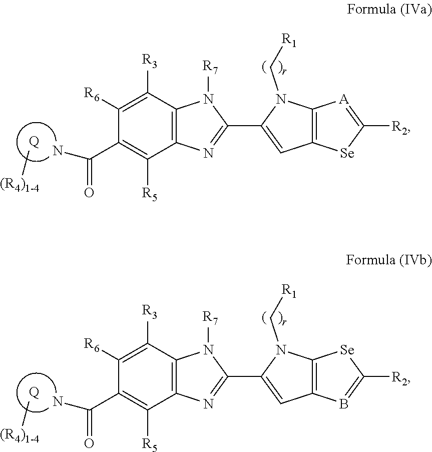

wherein when the compound has the Formula (IVa), A is N or CH; when the compound has the Formula (IVb), B is N or CH;
Ring Q with R₄ substitution is selected from the group consisting of

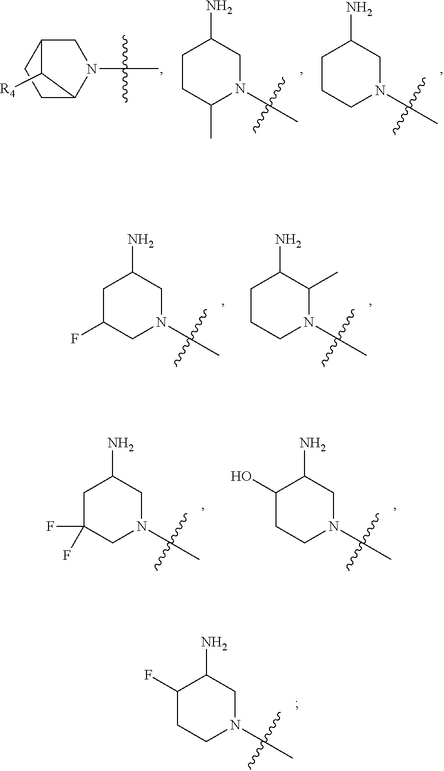

wherein R₄ in

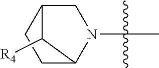

is selected from the group consisting of H, F, Cl, Br, —CN, —NH₂, —OH, —NO₂, C₁-C₄ alkyl, —(CH₂)ₚNRₐRₐ, —(CH₂)ₚOR_{b}; R₁, R₂, R₃, R₄, R₅, R₆, R₇, Rₐ, R_{b}, R_{c}, r, and p have the meanings as defined above.

In a preferred embodiment, the present disclosure provides compounds, its pharmaceutically acceptable salt, stereoisomer or solvate, the compound has the Formula (IVa) or (IVb):

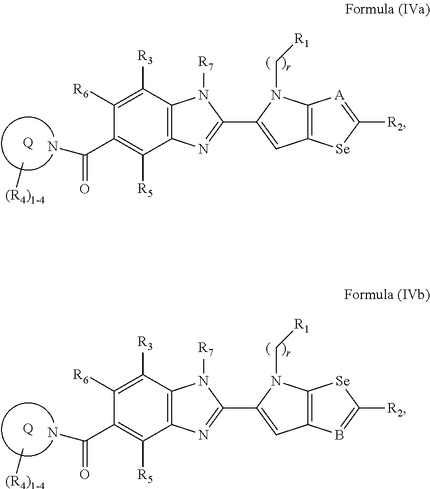

wherein when the compound has the Formula (IVa), A is N or CH; when the compound has the Formula (IVb), B is N or CH;
Ring Q with R₄ substitution is selected from the group consisting of

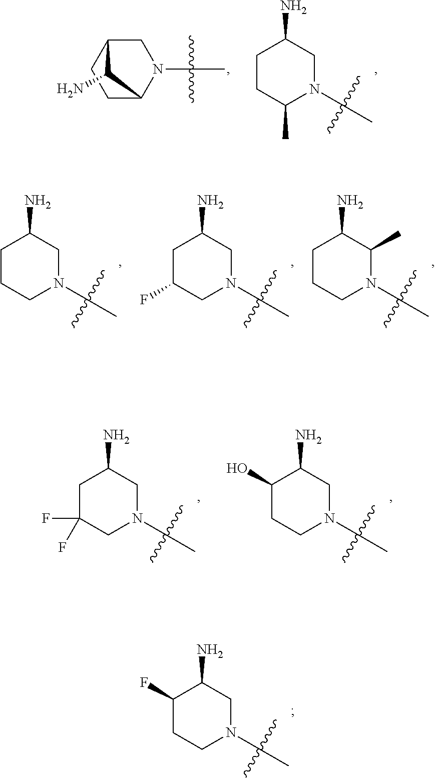

R₁ is selected from the group consisting of H, C₁-C₈ alkyl substituted with 0-5 R_{c}, C₃-C₆ cycloalkyl substituted with 0-5 R_{c}, C₆-C₁₀ aryl substituted with 0-5 R_{c}, and C₅-C₁₀ heteroaryl substituted with 0-4 R_{c};
R₂ is selected from the group consisting of H, C₁-C₈ alkyl substituted with 0-5 R_{c}, C₃-C₆ cycloalkyl substituted with 0-5 R_{c}, C₅-C₉ heterocycloalkyl substituted with 0-5 R_{c}, C₆-C₁₀ aryl substituted with 0-5 R_{c}, and C₅-C₁₀ heteroaryl substituted with 0-4 R_{c};
R₃ is selected from the group consisting of H, F, Cl, Br, C₁-C₄ alkyl, and C₁-C₄ alkoxy, and is preferably H, F, or C₁-C₃ alkoxy;
R₅ and R₆ are independently selected from the group consisting of H, F, Cl, Br, C₁-C₄ alkyl, and C₁-C₄ alkoxy, and are preferably H, F or C₁-C₃ alkyl;
R₇ is selected from the group consisting of hydrogen and C₁-C₈ alkyl, and is preferably H or C₁-C₃ alkyl;
R_{c} at each occurrence is independently selected from the group consisting of F, Cl, Br, —CN, —NO₂, —OH, —NH₂, C₁-C₃ alkyl, C₁-C₃ alkoxy and C₁-C₃ haloalkyl;
r is 1 or 2.

In a preferred embodiment, the present disclosure provides compounds, its pharmaceutically acceptable salt, stereoisomer or solvate, wherein
R₁ is selected from cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, pyridinyl substituted with 0-2 R_{c};
R₂ is selected from H, F, Cl, Br, methyl, ethyl, cyclopropyl substituted with 0-2 R_{c}, phenyl substituted with 0-2 R_{c}, pyrrolidinyl substituted with 0-2 R_{c}, pyridinyl substituted with 0-2 R_{c}; piperidinyl substituted with 0-2 R_{c};
R_{c} is at each occurrence is independently selected from the group consisting of F, C₁-C₃ alkyl, C₁-C₃ alkoxy and C₁-C₃ haloalkyl, and is preferably methyl, —OMe, or —CF₃;
r is 1.

In another embodiment, the present disclosure provides compounds of the formula (V), its pharmaceutically acceptable salt, stereoisomer or solvate,

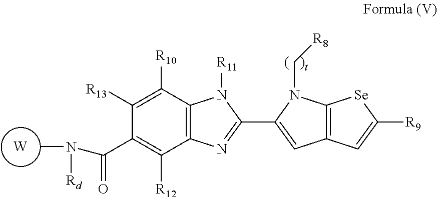

wherein
Ring W is 4- to 15-membered heterocyclyl, preferably 4- to 7-membered monoheterocyclyl, more preferably 5- to 7-membered monoheterocyclyl, and Ring W is optionally substituted with 1-4 R₁₄;
t, at each occurrence, is independently selected from 0, 1, 2, 3 and 4;
R₈ and R₉ are independently selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl and C₅-C₁₀ heteroaryl; which is optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy or C₁-C₈ haloalkyl;
R₁₀, R₁₂ and R₁₃ are independently selected from the group consisting of H, F, Cl, Br, C₁-C₄ alkyl, and C₁-C₄ alkoxy;
R₁₁ is selected from the group consisting of hydrogen, and C₁-C₈ alkyl;
R₁₄ is selected from the group consisting of H, F, Cl, Br, —CN, —NH₂, —OH, —NO₂, C₁-C₄ alkyl;
R_{d} is independently selected from H and C₁-C₄ alkyl.

In a preferred embodiment, the present disclosure provides compounds of the formula (V), its pharmaceutically acceptable salt, stereoisomer or solvate, wherein Ring W is 6-membered monoheterocyclyl, preferably piperidinyl, and Ring W is optionally substituted with 1 or 2 R₁₄.

In a preferred embodiment, the present disclosure provides compounds of the formula (V), its pharmaceutically acceptable salt, stereoisomer or solvate, wherein
t is 1 or 2;
R₈ is selected from the group consisting of H, C₁-C₅ alkyl substituted with 0-5 R_{c}, C₃-C₆ cycloalkyl substituted with 0-5 Re, C₆-C₁₀ aryl substituted with 0-5 R_{c}, and C₅-C₁₀ heteroaryl substituted with 0-4 R_{c};
R₉ is selected from the group consisting of H, C₁-C₅ alkyl substituted with 0-5 R_{c}, C₃-C₆ cycloalkyl substituted with 0-5 R_{c}, C₅-C₉ heterocycloalkyl substituted with 0-5 R_{c}, C₆-C₁₀ aryl substituted with 0-5 R_{c}, and C₅-C₁₀ heteroaryl substituted with 0-4 R_{c};
R₁₀ is selected from the group consisting of H, F, Cl, Br, C₁-C₄ alkyl, and C₁-C₄ alkoxy, and is preferably H, F, or C₁-C₃ alkoxy;
R₁₂ and R₁₃ are independently selected from the group consisting of H, F, Cl, Br, C₁-C₄ alkyl, and C₁-C₄ alkoxy, and are preferably H, F or C₁-C₃ alkyl;
R₁₁ is selected from the group consisting of hydrogen and C₁-C₈ alkyl, and is preferably H or C₁-C₃ alkyl; and R_{c} at each occurrence is independently selected from the group consisting of F, Cl, Br, —CN, —NO₂, —OH, —NH₂, C₁-C₃ alkyl, C₁-C₃ alkoxy and C₁-C₃ haloalkyl.

In a preferred embodiment, the present disclosure provides compounds of the formula (V), its pharmaceutically acceptable salt, stereoisomer or solvate, wherein
R₈ is selected from cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, pyridinyl substituted with 0-2 R_{c};
R₉ is selected from H, F, Cl, Br, methyl, ethyl, cyclopropyl substituted with 0-2 R_{c}, phenyl substituted with 0-2 R_{c}, pyrrolidinyl substituted with 0-2 R_{c}, pyridinyl substituted with 0-2 R_{c}; piperidinyl substituted with 0-2 R_{c};
R_{c} is at each occurrence is independently selected from the group consisting of F, C₁-C₃ alkyl, C₁-C₃ alkoxy and C₁-C₃ haloalkyl, and is preferably methyl, —OMe, or —CF₃;
R_{d} is H; and
t is 1.

In another embodiment, the present disclosure provides compounds having the structures as shown in Scheme 1.

In another embodiment, the present disclosure provides a pharmaceutical composition comprising the above compound, its pharmaceutically acceptable salt, stereoisomer or solvate, and one or more pharmaceutically acceptable carriers.

In another embodiment, the present disclosure provides a method of treating PAD4-mediated or NET-mediated disease, disorder or condition in a subject, the method comprising administering to the subject a therapeutically effective amount of the above compound, its pharmaceutically acceptable, stereoisomer or solvate, wherein said disease, disorder or condition are selected from the group consisting of cancers, rheumatoid arthritis, multiple sclerosis, vasculitis, systemic lupus erythematosus, ulcerative colitis, cystic fibrosis, asthma, cutaneous lupus erythematosis, psoriasis, Alzheimer's disease, ischemia-reperfusion injury, and immune responses induced during transplant rejection.

In a preferred embodiment, the present disclosure provides a method of treating PAD4-mediated or NET-mediated disease, disorder or condition in a subject, wherein the disease, disorder or condition is selected from the group consisting of lung cancer, liver cancer, blood cancer, esophageal cancer, breast cancer and colon cancer.

In another aspect, the present disclosure provides a combination therapy, comprising administering to the subject a first therapeutic agent and one or more additional therapeutics, the first therapeutic agent comprises the above compound, its pharmaceutically acceptable, stereoisomer or solvate, said one or more additional therapeutics is selected from the group consisting of radiotherapy, chemotherapy, cell therapy such as CAR-T, CAR-NK, CAR-NKT, CAR-M, CAR-Treg, CAR-γδT, TIL, TCR-T et al, and/or immune checkpoint inhibitor, and preferably the immune checkpoint inhibitor includes PD-1 inhibitor, PD-L1 inhibitor, CTLA-4 inhibitor, B7-H3 inhibitor, LAG3 inhibitor, TIM3 inhibitor, TIGIT inhibitor, anti-PDL1/TGFβ bispecific antibody, anti-EpCAM-CD3 bispecific antibody, and/or CD40 agonists.

In a preferred embodiment, the present disclosure provides a combination therapy, wherein the first therapeutic agent and the one or more additional therapeutics are administered together, simultaneously, sequentially or alternately.

In a preferred embodiment, the present disclosure provides a combination therapy, wherein the first therapeutic agent or the one or more additional therapeutics are administrated in a lower dose in comparison with the dose of the therapeutic agent or therapeutic when it is administered alone.

In another aspect, the present disclosure provides a method of treating PAD4-mediated or NET-mediated disease, disorder or condition in a subject, the method comprising administering to the subject a therapeutically effective amount of the above compound, its pharmaceutically acceptable, stereoisomer or solvate, wherein the subject is also administered with one or more additional therapeutics selected from the group consisting of radiotherapy, chemotherapy, cell therapy such as CAR-T, CAR-NK, CAR-NKT, CAR-M, CAR-Treg, CAR-γδT, TIL, TCR-T et al, and/or immune checkpoint inhibitor, and preferably the immune checkpoint inhibitor includes PD-1 inhibitor, PD-L1 inhibitor, CTLA-4 inhibitor, B7-H3 inhibitor, LAG3 inhibitor, TIM3 inhibitor, TIGIT inhibitor, anti-PDL1/TGFβ bispecific antibody, anti-EpCAM-CD3 bispecific antibody, and/or CD40 agonists.

In a preferred embodiment, the present disclosure provides a method of treating PAD4-mediated or NET-mediated disease, disorder or condition in a subject, wherein said disease, disorder or condition are selected from the group consisting of cancers, rheumatoid arthritis, multiple sclerosis, vasculitis, systemic lupus erythematosus, ulcerative colitis, cystic fibrosis, asthma, cutaneous lupus erythematosis, psoriasis, Alzheimer's disease, ischemia-reperfusion injury, and immune responses induced during transplant rejection, and preferably the disease, disorder or condition is selected from the group consisting of lung cancer, liver cancer, blood cancer, esophageal cancer, breast cancer and colon cancer.

In a preferred embodiment, the present disclosure provides a method of treating PAD4-mediated or NET-mediated disease, disorder or condition in a subject, wherein said above compound and the one or more additional therapeutics are administered together, simultaneously, sequentially or alternately.

In another aspect, the present disclosure provides a method for preparing a medicament for treating PAD4-mediated or NET-mediated disease, disorder or condition in a subject with the above compound, the method comprises administering to the subject a therapeutically effective amount of the above compound, its pharmaceutically acceptable, stereoisomer or solvate.

In a preferred embodiment, the present disclosure provides a method for preparing a medicament for treating PAD4-mediated or NET-mediated disease, disorder or condition in a subject with the above compound, wherein the subject is also administered with one or more additional therapeutics selected from the group consisting of radiotherapy, chemotherapy, cell therapy such as CAR-T, CAR-NK, CAR-NKT, CAR-M, CAR-Treg, CAR-γδT, TIL, TCR-T et al, and/or immune checkpoint inhibitor, and preferably the immune checkpoint inhibitor includes PD-1 inhibitor, PD-L1 inhibitor, CTLA-4 inhibitor, B7-H3 inhibitor, LAG3 inhibitor, TIM3 inhibitor, TIGIT inhibitor, anti-PDL1/TGFβ bispecific antibody, anti-EpCAM-CD3 bispecific antibody, and/or CD40 agonists.

In another embodiment, the present disclosure provides a method for preparing selenazole compound S-4, comprising the following steps:

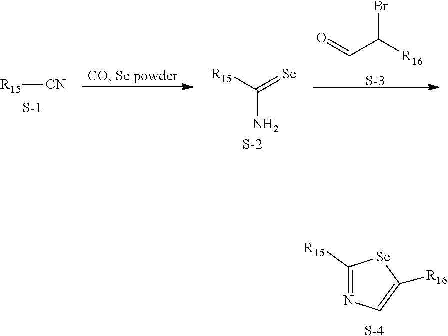

react the nitrile compound S-1 with CO and Se powder to yield compound S-2, which is further reacted with compound S-3 to yield compound S-4, wherein,
R₁₅ is selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl and C₅-C₁₀ heteroaryl; which is optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy or C₁-C₈ haloalkyl; is preferably selected from the group consisting of H, C₁-C₅ alkyl substituted with 0-5 R_{c}, C₃-C₆ cycloalkyl substituted with 0-5 R_{c}, C₆-C₁₀ aryl substituted with 0-5 R_{c}, and C₅-C₁₀ heteroaryl substituted with 0-4 R_{c}; and is more preferably selected from the group consisting of cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, pyridinyl substituted with 0-2 R_{c};
R_{c} is at each occurrence is independently selected from the group consisting of F, C₁-C₃ alkyl, C₁-C₃ alkoxy and C₁-C₃ haloalkyl, and is preferably methyl, —OMe, or —CF₃;
R₁₆ is CHO.

In a preferred embodiment, the present disclosure provides a method for preparing selenazole compound S-4, comprising the above steps, wherein the two reacting steps are performed at elevated temperature.

In another aspect, the present disclosure provides a method of synthesizing selenium containing heterocycle compounds, such as selenazoles, and others.

### DETAILED DESCRIPTION OF THE EMBODIMENTS

### Definitions

Unless otherwise defined below, all technical and scientific terms used herein have the same meanings as commonly understood by one of ordinary skill in the art. References to techniques used herein are intended to refer to techniques that are generally understood in the art, including those obvious changes or equivalent replacements of the techniques for those skilled in the art. While it is believed that the following terms are well understood by those skilled in the art, the following definitions are set forth to better explain the present disclosure.

As used herein, the terms “including”, “comprising”, “having”, “containing” or “comprising”, and other variants thereof, are inclusive or open, and do not exclude other unlisted elements or method steps.

Unless otherwise indicated, when a range of any type is disclosed or claimed, it is intended to cover each possible value that the range could reasonably cover, including any sub-ranges contained therein. For example, the number of groups from 1 to 6 indicates an integer within this range, wherein 1 to 6 should be understood to include 1, 2, 3, 4, 5, 6, and also include the sub-ranges 1 to 5, 1 to 4 and 1 to 3.

The term “alkyl” as used herein, alone or as part of another group, refers to an unsubstituted straight or branched aliphatic hydrocarbon containing from 1 to 12 carbon atoms (ie, C₁₋₁₂ alkyl) or an indicated number of carbon atoms, for example, C₁ alkyl such as methyl, C₂ alkyl such as ethyl, C₃ alkyl such as n-propyl or isopropyl, C₁₋₃ alkyl such as methyl, ethyl, n-propyl or isopropyl, or the like. In one embodiment, the alkyl is C₁₋₄ alkyl. Non-limiting examples of C₁₋₁₂ alkyl include methyl, ethyl, n-propyl, isopropyl, n-butyl, sec-butyl, tert-butyl, isobutyl, 3-pentyl, hexyl, heptyl, octyl, nonyl and decyl. Examples of C₁₋₄ alkyl include methyl, ethyl, n-propyl, isopropyl, n-butyl, sec-butyl, tert-butyl, and isobutyl.

The term “alkenyl” is intended to include hydrocarbon chains of either straight or branched configuration having the specified number of carbon atoms and one or more, preferably one to two, carbon-carbon double bonds that may occur in any stable point along the chain. For example, “C₂-C₆ alkenyl” is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3, pentenyl, 4-pentenyl, 2-hexenyl, 3-hexenyl, 4-hexenyl, 5-hexenyl, 2-methyl-2-propenyl, and 4-methyl-3-pentenyl.

The term “alkynyl” is intended to include hydrocarbon chains of either straight or branched configuration having one or more, preferably one to three, carbon-carbon triple bonds that may occur in any stable point along the chain. For example, “C₂-C₆ alkynyl” is intended to include C₂, C₃, C₄, C₅, and C₆ alkynyl groups; such as ethynyl, propynyl, butynyl, pentynyl, and hexynyl.

The term “cycloalkyl” or “carbocyclyl” as used herein, alone or as part of another group, refers to a saturated or partially unsaturated (containing one or two double bonds) cyclic aliphatic hydrocarbon, which comprises 1 or 2 rings having 3 to 12 carbon atoms or an indicated number of carbon atoms (i.e., C₃₋₁₂ cycloalkyl). In one embodiment, the cycloalkyl has two rings. In one embodiment, the cycloalkyl has one ring. In another embodiment, the cycloalkyl group is selected from the group consisting of C₃₋₈ cycloalkyl groups. In another embodiment, the cycloalkyl group is selected from the group consisting of C₃₋₆ cycloalkyl groups. Non-limiting examples of cycloalkyl include cyclopropyl, cyclobutyl, cyclopentyl, cyclohexyl, cycloheptyl, cyclooctyl, norbornyl, decahydronaphthyl, adamantyl, cyclohexenyl, and cyclopentenyl.

The term “alkoxyl” or “alkoxy” as used herein, alone or as part of another group, refers to an oxygen linked to an alkyl group as defined above.

The term “heteroalkyl” as used herein, alone or as part of another group, refers to an alkyl group wherein one carbon atom is replaced with a heteroatom (such as N, O or S) or a carbonyl group (C═O).

The term “aryl” and “heteroaryl” and “aromatic heterocyclic”, as used herein, refer to stable mono- or polycyclic, carbocyclic and heterocyclic unsaturated moieties having preferably 3-14 carbon atoms, each of which may be substituted or unsubstituted. In certain embodiments of the present disclosure, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present disclosure, the term “heteroaryl”, as used herein, refers to a monocyclic, bicyclic or tricyclic aromatic radical having from five to fifteen, preferably five to ten ring atoms of which one ring atom is selected from S, O, N or Se; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, furanyl, thienyl, quinolinyl, isoquinolinyl, and the like.

The term “heterocyclyl”, “heterocyclic”, “heterocycle” or “heterocycloalkyl”, as used herein, refers to a non-aromatic 5-, 6-, or 7-membered ring or a polycyclic group, including, but not limited to a bi- or tri-cyclic group comprising fused, spiro or bridged three-membered, four-membered, five-membered, six-membered rings or seven-membered rings, wherein at least one carbon atom of one of the rings is replaced by a heteroatom. Each heteroatom is independently selected from the group consisting of atoms of oxygen, sulfur (including sulfoxide and sulfone) and/or nitrogen (which may be oxidized or quaternized). The term “heterocyclyl”, “heterocyclic”, “heterocycle” or “heterocycloalkyl” is intended to include a group wherein —CH₂— in the ring is replaced by —C(═O)—, for example, cyclic ureido (such as 2-imidazolidinone) and cyclic amido (such as β-lactam, γ-lactam, δ-lactam, ε-lactam) and piperazin-2-one; wherein (i) each 5-membered ring has 0 to 1 double bond and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring, such as a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

In certain embodiments, a “substituted aryl, heteroaryl, heterocyclyl or heterocycle” group is utilized and as used herein, refers to an aryl, heteroaryl, heterocyclyl or heterocycle group, as defined above, substituted by the independent replacement of one, two or three of the hydrogen atoms thereon with but are not limited to alkyl; heteroalkyl; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, alkyl, heteroalkyl, aryl, heteroaryl, arylalkyl, or heteroarylalkyl, wherein any of the alkyl, heteroalkyl, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

The term “halogen”, as used herein, comprises F, Cl, Br, I, etc.

The term “pharmaceutically acceptable salt”, as used herein, includes both acid addition salts and base addition salts of a compound.

Suitable acid addition salts are formed from acids which form non-toxic salts. Examples include acetate, adipate, aspartate, benzoate, besylate, bicarbonate/carbonate, bisulfate/sulfate, borate, camphorsulfonate, citrate, cyclohexylaminosulfonate, ethanedisulfonate, ethanesulfonate, formate, fumarate, glucoheptonate, gluconate, glucuronate, hexafluorophosphate, hibenzate, hydrochloride/chloride, hydrobromide/bromide, hydroiodide/iodide, isethionate, lactate, malate, maleate, malonate, methanesulfonate, methylsulfate, naphthylate, 2-naphthalenesulfonate, nicotinate, nitrate, orotate, oxalate, palmitate, pamoate, phosphate/hydrogen phosphate/dihydrogen phosphate, pyroglutamate, aldarate, stearate, succinate, tannate, tartrate, tosylate, trifluoroacetate and xinofoate.

Suitable base addition salts are formed from bases which form non-toxic salts. Examples include aluminum salts, arginine salts, benzathine benzylpenicillin salts, calcium salts, choline salts, diethylamine salts, diethanolamine salts, glycine salts, lysine salts, magnesium salts, meglumine salts, ethanolamine salts, potassium salts, sodium salts, tromethamine salts and zinc salts.

For a review of suitable salts, see “Handbook of Pharmaceutical Salts: Properties, Selection, and Use” by Stahl and Wermuth (Wiley-VCH, 2002). Methods for preparing the pharmaceutically acceptable salts of the compounds of the present disclosure are known to those skilled in the art.

The term “solvate” as used herein is a substance formed by combination, physical binding and/or solvation of a compound of the present disclosure with a solvent molecule, such as a desolvate, a monosolvate or a hemisolvate, wherein the ratio of the solvent molecule to the compound of the present disclosure is about 2:1, about 1:1 or about 1:2, respectively. This kind of physical bonding involves ionization and covalent bonding (including hydrogen bonding) in different degrees. In some cases (e.g., when one or more solvent molecules are incorporated into crystal lattice of crystalline solid), the solvate can be isolated. Thus, the solvate comprises both solution phase and isolatable solvates. The compounds of the present disclosure may be in solvated forms with pharmaceutically acceptable solvents (such as water, methanol and ethanol), and the present application is intended to encompass both solvated and unsolvated forms of the compounds of the present disclosure.

One type of solvate is a hydrate. “Hydrate” relates to a specific subset of solvates wherein the solvent molecule is water. Solvates generally function in the form of pharmacological equivalents. The preparation of solvates is known in the art, see for example, M. Caira et al, J. Pharmaceut. Sci., 93(3): 601-611 (2004), which describes the preparation of a solvate of fluconazole with ethyl acetate and water. Similar methods for the preparation of solvates, hemisolvates, hydrates and the like are described by van Tonder et al, AAPS Pharm. Sci. Tech., 5 (1): Article 12 (2004) and A. L. Bingham et al, Chem. Commun. 603-604 (2001). A representative and non-limiting method for the preparation of solvate involves dissolving a compound of the present disclosure in a desired solvent (organic solvent, water or a mixture thereof) at a temperature above 20° C. to about 25° C., and then the solution is cooled at a rate sufficient to form a crystal, and the crystal is separated by a known method such as filtration. Analytical techniques such as infrared spectroscopy can be used to confirm the presence of the solvent in the crystal of the solvate.

“Pharmaceutically acceptable carrier” in the context of the present disclosure refers to a diluent, adjuvant, excipient or vehicle together with which the therapeutic agent is administered, and which is suitable for contacting a tissue of human and/or other animals within the scope of reasonable medical judgment, and without excessive toxicity, irritation, allergic reactions, or other problems or complications corresponding to a reasonable benefit/risk ratio.

The pharmaceutically acceptable carriers that can be used in the pharmaceutical compositions of the present disclosure include, but are not limited to, sterile liquids such as water and oils, including those oils derived from petroleum, animals, vegetables or synthetic origins, for example, peanut oil, soybean oil, mineral oil, sesame oil, etc. Water is an exemplary carrier when the pharmaceutical composition is administered intravenously. It is also possible to use physiological saline and an aqueous solution of glucose and glycerin as a liquid carrier, particularly for injection. Suitable pharmaceutical excipients include starch, glucose, lactose, sucrose, gelatin, maltose, chalk, silica gel, sodium stearate, glyceryl monostearate, talc, sodium chloride, skimmed milk powder, glycerin, propylene glycol, water, ethanol and the like. The pharmaceutical composition may further contain a small amount of a wetting agent, an emulsifier or a pH buffering agent as needed. Oral formulations may contain standard carriers such as pharmaceutical grades of mannitol, lactose, starch, magnesium stearate, sodium saccharin, cellulose, magnesium carbonate, and the like. Examples of suitable pharmaceutically acceptable carriers are as described in Remington's Pharmaceutical Sciences (1990).

The pharmaceutical compositions of the present disclosure may act systemically and/or locally. For this purpose, they may be administered via a suitable route, for example by injection (e.g., intravenous, intraarterial, subcutaneous, intraperitoneal, intramuscular administration, including instillation) or transdermal administration; or by oral, buccal, nasal, transmucosal, topical administration, in form of ophthalmic preparation or by inhalation.

For these routes of administration, the pharmaceutical compositions of the present disclosure may be administered in a suitable dosage form.

The dosage forms include, but are not limited to, tablets, capsules, troches, hard candy, pulvis, sprays, creams, ointments, suppositories, gels, pastes, lotions, ointments, aqueous suspensions, injectable solutions, elixirs, syrups.

The term “effective amount” as used herein refers to an amount of active ingredient that, after administration, will relieve to some extent one or more symptoms of the condition being treated.

As used herein, “individual” includes a human or a non-human animal. Exemplary human individual includes a human individual (referred to as a patient) suffering from a disease (such as the disease described herein) or a normal individual. “Non-human animal” in the present disclosure includes all vertebrates, such as non-mammals (e.g., birds, amphibians, reptiles) and mammals, such as non-human primates, domestic animals, and/or domesticated animals (e.g., sheep, dogs, cats, cows, pigs, etc.).

The compounds of the present disclosure may contain one or more asymmetric centers, depending on the location and nature of the various substituents desired. Asymmetric carbon atoms can exist in the (R) or (S) configuration, giving racemic mixtures in the case of one asymmetric center, and diastereomeric mixtures in the case of multiple asymmetric centers.

Preferred compounds are those that produce the more desirable biological activity. Isolated, purified or partially purified isomers and stereoisomers, or racemic or diastereomeric mixtures of the disclosed compounds are included within the scope of the present disclosure. Purification and isolation of such substances can be accomplished by standard techniques known in the art.

### EXAMPLES

In order to make the objects and technical solutions of the present disclosure clearer, the present disclosure will be further described below in conjunction with specific example. It should be understood that the examples are not intended to limit the scope of the present disclosure. Further, specific experimental methods not mentioned in the following examples were carried out in accordance with a conventional experimental method.

### Synthesis

The prior art compounds Cpd-118 and Cpd-27 are synthesized according to the prior art methods such as those recorded in WO2021057910A1 or WO2020033490A1.

Compounds of the disclosure are synthesized and characterized with the following procedures.
LCMS Conditions: Agilent 6125, Column: Waters CORTECS C18+, 2.7 μm 4.6*30 mm, Mobile phase: ACN (0.05% FA)-Water (0.05% FA), Gradient: 5% ACN to 95% ACN in 1.0 min, hold 1.0 min, total 2.5 min, Flow rate: 1.8 mL/min
HPLC Conditions: Shimadzu LC-2030, Column: XBRIDGE, 2.1*50 mm, 3.5 μm, Mobile phase: ACN (0.05% TFA)-Water (0.05% TFA), Gradient: 0% ACN to 60% ACN over 7.0 min, 7-8 min, ACN from 60% to 100%, Flow rate: 0.8 mL/min

### Example 1. Synthesis of HH-1

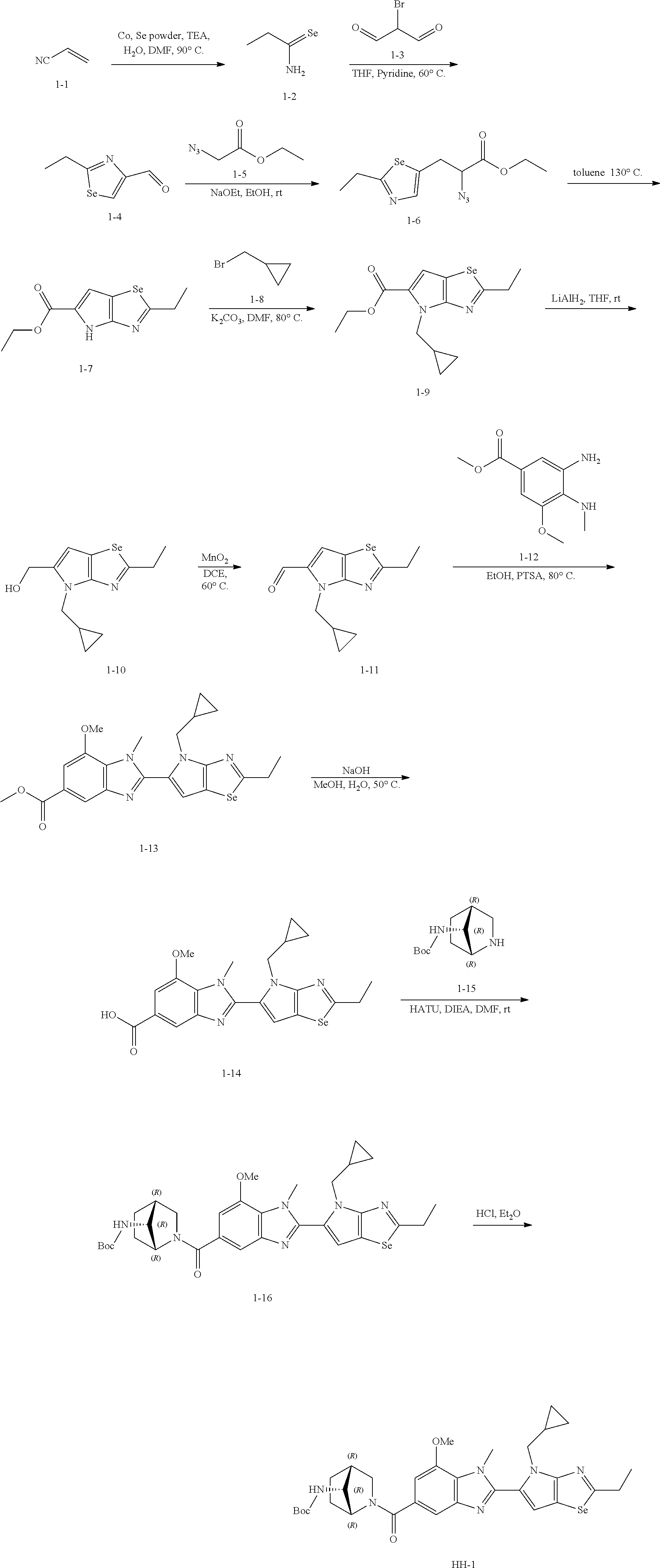

### Step 1

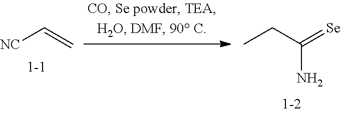

Compound 1-2: To a solution of acrylonitrile (5.0 g, 94.3 mmol) in DMF/H₂O (50/5 mL) was added Se powder (8.9 g, 113.2 mmol). The reaction mixture was stirred at 90° C. under CO atmosphere for 5 h. The mixture was diluted with water (100 mL), extracted with EtOAc (3×100 mL). The organic layers were washed with brine (100 mL), dried over anhydrous Na₂SO₄ and concentrated under vacuum to afford crude product, which was purified by silica gel chromatography (elution gradient: petroleum ether/EtOAc, 5/1, v/v) to afford compound 1-2 (7 g) as a brown solid, yield: 55.2%. LCMS: Rt=0.391 min, MS (ESI) m/z=138.0 [M+H]⁺.

### Step 2

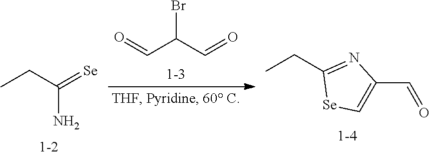

Compound 1-4: To a solution of compound 1-2 (3.5 g, 25.5 mmol) in THF (20 mL) was added 2-bromopropanedial (3.8 g, 25.5 mmol) and pyridine (4.0 g, 51.1 mmol). The reaction mixture was stirred at 60° C. for 16 h. The solvent was evaporated and diluted with water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 1-4 (1.42 g) as a yellow solid, yield: 29%. LCMS: Rt=1.047 min, MS (ESI) m/z=190.1 [M+H]⁺.

### Step 3

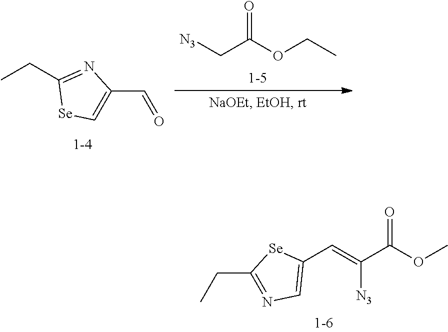

Compound 1-6: To a solution of sodium ethoxide (20% wt. in EtOH) (5.4 g, 15.87 mmol) in EtOH (50 mL) was slowly added compound 1-4 (710 mg, 3.76 mmol) and compound 1-5 (2.0 g, 15.87 mmol) in EtOH (50 mL) at −15° C. under N₂. The reaction mixture was stirred at −15° C. for 1 h and 0° C. for further 1 h. The mixture was quenched with ice water (50 mL) and extracted with ethyl acetate (50 mL×3). The organic layers were washed with water (100 mL), brine (100 mL), dried over anhydrous Na₂SO₄ and concentrated under vacuum to afford crude product, which was purified by silica gel chromatography (petroleum ether/EtOAc, 5/1, v/v) to afford compound 1-6 (560 mg) as a yellow oil, yield: 49.8%. LCMS: Rt=1.378 min, MS (ESI) m/z=301.0 [M+H]⁺.

### Step 4

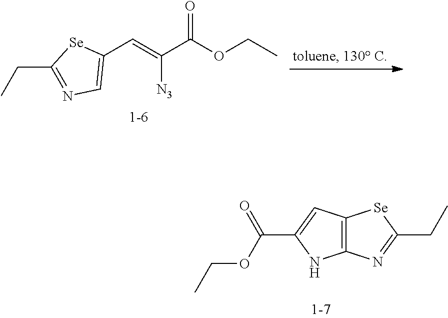

Compound 1-7: A solution of compound 1-6 (560 mg, 1.86 mmol) in toluene (40 mL) mixture was stirred at 130° C. for 2 h. The reaction mixture was concentrated under vacuum to give the crude product, which was purified by silica gel chromatography (elution gradient: petroleum ether/EtOAc, 5/1, v/v) to afford compound 1-7 (440 mg) as yellow oil, yield: 86.6%. LCMS: Rt=1.282 min, MS (ESI) m/z=273.0 [M+H]⁺.

### Step 5

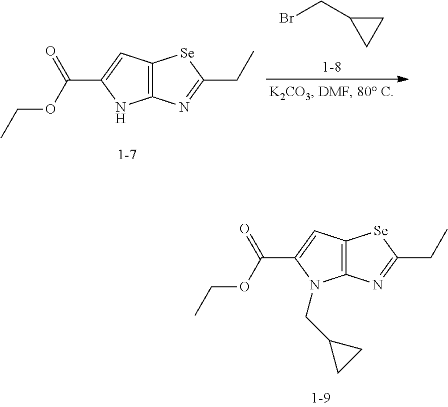 [SMILES: BrCC1CC1 ; CCOC(=O)c1cc2[se]c(CC)nc2[nH]1 ; CCOC(=O)c1cc2[se]c(CC)nc2n1CC1CC1]

Compound 1-9: To a solution of compound 1-7 (440 mg, 1.62 mmol) and compound 1-8 (260.5 mg, 1.94 mmol) in DMF (18 mL) was added potassium carbonate (670.7 mg, 4.86 mmol). The mixture was stirred at 50° C. for 16 h. The mixture was diluted with water (30 mL) and extracted with ethyl acetate (30 mL×3). The organic layers were washed with brine (30 mL), dried over anhydrous Na₂SO₄ and concentrated under vacuum to afford crude product, which was purified by silica gel chromatography (elution gradient: petroleum ether/EtOAc, 1/1, v/v) to afford compound 1-9 (360 mg) as a yellow oil, yield: 68.4%. LCMS: Rt=1.534 min, MS (ESI) m/z=327.0 [M+H]⁺.

### Step 6

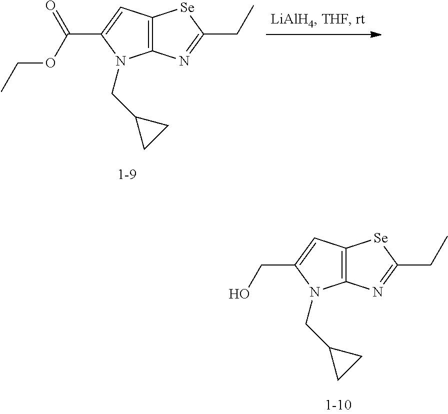 [SMILES: CCOC(=O)c1cc2[se]c(CC)nc2n1CC1CC1 ; CCc1nc2c(cc(CO)n2CC2CC2)[se]1]

Compound 1-10: To a solution of compound 1-9 (360 mg, 1.1 mmol) in THF (10 mL), then was added LiAlH₄ (1M in THF, 3.2 mL, 3.2 mmol) at 0° C., The reaction mixture was stirred at 25° C. for 2 h. The mixture was quenched with water (18 mL) and filtered. The filtrate was extracted with ethyl acetate (20 mL×3). The organic layers were dried over anhydrous Na₂SO₄ and concentrated under vacuum to afford crude product (300 mg) as a yellow oil. LCMS: Rt=1.266 min, MS (ESI) m/z=285.1 [M+H]⁺.

### Step 7

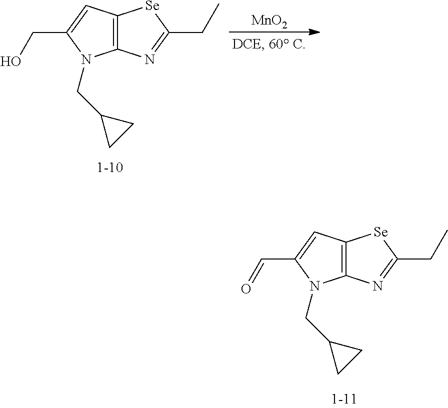

Compound 1-11: To a solution of compound 1-10 (300 mg, 1.06 mmol) in DCE (10 mL) was added MnO₂ (916 mg, 10.5 mmol). The reaction mixture was stirred at 60° C. for 16 h. The mixture was filtered and the filtrate was concentrated under vacuum to afford crude product 1-11 (280 mg) as a brown oil. LCMS: Rt=1.403 min, MS (ESI) m/z=283.1 [M+H]⁺.

### Step 8

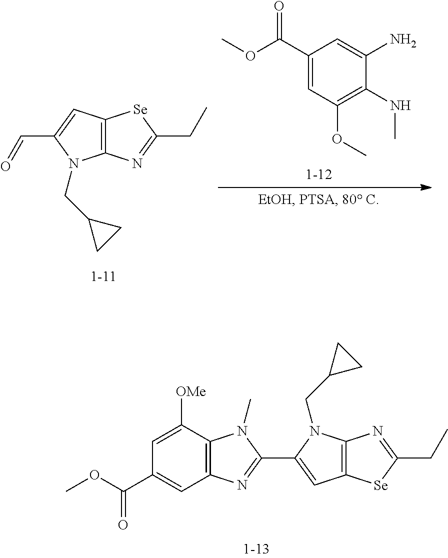

Compound 1-13: To a solution of compound 1-11 (280 mg, 1 mmol) in EtOH (20 mL) was added compound 1-12 (208 mg, 1 mmol) and PTSA (17.2 mg, 0.1 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 1-13 (340 mg) as a yellow oil, yield: 84.8%. LCMS: Rt=1.497 min, MS (ESI) m/z=473.0 [M+H]⁺.

### Step 9

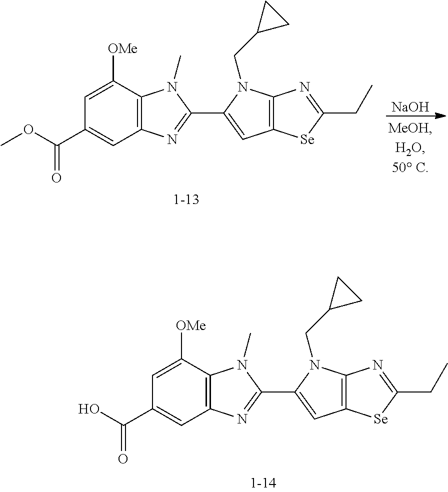

Compound 1-14: To a solution of compound 1-13 (340 mg, 0.72 mmol) in MeOH/H₂O (10/2 mL) was added NaOH (86 mg, 2.16 mmol), The reaction mixture was stirred at 50° C. for 16 h. The mixture was concentrated and adjusted pH to 5-6 with 1N hydrochloric acid aqueous solution, then extracted with ethyl acetate (20 mL×3). The organic phase was washed with water (10 mL), brine (10 mL), dried over anhydrous Na₂SO₄ and concentrated under vacuum to give compound 1-14 (280 mg) as a yellow solid, yield: 84.8%. LCMS: Rt=1.342 min, MS (ESI) m/z=459.1 [M+H]⁺.

### Step 10

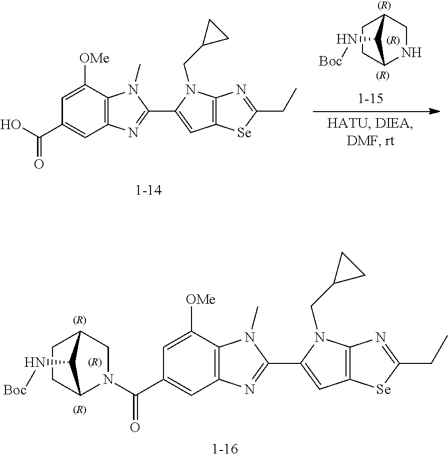

Compound 1-16: To a solution of compound 1-14 (200 mg, 0.44 mmol) in DMF (10 mL) was added compound 1-15 (92 mg, 0.44 mmol), HATU (168 mg, 0.44 mmol) and DIEA (170 mg, 1.32 mmol). The reaction mixture was stirred at 25° C. for 16 h. The mixture was diluted with water (20 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 1-16 (200 mg) as a brown solid, yield: 70.4%. LCMS: Rt=1.421 min, MS (ESI) m/z=653.2 [M+H]⁺.

### Step 11

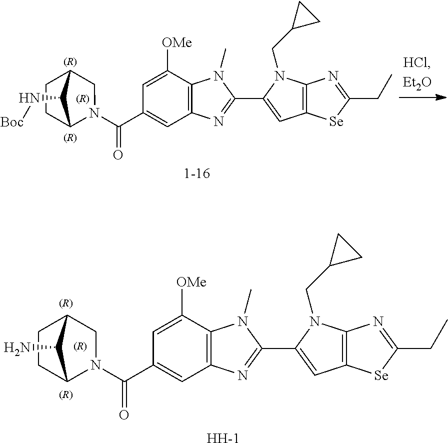

HH-1: To a solution of compound 1-16 (200 mg, 0.3 mmol) in Et₂O (10 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-1 (120 mg, hydrochloric acid salt form) as a yellow solid. yield: 70.6%. ¹H NMR (400 MHz, DMSO-d₆) δ ppm 7.34 (s, 1H), 6.92-6.87 (m, 2H), 4.38 (d, J=7.0 Hz, 2H), 4.19-3.99 (m, 7H), 3.56 (s, 1H), 3.35 (s, 1H), 3.11-3.04 (m, 3H), 2.39 (s, 1H), 2.05-1.90 (m, 2H), 1.79 (t, J=9.2 Hz, 1H), 1.54-1.46 (m, 1H), 1.33 (t, J=7.6 Hz, 3H), 1.20-1.12 (m, 1H), 0.34-0.27 (m, 2H), 0.19-0.13 (m, 2H). LCMS: Rt=1.121 min, MS (ESI) m/z=553.0 [M+H]⁺. HPLC: Rt=4.78 min, Purity: 95.57% (214 nm) and 95.26% (254 nm).

### Example 2. Synthesis of HH-2

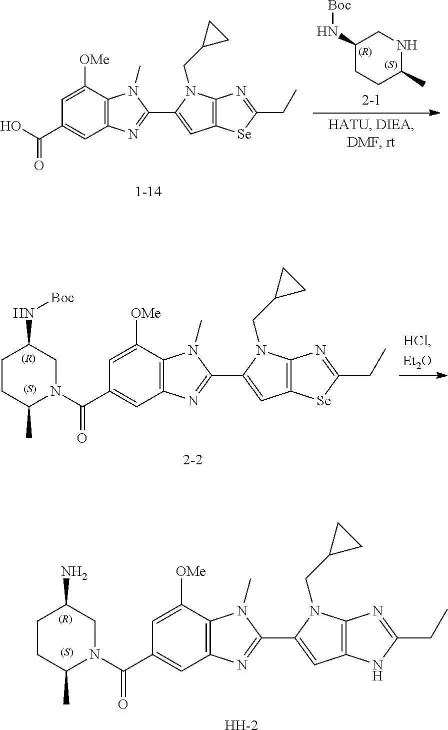

### Step 1

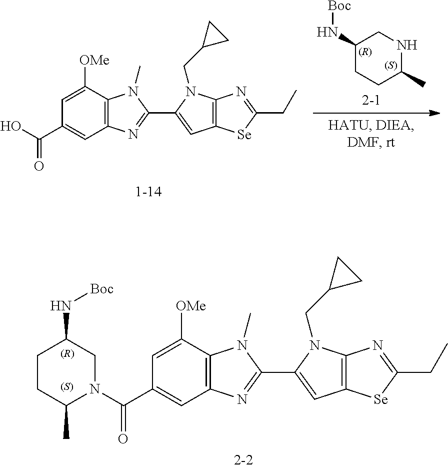

Compound 2-2: To a solution of compound 1-14 (180 mg, 0.40 mmol) in DMF (8 mL) was added compound 2-1 (86 mg, 0.40 mmol), HATU (152 mg, 0.4 mmol) and DIEA (156 mg, 1.2 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 2-2 (180 mg) as a brown solid, yield: 70.3%. LCMS: Rt=1.454 min, MS (ESI) m/z=655.3 [M+H]⁺.

### Step 2

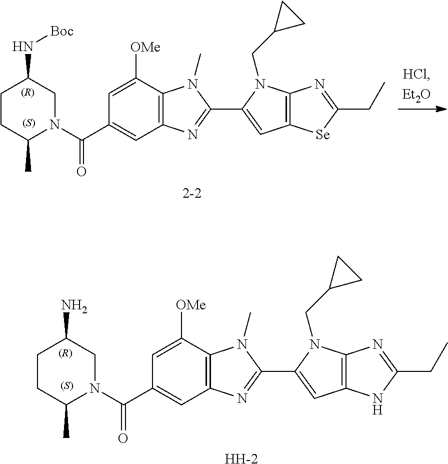

HH-2: To a solution of compound 2-2 (180 mg, 0.28 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-2 (76 mg, hydrochloric acid salt form) as a yellow solid. yield: 50.0%. ¹H NMR (400 MHz, DMSO) δ 8.26 (s, 3H), 7.33 (s, 1H), 7.04 (s, 1H), 6.93 (s, 1H), 4.99 (s, 2H), 4.39 (d, J=7.1 Hz, 2H), 4.13 (s, 3H), 4.01 (s, 3H), 3.22-3.03 (m, 4H), 1.94-1.72 (m, 3H), 1.59 (s, 1H), 1.35 (t, J=7.5 Hz, 3H), 1.19 (dd, J=18.8, 6.9 Hz, 4H), 0.39-0.30 (m, 2H), 0.19 (q, J=4.8 Hz, 2H). LCMS: Rt=1.150 min, MS (ESI) m/z=555.0 [M+H]⁺. HPLC: Rt=5.01 min, Purity: 99.89% (214 nm) and 99.89% (254 nm).

### Example 3. Synthesis of HH-3

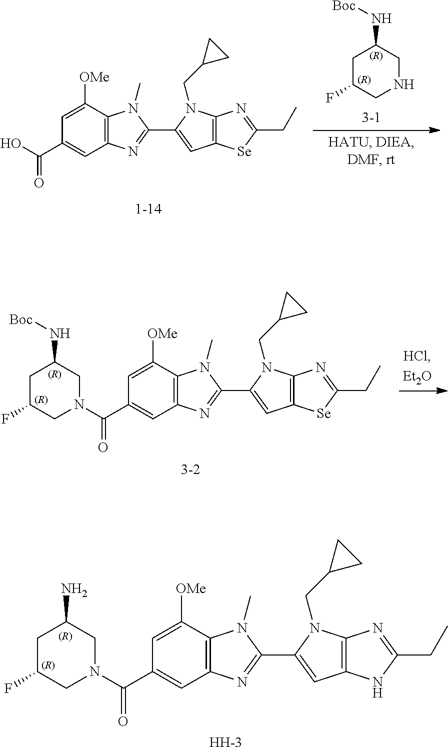

### Step 1

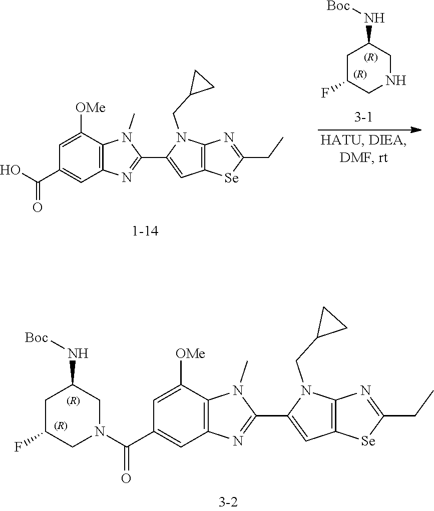

Compound 16: To a solution of compound 1-14 (180 mg, 0.40 mmol) in DMF (8 mL) was added compound 3-1 (86 mg, 0.40 mmol), HATU (152 mg, 0.4 mmol) and DIEA (156 mg, 1.2 mmol). The reaction mixture was stirred at 25° C. for 16 h. The mixture was diluted with water (10 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 3-2 (180 mg) as a brown solid, yield: 69.8%. LCMS: Rt=1.405 min, MS (ESI) m/z=659.2 [M+H]⁺.

### Step 2

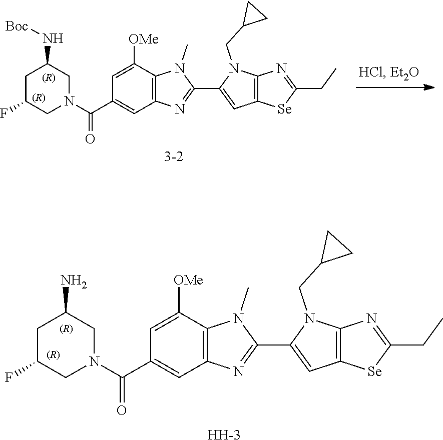

HH-3: To a solution of compound 3-2 (180 mg, 0.28 mmol) in Et₂O (10 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HHBP-9449 (112 mg, hydrochloric acid salt form) as a yellow solid. yield: 73.6%. ¹H NMR (400 MHz, DMSO) δ 8.51 (s, 3H), 7.37 (s, 1H), 7.07 (d, J=13.6 Hz, 1H), 6.99 (s, 1H), 5.09-4.66 (m, 2H), 4.39 (d, J=7.0 Hz, 2H), 4.14 (s, 3H), 4.01 (s, 3H), 3.38 (d, J=4.8 Hz, 2H), 3.12 (dd, J=9.6, 5.4 Hz, 2H), 2.40 (s, 1H), 2.08-1.84 (m, 1H), 1.31 (ddd, J=14.6, 12.0, 7.0 Hz, 5H), 1.14 (s, 1H), 0.34 (d, J=7.9 Hz, 2H), 0.18 (s, 2H). LCMS: Rt=1.089 min, MS (ESI) m/z=559.0 [M+H]⁺. HPLC: Rt=4.93 min, Purity: 99.87% (214 nm) and 99.69% (254 nm).

### Example 4. Synthesis of HH-4

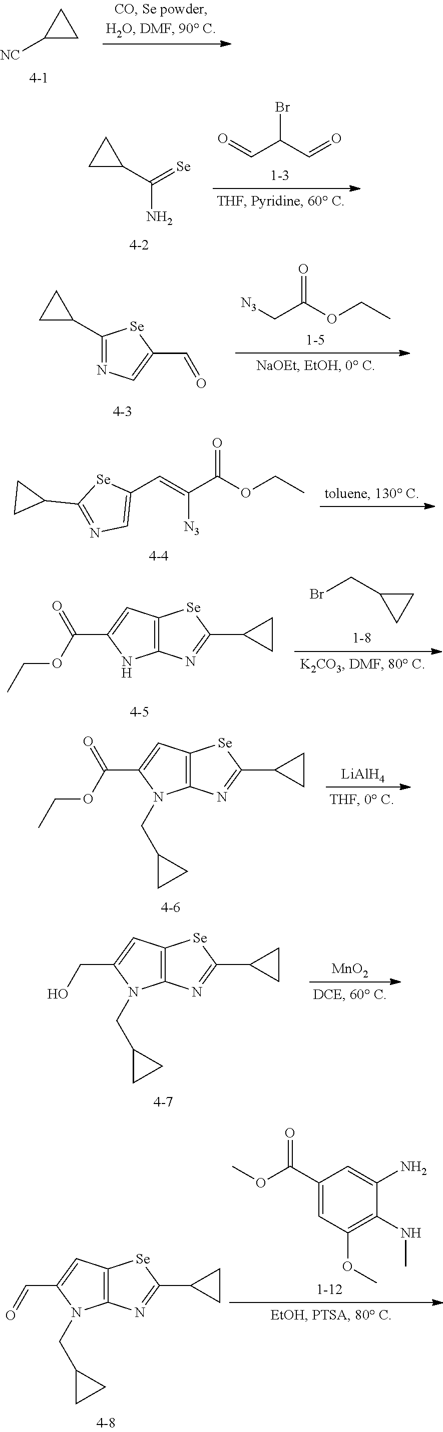

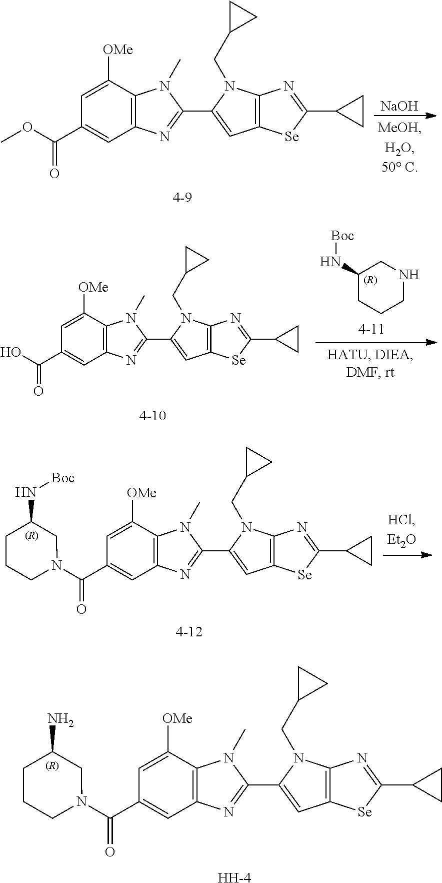

### Step 1

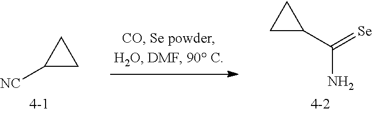 [SMILES: N#CC1CC1 ; NC(=[Se])C1CC1]

Compound 4-2: To a solution of compound 4-1 (10 g, 0.15 mol) in DMF/H₂O (150/15 mL) was added Se powder (14.1 g, 0.18 mol). The reaction mixture was stirred at 90° C. under CO atmosphere for 5 h. The mixture was diluted with water (200 mL), extracted with EtOAc (3×150 mL). The organic layers were washed with brine (200 mL), dried over anhydrous Na₂SO₄ and concentrated under vacuum to afford crude product, which was purified by silica gel chromatography (elution gradient: petroleum ether/EtOAc, 5/1, v/v) to afford compound 4-2 (13.5 g) as a brown solid, yield: 61.4%. LCMS: Rt=0.640 min, MS (ESI) m/z=150.0 [M+H]⁺.

### Step 2

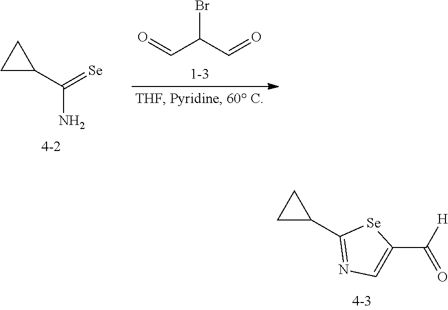 [SMILES: NC(=[Se])C1CC1 ; O=CC(Br)C=O ; O=Cc1cnc(C2CC2)[se]1]

Compound 4-3: To a solution of compound 4-2 (9 g, 60.4 mmol) in THF (50 mL) was added 2-bromopropanedial (9.1 g, 60.4 mmol) and pyridine (9.5 g, 120.8 mmol). The reaction mixture was stirred at 60° C. for 16 h. The solvent was evaporated and diluted with water (50 mL). The mixture was extracted with ethyl acetate (50 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 4-3 (6 g) as a yellow solid, yield: 50%. LCMS: Rt=1.081 min, MS (ESI) m/z=202.0 [M+H]⁺.

### Step 3

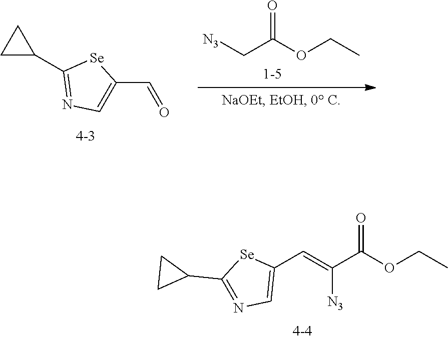

Compound 4-4: To a solution of sodium ethoxide (20% wt. in EtOH) (20.3 g, 59.7 mmol) in EtOH (100 mL) was slowly added compound 4-3 (4 g, 19.9 mmol) and compound 1-5 (7.7 g, 59.7 mmol) in EtOH (50 mL) at −15° C. under N₂. The reaction mixture was stirred at −15° C. for 1 h and 0° C. for further 1 h. The mixture was quenched with ice water (100 mL) and extracted with ethyl acetate (100 mL×3). The organic layers were washed with water (100 mL), brine (100 mL), dried over anhydrous Na₂SO₄ and concentrated under vacuum to afford crude product, which was purified by silica gel chromatography (petroleum ether/EtOAc, 5/1, v/v) to afford compound 4-4 (2.7 g) as a yellow oil, yield: 43.5%. LCMS: Rt=1.366 min, MS (ESI) m/z=313.0 [M+H]⁺.

### Step 4

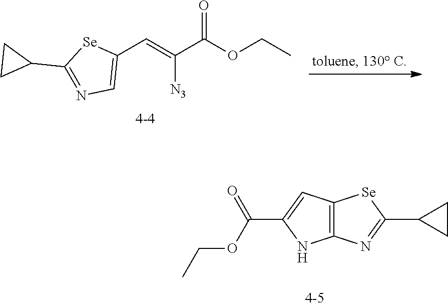

Compound 4-5: A solution of compound 4-4 (2.7 mg, 8.64 mmol) in toluene (60 mL) mixture was stirred at 130° C. for 2 h. The reaction mixture was concentrated under vacuum to give the crude product, which was purified by silica gel chromatography (elution gradient: petroleum ether/EtOAc, 5/1, v/v) to afford compound 4-5 (1.8 g) as yellow oil, yield: 73.0%. LCMS: Rt=1.278 min, MS (ESI) m/z=285.0 [M+H]⁺.

### Step 5

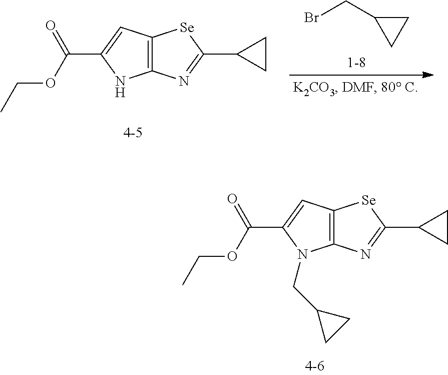 [SMILES: CCOC(=O)c1cc2[se]c(C3CC3)nc2[nH]1 ; BrCC1CC1 ; CCOC(=O)c1cc2[se]c(C3CC3)nc2n1CC1CC1]

Compound 4-6: To a solution of compound 4-5 (1.2 g, 4.22 mmol) and compound 1-8 (677 mg, 5.06 mmol) in DMF (16 mL) was added potassium carbonate (1.165 g, 8.44 mmol). The mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 4-6 (800 mg) as a yellow oil, yield: 56.3%. LCMS: Rt=1.557 min, MS (ESI) m/z=339.0 [M+H]⁺.

### Step 6

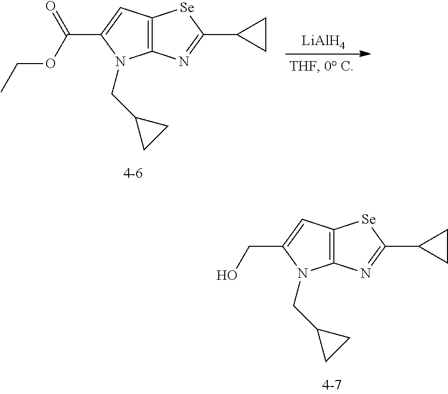

Compound 4-7: To a solution of compound 4-6 (800 mg, 2.36 mmol) in THF (10 mL), then was added LiAlH₄ (1M in THF, 7 mL, 7 mmol) at 0° C., The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product (640 mg) as a yellow oil. LCMS: Rt=1.275 min, MS (ESI) m/z=297.0 [M+H]⁺.

### Step 7

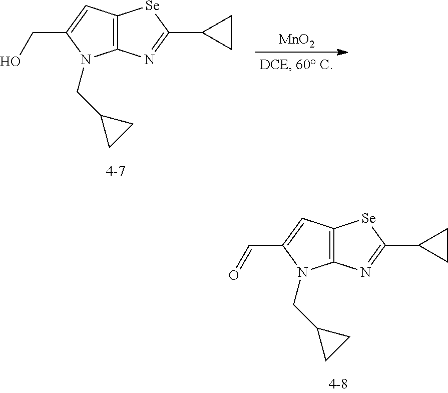

Compound 4-8: To a solution of compound 4-7 (640 mg, 2.16 mmol) in DCE (20 mL) was added MnO₂ (1.88 g, 21.6 mmol). The reaction mixture was stirred at 60° C. for 16 h. The mixture was filtered and the filtrate was concentrated under vacuum to afford crude product (600 mg) as a brown oil. LCMS: Rt=1.404 min, MS (ESI) m/z=295.0 [M+H]⁺.

### Step 8

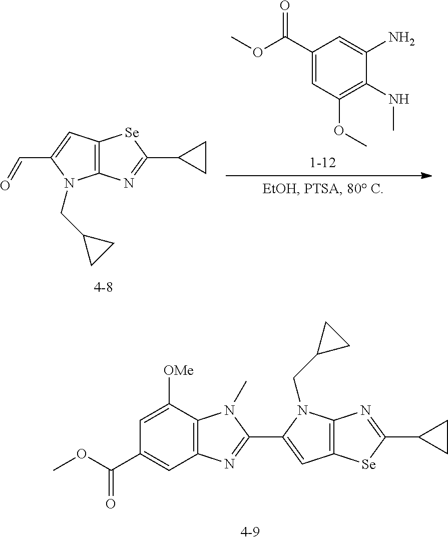

Compound 4-9: To a solution of compound 4-8 (600 mg, 2.12 mmol) in EtOH (20 mL) was added compound 12 (446 mg, 2.12 mmol) and PTSA (38 mg, 0.22 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 4-9 (840 mg) as a yellow oil, yield: 85.2%. LCMS: Rt=1.490 min, MS (ESI) m/z=485.1 [M+H]⁺.

### Step 9

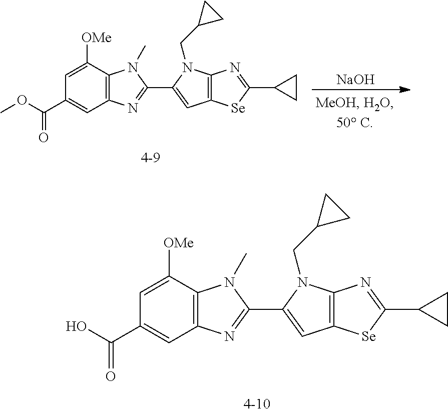

Compound 4-10: To a solution of compound 4-9 (840 mg, 0.87 mmol) in MeOH/H₂O (10/2 mL) was added NaOH (208.8 mg, 5.22 mmol), The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 4-10 (800 mg) as a yellow solid, yield: 98.0%. LCMS: Rt=1.349 min, MS (ESI) m/z=471.1 [M+H]⁺.

### Step 10

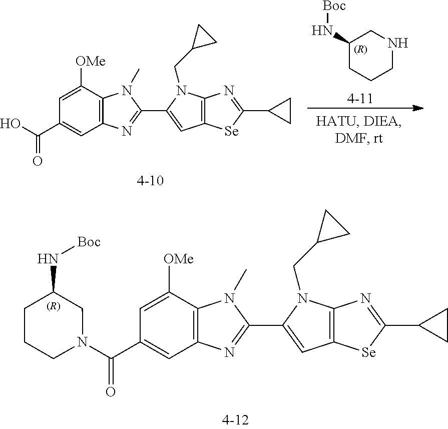

Compound 4-12: To a solution of compound 4-10 (100 mg, 0.21 mmol) in DMF (5 mL) was added compound 4-11 (42 mg, 0.21 mmol), HATU (79.8 mg, 0.21 mmol) and DIEA (81.3 mg, 0.63 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 4-12 (100 mg) as a brown solid, yield: 71.9%. LCMS: Rt=1.428 min, MS (ESI) m/z=653.2 [M+H]⁺.

### Step 11

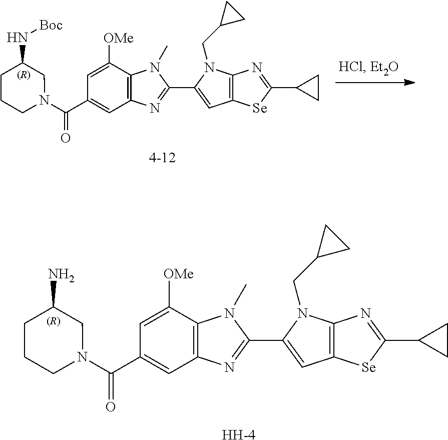

HH-4: To a solution of compound 4-12 (100 mg, 0.15 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-4 (64 mg, hydrochloric acid salt form) as a yellow solid. yield: 73.6%. ¹H NMR (400 MHz, DMSO-d₆) δ ppm 8.30 (s, 3H), 7.37 (s, 1H), 7.02 (s, 2H), 4.34 (d, J=6.8 Hz, 4H), 4.21 (s, 3H), 4.01 (s, 3H), 3.28-3.01 (m, 3H), 2.60-2.48 (m, 1H), 2.05-1.98 (m, 1H), 1.79-1.45 (m, 3H), 1.21-0.96 (m, 5H), 0.32-0.22 (m, 2H), 0.12 (t, J=4.8 Hz, 2H). LCMS: Rt=1.173 min, MS (ESI) m/z=552.9 [M+H]⁺. HPLC: Rt=4.96 min, Purity: 97.52% (214 nm) and 97.37% (254 nm).

### Example 5. Synthesis of HH-5

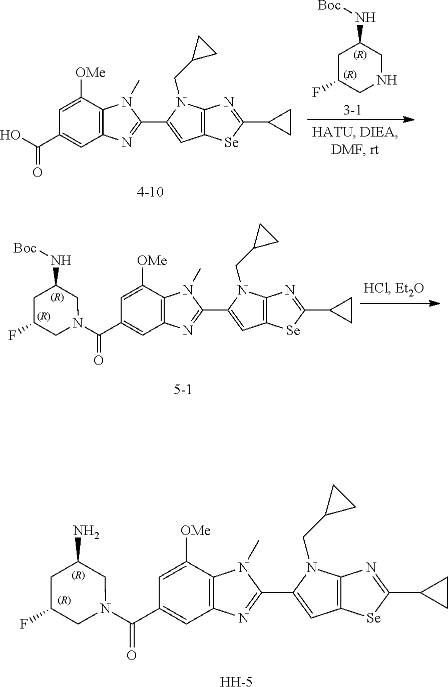

### Step 1

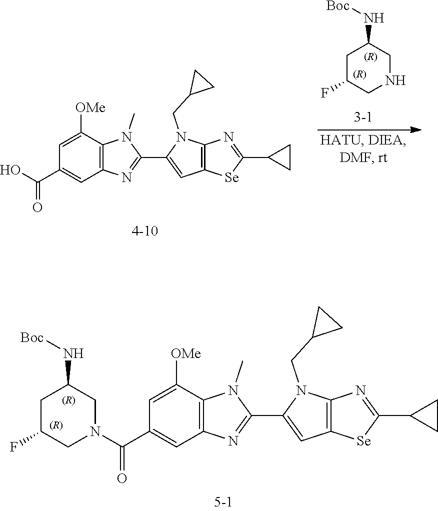

Compound 5-1: To a solution of compound 4-10 (100 mg, 0.21 mmol) in DMF (5 mL) was added compound 3-1 (46 mg, 0.21 mmol), HATU (79.8 mg, 0.21 mmol) and DIEA (81.3 mg, 0.63 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 5-1 (90 mg) as a brown solid, yield: 62.9%. LCMS: Rt=1.412 min, MS (ESI) m/z=671.2 [M+H]⁺.

### Step 2

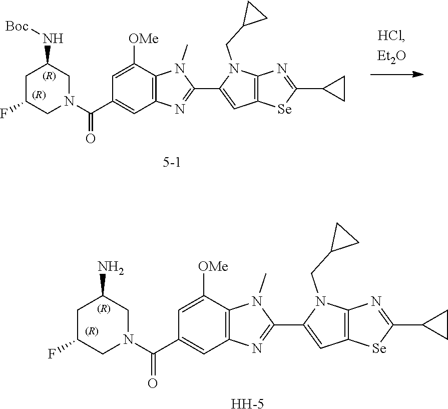

HH-5: To a solution of compound 5-1 (90 mg, 0.13 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-5 (36 mg, hydrochloric acid salt form) as a yellow solid. yield: 47%. ¹H NMR (400 MHz, DMSO-d₆) δ ppm 8.49 (s, 3H), 7.34 (s, 1H), 7.05-6.98 (m, 2H), 5.08-4.66 (m, 3H), 4.29 (d, J=7.0 Hz, 2H), 4.09 (s, 3H), 3.98 (s, 3H), 3.41-3.30 (m, 2H), 3.13-2.88 (m, 1H), 2.57-2.50 (m, 1H), 2.41-2.31 (m, 1H), 2.01-1.84 (m, 1H), 1.25-1.12 (m, 2H), 1.10-0.96 (m, 3H), 0.31-0.25 (m, 2H), 0.15-0.07 (m, 2H). LCMS: Rt=1.187 min, MS (ESI) m/z=570.9 [M+H]⁺. HPLC: Rt=5.047 min, Purity: 97.23% (214 nm) and 97.12% (254 nm).

### Example 6. Synthesis of HH-6

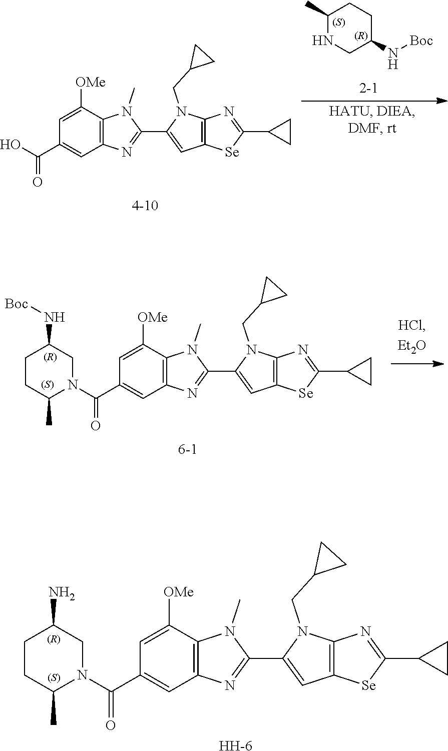

Step 1

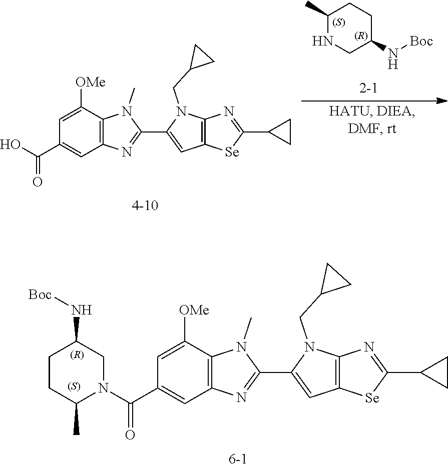

Compound 6-1: To a solution of compound 4-10 (100 mg, 0.21 mmol) in DMF (5 mL) was added compound 2 (45 mg, 0.21 mmol), HATU (79.8 mg, 0.21 mmol) and DIEA (81.3 mg, 0.63 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 6-1 (95 mg) as a brown solid, yield: 66.9%. LCMS: Rt=1.453 min, MS (ESI) m/z=667.2 [M+H]⁺.

### Step 2

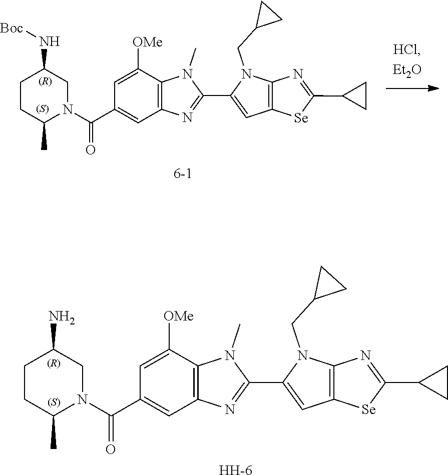

HH-6: To a solution of compound 6-1 (95 mg, 0.14 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-6 (48 mg, hydrochloric acid salt form) as a yellow solid. yield: 59.1%. ¹H NMR (400 MHz, DMSO-d₆) δ ppm 8.17 (s, 3H), 7.28 (s, 1H), 7.02-6.96 (m, 1H), 6.94-6.87 (m, 1H), 4.30 (d, J=6.4 Hz, 2H), 4.08 (s, 5H), 3.96 (s, 4H), 3.16-2.98 (m, 2H), 1.91-1.67 (m, 3H), 1.55 (s, 1H), 1.21-1.05 (m, 6H), 1.04-0.98 (m, 2H), 0.34-0.25 (m, 2H), 0.16-0.11 (m, 2H). LCMS: Rt=1.107 min, MS (ESI) m/z=567.0 [M+H]⁺. HPLC: Rt=5.138 min, Purity: 98.10% (214 nm) and 98.07% (254 nm).

### Example 7. Synthesis of HH-7

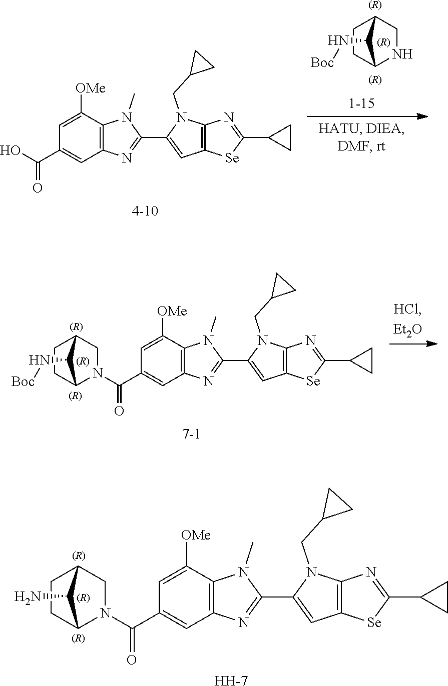

### Step 1

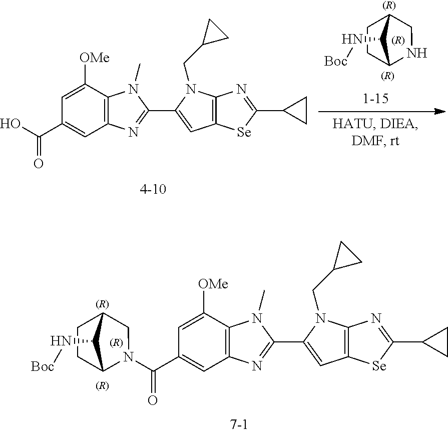

Compound 7-1: To a solution of compound 4-10 (100 mg, 0.21 mmol) in DMF (5 mL) was added compound 1-15 (45 mg, 0.21 mmol), HATU (79.8 mg, 0.21 mmol) and DIEA (81.3 mg, 0.63 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 3 (95 mg) as a brown solid, yield: 67.4%. LCMS: Rt=1.426 min, MS (ESI) m/z=665.2 [M+H]⁺.

### Step 2

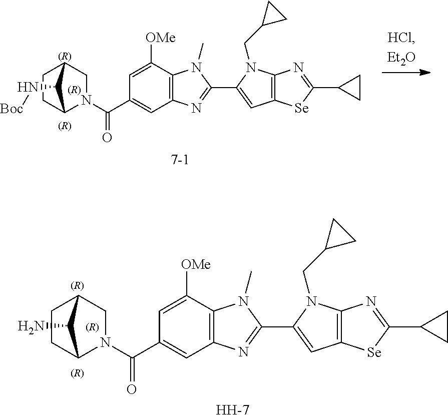

HH-7: To a solution of compound 7-1 (95 mg, 0.14 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HHBP-9526 (51 mg, hydrochloric acid salt form) as a yellow solid. yield: 63.3%. ¹H NMR (400 MHz, DMSO-d₆) δ ppm 8.32 (s, 3H), 7.38 (s, 1H), 6.95 (s, 1H), 6.90 (s, 1H), 4.39-4.30 (m, 3H), 4.07 (s, 3H), 3.98 (s, 3H), 3.65-3.45 (m, 2H), 3.15 (d, J=10.4 Hz, 1H), 2.68-2.58 (m, 1H), 2.52-2.47 (m, 1H), 2.01-1.84 (m, 3H), 1.64-1.56 (m, 1H), 1.25-1.13 (m, 3H), 1.05-0.96 (m, 2H), 0.33-0.29 (m, 2H), 0.17-0.13 (m, 2H). LCMS: Rt=1.110 min, MS (ESI) m/z=565.2 [M+H]⁺. HPLC: Rt=4.920 min, Purity: 96.99% (214 nm) and 96.85% (254 nm).

### Example 8. Synthesis of HH-8

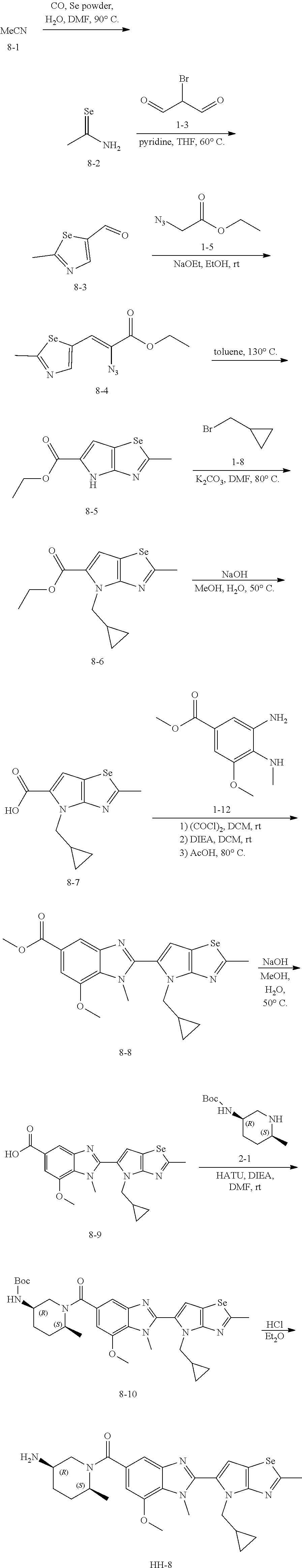

### Step 1

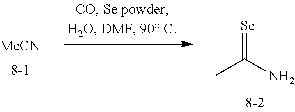

Compound 8-2: To a solution of MeCN (20 g, 487.8 mmol) in DMF/H₂O (200/20 mL) was added Se powder (44.5 g, 566 mmol). The reaction mixture was stirred at 90° C. under CO atmosphere for 5 h. The mixture was diluted with water (500 mL), extracted with EtOAc (3×300 mL). The organic layers were washed with brine (300 mL), dried over anhydrous Na₂SO₄ and concentrated under vacuum to afford crude product, which was purified by silica gel chromatography (elution gradient: petroleum ether/EtOAc, 5/1, v/v) to afford compound 8-2 (33.7 g) as a brown solid, yield: 56%. LCMS: Rt=0.25 min, MS (ESI) m/z=124.0 [M+H]⁺.

### Step 2

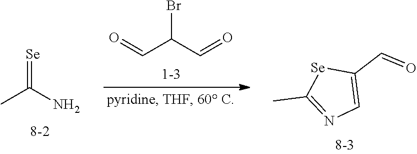

Compound 8-3: To a solution of compound 8-2 (28 g, 227.6 mmol) in THF (500 mL) was added compound 3 (35 g, 233.5 mmol) and pyridine (36 g, 455.7 mmol). The mixture was stirred at 60° C. for 16 h. The mixture was filtered and concentrated under reduced pressure to afford residue. The residue was diluted with water (300 mL), extracted with ethyl acetate (300 mL×3). The combined organic layers were dried over anhydrous sodium sulfate and concentrated under vacuum to afford crude product, which was purified by flash chromatography (elution gradient: petroleum ether/ethyl acetate, 5/1, v/v) to afford compound 8-3 (22.8 g) as yellow solid, yield: 57.3%. LCMS: Rt=0.85 min, MS (ESI) m/z=175.9 [M+H]⁺.

### Step 3

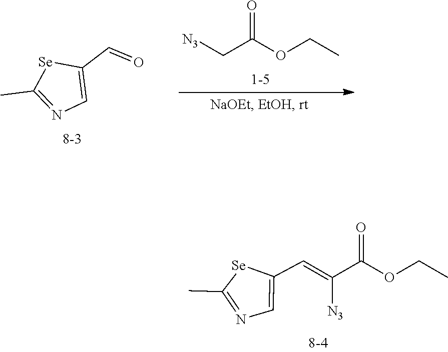

Compound 8-4: To a solution of sodium ethoxide (44.6 g, 131.2 mmol, 20% wt) in EtOH (200 mL) was added compound 8-3 (15.2 g, 87.4 mmol) and compound 1-5 (33.8 g, 262.2 mmol) at −10° C. under N₂. The mixture was stirred at −10° C. under N₂ for 1 h and at 10° C. for further 1 h. The mixture was quenched with saturated ammonium chloride aqueous solution (80 mL), extracted with ethyl acetate (300 mL×3). The organic layers were combined, dried over anhydrous sodium sulfate, filtered and concentrated under vacuum to afford crude product, which was purified by flash chromatography (elution gradient: petroleum ether/ethyl acetate, 5/1, v/v) to afford compound 6 (14.4 g) as yellow solid, yield: 58.0%. LCMS: Rt=1.33 min, MS (ESI) m/z=286.9 [M+H]⁺.

### Step 4

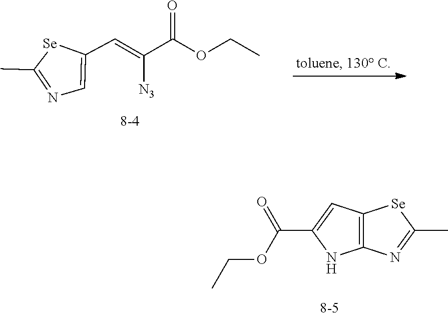 [SMILES: CCOC(=O)/C(=C/c1cnc(C)[se]1)N=[N+]=[N-] ; CCOC(=O)c1cc2[se]c(C)nc2[nH]1]

Compound 8-5: A solution of compound 8-4 (7.2 g, 25.2 mmol) in toluene (80 mL) was stirred at 130° C. for 5 h. The mixture was concentrated under vacuum to afford crude product, which was purified by flash chromatography (elution gradient: petroleum ether/ethyl acetate, 5/1, v/v) to afford compound 7 (5.2 g) as yellow solid, yield: 81.3%. LCMS: Rt=1.19 min, MS (ESI) m/z=259.0 [M+H]⁺.

### Step 5

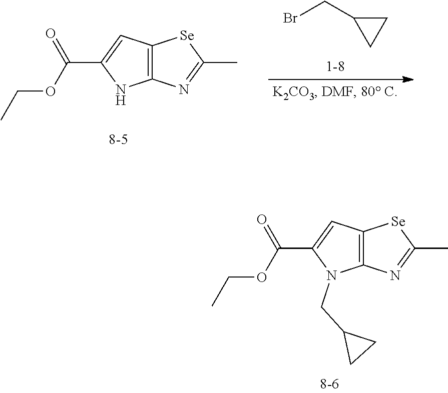 [SMILES: CCOC(=O)c1cc2[se]c(C)nc2[nH]1 ; BrCC1CC1 ; CCOC(=O)c1cc2[se]c(C)nc2n1CC1CC1]

Compound 8-6: To a solution of compound 8-5 (5.2 g, 20.2 mmol) in DMF (50 mL) was added compound 1-8 (4.2 g, 31.4 mmol) and K₂CO₃ (8.4 g, 61 mmol). The mixture was stirred at 80° C. for 5 h. The mixture was diluted with ethyl acetate (200 mL), washed with water (200 mL×2) and brine (150 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 9 (5.6 g) as a yellow solid, yield: 90.3%. LCMS: Rt=1.49 min, MS (ESI) m/z=313.0 [M+H]⁺.

### Step 6

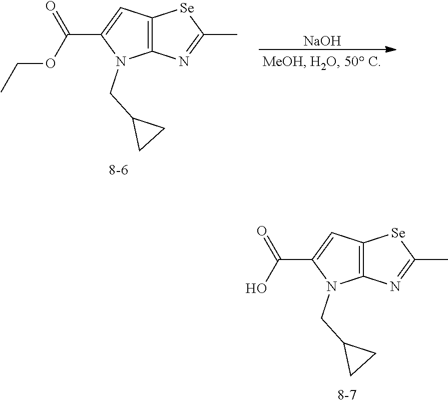 [SMILES: CCOC(=O)c1cc2[se]c(C)nc2n1CC1CC1 ; "NaOH" ; Cc1nc2c(cc(C(=O)O)n2CC2CC2)[se]1]

Compound 8-7: To a solution of compound 8-6 (5.6 g, 78 mmol) in MeOH (40 mL) was added 2N sodium hydroxide aqueous solution (36 mL, 78 mmol). The mixture was stirred at 50° C. for 12 h. The mixture was concentrated and adjusted pH to 3-4 with 1N hydrochloric acid aqueous solution. The precipitate was collected by filtration, washed with ethyl ether (15 mL), dried under vacuum to afford compound 10 (4.6 g) as a yellow solid, yield: 92.0%. LCMS: Rt=1.23 min, MS (ESI) m/z=284.9 [M+H]⁺.

### Step 7

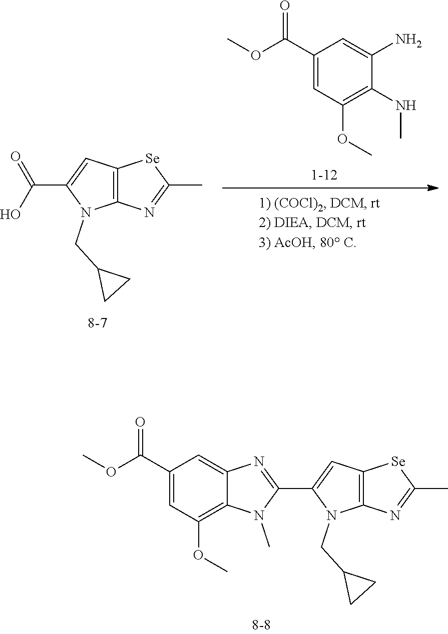

Compound 12: To a solution of compound 10 (4.6 g, 16.2 mmol) in DCM (80 mL) was added oxalyl chloride (4.2 g, 33.4 mmol) and DMF (0.1 mL). The mixture was stirred at room temperature for 1.5 h. The mixture was concentrated under vacuum to afford residue. The residue was dissolved in DCM (80 mL), DIEA (6.4 g, 49.6 mmol) and compound 1-12 (4 g, 19 mmol) was added. The mixture was stirred at room temperature for 2 h. The mixture was concentrated under vacuum to afford residue. The residue was dissolved in AcOH (80 mL) and stirred at 80° C. for 2 h. The mixture was concentrated under reduced pressure to afford residue. The residue was diluted with water (80 mL), extracted with ethyl acetate (100 mL×3). The combined organic layers were dried over anhydrous sodium sulfate and concentrated under vacuum to afford crude product, which was purified by flash chromatography (elution gradient: petroleum ether/ethyl acetate, 1/1, v/v) to afford compound 12 (6.4 g) as yellow solid, yield: 86.5%. LCMS: Rt=1.44 min, MS (ESI) m/z=458.9 [M+H]⁺.

### Step 8

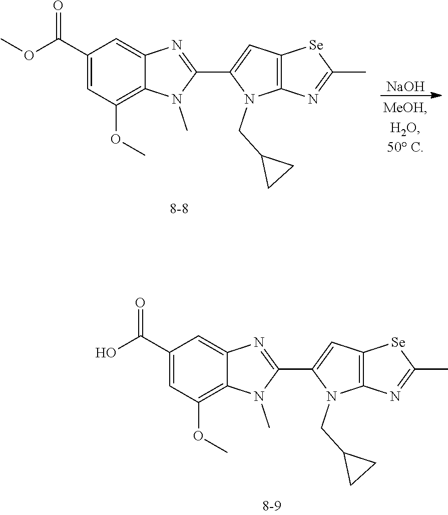

Compound 8-9: To a solution of compound 8-8 (6.4 g, 14 mmol) in MeOH (50 mL) was added 2N sodium hydroxide aqueous solution (28 mL, 56 mmol). The mixture was stirred at 50° C. for 12 h. The mixture was concentrated and adjusted pH to 3-4 with 1N hydrochloric acid aqueous solution. The precipitate was collected by filtration, washed with water (10 mL) and ethyl ether (10 mL), dried under vacuum to afford compound 13 (5.6 g) as a yellow solid, yield: 90.3%. LCMS: Rt=1.27 min, MS (ESI) m/z=445.1 [M+H]⁺.

### Step 9

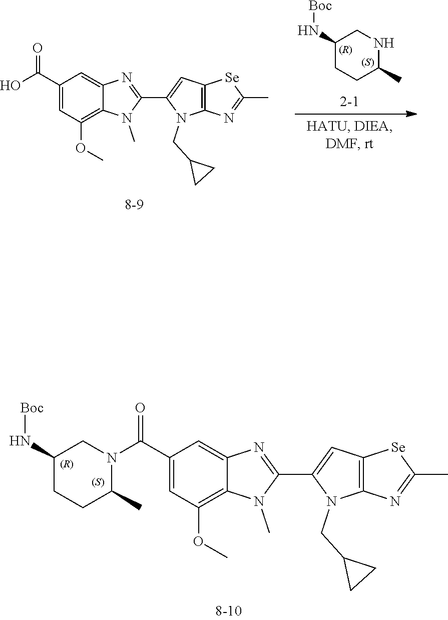

Compound 8-10: To a solution of compound 8-9 (1.8 g, 4.1 mmol), DIEA (1.6 g, 12.4 mmol), HATU (2 g, 5.3 mmol) in DMF (40 mL) was added compound 2-1 (1.1 g, 5.1 mmol). The mixture was stirred at room temperature for 3 h. The residue was diluted with ethyl acetate (100 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 15 (1.9 g) as a yellow solid, yield: 73.1%. LCMS: Rt=1.40 min, MS (ESI) m/z=641.2 [M+H]⁺.

### Step 10

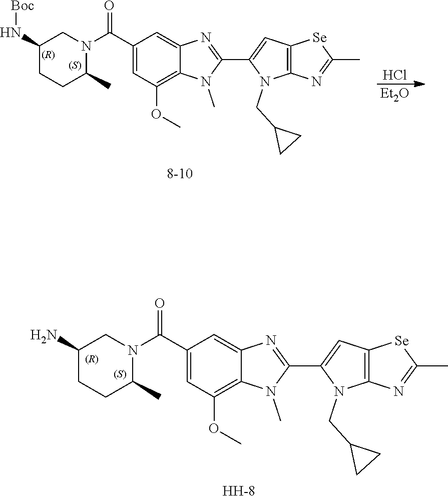

HH-8: To a solution of compound 8-10 (1.9 g, 3.0 mmol) in Et₂O (50 mL) was added 2N HCl in Et₂O (25 mL). The mixture was stirred at room temperature for 1 h. The mixture was concentrated and diluted with water (50 mL). The mixture was adjusted pH to 10 with 2N sodium hydroxide aqueous solution, and then extracted with dichloromethane (100 mL×3). The combined organic layers were dried over anhydrous sodium sulfate and concentrated under vacuum to afford crude product, which was purified by flash chromatography (elution gradient: dichloromethane/methanol, 10/1, v/v) to afford HH-8 (1.0173 g) as yellow solid, yield: 63.6%. ¹H NMR (400 MHz, DMSO-d₆): δ ppm 7.21 (s, 1H), 6.93 (s, 1H), 6.78 (s, 1H), 4.41 (d, J=7.2 Hz, 2H), 4.10 (s, 3H), 3.97 (s, 3H), 2.81 (s, 3H), 2.61 (br. s, 2H), 2.03 (br. s, 2H), 1.70-1.64 (m, 2H), 1.52-1.41 (m, 2H), 1.23-1.18 (m, 4H), 0.36-0.31 (m, 2H), 0.21-0.17 (m, 2H). LCMS: Rt=1.12 min, MS (ESI) m/z=541.0 [M+H]⁺. HPLC: Rt=4.94 min, Purity: 99.0% (214 nm and 254 nm).

### Example 9. Synthesis of HH-9

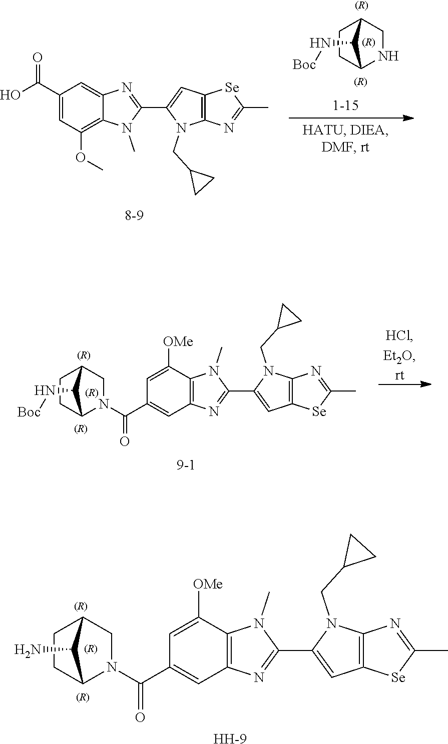

### Step 1

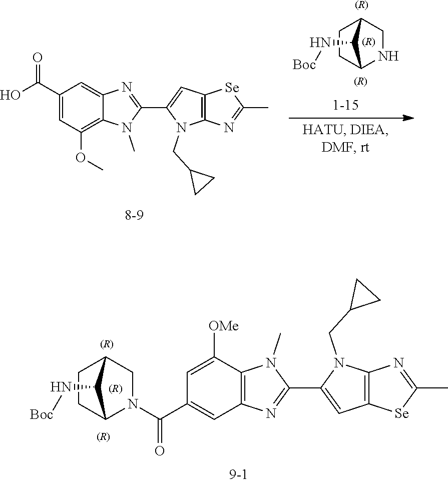

Compound 9-1: To a solution of compound 8-9 (500 mg, 1.1 mmol), DIEA (438 mg, 3.4 mmol), HATU (558 mg, 5.3 mmol) in DMF (10 mL) was added compound 1-15 (265 mg, 1.2 mmol). The mixture was stirred at room temperature for 2 h. The residue was diluted with ethyl acetate (100 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 9-1 (570 mg) as a yellow solid, yield: 79.3%. LCMS: Rt=1.38 min, MS (ESI) m/z=639.0 [M+H]⁺.

### Step 2

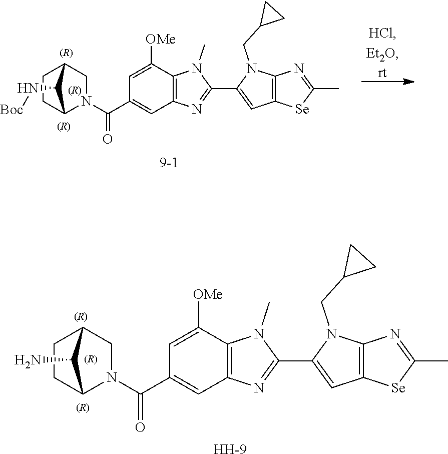

HH-9: To a solution of compound 9-1 (570 mg, 0.89 mmol) in ethyl ether (4 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 23-34%) to afford HH-9 (321.3 mg, hydrochloric acid salt form) as a brown solid. yield: 62.7%. ¹H NMR (400 MHz, DMSO-d₆): δ ppm 8.40 (s, 3H), 7.42 (s, 1H), 7.00 (s, 1H), 6.96 (s, 1H), 4.39 (d, J=6.8 Hz, 3H), 4.10 (s, 3H), 4.01 (s, 3H), 3.64 (br. s, 1H), 3.51 (s, 1H), 3.17 (d, J=10.0 Hz, 1H), 2.81 (s, 3H), 2.66 (s, 1H), 2.02-1.89 (m, 3H), 1.65-1.60 (m, 1H), 1.21-1.11 (m, 1H), 0.35-0.31 (m, 2H), 0.18-0.14 (m, 2H). LCMS: Rt=1.10 min, MS (ESI) m/z=539.0 [M+H]⁺. HPLC: Rt=5.11 min, Purity: 99.7% (214 nm and 254 nm)

### Example 10. Synthesis of HH-10

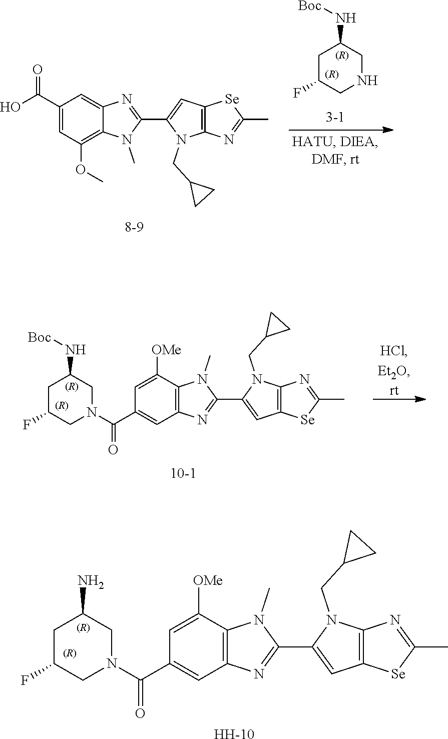

### Step 1

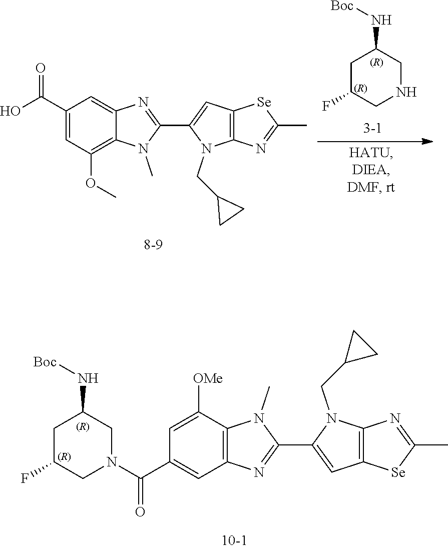

Compound 10-1: To a solution of compound 8-9 (500 mg, 1.1 mmol), DIEA (438 mg, 3.4 mmol), HATU (558 mg, 5.3 mmol) in DMF (10 mL) was added compound 3-1 (272 mg, 1.2 mmol). The mixture was stirred at room temperature for 2 h. The residue was diluted with ethyl acetate (100 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 3 (520 mg) as a yellow solid, yield: 71.7%. LCMS: Rt=1.36 min, MS (ESI) m/z=645.0 [M+H]⁺.

### Step 2

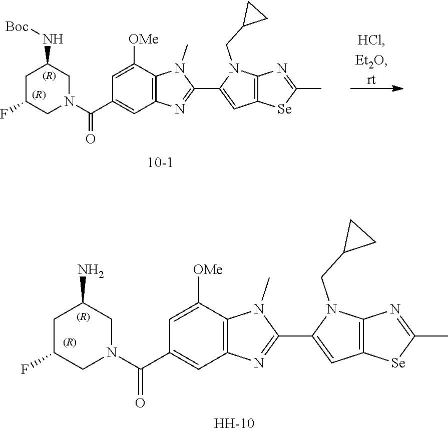

HH-10: To a solution of compound 10-1 (520 mg, 0.81 mmol) in ethyl ether (4 mL) was added hydrochloric acid solution (4 mL, 2M in ethyl ether). The mixture was stirred at room temperature for 2 h. The mixture was concentrated and diluted with water (50 mL). The mixture was adjusted pH to 10 with 2N sodium hydroxide aqueous solution, and then extracted with dichloromethane (100 mL×3). The combined organic layers were dried over anhydrous sodium sulfate and concentrated under vacuum to afford crude product, which was purified by flash chromatography (elution gradient: dichloromethane/methanol, 10/1, v/v). Fractions containing the desired compound was concentrated, diluted with water (10 mL) and hydrochloric acid aqueous solution (0.5 mL, 1N), which was freeze-dried under vacuum to afford HH-10 (305.6 mg, hydrochloric acid salt form) as a yellow solid. yield: 65.3%. ¹H NMR (400 MHz, DMSO-d₆): δ ppm 8.47 (s, 3H), 7.38 (s, 1H), 7.08 (s, 1H), 7.00 (s, 1H), 5.06-4.94 (m, 1H), 4.71 (br. s, 1H), 4.38 (d, J=6.8 Hz, 2H), 4.14-4.02 (m, 7H), 3.46-3.06 (m, 3H), 2.82 (s, 3H), 2.43-2.33 (m, 1H), 2.04-1.87 (m, 1H), 1.15-1.10 (m, 1H), 0.34-0.32 (m, 2H), 0.16-0.15 (m, 2H). LCMS: Rt=1.11 min, MS (ESI) m/z=545.0 [M+H]⁺. HPLC: Rt=4.77 min, Purity: 98.3% (214 nm) and 98.4% (254 nm).

### Example 11. Synthesis of HH-11

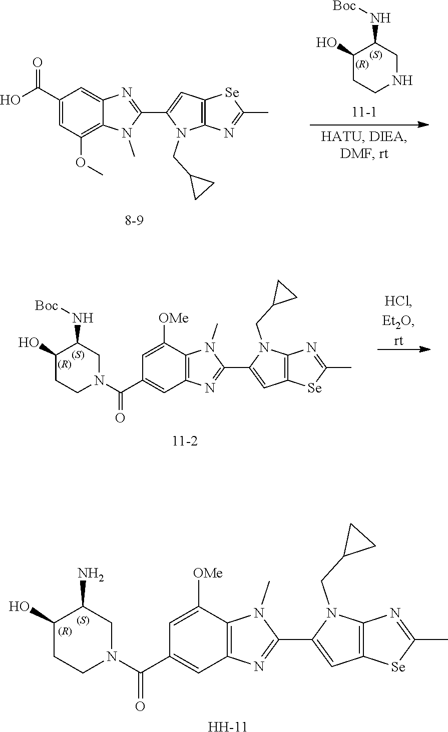

### Step 1

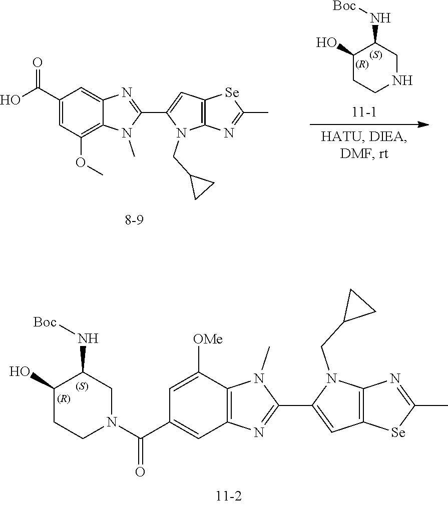

Compound 11-2: To a solution of compound 8-9 (70 mg, 0.16 mmol), DIEA (61 mg, 0.47 mmol), HATU (78 mg, 0.21 mmol) in DMF (5 mL) was added compound 11-1 (38 mg, 0.18 mmol). The mixture was stirred at room temperature for 2 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 11-2 (85 mg) as a yellow solid, yield: 84.0%. LCMS: Rt=1.28 min, MS (ESI) m/z=643.0 [M+H]⁺.

### Step 2

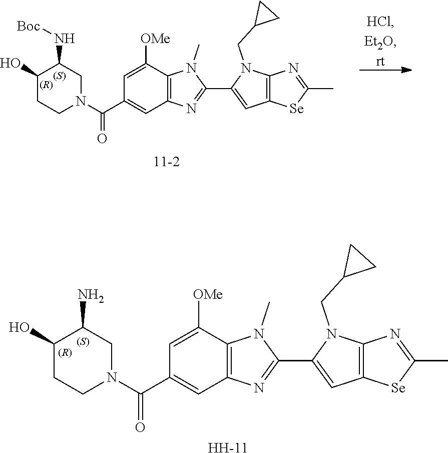

HH-11: To a solution of compound 11-2 (85 mg, 0.13 mmol) in ethyl ether (2 mL) was added hydrochloric acid solution (2 mL, 2M in ethyl ether). The mixture was stirred at room temperature for 2 h. The mixture was concentrated under vacuum to afford crude product, which was purified by preparative HPLC (Gemini-C18 column, 5μ silica, 21.2 mm diameter, 150 mm length), using decreasingly polar mixtures of water (containing 0.05% FA) and MeCN as eluents 10-40%) to afford HH-11 (42.4 mg, hydrochloric acid salt form) as a yellow solid. yield: 55.4%. ¹H NMR (400 MHz, DMSO-d₆): δ ppm 8.16 (s, 3H), 7.43 (s, 1H), 7.07 (s, 2H), 4.39 (d, J=6.8 Hz, 2H), 4.14 (s, 3H), 4.08-4.07 (m, 1H), 4.02 (s, 3H), 3.61-3.36 (m, 4H), 2.82 (s, 3H), 1.82-1.68 (m, 2H), 1.16-1.07 (m, 1H), 0.35-0.31 (m, 2H), 0.17-0.13 (m, 2H). LCMS: Rt=1.10 min, MS (ESI) m/z=543.0 [M+H]⁺. HPLC: Rt=4.68 min, Purity: 100% (214 nm and 254 nm).

### Example 12. Synthesis of HH-12

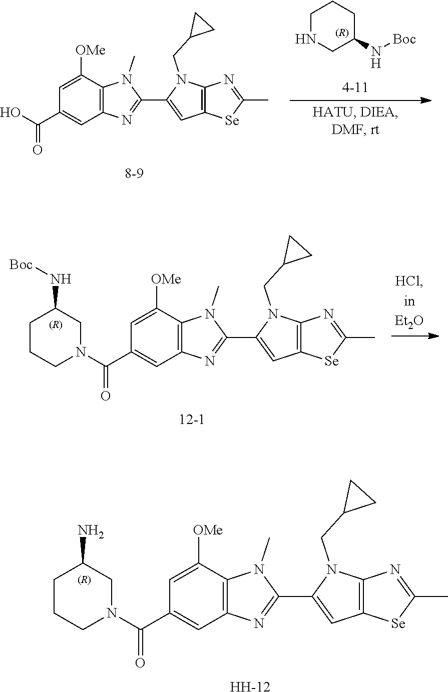

### Step 1

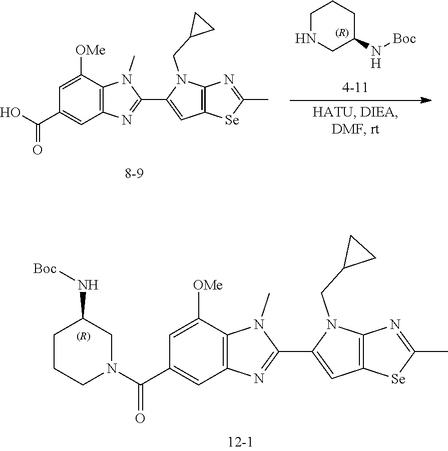

Compound 12-1: To a solution of compound 8-9 (60 mg, 0.14 mmol), DIEA (53 mg, 0.41 mmol), HATU (67 mg, 0.18 mmol) in DMF (5 mL) was added compound 4-11 (30 mg, 0.15 mmol). The mixture was stirred at room temperature for 2 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 12-1 (75 mg) as a yellow solid, yield: 88.7%. LCMS: Rt=1.37 min, MS (ESI) m/z=627.0 [M+H]⁺.

### Step 2

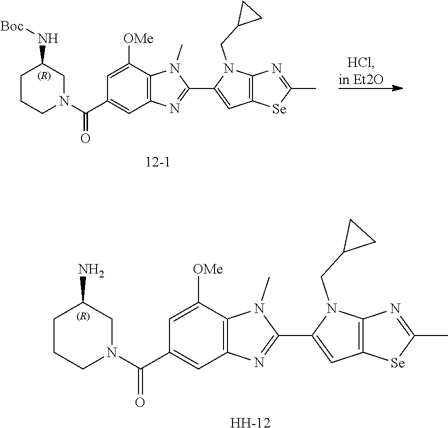

HH-12: To a solution of compound 12-1 (75 mg, 0.12 mmol) in ethyl ether (2 mL) was added hydrochloric acid solution (2 mL, 2M in ethyl ether). The mixture was stirred at room temperature for 2 h. The mixture was concentrated under vacuum to afford crude product, which was purified by preparative HPLC (Gemini-C18 column, 5μ silica, 21.2 mm diameter, 150 mm length), using decreasingly polar mixtures of water (containing 0.05% FA) and MeCN as eluents 10-40%) to afford HH-12 (37.9 mg, hydrochloric acid salt form) as a yellow solid. yield: 56.3%. ¹H NMR (400 MHz, DMSO-d₆): δ ppm 8.39 (s, 3H), 7.42 (s, 1H), 7.08 (s, 2H), 4.38 (d, J=7.2 Hz, 2H), 4.14 (s, 3H), 4.26-4.14 (m, 1H), 4.03 (s, 3H), 3.27-3.13 (m, 3H), 2.82 (s, 3H), 2.07-2.03 (m, 1H), 1.80-1.53 (m, 3H), 1.15-1.09 (m, 1H), 0.35-0.31 (m, 2H), 0.16-0.13 (m, 2H). LCMS: Rt=1.10 min, MS (ESI) m/z=527.0 [M+H]⁺. HPLC: Rt=4.84 min, Purity: 100% (214 nm and 254 nm)

### Example 13. Synthesis of HH-13

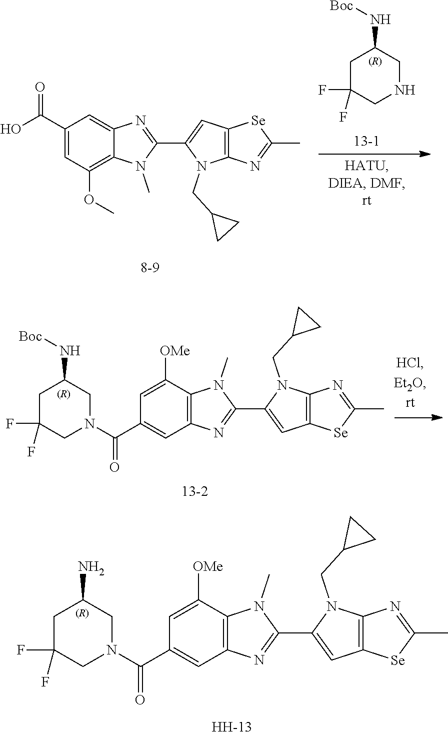

### Step 1

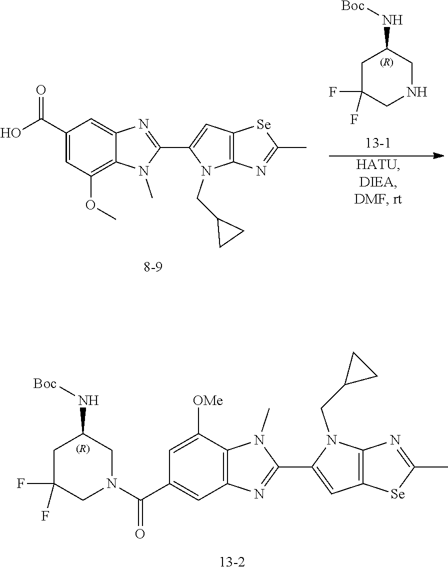

Compound 13-2: To a solution of compound 8-9 (50 mg, 0.11 mmol), DIEA (44 mg, 0.34 mmol), HATU (56 mg, 0.15 mmol) in DMF (5 mL) was added compound 13-1 (30 mg, 0.13 mmol). The mixture was stirred at room temperature for 2 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 13-2 (60 mg) as a yellow solid, yield: 80.4%. LCMS: Rt=1.42 min, MS (ESI) m/z=663.0 [M+H]⁺.

### Step 2

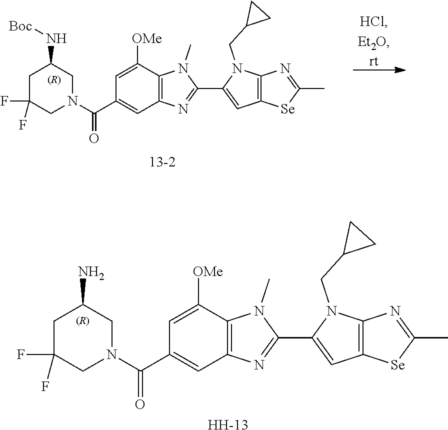

HH-13: To a solution of compound 13-2 (60 mg, 0.091 mmol) in ethyl ether (2 mL) was added hydrochloric acid solution (2 mL, 2M in ethyl ether). The mixture was stirred at room temperature for 2 h. The mixture was concentrated under vacuum to afford crude product, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-13 (20.8 mg, hydrochloric acid salt form) as a yellow solid. yield: 38.4%. ¹H NMR (400 MHz, DMSO-d₆): δ ppm 8.65 (s, 3H), 7.43 (s, 1H), 7.07 (s, 1H), 7.03 (s, 1H), 4.39 (d, J=7.2 Hz, 2H), 4.14 (s, 3H), 4.02 (s, 3H), 3.76-3.57 (m, 1H), 3.43-3.38 (m, 2H), 3.09-3.02 (m, 1H), 2.82 (s, 3H), 2.68-2.59 (m, 1H), 2.47-2.30 (m, 1H), 1.16-1.11 (m, 1H), 0.35-0.31 (m, 2H), 0.18-0.15 (m, 2H). LCMS: Rt=1.13 min, MS (ESI) m/z=563.1 [M+H]⁺. HPLC: Rt=4.96 min, Purity: 99.2% (214 nm) and 99.1% (254 nm).

### Example 14. Synthesis of HH-14

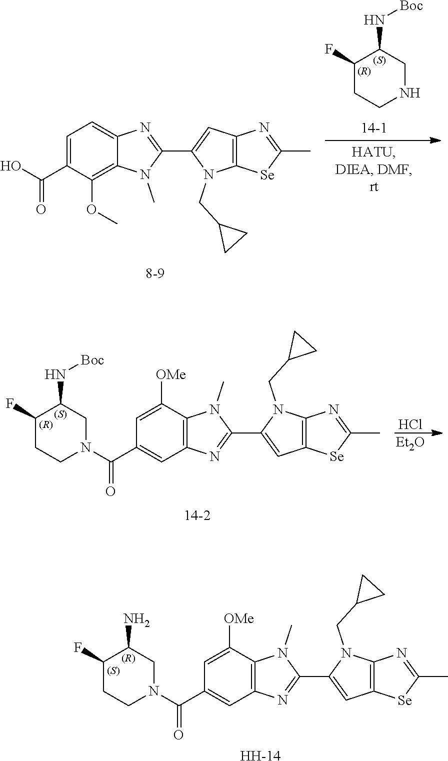

### Step 1

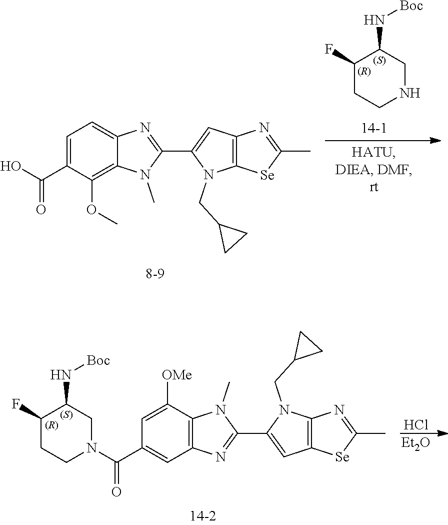

Compound 14-2: To a solution of compound 8-9 (40 mg, 0.09 mmol), DIEA (35 mg, 0.27 mmol), HATU (45 mg, 0.12 mmol) in DMF (5 mL) was added compound 2 (30 mg, 0.13 mmol). The mixture was stirred at room temperature for 2 h. The residue was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 14-2 (38 mg) as a yellow solid, yield: 65.5%. LCMS: Rt=1.39 min, MS (ESI) m/z=645.0 [M+H]⁺.

### Step 2

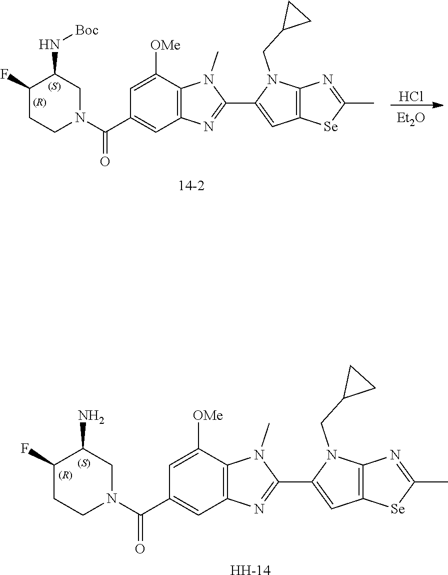

HH-14: To a solution of compound 14-2 (38 mg, 0.059 mmol) in ethyl ether (2 mL) was added hydrochloric acid solution (2 mL, 2M in ethyl ether). The mixture was stirred at room temperature for 2 h. The mixture was concentrated under vacuum to afford crude product, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-14 (22.8 mg, hydrochloric acid salt form) as a yellow solid. yield: 66.7%. ¹H NMR (400 MHz, DMSO-d₆): δ ppm 8.75 (s, 3H), 7.47 (s, 1H), 7.13 (s, 2H), 5.28-5.15 (m, 1H), 4.51-4.29 (m, 3H), 4.15 (s, 3H), 4.04 (s, 3H), 3.68-3.61 (m, 1H), 3.36-3.17 (m, 2H), 2.83 (s, 3H), 2.08-1.97 (m, 2H), 1.11-1.08 (m, 1H), 0.33-0.31 (m, 2H), 0.15-0.12 (m, 2H). LCMS: Rt=1.12 min, MS (ESI) m/z=545.0 [M+H]⁺. HPLC: Rt=4.80 min, Purity: 100% (214 nm and 254 nm).

### Example 15. Synthesis of HH-15

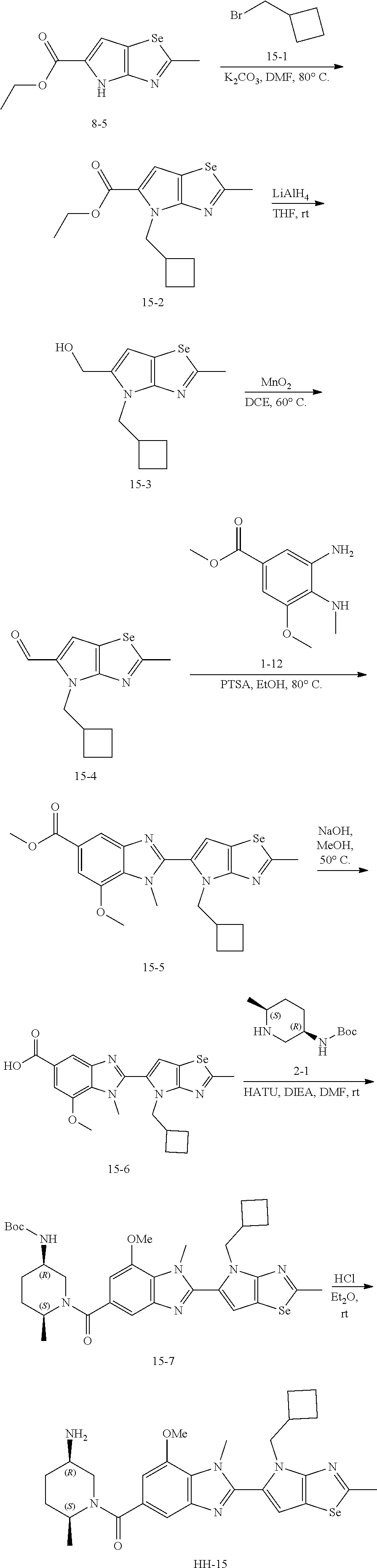

### Step 1

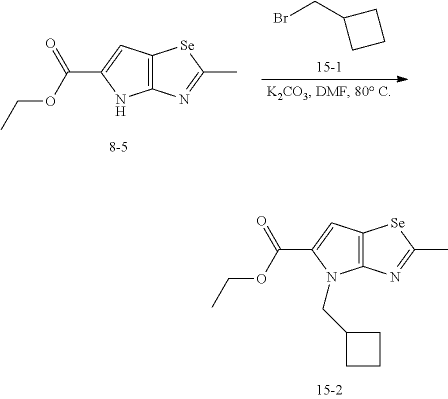 [SMILES: CCOC(=O)c1cc2[se]c(C)nc2[nH]1 ; BrCC1CCC1 ; CCOC(=O)c1cc2[se]c(C)nc2n1CC1CCC1]

Compound 15-2: To a solution of compound 8-5 (1.5 g, 5.82 mmol) in DMF (25 mL) was added compound 15-1 (1.304 g, 8.76 mmol) and K₂CO₃ (1.612 g, 11.67 mmol). The mixture was stirred at 80° C. for 8 h. The mixture was diluted with ethyl acetate (100 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 3 (1.53 g) as a yellow solid, yield: 80.6%. LCMS: Rt=1.544 min, MS (ESI) m/z=327.1 [M+H]⁺.

### Step 2

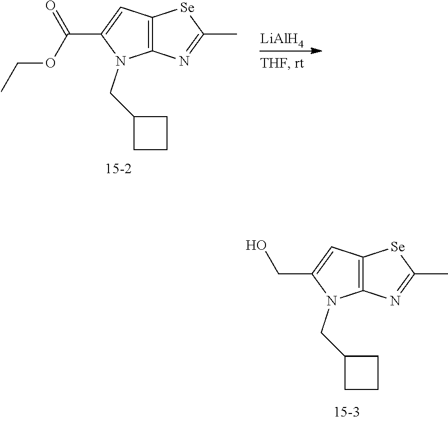

Compound 15-3: To a solution of compound 15-2 (1.53 g, 5.16 mmol) in THF (25 mL), then was added LiAlH₄ (1M in THF, 10.3 mL, 10.3 mmol) at 0° C., The reaction mixture was stirred at 25° C. for 3 h. The mixture was quenched with water (20 mL) and filtered. The filtrate was extracted with ethyl acetate (20 mL×3). The organic layers were dried over anhydrous Na₂SO₄ and concentrated under vacuum to afford crude product (1.05 g) as a yellow oil. LCMS: Rt=1.243 min, MS (ESI) m/z=285.1 [M+H]⁺.

### Step 3

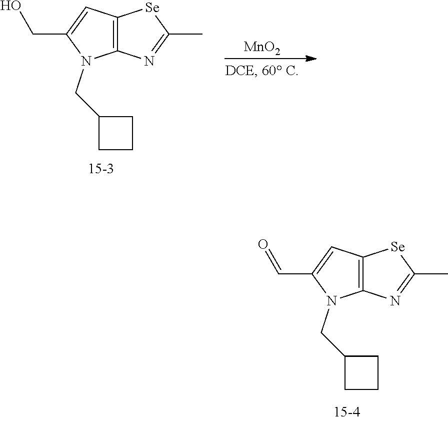

Compound 15-4: To a solution of compound 15-3 (1.05 g, 3.72 mmol) in DCE (50 mL) was added MnO₂ (3.21 g, 36.9 mmol). The reaction mixture was stirred at 60° C. for 16 h. The mixture was filtered and the filtrate was concentrated under vacuum to afford crude product 15-4 (840 mg) as a brown oil. LCMS: Rt=1.388 min, MS (ESI) m/z=282.9 [M+H]⁺.

### Step 4

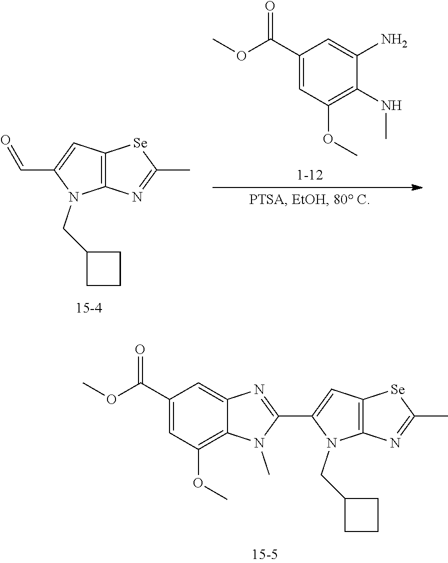

Compound 15-5: To a solution of compound 15-4 (840 mg, 2.97 mmol) in EtOH (25 mL) was added compound 1-12 (627 mg, 2.97 mmol) and PTSA (51 mg, 0.27 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 15-5 (900 mg) as a yellow oil, yield: 63.9%. LCMS: Rt=1.485 min, MS (ESI) m/z=473.0 [M+H]⁺.

### Step 5

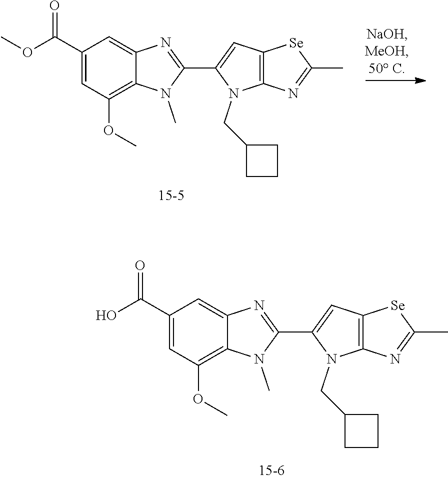

Compound 15-6: To a solution of compound 15-5 (900 mg, 1.92 mmol) in MeOH (25 mL) was added sodium hydroxide (2 N, 1.9 mL). The mixture was stirred at 50° C. for 3 h. The mixture was concentrated and adjusted pH to 5-6 with hydrochloric acid aqueous solution (2N), then extracted with ethyl acetate (50 mL×3). The organic phase was washed with water (50 mL), brine (50 mL), and concentrated under vacuum to give compound 8 (600 mg) as a yellow solid, yield: 71.5%. LCMS: Rt=1.336 min, MS (ESI) m/z=458.9 [M+H]⁺.

### Step 6

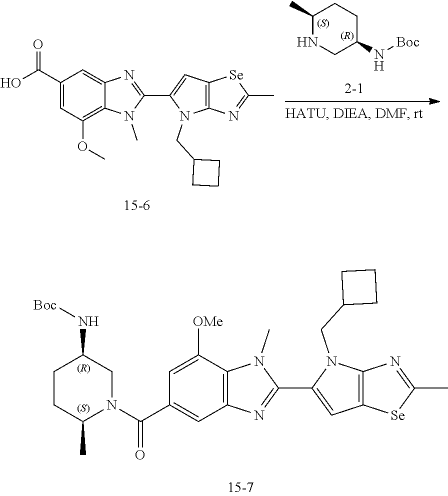

Compound 15-7: To a solution of compound 15-6 (100 mg, 0.22 mmol) in DMF (8 mL) was added compound 2-1 (51.4 mg, 0.24 mmol), HATU (99.6 mg, 0.26 mmol) and DIEA (84.8 mg, 0.64 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1, v/v) to afford compound 15-7 (100 mg) as a brown solid, yield: 69.9%. LCMS: Rt=1.431 min, MS (ESI) m/z=655.2 [M+H]⁺.

### Step 7

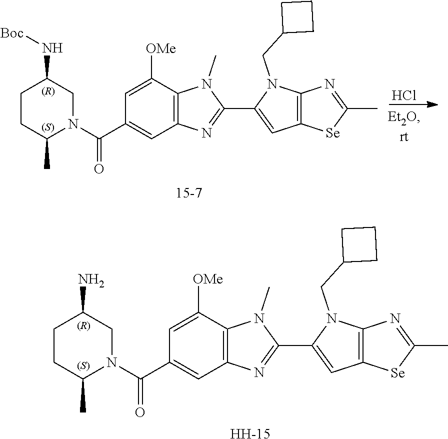

HH-15: To a solution of compound 15-7 (100 mg, 0.16 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-15 (44.0 mg, hydrochloric acid salt form) as a yellow solid. yield: 52%. ¹H NMR (400 MHz, DMSO-d₆) δ ppm 8.28 (s, 3H), 7.35 (s, 1H), 7.00 (s, 1H), 6.94 (s, 1H), 4.59 (d, J=7.2 Hz, 2H), 4.11 (s, 4H), 4.01 (s, 3H), 3.19-3.06 (m, 2H), 2.82 (s, 3H), 2.56-2.60 (m, 2H), 1.95-1.40 (m, 10H), 1.22 (d, J=6.8 Hz, 3H). LCMS: Rt=1.122 min, MS (ESI) m/z=555.2 [M+H]⁺. HPLC: Rt=4.831 min, Purity: 98.2% (214 nm) and 98.5% (254 nm).

### Example 16. Synthesis of HH-16

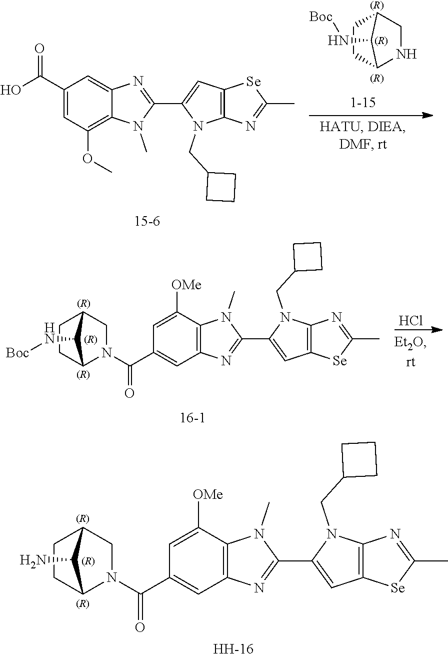

### Step 1

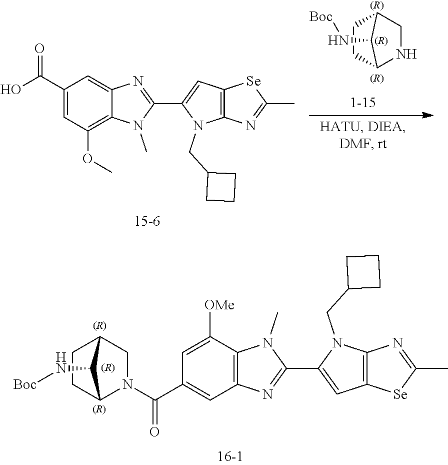

Compound 16-1: To a solution of compound 15-6 (100 mg, 0.22 mmol) in DMF (8 mL) was added compound 1-15 (51 mg, 0.24 mmol), HATU (99.6 mg, 0.26 mmol) and DIEA (84.8 mg, 0.64 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1, v/v) to afford compound 16-1 (100 mg) as a brown solid, yield: 70.2%. LCMS: Rt=1.399 min, MS (ESI) m/z=653.2 [M+H]⁺.

### Step 2

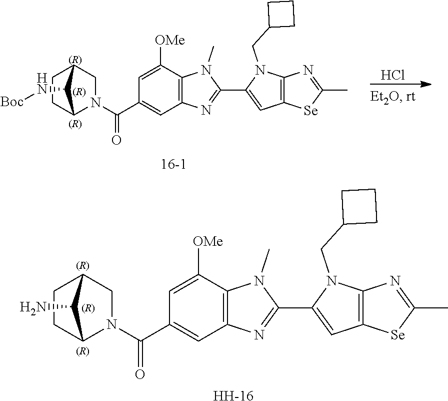

HH-16: To a solution of compound 16-1 (100 mg, 0.16 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-16 (60 mg, hydrochloric acid salt form) as a yellow solid. yield: 70.8%. ¹H NMR (400 MHz, DMSO-d₆) δ ppm 8.38 (s, 3H), 7.42 (s, 1H), 6.98 (s, 1H), 6.92 (s, 1H), 4.59 (d, J=7.2 Hz, 2H), 4.35 (br. s, 1H), 4.08 (s, 3H), 4.01 (s, 3H), 3.65 (s, 1H), 3.51 (s, 1H), 3.18 (d, J=10.4 Hz, 1H), 2.80 (s, 3H), 2.66-2.59 (m, 2H), 2.05-1.88 (m, 3H), 1.81-1.61 (m, 5H), 1.58-1.48 (m, 2H). LCMS: Rt=1.096 min, MS (ESI) m/z=553.2 [M+H]⁺. HPLC: Rt=4.628 min, Purity: 95.39% (214 nm) and 97.04% (254 nm).

### Example 17. Synthesis of HH-17

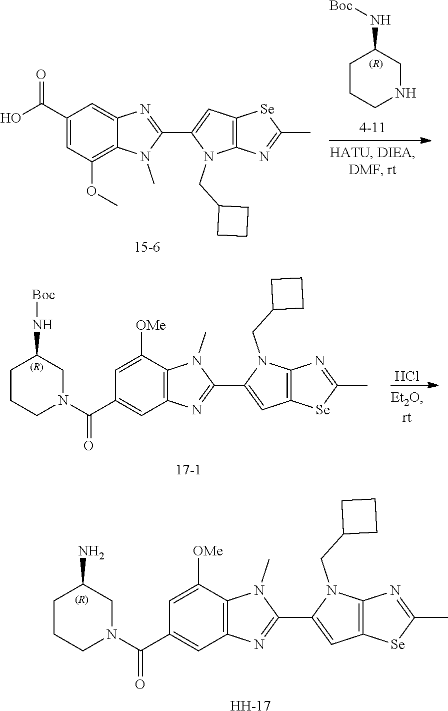

### Step 1

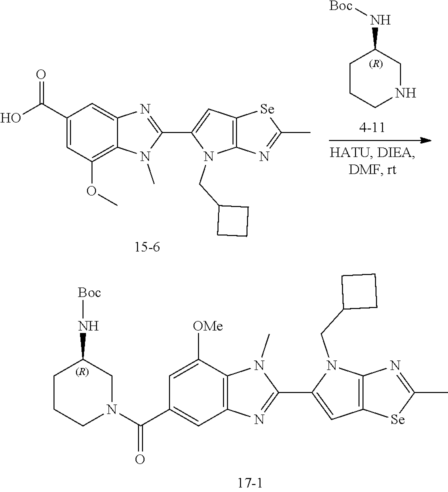

Compound 17-1: To a solution of compound 15-6 (100 mg, 0.22 mmol) in DMF (8 mL) was added compound 4-11 (48.2 mg, 0.24 mmol), HATU (99.6 mg, 0.26 mmol) and DIEA (84.8 mg, 0.64 mmol). The reaction mixture was stirred at 25° C. for 5 h. The mixture was diluted with water (30 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1, v/v) to afford compound 17-1 (100 mg) as a brown solid, yield: 71.5%. LCMS: Rt=1.397 min, MS (ESI) m/z=641.2 [M+H]⁺.

### Step 2

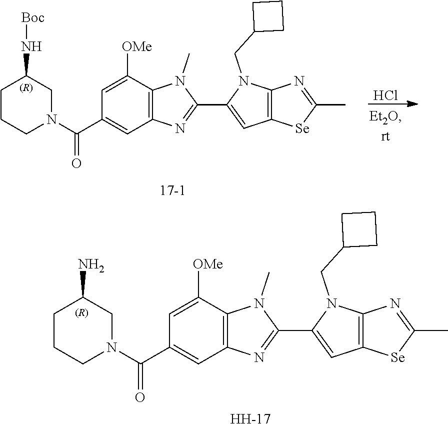

HH-17: To a solution of compound 17-1 (100 mg, 0.16 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-17 (60 mg, hydrochloric acid salt form) as a yellow solid, yield: 47.4%. ¹H NMR (400 MHz, DMSO-d₆) δ ppm 8.33 (s, 3H), 7.42 (s, 1H), 7.02 (s, 2H), 4.59 (d, J=7.2 Hz, 2H), 4.11 (s, 4H), 4.01 (s, 3H), 3.28-3.10 (m, 3H), 2.82 (s, 3H), 2.08-2.01 (m, 1H), 1.72-1.45 (m, 10H). LCMS: Rt=1.102 min, MS (ESI) m/z=541.2 [M+H]⁺. HPLC: Rt=4.670 min, Purity: 98.65% (214 nm) and 98.95% (254 nm).

### Example 18. Synthesis of HH-18

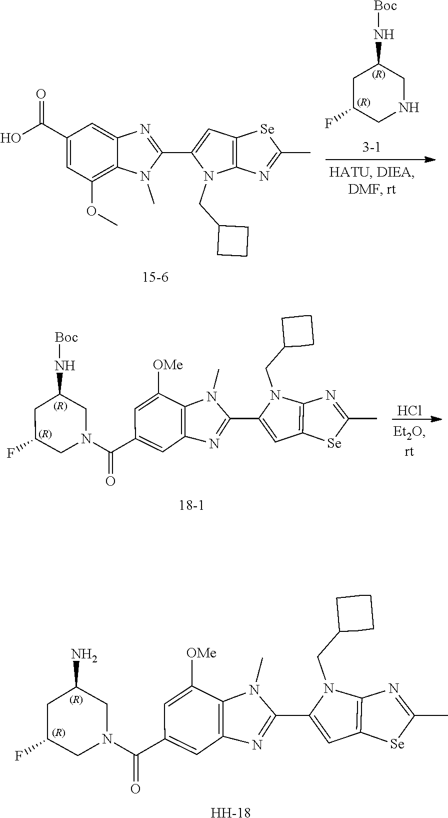

### Step 1

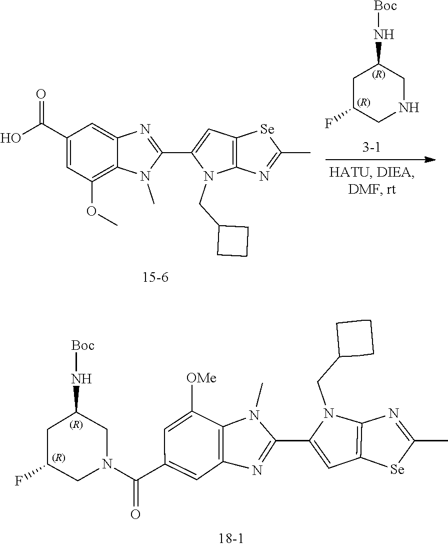

Compound 18-1: To a solution of compound 15-6 (100 mg, 0.22 mmol) in DMF (8 mL) was added compound 3-1 (52.4 mg, 0.24 mmol), HATU (99.6 mg, 0.26 mmol) and DIEA (84.8 mg, 0.64 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1, v/v) to afford compound 18-1 (100 mg) as a brown solid, yield: 69.5%. LCMS: Rt=1.354 min, MS (ESI) m/z=659.2 [M+H]⁺.

### Step 2

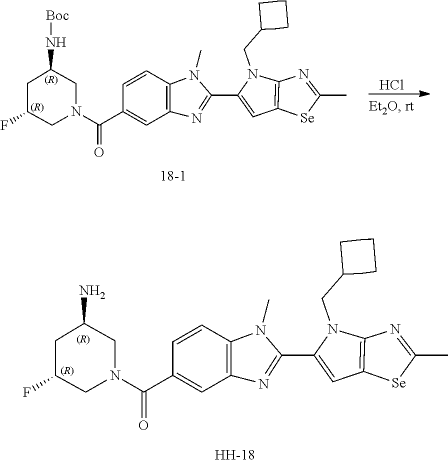

HH-18: To a solution of compound 18-1 (100 mg, 0.16 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-18 (54 mg, hydrochloric acid salt form) as a yellow solid. yield: 63.9%. ¹H NMR (400 MHz, DMSO-d₆) δ ppm 8.36 (s, 3H), 7.36 (s, 1H), 6.98 (s, 1H), 6.93 (s, 1H), 5.05-4.94 (m, 1H), 4.60 (d, J=7.2 Hz, 3H), 4.11 (s, 3H), 3.99 (s, 3H), 3.39 (s, 2H), 3.03 (s, 1H), 2.82 (s, 3H), 2.60-2.56 (m, 2H), 2.45-2.31 (m, 1H), 2.03-1.80 (m, 1H), 1.78-1.59 (m, 4H), 1.55-1.42 (m, 2H). LCMS: Rt=1.117 min, MS (ESI) m/z=559.1 [M+H]⁺. HPLC: Rt=4.757 min, Purity: 98.89% (214 nm) and 98.28% (254 nm).

### Example 19. Synthesis of HH-19

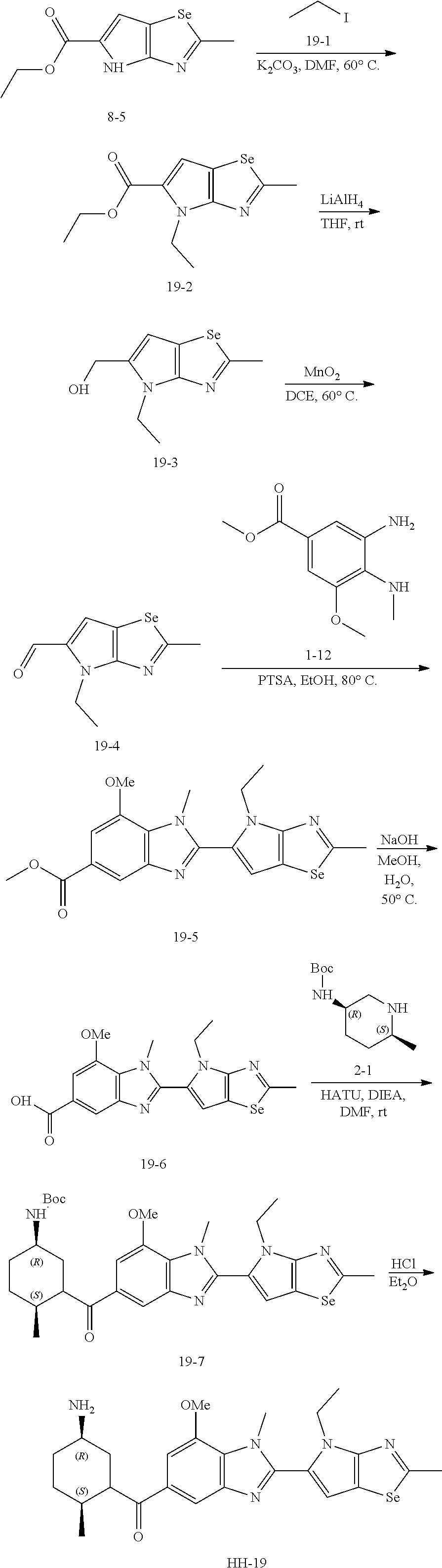

### Step 1

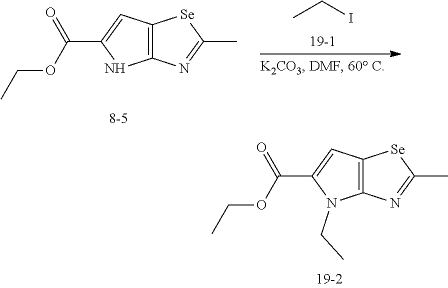

Compound 19-2: To a solution of compound 8-5 (1.5 g, 5.82 mmol) and compound 19-1 (1.214 g, 7.78 mmol) in DMF (20 mL) was added potassium carbonate (2.012 g, 14.57 mmol). The mixture was stirred at 60° C. for 16 h. The mixture was diluted with water (40 mL) and extracted with ethyl acetate (50 mL×3). The organic layers were washed with brine (50 mL), dried over anhydrous Na₂SO₄ and concentrated under vacuum to afford crude product, which was purified by silica gel chromatography (elution gradient: petroleum ether/EtOAc, 1/1, v/v) to afford compound 19-2 (1.5 g) as a yellow oil, yield: 90.3%. LCMS: Rt=1.424 min, MS (ESI) m/z=286.9 [M+H]⁺.

### Step 2

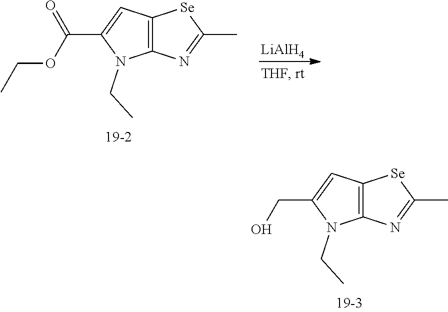

Compound 19-3: To a solution of compound 19-2 (1.5 g, 5.25 mmol) in THF (15 mL), then was added LiAlH₄ (1M in THF, 15.8 mL, 15.8 mmol) at 0° C. The reaction mixture was stirred at 25° C. for 2 h. The mixture was quenched with water (30 mL) and filtered. The filtrate was extracted with ethyl acetate (20 mL×3). The organic layers were dried over anhydrous Na₂SO₄ and concentrated under vacuum to afford crude product (1.05 g) as a yellow oil. LCMS: Rt=1.106 min, MS (ESI) m/z=245.0 [M+H]⁺.

### Step 3

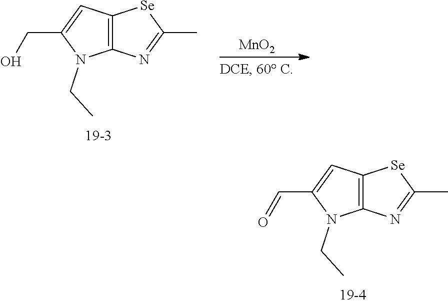

Compound 19-4: To a solution of compound 19-3 (1.05 mg, 4.32 mmol) in DCE (25 mL) was added MnO₂ (3.756 g, 43.17 mmol). The reaction mixture was stirred at 60° C. for 16 h. The mixture was filtered and the filtrate was concentrated under vacuum to afford crude product (960 mg) as a brown oil. LCMS: Rt=1.238 min, MS (ESI) m/z=242.9 [M+H]⁺.

### Step 4

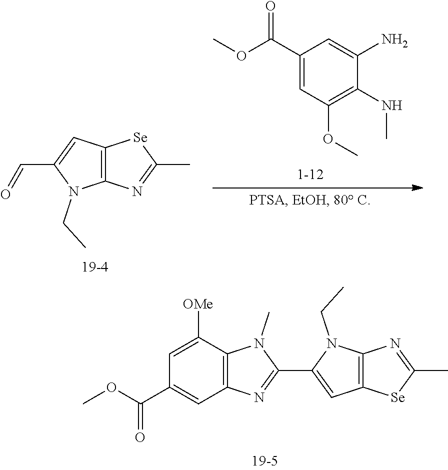

Compound 19-5: To a solution of compound 19-4 (960 mg, 3.99 mmol) in EtOH (25 mL) was added compound 1-12 (921 mg, 4.38 mmol) and PTSA (69 mg, 0.39 mmol). The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 19-5 (960 mg) as a yellow oil, yield: 56.0%. LCMS: Rt=1.376 min, MS (ESI) m/z=432.9 [M+H]⁺.

### Step 5

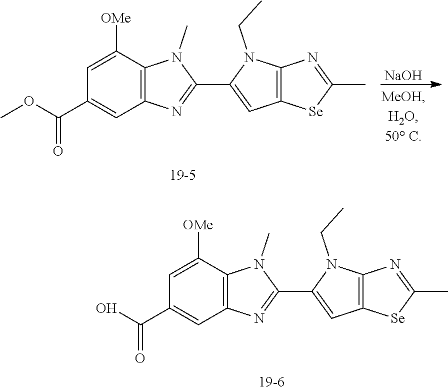

Compound 19-6: To a solution of compound 19-5 (960 mg, 2.22 mmol) in MeOH/H₂O (15/3 mL) was added NaOH (267 mg, 6.69 mmol). The reaction mixture was stirred at 50° C. for 16 h. The mixture was concentrated and adjusted pH to 5-6 with 1N hydrochloric acid aqueous solution, then extracted with ethyl acetate (30 mL×3). The organic phase was washed with water (20 mL), brine (20 mL), dried over anhydrous Na₂SO₄ and concentrated under vacuum to give compound 19-6 (720 mg) as a yellow solid, yield: 77.7%. LCMS: Rt=1.233 min, MS (ESI) m/z=418.9 [M+H]⁺.

### Step 6

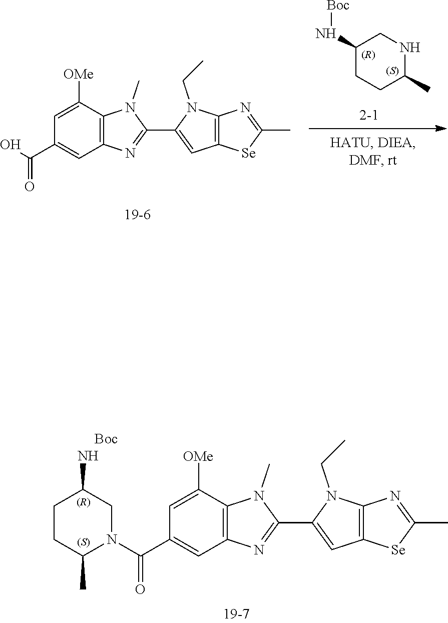

Compound 19-7: To a solution of compound 19-6 (160 mg, 0.38 mmol) in DMF (8 mL) was added compound 2-1 (100 mg, 0.46 mmol), HATU (146 mg, 0.38 mmol) and DIEA (148 mg, 1.16 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 19-7 (180 mg) as a brown solid, yield: 76.3%. LCMS: Rt=1.349 min, MS (ESI) m/z=615.0 [M+H]⁺.

### Step 7

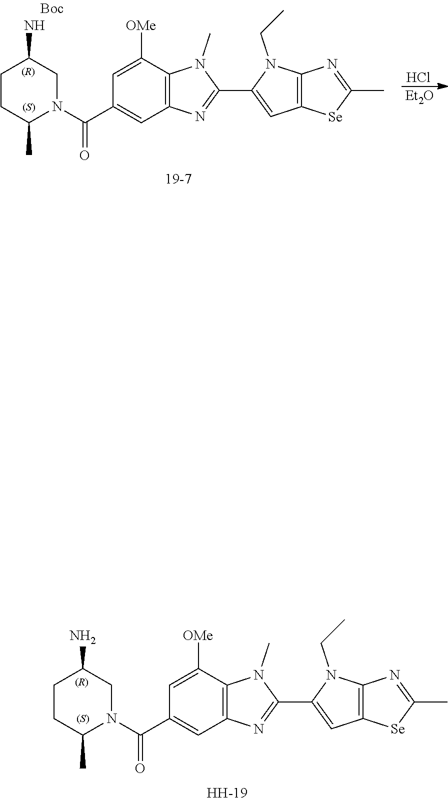

HH-19: To a solution of compound 19-7 (180 mg, 0.3 mmol) in Et₂O (8 mL) was added 2M HCl in Et₂O (12 mL, 24 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-19 (64 mg, hydrochloric acid salt form) as a yellow solid. yield: 42.6%. ¹H NMR (400 MHz, DMSO-d₆) δ ppm 8.27 (s, 3H), 7.35 (s, 1H), 7.03 (s, 1H), 6.96 (s, 1H), 4.69-4.49 (m, 3H), 4.05-3.73 (m, 7H), 3.22-3.12 (m, 2H), 2.83 (s, 3H), 1.97-1.71 (m, 3H), 1.59 (s, 1H), 1.35-1.07 (m, 6H). LCMS: Rt=1.100 min, MS (ESI) m/z=514.9 [M+H]⁺. HPLC: Rt=4.142 min, Purity: 99.65% (214 nm) and 99.66% (254 nm).

### Example 20. Synthesis of HH-20

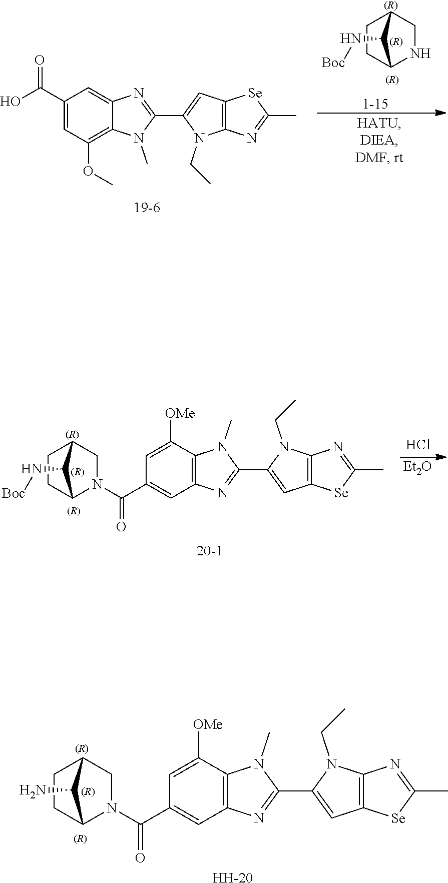

### Step 1

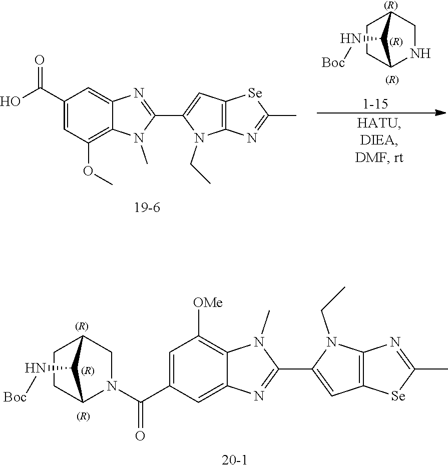

Compound 20-1: To a solution of compound 19-6 (80 mg, 0.19 mmol) in DMF (5 mL) was added compound 1-15 (50 mg, 0.23 mmol), HATU (73 mg, 0.19 mmol) and DIEA (74 mg, 0.58 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 20-1 (90 mg) as a brown solid, yield: 76.3%. LCMS: Rt=1.320 min, MS (ESI) m/z=613.0 [M+H]⁺.

### Step 2

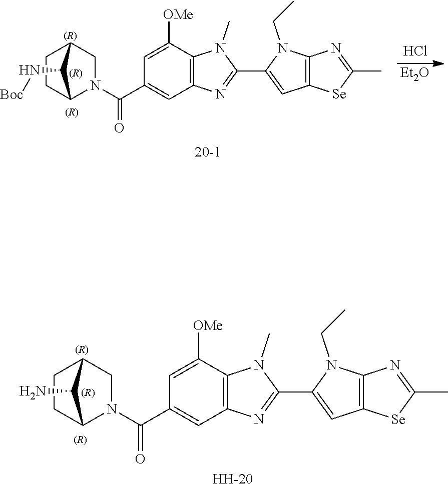

Compound HH-20: To a solution of compound 20-1 (80 mg, 0.19 mmol) in DMF (5 mL) was added compound 2 (50 mg, 0.23 mmol), HATU (73 mg, 0.19 mmol) and DIEA (74 mg, 0.58 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound HH-20 (90 mg) as a brown solid, yield: 76.3%. LCMS: Rt=1.320 min, MS (ESI) m/z=613.0 [M+H]⁺.

### Example 21. Synthesis of HH-21

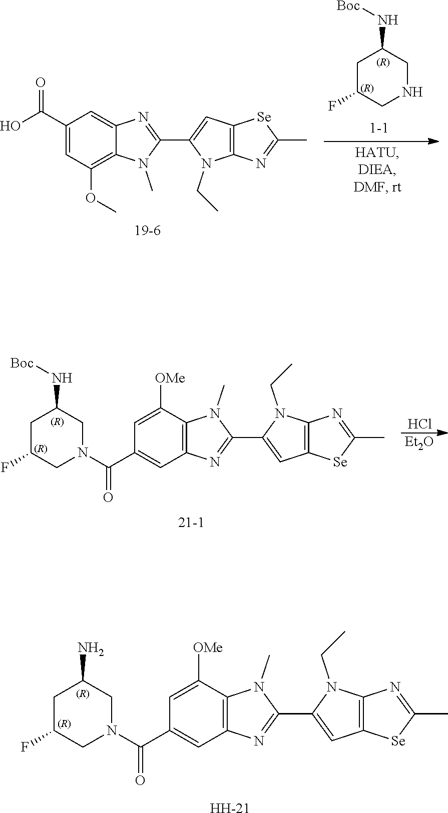

### Step 1

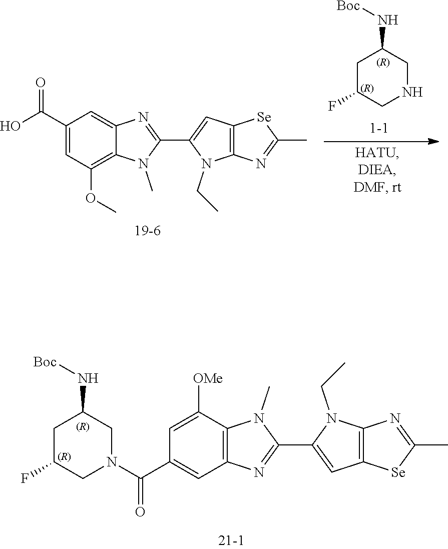

Compound 21-1: To a solution of compound 19-6 (80 mg, 0.19 mmol) in DMF (5 mL) was added compound 2 (50 mg, 0.23 mmol), HATU (73 mg, 0.19 mmol) and DIEA (74 mg, 0.58 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 21-1 (90 mg) as a brown solid, yield: 76.3%. LCMS: Rt=1.306 min, MS (ESI) m/z=619.0 [M+H]⁺.

### Step 2

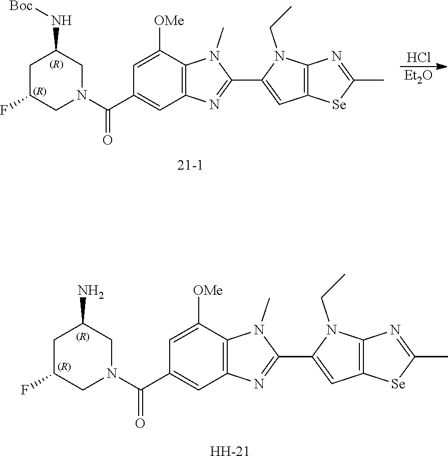

HH-21: To a solution of compound 21-1 (90 mg, 0.15 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-21 (51 mg, hydrochloric acid salt form) as a yellow solid. yield: 63.9%. ¹H NMR (400 MHz, DMSO-d₆) δ ppm 8.62-8.20 (m, 3H), 7.34 (d, J=8.0 Hz, 1H), 6.99-6.91 (m, 2H), 5.36-4.90 (m, 3H), 4.47 (d, J=7.2 Hz, 2H), 4.20-3.82 (m, 7H), 3.31-2.99 (m, 2H), 2.79 (s, 3H), 2.35-2.28 (m, 1H), 2.03-1.78 (m, 1H), 1.23 (td, J=7.0, 3.2 Hz, 3H). LCMS: Rt=1.083 min, MS (ESI) m/z=518.9 [M+H]⁺. HPLC: Rt=4.810 min, Purity: 100% (214 nm) and 100% (254 nm).

### Example 22. Synthesis of HH-22

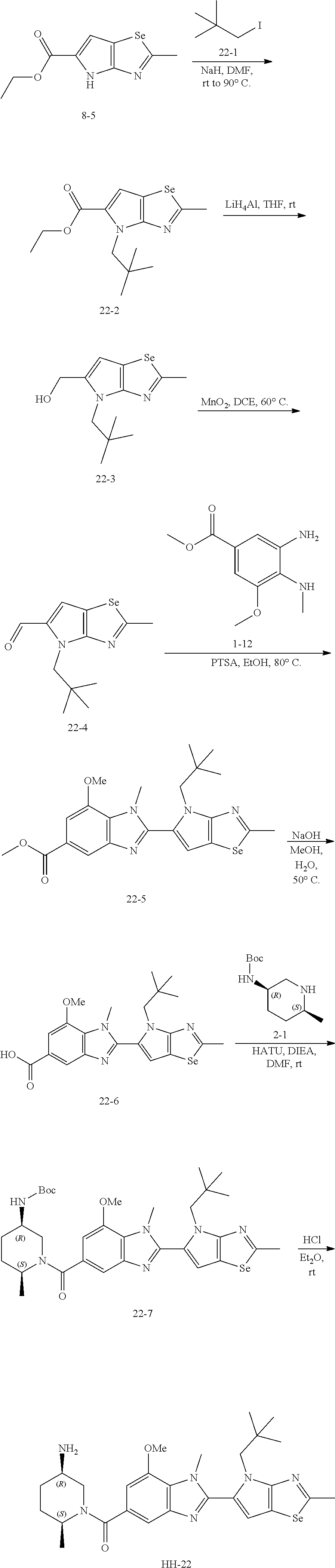

### Step 1

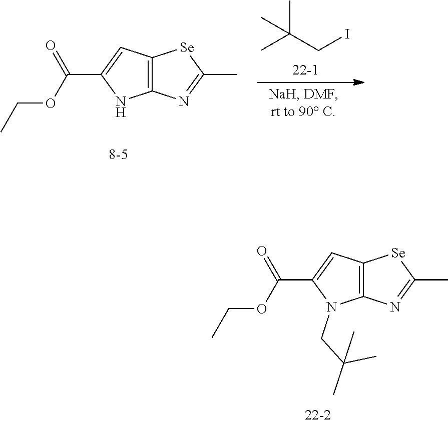

Compound 22-2: To a solution of compound 8-5 (320 mg, 1.24 mmol) in DMF (15 mL), was added NaH (60 mg, 1.48 mmol) at 0° C. The mixture was stirred at 0° C. for 30 mins. Then compound 22-1 (294 mg, 1.48 mmol) was added. The mixture was stirred at 90° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 22-2 (280 mg) as a yellow oil, yield: 69%. LCMS: Rt=1.582 min, MS (ESI) m/z=329.0 [M+H]⁺.

### Step 2

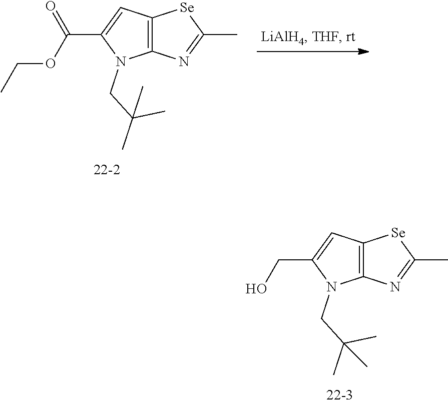 [SMILES: CCOC(=O)c1cc2[se]c(C)nc2n1CC(C)(C)C ; Cc1nc2c(cc(CO)n2CC(C)(C)C)[se]1]

Compound 22-3: To a solution of compound 22-2 (280 mg, 0.84 mmol) in THF (15 mL) was added LiAlH₄ (1M in THF, 2.5 mL, 2.5 mmol) at 0° C., The reaction mixture was stirred at 25° C. for 2 h. The mixture was quenched with water (20 mL) and filtered. The filtrate was extracted with ethyl acetate (20 mL×3). The organic layers were dried over anhydrous Na₂SO₄ and concentrated under vacuum to afford crude product (240 mg) as a yellow oil. LCMS: Rt=1.286 min, MS (ESI) m/z=287.0 [M+H]⁺.

### Step 3

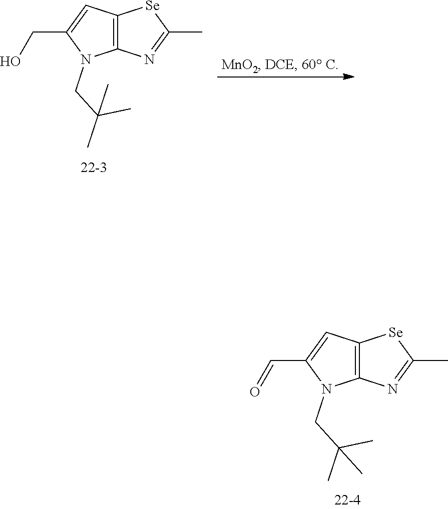

Compound 22-4: To a solution of compound 22-3 (240 mg, 0.84 mmol) in DCE (30 mL) was added MnO₂ (730 mg, 8.4 mmol). The reaction mixture was stirred at 60° C. for 16 h. The mixture was filtered and the filtrate was concentrated under vacuum to afford crude product 22-4 (200 mg) as a brown oil. LCMS: Rt=1.419 min, MS (ESI) m/z=285.0 [M+H]⁺.

### Step 4

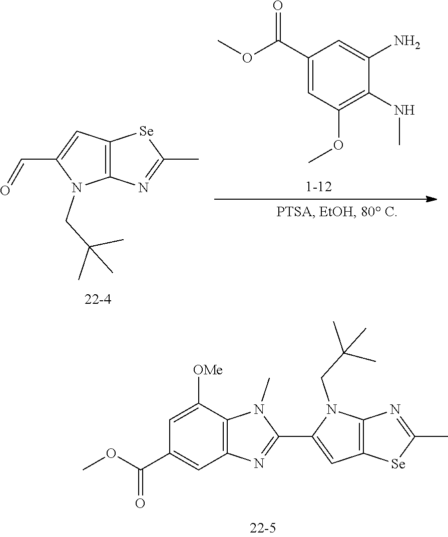

Compound 22-5: To a solution of compound 5 (200 mg, 0.72 mmol) in EtOH (30 mL) was added compound 1-12 (152 mg, 0.72 mmol) and PTSA (20 mg, 0.08 mmol). The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 22-5 (240 mg) as a yellow oil, yield: 71.4%. LCMS: Rt=1.457 min, MS (ESI) m/z=475.0 [M+H]⁺.

### Step 5

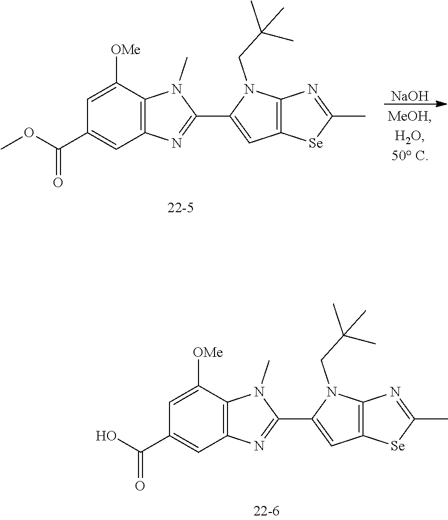

Compound 22-6: To a solution of compound 22-5 (240 mg, 0.42 mmol) in MeOH/H₂O (15/3 mL) was added NaOH (60 mg, 1.52 mmol). The reaction mixture was stirred at 50° C. for 16 h. The mixture was concentrated and adjusted pH to 5-6 with 1N hydrochloric acid aqueous solution, then extracted with ethyl acetate (20 mL×3). The organic phase was washed with water (10 mL), brine (10 mL), dried over anhydrous Na₂SO₄ and concentrated under vacuum to give compound 8 (160 mg) as a yellow solid, yield: 69.0%. LCMS: Rt=1.318 min, MS (ESI) m/z=461.1 [M+H]⁺.

### Step 6

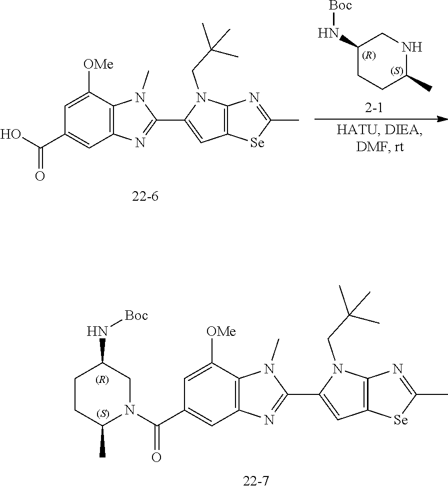

Compound 22-7: To a solution of compound 22-6 (80 mg, 0.174 mmol) in DMF (8 mL) was added compound 2-1 (46 mg, 0.20 mmol), HATU (66 mg, 0.174 mmol) and DIEA (68 mg, 0.52 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 22-7 (75 mg) as a brown solid, yield: 66%. LCMS: Rt=1.428 min, MS (ESI) m/z=657.2 [M+H]⁺.

### Step 7

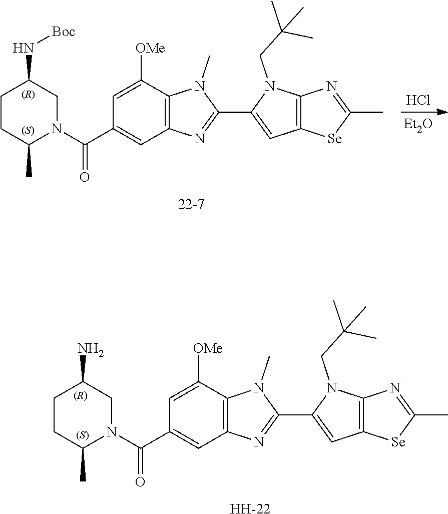

HH-22: To a solution of compound 22-7 (75 mg, 0.114 mmol) in Et₂O (10 mL) was added 2M HCl in Et₂O (18 mL, 36 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 20-40%) to afford HHBP-9527 (40.5 mg, hydrochloric acid salt form) as a yellow solid. yield: 64.5%. ¹H NMR (400 MHz, DMSO-d₆) δ ppm 8.14 (s, 3H), 7.32 (s, 1H), 6.99 (s, 1H), 6.87 (s, 1H), 4.60-4.53 (m, 3H), 4.13 (s, 3H), 3.99 (s, 3H), 3.25-3.10 (m, 3H), 2.80 (s, 3H), 1.90-1.59 (m, 4H), 1.21 (d, J=6.8 Hz, 3H), 0.59 (s, 9H). LCMS: Rt=1.140 min, MS (ESI) m/z=557.3 [M+H]⁺. HPLC: Rt=6.537 min, Purity: 100% (214 nm) and 99.30% (254 nm).

### Example 23. Synthesis of HH-23

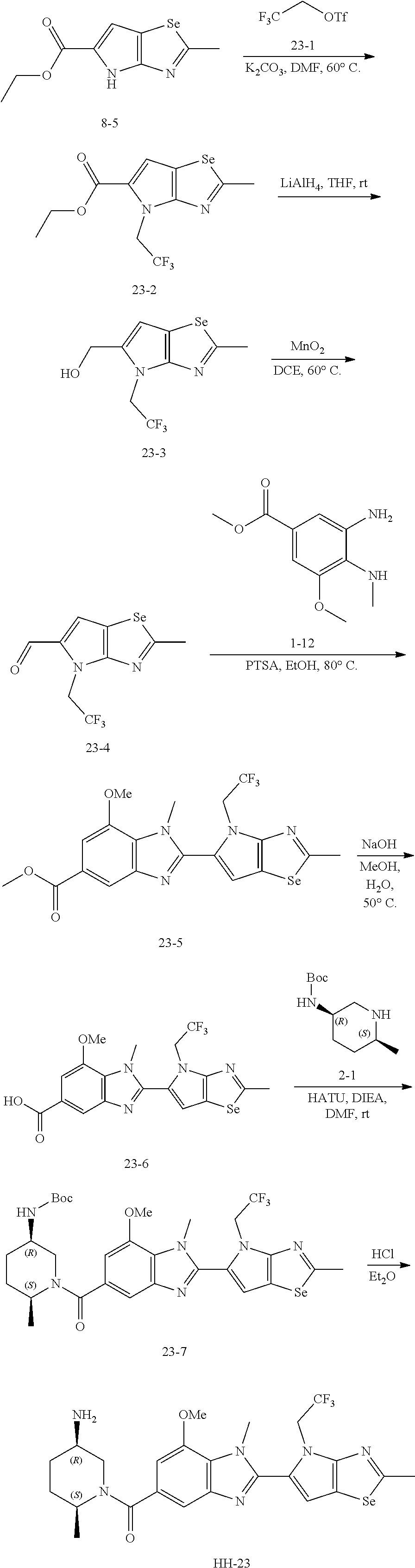

### Step 1

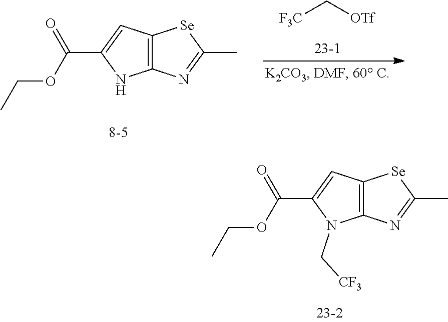 [SMILES: O=S(=O)(OCC(F)(F)F)C(F)(F)F ; CCOC(=O)c1cc2[se]c(C)nc2[nH]1 ; CCOC(=O)c1cc2[se]c(C)nc2n1CC(F)(F)F]

Compound 23-2: To a solution of compound 8-5 (180 mg, 0.70 mmol) and compound 23-1 (195 mg, 0.84 mmol) in DMF (10 mL) was added potassium carbonate (290 mg, 2.1 mmol). The mixture was stirred at 60° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 23-2 (220 mg) as a yellow oil, yield: 84.0%. LCMS: Rt=1.402 min, MS (ESI) m/z=340.9 [M+H]⁺.

### Step 2

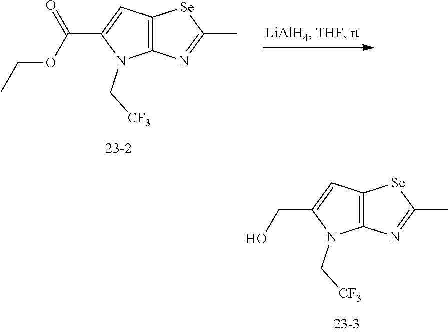 [SMILES: CCOC(=O)c1cc2[se]c(C)nc2n1CC(F)(F)F ; Cc1nc2c(cc(CO)n2CC(F)(F)F)[se]1]

Compound 23-3: To a solution of compound 23-2 (220 mg, 0.65 mmol) in THF (5 mL), then was added LiAlH₄ (1M in THF, 1.94 mL, 1.94 mmol) at 0° C. The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product (180 mg) as a yellow oil. LCMS: Rt=1.168 min, MS (ESI) m/z=298.9 [M+H]⁺.

### Step 3

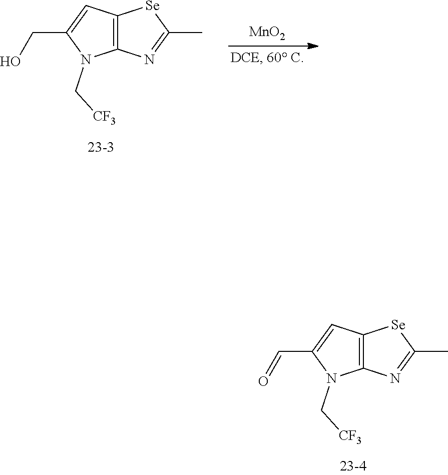

Compound 23-4: To a solution of compound 23-3 (180 mg, 0.60 mmol) in DCE (10 mL) was added MnO₂ (526 mg, 6.04 mmol). The reaction mixture was stirred at 60° C. for 16 h. The mixture was filtered and the filtrate was concentrated under vacuum to afford crude product (170 mg) as a brown oil. LCMS: Rt=1.249 min, MS (ESI) m/z=297.0 [M+H]⁺.

### Step 4

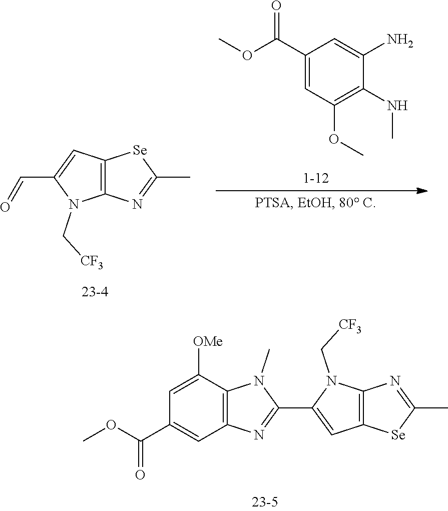

Compound 23-5: To a solution of compound 23-4 (170 mg, 0.57 mmol) in EtOH (10 mL) was added compound 1-12 (122 mg, 0.57 mmol) and PTSA (10 mg, 0.06 mmol). The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 7 (170 mg) as a yellow oil, yield: 60.5%. LCMS: Rt=1.399 min, MS (ESI) m/z=487.0 [M+H]⁺.

### Step 5

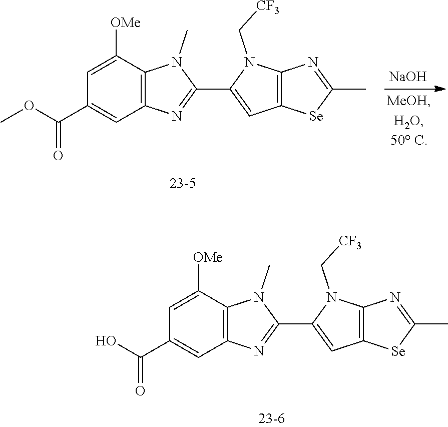

Compound 23-6: To a solution of compound 23-5 (170 mg, 0.35 mmol) in MeOH/H₂O (5/1 mL) was added NaOH (15 mg, 1.05 mmol). The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 23-6 (150 mg) as a yellow solid, yield: 90.9%. LCMS: Rt=1.289 min, MS (ESI) m/z=472.9 [M+H]⁺.

### Step 6

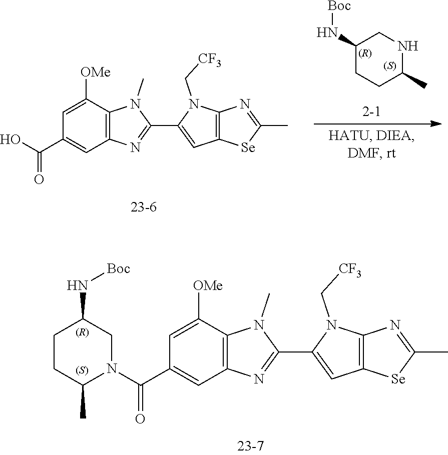

Compound 23-7: To a solution of compound 23-6 (150 mg, 0.32 mmol) in DMF (5 mL) was added compound 2-1 (68 mg, 0.32 mmol), HATU (122 mg, 0.32 mmol) and DIEA (124 mg, 0.96 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 23-7 (150 mg) as a brown solid, yield: 70.8%. LCMS: Rt=1.402 min, MS (ESI) m/z=669.0 [M+H]⁺.

### Step 7

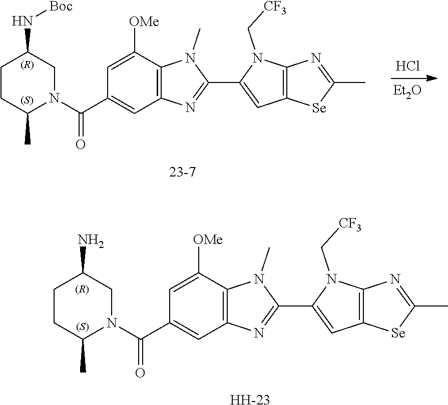

HH-23: To a solution of compound 23-7 (150 mg, 0.22 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 20-40%) to afford HH23-7 (66 mg, hydrochloric acid salt form) as a yellow solid. yield: 51.9%. ¹H NMR (400 MHz, DMSO-d₆) δ ppm 8.10 (s, 3H), 7.31 (d, J=1.0 Hz, 1H), 7.17 (s, 1H), 6.86 (s, 1H), 5.77-5.62 (m, 2H), 4.14-3.99 (m, 7H), 3.15-3.01 (m, 3H), 2.83 (s, 3H), 1.94-1.74 (m, 3H), 1.61-1.51 (m, 1H), 1.21 (d, J=6.8 Hz, 3H). LCMS: Rt=1.101 min, MS (ESI) m/z=569.1 [M+H]⁺. HPLC: Rt=5.125 min, Purity: 96.79% (214 nm) and 100% (254 nm).

### Example 24. Synthesis of HH-24

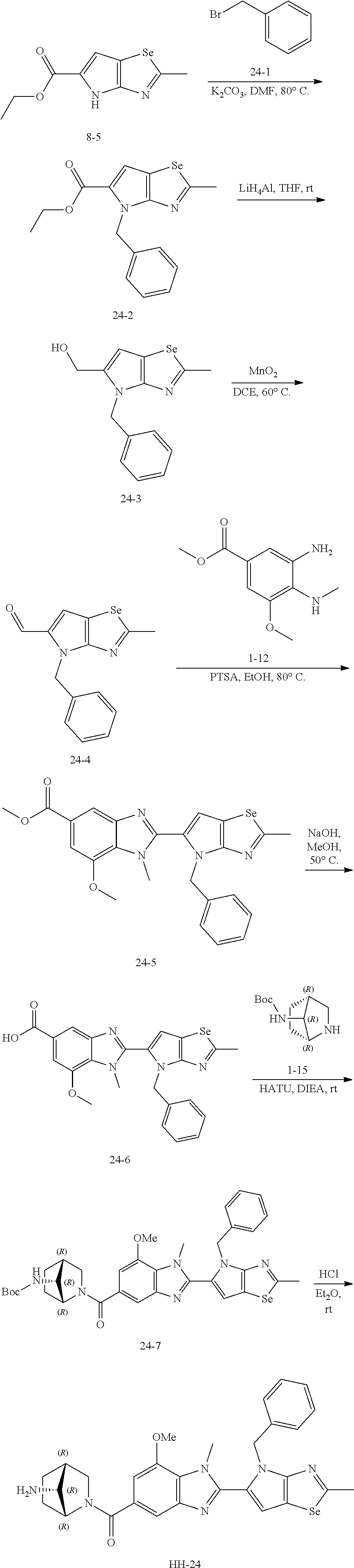

### Step 1

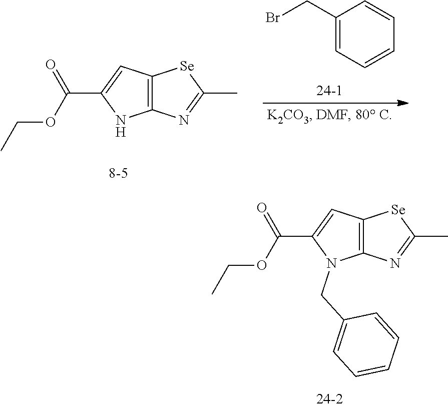 [SMILES: CCOC(=O)c1cc2[se]c(C)nc2[nH]1 ; BrCc1ccccc1 ; CCOC(=O)c1cc2[se]c(C)nc2n1Cc1ccccc1]

Compound 24-2: To a solution of compound 8-5 (350 mg, 1.36 mmol) in DMF (10 mL) was added compound 24-1 (349.2 mg, 2.04 mmol) and K₂CO₃ (376.2 mg, 2.72 mmol). The mixture was stirred at 80° C. for 3 h. The mixture was diluted with ethyl acetate (50 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 3 (400 mg) as a yellow solid, yield: 84.6%. LCMS: Rt=1.472 min, MS (ESI) m/z=349.1[M+H]⁺.

### Step 2

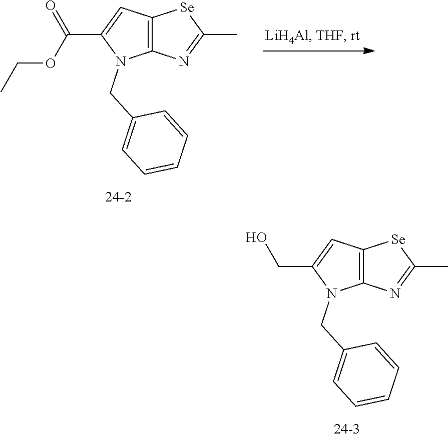 [SMILES: CCOC(=O)c1cc2[se]c(C)nc2n1Cc1ccccc1 ; Cc1nc2c(cc(CO)n2Cc2ccccc2)[se]1]

Compound 24-3: To a solution of compound 24-2 (400 mg, 1.15 mmol) in THE (10 mL), then was added LiAlH₄ (1M in THF, 2.3 mL, 2.30 mmol) at 0° C. The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product 24-3 (330 mg) as a yellow oil. LCMS: Rt=1.229 min, MS (ESI) m/z=307.0 [M+H]⁺.

### Step 3

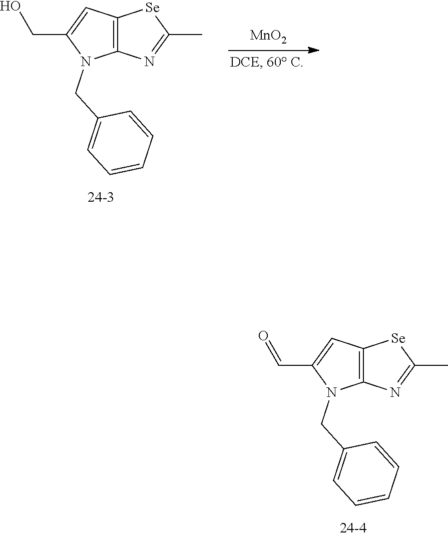

Compound 24-4: To a solution of compound 24-3 (330 mg, 1.08 mmol) in DCE (20 mL) was added MnO₂ (939.8 mg, 10.8 mmol). The reaction mixture was stirred at 60° C. for 16 h. The mixture was filtered and the filtrate was concentrated under vacuum to afford crude product (200 mg) as a brown oil. LCMS: Rt=1.347 min, MS (ESI) m/z=304.9 [M+H]⁺.

### Step 4

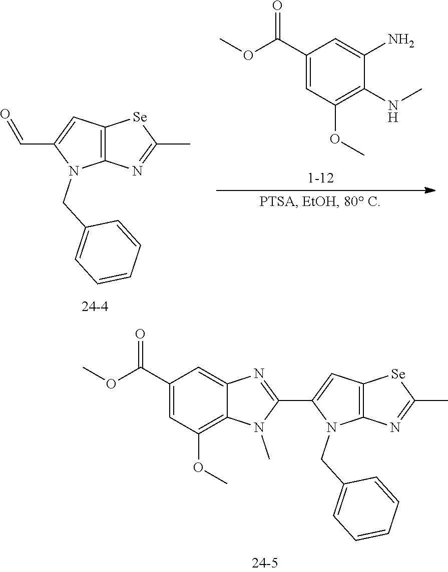

Compound 24-5: To a solution of compound 24-4 (200 mg, 0.66 mmol) in EtOH (10 mL) was added compound 1-12 (138.7 mg, 0.66 mmol) and PTSA (11.4 mg, 0.06 mmol), The reaction mixture was stirred at 80° C. for 12 h. The mixture was concentrated under vacuum to give the crude product, which was purified by silica gel chromatography (elution gradient: petroleum ether/EtOAc, 5/1, v/v) to afford compound 24-5 (280 mg) as a yellow oil, yield: 86.0%. LCMS: Rt=1.439 min, MS (ESI) m/z=494.9 [M+H]⁺.

### Step 5

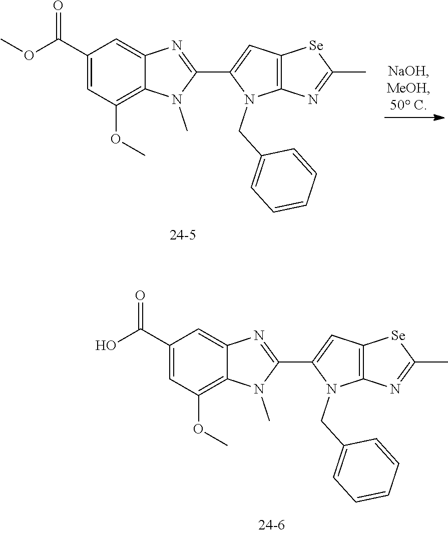

Compound 24-6: To a solution of compound 24-5 (280 mg, 0.57 mmol) in MeOH (10 mL) was added sodium hydroxide (2 N, 0.6 mL). The mixture was stirred at 50° C. for 3 h. The mixture was concentrated and adjusted pH to 5-6 with hydrochloric acid aqueous solution (2N), then extracted with ethyl acetate (50 mL×3). The organic phase was washed with water (50 mL), brine (50 mL), and concentrated under vacuum to give compound 24-6 (200 mg) as a yellow solid, yield: 73.5%. LCMS: Rt=1.310 min, MS (ESI) m/z=480.9 [M+H]⁺.

### Step 6

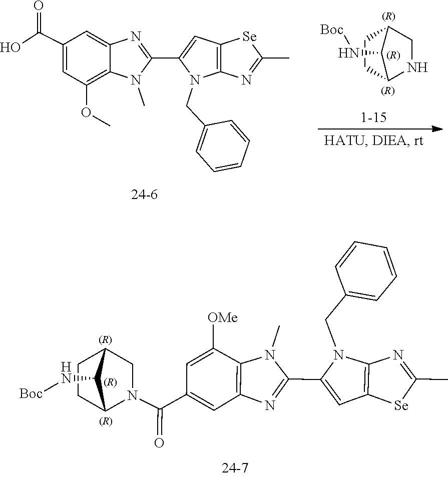

Compound 24-7: To a solution of compound 24-6 (100 mg, 0.21 mmol) in DMF (10 mL) was added compound 1-15 (49.2 mg, 0.23 mmol), HATU (95.2 mg, 0.25 mmol) and DIEA (80.9 mg, 0.62 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/10, v/v) to afford compound 24-7 (100 mg) as a brown solid, yield: 70.9%. LCMS: Rt=1.401 min, MS (ESI) m/z=677.2 [M+H]⁺.

### Step 7

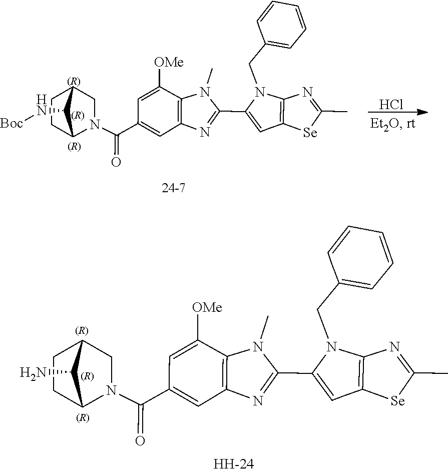

HH-24: To a solution of compound 24-7 (140 mg, 0.20 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-24 (11.0 mg, hydrochloric acid salt form) as a yellow solid. yield: 33.3%. ¹H NMR (400 MHz, DMSO-d₆) δ ppm 8.21 (s, 3H), 7.31 (s, 1H), 7.21-7.11 (m, 3H), 7.09 (s, 1H), 6.92-6.83 (m, 3H), 5.84 (s, 2H), 3.99 (s, 3H), 3.97 (s, 3H), 3.26-2.95 (m, 3H), 2.80 (s, 3H), 1.95-1.75 (m, 3H), 1.59-1.51 (m, 1H), 1.20 (d, J=7.2 Hz, 3H). LCMS: Rt=1.145 min, MS (ESI) m/z=577.1 [M+H]⁺. HPLC: Rt=5.553 min, Purity: 100% (214 nm) and 100% (254 nm).

### Example 25. Synthesis of HH-25

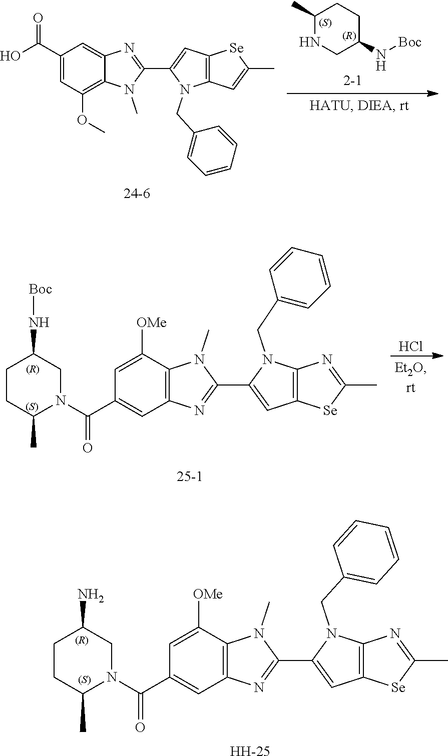

### Step 1

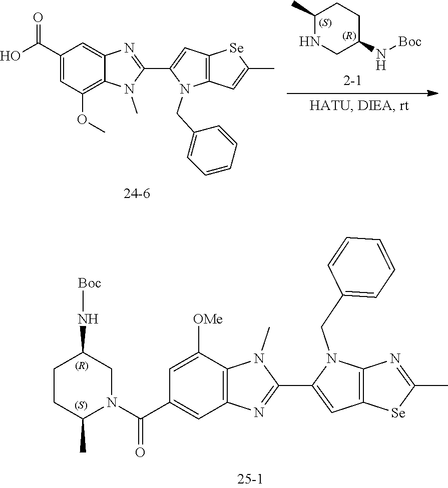

Compound 25-1: To a solution of compound 24-6 (100 mg, 0.21 mmol) in DMF (10 mL) was added compound 2-1 (48.1 mg, 0.23 mmol), HATU (95.2 mg, 0.25 mmol) and DIEA (80.2 mg, 0.62 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/10, v/v) to afford compound 25-1 (100 mg) as a brown solid, yield: 71.2%. LCMS: Rt=1.386 min, MS (ESI) m/z=675.0 [M+H]⁺.

### Step 2

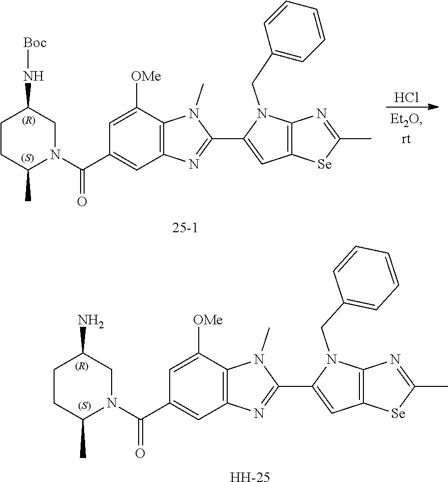

HH-25: To a solution of compound 25-1 (100 mg, 0.15 mmol) in Et₂O (10 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HHBP-9518 (60 mg, hydrochloric acid salt form) as a yellow solid. yield: 71.4%. ¹H NMR (400 MHz, DMSO-d₆) δ ppm 8.31 (s, 3H), 7.40 (s, 1H), 7.16-7.10 (m, 3H), 7.00 (s, 1H), 6.95-6.89 (m, 3H), 5.81 (s, 2H), 3.97 (s, 3H), 3.93 (s, 3H), 3.63-3.50 (m, 1H), 3.16 (d, J=10.4 Hz, 1H), 2.80 (s, 3H), 2.02-1.87 (m, 3H), 1.65-1.60 (m, 1H). LCMS: Rt=1.115 min, MS (ESI) m/z=575.1 [M+H]⁺. HPLC: Rt=5.366 min, Purity: 100% (214 nm) and 100% (254 nm).

### Example 26. Synthesis of HH-26

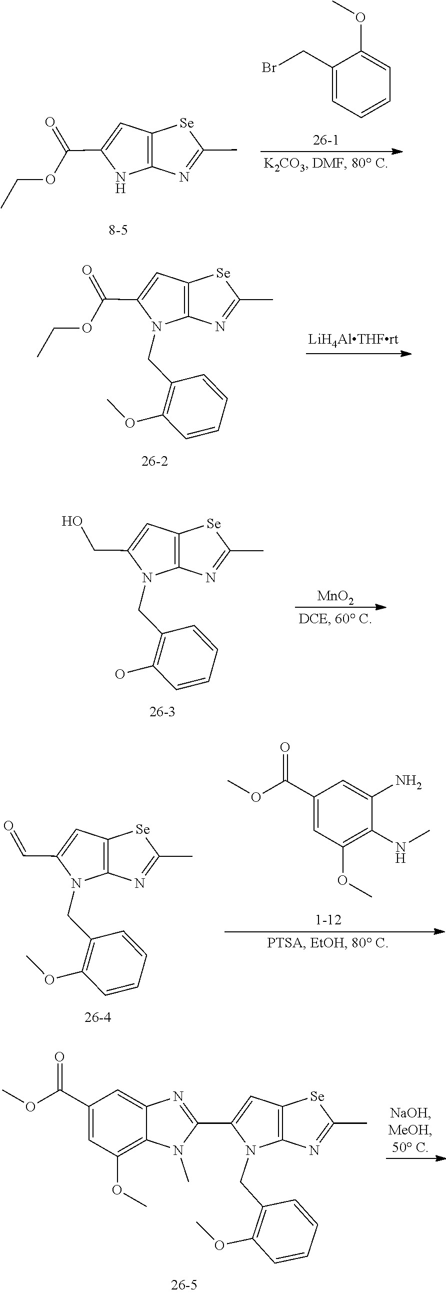

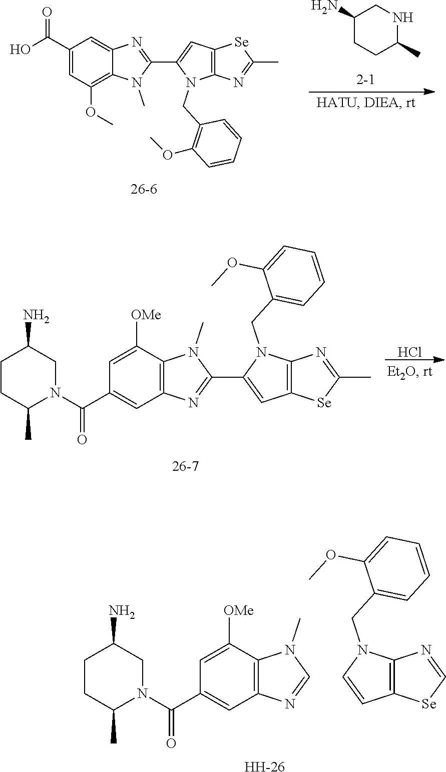

### Step 1

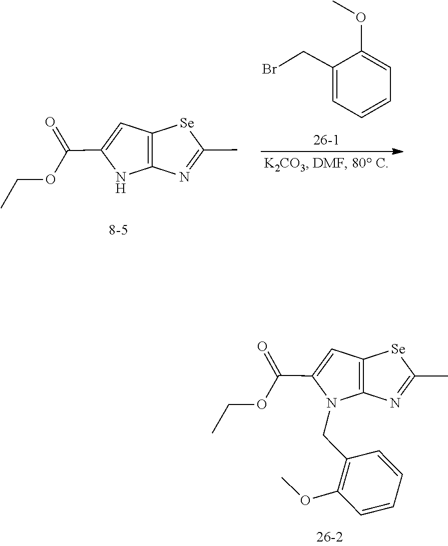

Compound 26-2: To a solution of compound 8-5 (180 mg, 0.70 mmol) in DMF (5 mL) was added compound 26-1 (211 mg, 1.06 mmol) and K₂CO₃ (194 mg, 1.41 mmol). The mixture was stirred at 50° C. for 3 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 26-2 (250 mg) as a yellow solid, yield: 94.8%. LCMS: Rt=1.44 min, MS (ESI) m/z=379.0 [M+H]⁺.

### Step 2

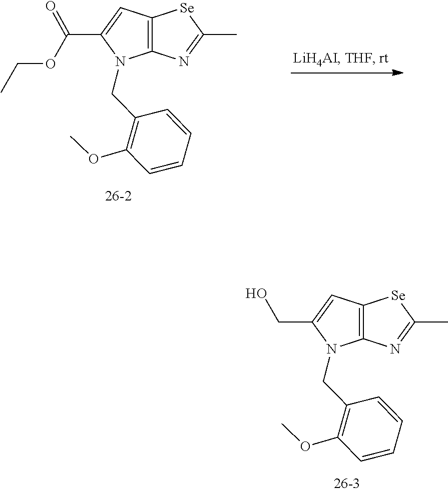 [SMILES: CCOC(=O)c1cc2[se]c(C)nc2n1Cc1ccccc1OC ; COc1ccccc1Cn1c(CO)cc2[se]c(C)nc21]

Compound 26-3: To a solution of compound 26-2 (250 mg, 0.66 mmol) in THE (10 mL) was added LiAlH₄ (1M in THF, 2 mL, 2 mmol). The mixture was stirred at room temperature for 2 h. The mixture was quenched with 2N sodium hydroxide aqueous solution (2 mL) and diluted with H₂O (10 mL), filtered and extracted with ethyl acetate (30 mL×2). The organic layers were combined, dried over anhydrous sodium sulfate, filtered and concentrated under vacuum to afford compound 26-3 (180 mg) as a yellow oil, yield: 81.1%. LCMS: Rt=1.26 min, MS (ESI) m/z=337.1 [M+H]⁺.

### Step 3

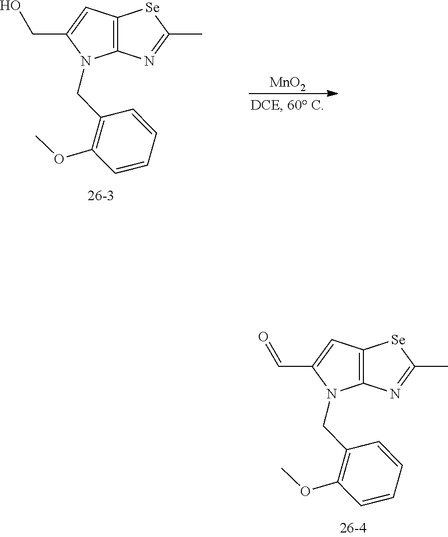 [SMILES: COc1ccccc1Cn1c(CO)cc2[se]c(C)nc21 ; "MnO2" ; COc1ccccc1Cn1c(C=O)cc2[se]c(C)nc21]

Compound 26-4: To a solution of compound 26-3 (180 mg, 0.54 mmol) in DCE (10 mL) was added MnO₂ (467 mg, 5.38 mmol). The mixture was stirred at 60° C. for 16 h. The mixture was filtered and concentrated under reduced pressure to afford crude product, which was purified by flash chromatography (elution gradient: petroleum ether/ethyl acetate, 5/1, v/v) to afford compound 26-4 (130 mg) as a yellow solid, yield: 72.6%. LCMS: Rt=1.34 min, MS (ESI) m/z=335.0 [M+H]⁺.

### Step 4

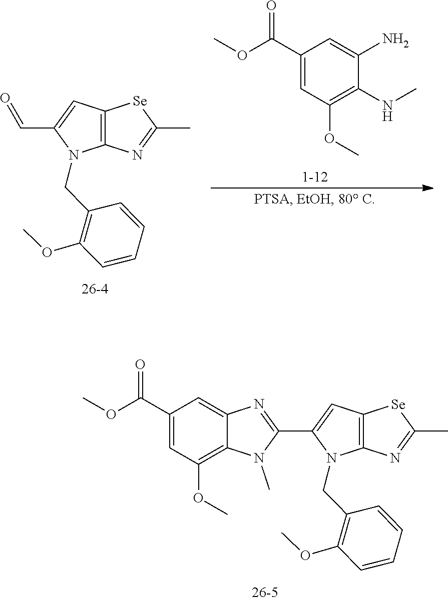

Compound 26-5: To a solution of compound 26-4 (130 mg, 0.39 mmol) in EtOH (5 mL) was added compound 1-12 (90 mg, 0.43 mmol) and PTSA (7 mg, 0.041 mmol). The mixture was stirred at 80° C. for 4 h. The mixture was concentrated under reduced pressure to afford compound 26-5 (180 mg, crude) as a yellow solid. The crude product was used into next step without further purification. LCMS: Rt=1.41 min, MS (ESI) m/z=525.1 [M+H]⁺.

### Step 5

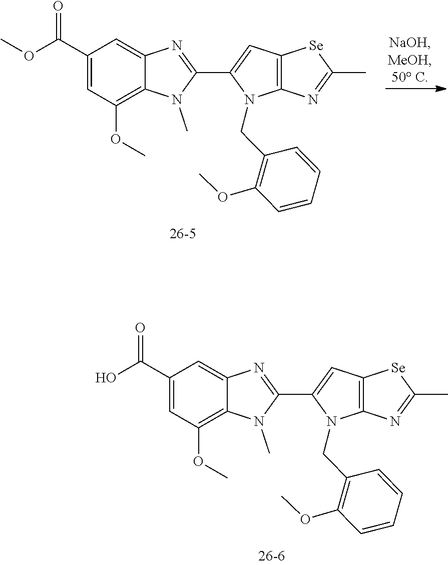

Compound 26-6: To a solution of compound 26-5 (180 mg, crude) in MeOH (5 mL) was added 2N NaOH (0.5 mL, 1 mmol). The mixture was stirred at 50° C. for 5 h. The mixture was concentrated and adjusted pH to 5-6 with 1N hydrochloric acid aqueous solution and diluted with H₂O (10 mL), then extracted with dichloromethane (20 mL×3). The organic phases were combined, dried over Na₂SO₄, filtered and concentrated under vacuum to give compound 26-6 (160 mg) as a yellow oil, yield: 80.4%. LCMS: Rt=1.28 min, MS (ESI) m/z=511.1 [M+H]⁺.

### Step 6

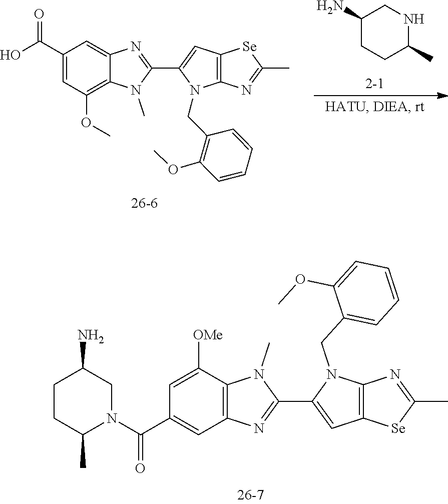

Compound 26-7: To a solution of compound 26-6 (160 mg, 0.31 mmol), DIEA (122 mg, 0.94 mol), HATU (155 mg, 0.41 mmol) in DMF (5 mL) was added compound 2-1 (75 mg, 0.35 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 26-7 (170 mg) as a yellow solid, yield: 76.37%. LCMS: Rt=1.38 min, MS (ESI) m/z=707.2 [M+H]⁺.

### Step 7

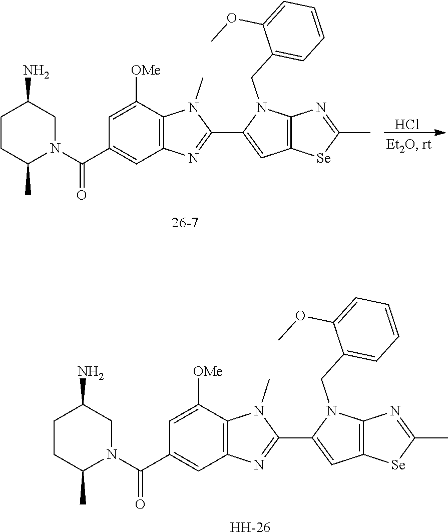

HH-26: To a solution of compound 26-7 (170 mg, 0.24 mmol) in ethyl ether (5 mL) was added hydrochloric acid solution (5 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-40%) to afford HH-26 (99.9 mg, hydrochloric acid salt form) as a yellow solid. yield: 64.6%. ¹H NMR (400 MHz, DMSO-d₆): δ ppm 8.34 (s, 3H), 7.35 (s, 1H), 7.16-7.12 (m, 2H), 6.99 (s, 1H), 6.84 (d, J=8.0 Hz, 1H), 6.70 (d, J=7.4 Hz, 1H), 6.57-6.55 (m, 1H), 5.71 (s, 2H), 4.00 (d, J=4.8 Hz, 6H), 3.47 (s, 3H), 3.16-3.09 (m, 2H), 2.81 (s, 3H), 1.90-1.73 (m, 3H), 1.57-1.55 (m, 1H), 1.21 (d, J=6.8 Hz, 3H). LCMS: Rt=1.11 min, MS (ESI) m/z=607.1 [M+H]⁺. HPLC: Rt=5.00 min, Purity: 100% (214 nm and 254 nm).

### Example 27. Synthesis of HH-27

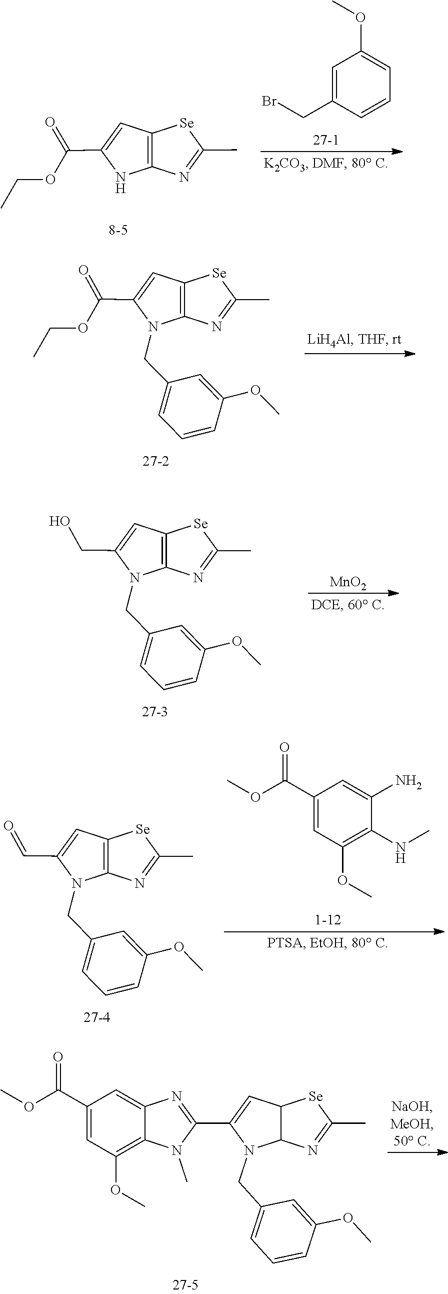

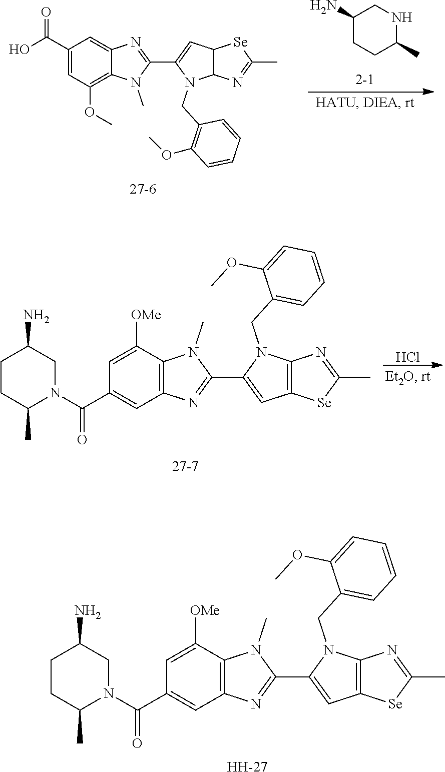

### Step 1

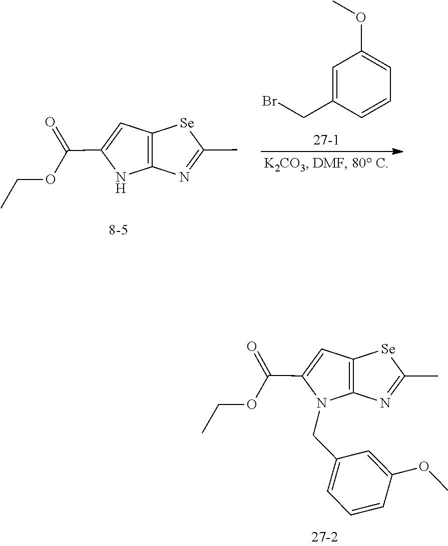

Compound 27-2: To a solution of compound 8-5 (250 mg, 0.97 mmol) in DMF (10 mL) was added compound 27-1 (293.2 mg, 1.46 mmol) and K₂CO₃ (268.7 mg, 1.94 mmol). The mixture was stirred at 80° C. for 3 h. The mixture was diluted with ethyl acetate (50 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 27-2 (300 mg) as a yellow solid, yield: 81.8%. LCMS: Rt=1.464 min, MS (ESI) m/z=378.9[M+H]⁺.

### Step 2

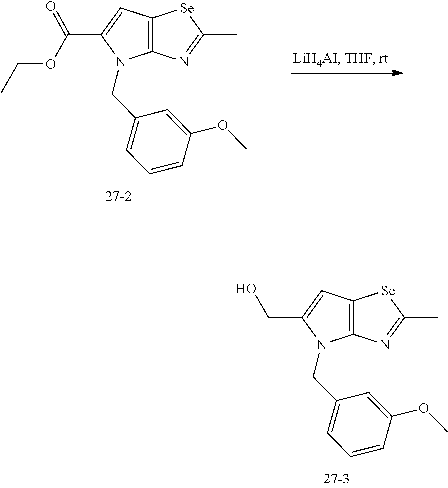 [SMILES: CCOC(=O)c1cc2[se]c(C)nc2n1Cc1cccc(OC)c1 ; COc1cccc(Cn2c(CO)cc3[se]c(C)nc32)c1]

Compound 27-3: To a solution of compound 27-2 (300 mg, 0.79 mmol) in THE (10 mL), then was added LiAlH₄ (1M in THF, 1.6 mL, 1.59 mmol) at 0° C. The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product 27-3 (200 mg) as a yellow oil. LCMS: Rt=1.228 min, MS (ESI) m/z=337.1 [M+H]⁺.

### Step 3

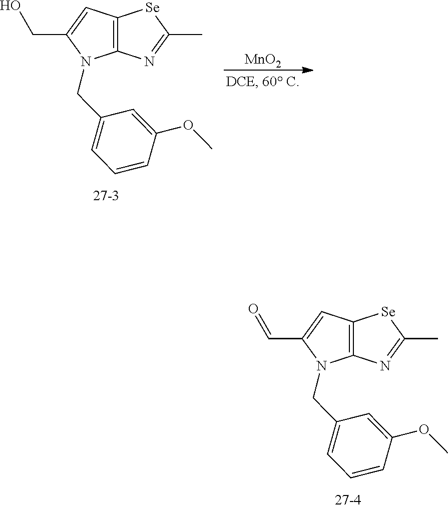

Compound 27-4: To a solution of compound 27-3 (200 mg, 0.59 mmol) in DCE (20 mL) was added MnO₂ (518.6 mg, 5.97 mmol). The reaction mixture was stirred at 60° C. for 16 h. The mixture was filtered and the filtrate was concentrated under vacuum to afford crude product (150 mg) as a brown oil. LCMS: Rt=1.338 min, MS (ESI) m/z=334.9 [M+H]⁺.

### Step 4

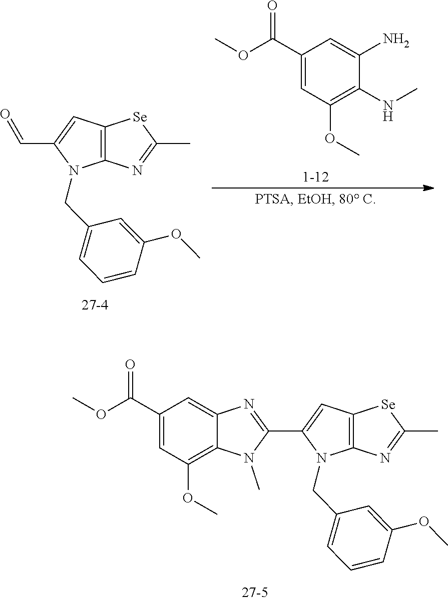

Compound 27-5: To a solution of compound 27-4 (150 mg, 0.45 mmol) in EtOH (10 mL) was added compound 1-12 (94.6 mg, 0.45 mmol) and PTSA (7.75 mg, 0.04 mmol). The reaction mixture was stirred at 80° C. for 12 h. The mixture was concentrated under vacuum to give the crude product, which was purified by silica gel chromatography (elution gradient: petroleum ether/EtOAc, 5/1, v/v) to afford compound 27-5 (180 mg) as a yellow oil, yield: 76.4%. LCMS: Rt=1.414 min, MS (ESI) m/z=525.0 [M+H]⁺.

### Step 5

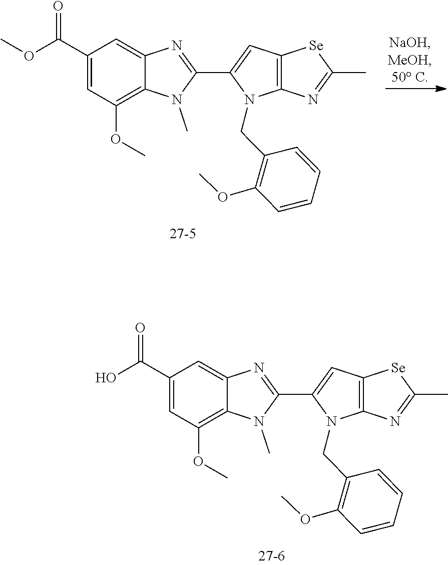

Compound 27-6: To a solution of compound 27-5 (180 mg, 0.34 mmol) in MeOH (10 mL) was added sodium hydroxide (2 N, 0.4 mL). The mixture was stirred at 50° C. for 3 h. The mixture was concentrated and adjusted pH to 5-6 with hydrochloric acid aqueous solution (2N), then extracted with ethyl acetate (50 mL×3). The organic phase was washed with water (50 mL), brine (50 mL), and concentrated under vacuum to give compound 27-6 (150 mg) as a yellow solid, yield: 85.6%. LCMS: Rt=1.307 min, MS (ESI) m/z=510.9 [M+H]⁺.

### Step 6

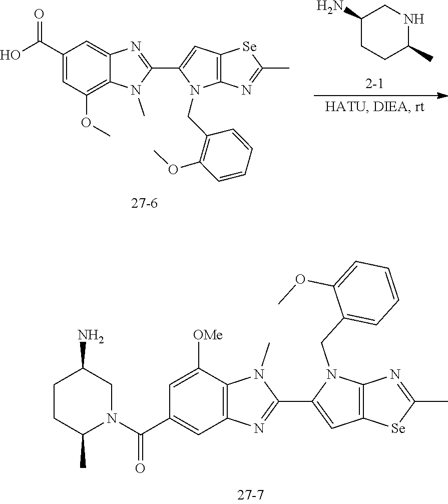

Compound 27-7: To a solution of compound 27-6 (150 mg, 0.29 mmol) in DMF (5 mL) was added compound 2-1 (69.4 mg, 0.32 mmol), HATU (134.3 mg, 0.35 mmol) and DIEA (114.2 mg, 0.88 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/99, v/v) to afford compound 27-7 (140 mg) as a brown solid, yield: 67.4%. LCMS: Rt=1.396 min, MS (ESI) m/z=707.0 [M+H]⁺.

### Step 7

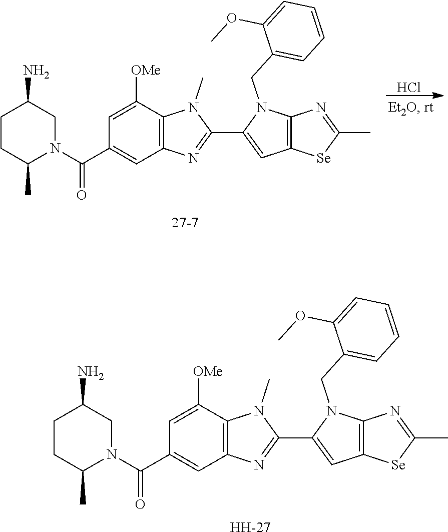

HH-27: To a solution of compound 27-7 (140 mg, 0.20 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-27 (11.0 mg, hydrochloric acid salt form) as a yellow solid. yield: 33.3%. ¹H NMR (400 MHz, DMSO-d₆) δ ppm 8.25 (s, 3H), 7.29 (s, 1H), 7.09-7.02 (m, 2H), 6.88 (s, 1H), 6.69-6.62 (m, 1H), 6.44-6.39 (m, 2H), 5.75 (s, 2H), 3.94 (s, 3H), 3.92 (s, 3H), 3.48 (s, 3H), 3.17-2.98 (m, 2H), 2.77 (s, 3H), 1.90-1.65 (m, 3H), 1.54 (s, 1H), 1.16 (d, J=6.8 Hz, 3H). LCMS: Rt=1.133 min, MS (ESI) m/z=607.1 [M+H]⁺. HPLC: Rt=4.863 min, Purity: 99.7% (214 nm) and 99.7% (254 nm).

### Example 28. Synthesis of HH-28

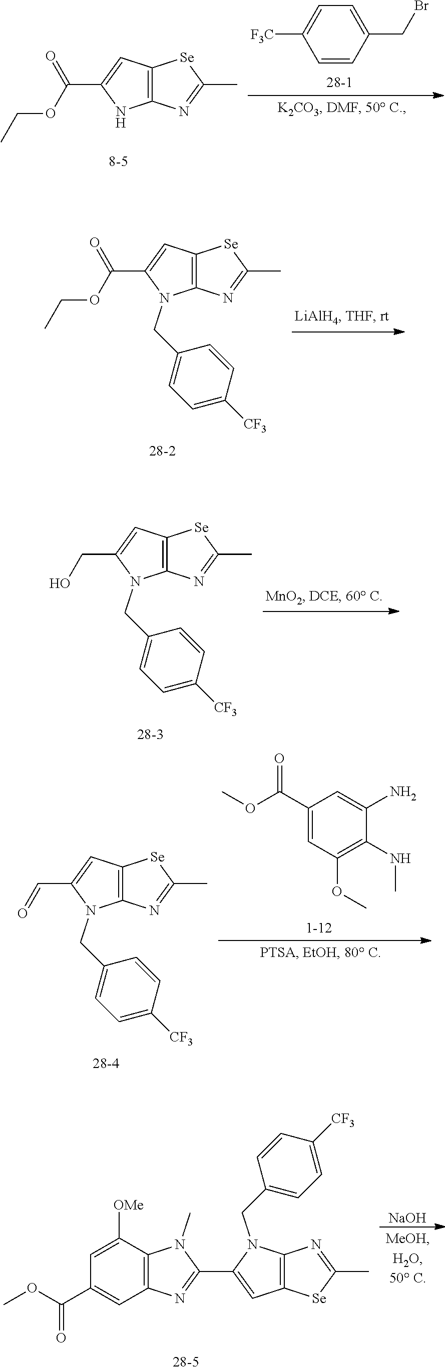

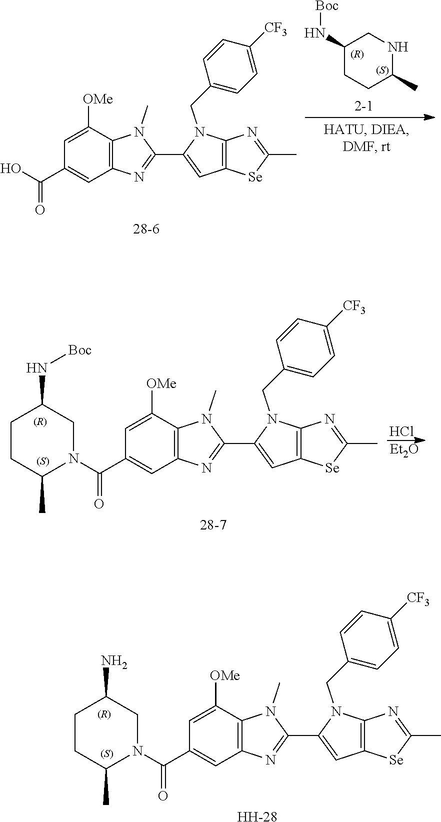

### Step 1

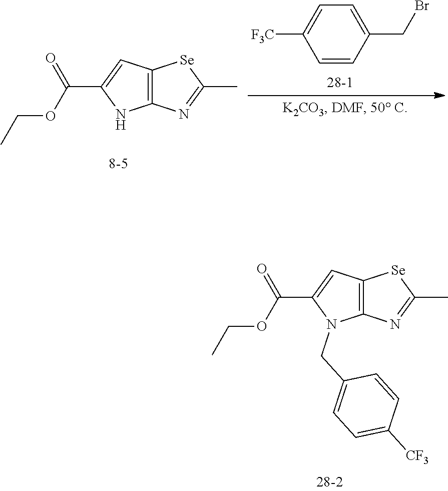 [SMILES: CCOC(=O)c1cc2[se]c(C)nc2[nH]1 ; FC(F)(F)c1ccc(CBr)cc1 ; CCOC(=O)c1cc2[se]c(C)nc2n1Cc1ccc(C(F)(F)F)cc1]

Compound 28-2: To a solution of compound 8-5 (180 mg, 0.70 mmol) in DMF (5 mL) was added compound 28-1 (250 mg, 1.05 mmol) and K₂CO₃ (194 mg, 1.41 mmol). The mixture was stirred at 50° C. for 3 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 28-2 (270 mg) as a yellow solid, yield: 93.1%. LCMS: Rt=1.53 min, MS (ESI) m/z=417.0 [M+H]⁺.

### Step 2

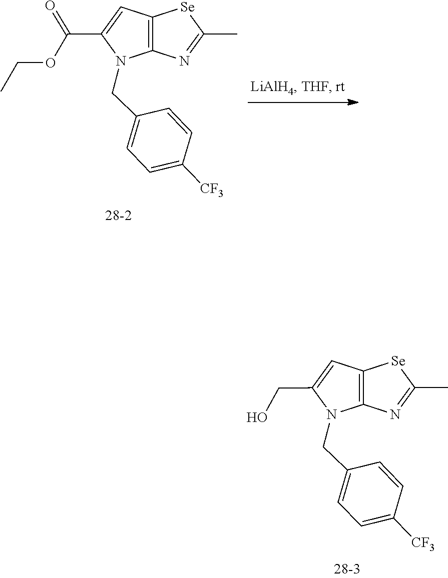 [SMILES: CCOC(=O)c1cc2[se]c(C)nc2n1Cc1ccc(C(F)(F)F)cc1 ; Cc1nc2c(cc(CO)n2Cc2ccc(C(F)(F)F)cc2)[se]1]

Compound 28-3: To a solution of compound 28-2 (270 mg, 0.65 mmol) in THF (10 mL) was added LiAlH₄ (1M in THF, 2 mL, 2 mmol). The mixture was stirred at room temperature for 2 h. The mixture was quenched with 2N sodium hydroxide aqueous solution (2 mL) and diluted with H₂O (10 mL), filtered and extracted with ethyl acetate (30 mL×2). The organic layers were combined, dried over anhydrous sodium sulfate, filtered and concentrated under vacuum to afford compound 28-3 (180 mg) as a yellow oil, yield: 74.2%. LCMS: Rt=1.32 min, MS (ESI) m/z=375.0 [M+H]⁺.

### Step 3

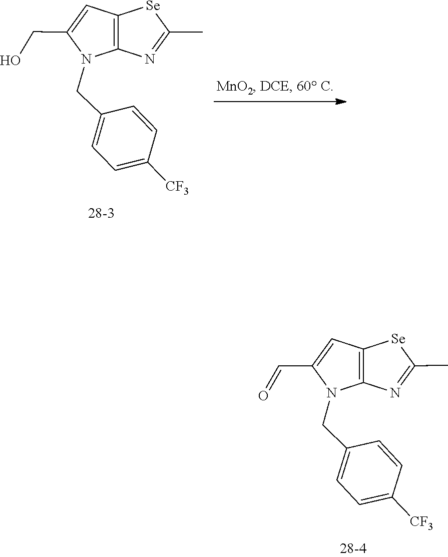 [SMILES: Cc1nc2c(cc(CO)n2Cc2ccc(C(F)(F)F)cc2)[se]1 ; Cc1nc2c(cc(C=O)n2Cc2ccc(C(F)(F)F)cc2)[se]1]

Compound 28-4: To a solution of compound 28-3 (180 mg, 0.48 mmol) in DCE (5 mL) was added MnO₂ (420 mg, 4.83 mmol). The mixture was stirred at 60° C. for 16 h. The mixture was filtered and concentrated under reduced pressure to afford crude product, which was purified by flash chromatography (elution gradient: petroleum ether/ethyl acetate, 5/1, v/v) to afford compound 28-4 (140 mg) as a yellow solid, yield: 78.2%. LCMS: Rt=1.39 min, MS (ESI) m/z=373.0 [M+H]⁺.

### Step 4

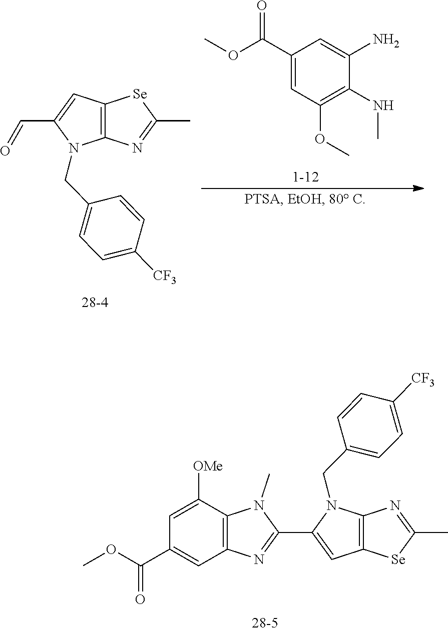

Compound 28-5: To a solution of compound 28-4 (140 mg, 0.38 mmol) in EtOH (5 mL) was added compound 1-12 (88 mg, 0.42 mmol) and PTSA (6.5 mg, 0.038 mmol). The mixture was stirred at 80° C. for 4 h. The mixture was concentrated under reduced pressure to compound 28-5 (180 mg, crude) as a yellow solid. The crude product was used into next step without further purification. LCMS: Rt=1.51 min, MS (ESI) m/z=562.9 [M+H]⁺.

### Step 5

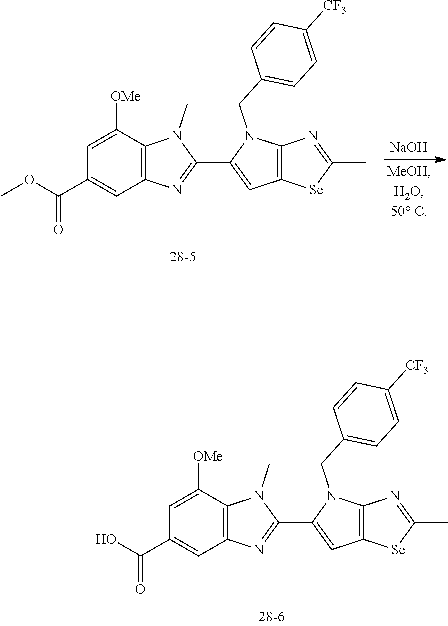

Compound 28-6: To a solution of compound 28-5 (180 mg, crude) in MeOH (5 mL) was added 2N NaOH (0.5 mL, 1 mmol). The mixture was stirred at 50° C. for 5 h. The mixture was concentrated and adjusted pH to 5-6 with 1N hydrochloric acid aqueous solution and diluted with H₂O (10 mL), then extracted with dichloromethane (20 mL×3). The organic phases were combined, dried over Na₂SO₄, filtered and concentrated under vacuum to give compound 28-6 (160 mg) as a yellow oil, yield: 77.7%. LCMS: Rt=1.38 min, MS (ESI) m/z=548.9 [M+H]⁺.

### Step 6

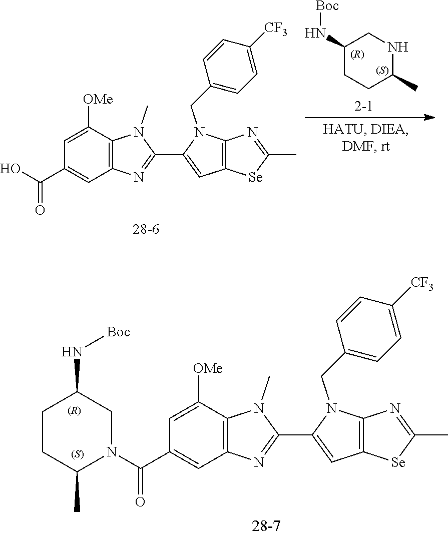

Compound 28-7: To a solution of compound 28-6 (160 mg, 0.29 mmol), DIEA (114 mg, 0.88 mol), HATU (145 mg, 0.38 mmol) in DMF (5 mL) was added compound 2-1 (70 mg, 0.33 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 28-7 (140 mg) as a yellow solid, yield: 64.5%. LCMS: Rt=1.47 min, MS (ESI) m/z=745.2 [M+H]⁺.

### Step 7

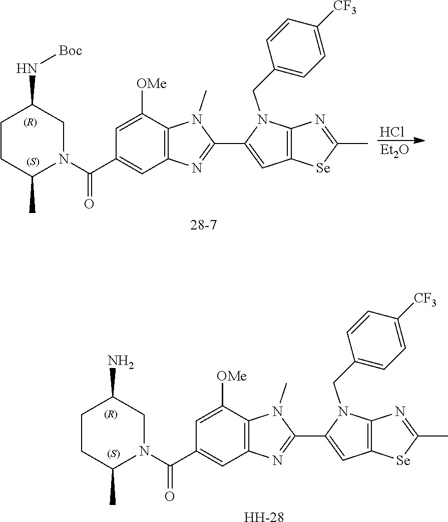

HH-28: To a solution of compound 28-7 (140 mg, 0.19 mmol) in ethyl ether (5 mL) was added hydrochloric acid solution (5 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 28-35%) to afford HH-28 (47.5 mg, hydrochloric acid salt form) as a yellow solid. yield: 37.1%. ¹H NMR (400 MHz, DMSO-d₆): δ ppm 8.30 (s, 3H), 7.57 (d, J=8.4 Hz, 2H), 7.29 (s, 1H), 7.17-7.16 (m, 3H), 6.90 (s, 1H), 5.94 (s, 2H), 4.05 (s, 3H), 4.01-3.98 (m, 1H), 3.97 (s, 3H), 3.15-3.07 (m, 2H), 2.79 (s, 3H), 1.89-1.72 (m, 3H), 1.57-1.55 (m, 1H), 1.20 (d, J=6.8 Hz, 3H). LCMS: Rt=1.16 min, MS (ESI) m/z=645.1 [M+H]⁺. HPLC: Rt=5.79 min, Purity: 99.60% (214 nm) 99.62% and (254 nm).

### Example 29. Synthesis of HH-29

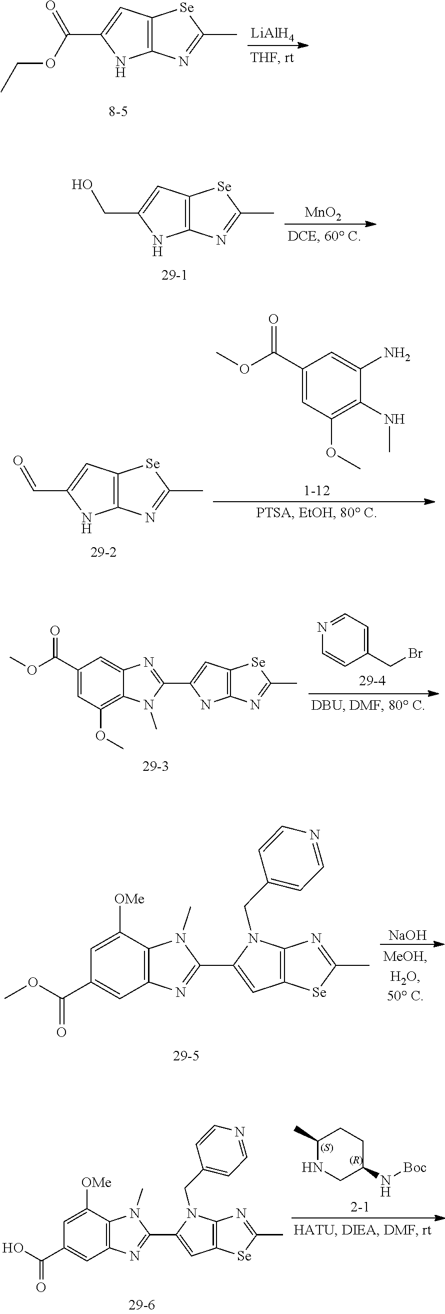

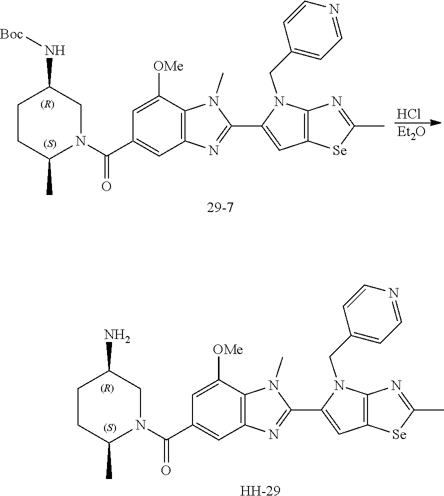

### Step 1

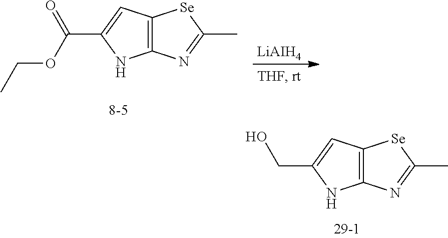

Compound 29-1: To a solution of compound 8-5 (0.9 g, 3.50 mmol) in THF (5 mL), then was added LiAlH₄ (1M in THF, 10.5 mL, 10.50 mmol) at 0° C., The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product (0.5 g) as a yellow oil. LCMS: Rt=0.993 min, MS (ESI) m/z=216.9 [M+H]⁺.

### Step 2

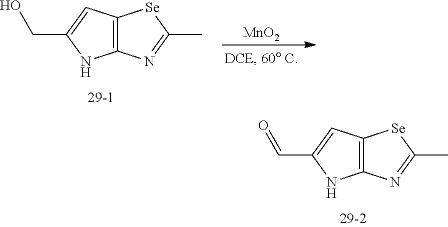

Compound 29-2: To a solution of compound 29-1 (500 mg, 2.32 mmol) in DCE (10 mL) was added MnO₂ (2.02 g, 23.24 mmol). The reaction mixture was stirred at 60° C. for 16 h. The mixture was filtered and the filtrate was concentrated under vacuum to afford crude product (380 mg) as a brown oil. LCMS: Rt=1.029 min, MS (ESI) m/z=215.1 [M+H]⁺.

### Step 3

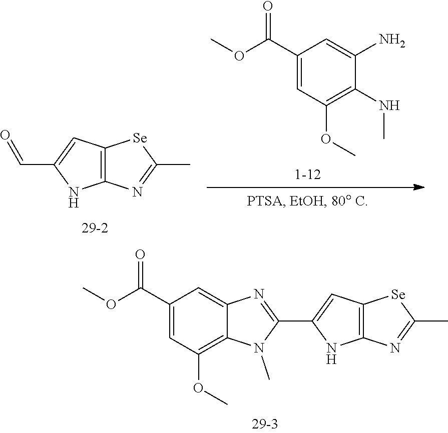

Compound 29-3: To a solution of compound 29-2 (380 mg, 1.77 mmol) in EtOH (10 mL) was added compound 1-12 (375 mg, 1.77 mmol) and PTSA (30.4 mg, 0.18 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 29-3 (350 mg) as a yellow oil, yield: 48.8%. LCMS: Rt=1.239 min, MS (ESI) m/z=404.9 [M+H]⁺.

### Step 4

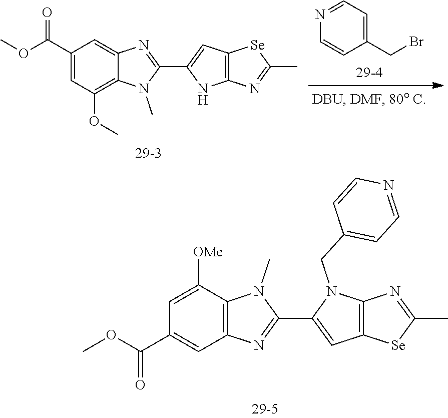

Compound 29-5: To a solution of compound 29-3 (200 mg, 0.50 mmol) and compound 29-4 (248.51 mg, 0.99 mmol) in DMF (10 mL) was added DBU (380 mg, 2.50 mmol). The mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: EtOAc) to afford compound 29-5 (200 mg) as a yellow oil, yield: 81.6%. LCMS: Rt=1.159 min, MS (ESI) m/z=495.9 [M+H]⁺.

### Step 5

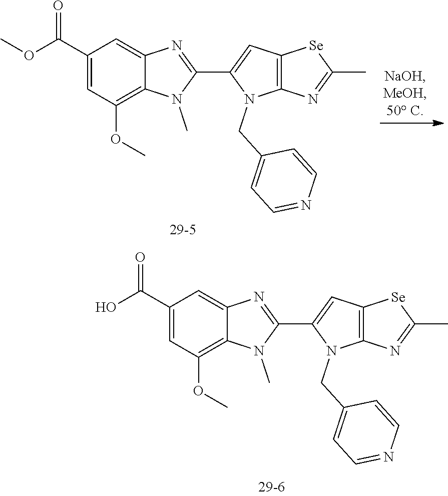

Compound 29-6: To a solution of compound 29-5 (200 mg, 0.40 mmol) in MeOH/H₂O (5/1 mL) was added NaOH (48.5 mg, 1.21 mmol), The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 29-6 (190 mg) as a yellow solid, yield: 97.9%. LCMS: Rt=1.076 min, MS (ESI) m/z=481.9 [M+H]⁺.

### Step 6

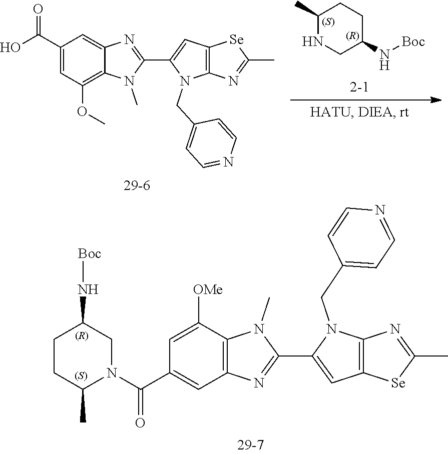

Compound 29-7: To a solution of compound 29-6 (95 mg, 0.20 mmol) in DMF (5 mL) was added compound 2-1 (42 mg, 0.20 mmol), HATU (76 mg, 0.20 mmol) and DIEA (77.4 mg, 0.60 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 29-7 (70 mg) as a brown solid, yield: 52.6%. LCMS: Rt=1.187 min, MS (ESI) m/z=678.1 [M+H]⁺.

### Step 7

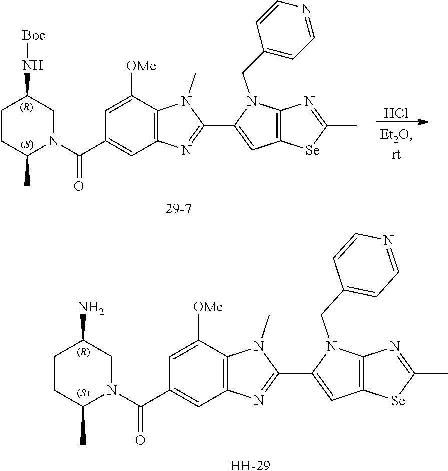

HH-29: To a solution of compound 29-7 (70 mg, 0.10 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-40%) to afford HH-29 (9 mg, hydrochloric acid salt form) as a yellow solid. yield: 15.0%. ¹H NMR (400 MHz, DMSO-d₆) δ ppm 8.82-8.70 (m, 2H), 8.49-8.16 (m, 3H), 7.61 (d, J=5.4 Hz, 2H), 7.23-7.08 (m, 2H), 6.80 (d, J=6.2 Hz, 1H), 6.07 (s, 2H), 4.15-3.90 (m, 7H), 3.07 (s, 2H), 2.78-2.68 (m, 3H), 1.95-1.36 (m, 4H), 1.14 (d, J=6.8 Hz, 3H). LCMS: Rt=1.011 min, MS (ESI) m/z=578.2 [M+H]⁺. HPLC: Rt=4.09 min, Purity: 97.98% (214 nm) and 97.25% (254 nm).

### Example 30. Synthesis of HH-30

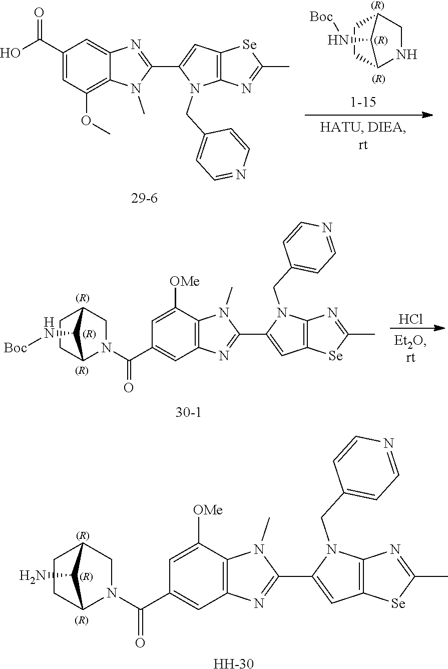

### Step 1

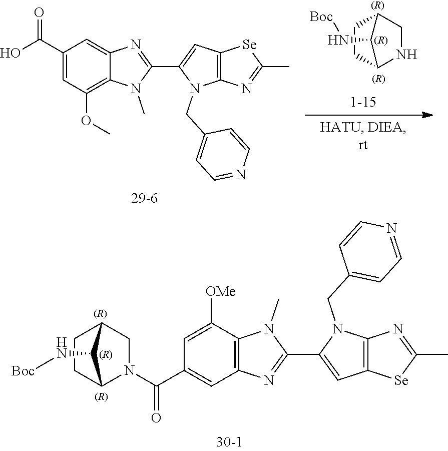

Compound 30-1: To a solution of compound 29-6 (95 mg, 0.20 mmol) in DMF (5 mL) was added compound 1-15 (42 mg, 0.20 mmol), HATU (76 mg, 0.20 mmol) and DIEA (77.4 mg, 0.60 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 30-1 (50 mg) as a brown solid, yield: 37.6%. LCMS: Rt=1.167 min, MS (ESI) m/z=676.2 [M+H]⁺.

### Step 2

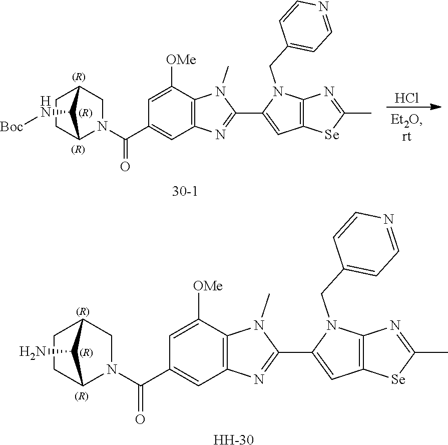

HH-30: To a solution of compound 30-1 (50 mg, 0.07 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-40%) to afford HH-30 (4.5 mg, hydrochloric acid salt form) as a yellow solid. yield: 9.57%. ¹H NMR (400 MHz, DMSO-d₆) δ ppm 8.62 (d, J=6.0 Hz, 2H), 8.48-8.18 (m, 3H), 7.45-7.39 (m, 2H), 7.28-7.23 (m, 1H), 7.12 (s, 1H), 6.88 (s, 1H), 6.01 (s, 2H), 4.09 (s, 4H), 3.94 (s, 3H), 3.63-3.54 (m, 1H), 3.48-3.41 (m, 1H), 3.16-3.08 (m, 1H), 2.75 (s, 3H), 2.65-2.59 (m, 1H), 2.02-1.76 (m, 4H). LCMS: Rt=0.997 min, MS (ESI) m/z=576.1 [M+H]⁺. HPLC: Rt=3.862 min, Purity: 93.56% (214 nm) and 91.04% (254 nm).

### Example 31. Synthesis of HH-31

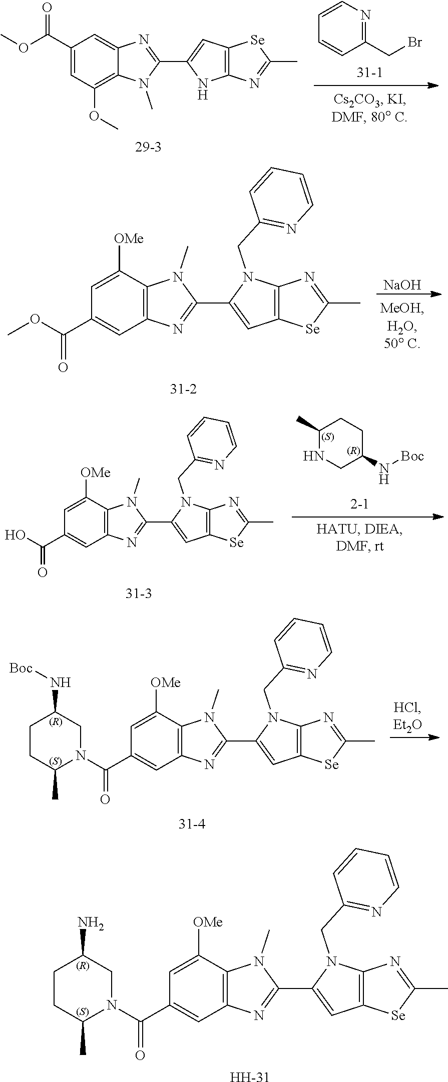

### Step 1

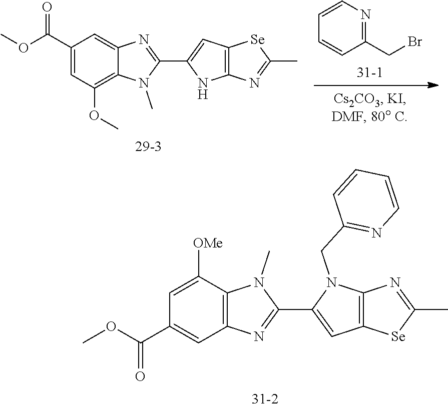

Compound 31-2: To a solution of compound 29-3 (150 mg, 0.37 mmol) and compound 31-1 (185.74 mg, 0.74 mmol) in DMF (10 mL) was added K₂CO₃ (255.3 mg, 1.85 mmol), KI (6.2 mg, 0.04 mmol). The mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: EtOAc) to afford compound 31-2 (150 mg) as a yellow oil, yield: 81.9%. LCMS: Rt=1.216 min, MS (ESI) m/z=496.1 [M+H]⁺.

### Step 2

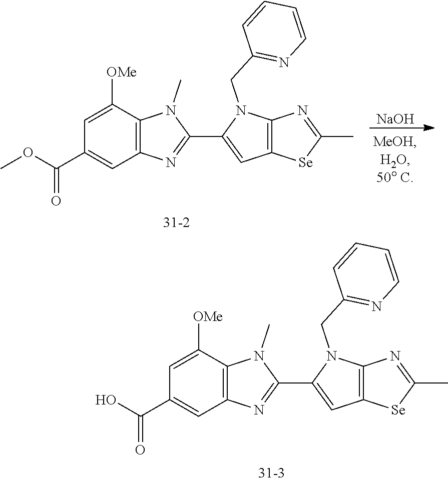

Compound 31-3: To a solution of compound 31-2 (150 mg, 0.30 mmol) in MeOH/H₂O (5/1 mL) was added NaOH (36.3 mg, 0.91 mmol), The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 31-3 (90 mg) as a yellow solid, yield: 62.5%. LCMS: Rt=1.147 min, MS (ESI) m/z=481.9 [M+H]⁺.

### Step 3

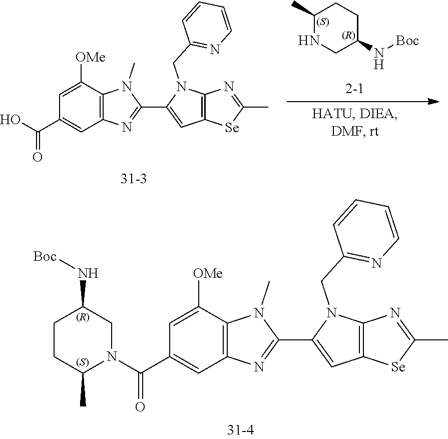

Compound 31-4: To a solution of compound 31-3 (90 mg, 0.19 mmol) in DMF (5 mL) was added compound 2-1 (40 mg, 0.19 mmol), HATU (72.2 mg, 0.19 mmol) and DIEA (73.5 mg, 0.57 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 31-4 (80 mg) as a brown solid, yield: 63.5%. LCMS: Rt=1.262 min, MS (ESI) m/z=678.0 [M+H]⁺.

### Step 4

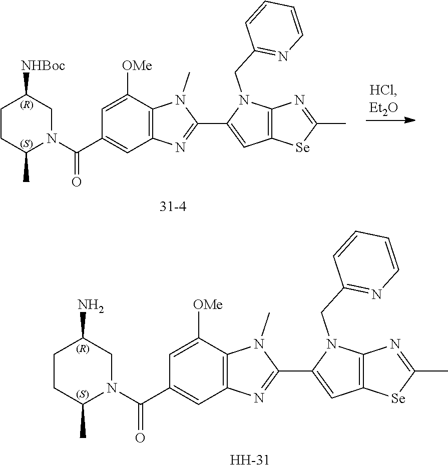

HH-31: To a solution of compound 31-4 (80 mg, 0.12 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-40%) to afford HH-31 (16 mg, hydrochloric acid salt form) as a yellow solid. yield: 23.5%. ¹H NMR (400 MHz) δ 8.76-8.60 (m, 1H), 8.47-8.25 (m, 3H), 8.05-7.95 (m, 1H), 7.55-7.45 (m, 1H), 7.42-7.33 (m, 2H), 7.27-7.21 (m, 1H), 6.95-6.90 (m, 1H), 5.94 (s, 2H), 4.20-3.98 (m, 7H), 3.27-3.01 (m, 2H), 2.76 (s, 3H), 1.94-1.52 (m, 4H), 1.20 (d, J=6.8 Hz, 3H). LCMS: Rt=1.044 min, MS (ESI) m/z=578.2 [M+H]⁺. HPLC: Rt=4.44 min, Purity: 95.01% (214 nm) and 95.16% (254 nm).

### Example 32. Synthesis of HH-32

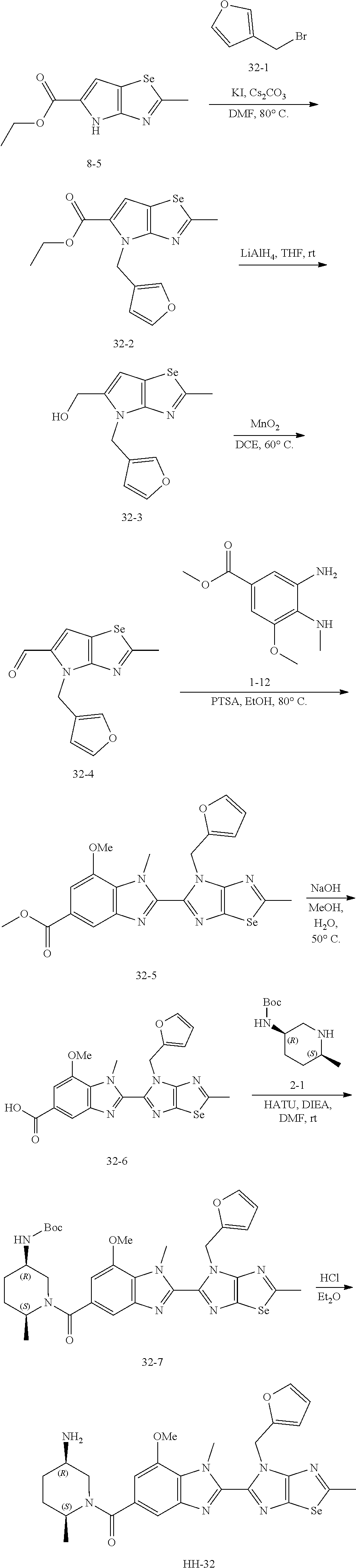

### Step 1

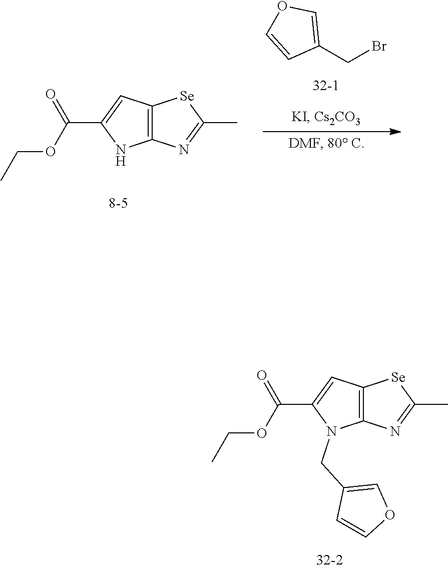 [SMILES: BrCc1ccoc1 ; CCOC(=O)c1cc2[se]c(C)nc2[nH]1 ; CCOC(=O)c1cc2[se]c(C)nc2n1Cc1ccoc1]

Compound 32-2: To a solution of compound 8-5 (400 mg, 1.56 mmol) and compound 32-1 (298 mg, 1.86 mmol) in DMF (10 mL) was added potassium carbonate (646 mg, 4.68 mmol). The mixture was stirred at 60° C. for 16 h. The mixture was diluted with water (30 mL) and extracted with ethyl acetate (30 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 32-2 (450 mg) as a yellow oil, yield: 82.1%. LCMS: Rt=1.413 min, MS (ESI) m/z=339.0 [M+H]⁺.

### Step 2

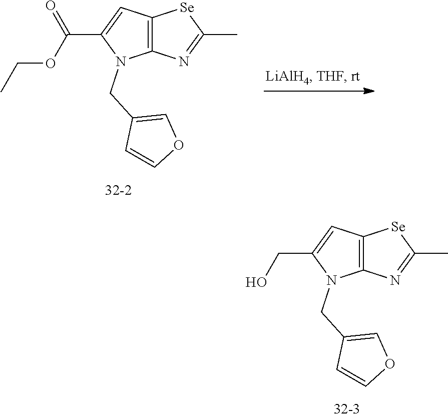 [SMILES: CCOC(=O)c1cc2[se]c(C)nc2n1Cc1ccoc1 ; Cc1nc2c(cc(CO)n2Cc2ccoc2)[se]1]

Compound 32-3: To a solution of compound 32-2 (450 mg, 1.32 mmol) in THF (12 mL) was added LiAlH₄ (1M in THF, 3.99 mL, 3.99 mmol) at 0° C. The reaction mixture was stirred at 25° C. for 5 h. The mixture was quenched with water (20 mL) and filtered. The filtrate was extracted with ethyl acetate (20 mL×3). The organic layers were dried over anhydrous Na₂SO₄ and concentrated under vacuum to afford crude product (390 mg) as a yellow oil. LCMS: Rt=1.181 min, MS (ESI) m/z=297.0 [M+H]⁺.

### Step 3

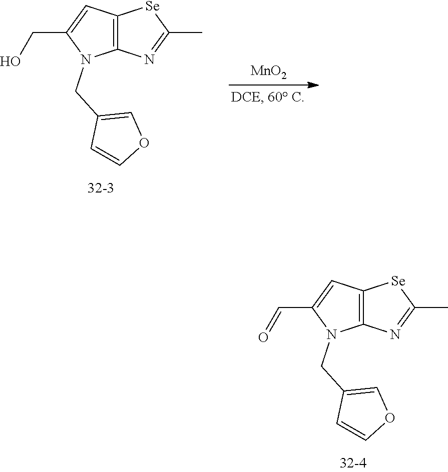

Compound 32-4: To a solution of compound 32-3 (390 mg, 1.32 mmol) in DCE (20 mL) was added MnO₂ (1.14 g, 13.14 mmol). The reaction mixture was stirred at 60° C. for 16 h. The mixture was filtered and the filtrate was concentrated under vacuum to afford crude product (360 mg) as a brown oil. LCMS: Rt=1.293 min, MS (ESI) m/z=294.9 [M+H]⁺.

### Step 4

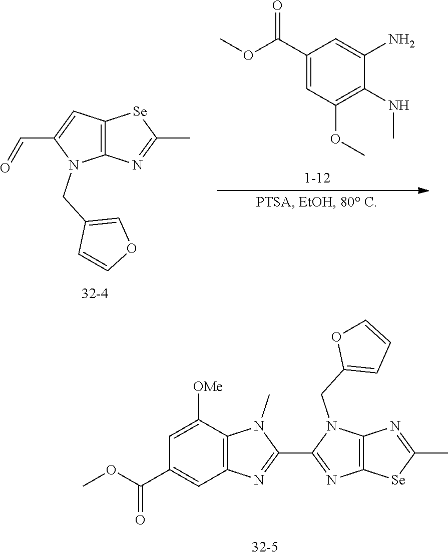

Compound 32-5: To a solution of compound 32-4 (360 mg, 1.23 mmol) in EtOH (25 mL) was added compound 1-12 (255 mg, 1.23 mmol) and PTSA (18 mg, 0.12 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 32-5 (390 mg) as a yellow oil, yield: 65.7%. LCMS: Rt=1.407 min, MS (ESI) m/z=484.9 [M+H]⁺.

### Step 5

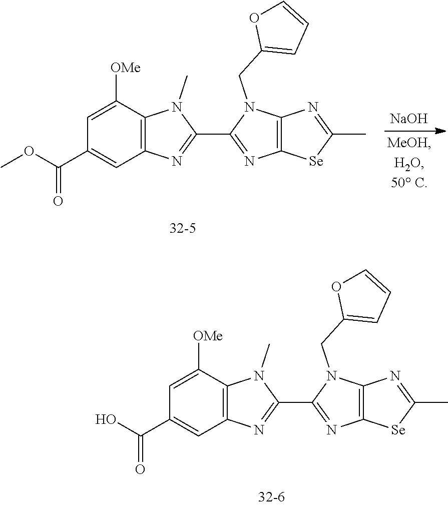

Compound 32-6: To a solution of compound 32-5 (390 mg, 0.81 mmol) in MeOH/H₂O (10/2 mL) was added NaOH (45 mg, 2.43 mmol). The reaction mixture was stirred at 50° C. for 16 h. The mixture was concentrated and adjusted pH to 5-6 with 1N hydrochloric acid aqueous solution, then extracted with ethyl acetate (20 mL×3). The organic phase was washed with water (10 mL), brine (10 mL), dried over anhydrous Na₂SO₄ and concentrated under vacuum to give compound 32-6 (300 mg) as a yellow solid, yield: 79.4%. LCMS: Rt=1.281 min, MS (ESI) m/z=470.9 [M+H]⁺.

### Step 6

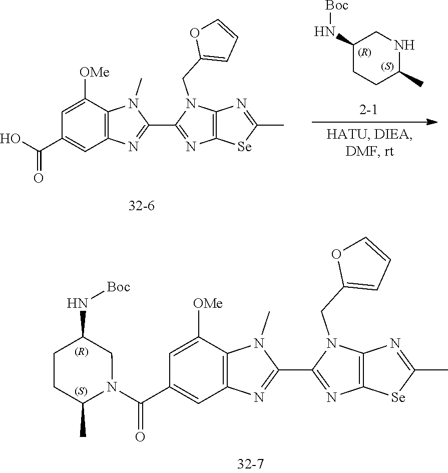

Compound 32-7: To a solution of compound 32-6 (100 mg, 0.21 mmol) in DMF (5 mL) was added compound 2-1 (46 mg, 0.21 mmol), HATU (80 mg, 0.21 mmol) and DIEA (82 mg, 0.63 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: EtOAc) to afford compound 32-7 (90 mg) as a brown solid, yield: 63.4%. LCMS: Rt=1.407 min, MS (ESI) m/z=667.0 [M+H]⁺.

### Step 7

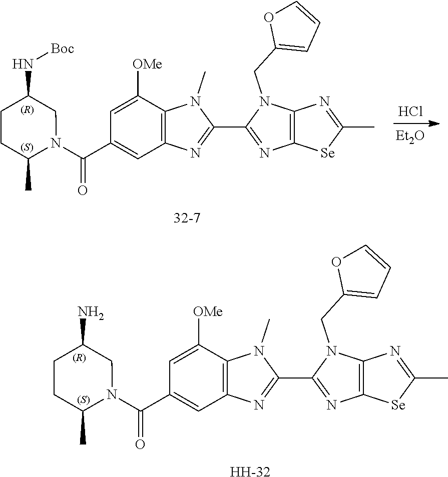

HH-32: To a solution of compound 32-7 (90 mg, 0.13 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 20-40%) to afford HH-32 (40 mg, hydrochloric acid salt form) as a yellow solid. yield: 52.6%. ¹H NMR (400 MHz, DMSO-d₆) δ ppm 8.32 (s, 3H), 7.43 (t, J=1.6 Hz, 1H), 7.36-7.30 (m, 2H), 7.10-7.04 (m, 1H), 6.94 (s, 1H), 6.12-6.06 (m, 1H), 5.58 (s, 2H), 5.22-5.03 (m, 2H), 4.03 (s, 3H), 3.97 (s, 3H), 3.22-2.97 (m, 2H), 2.85-2.73 (m, 3H), 1.92-1.67 (m, 3H), 1.62-1.52 (m, 1H), 1.18 (d, J=6.8 Hz, 3H). LCMS: Rt=1.135 min, MS (ESI) m/z=566.9 [M+H]⁺. HPLC: Rt=4.825 min, Purity: 100% (214 nm) and 100% (254 nm).

### Example 33. Synthesis of HH-33

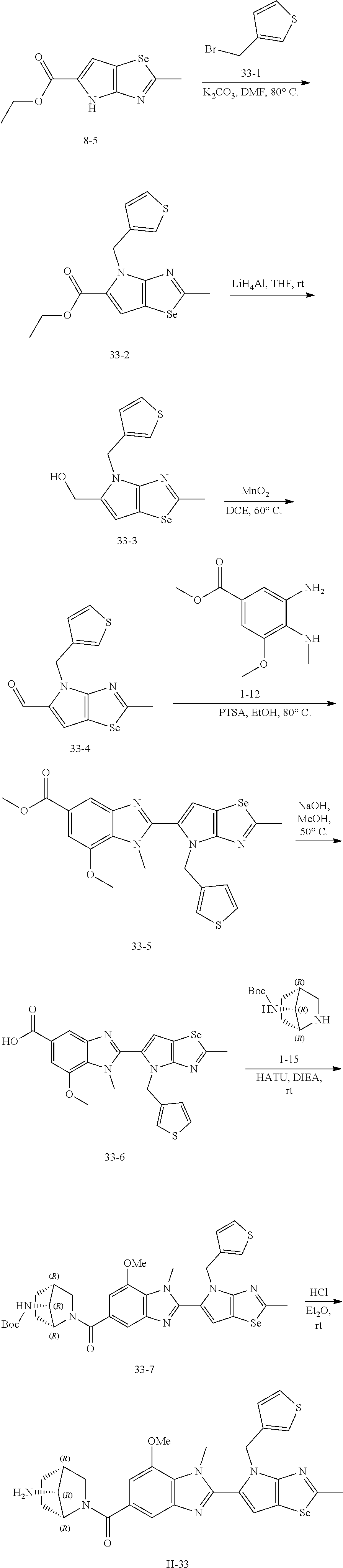

### Step 1

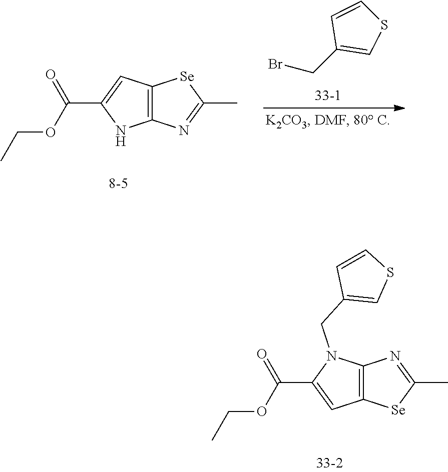 [SMILES: BrCc1ccsc1 ; CCOC(=O)c1cc2[se]c(C)nc2[nH]1 ; CCOC(=O)c1cc2[se]c(C)nc2n1Cc1ccsc1]

Compound 33-2: To a solution of compound 8-5 (400 mg, 1.56 mmol) in DMF (10 mL) was added compound 33-1 (413.1 mg, 2.33 mmol) and K₂CO₃ (429.9 mg, 3.11 mmol). The mixture was stirred at 80° C. for 3 h. The mixture was diluted with ethyl acetate (50 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 33-2 (400 mg) as a yellow solid, yield: 72.8%. LCMS: Rt=1.462 min, MS (ESI) m/z=355.0[M+H]⁺.

### Step 2

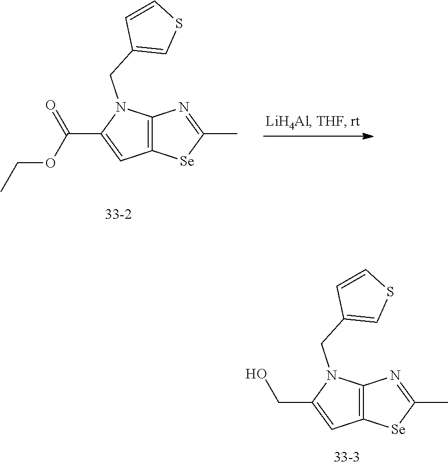 [SMILES: CCOC(=O)c1cc2[se]c(C)nc2n1Cc1ccsc1 ; Cc1nc2c(cc(CO)n2Cc2ccsc2)[se]1]

Compound 33-3: To a solution of compound 33-2 (0.4 g, 1.13 mmol) in THE (10 mL), then was added LiAlH₄ (1M in THF, 2.3 mL, 2.3 mmol) at 0° C. The reaction mixture was stirred at 25° C. for 5 h. The mixture was quenched with water (10 mL) and filtered. The filtrate was extracted with ethyl acetate (10 mL×3). The organic layers were dried over anhydrous Na₂SO₄ and concentrated under vacuum to afford crude product (0.2 g) as a yellow oil. LCMS: Rt=1.223 min, MS (ESI) m/z=312.9 [M+H]⁺.

### Step 3

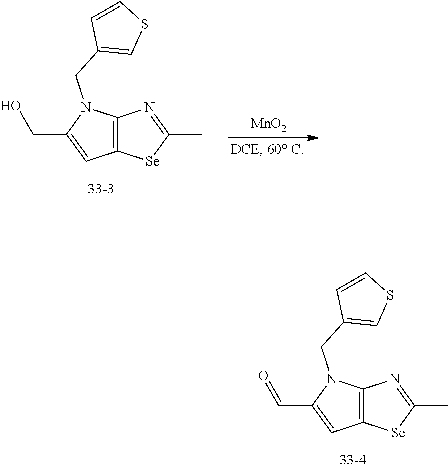

Compound 33-4: To a solution of compound 33-3 (330 mg, 1.06 mmol) in DCE (20 mL) was added MnO₂ (921.7 mg, 10.6 mmol). The reaction mixture was stirred at 60° C. for 16 h. The mixture was filtered and the filtrate was concentrated under vacuum to afford crude product (200 mg) as a brown oil. LCMS: Rt=1.319 min, MS (ESI) m/z=311.0 [M+H]⁺.

### Step 4

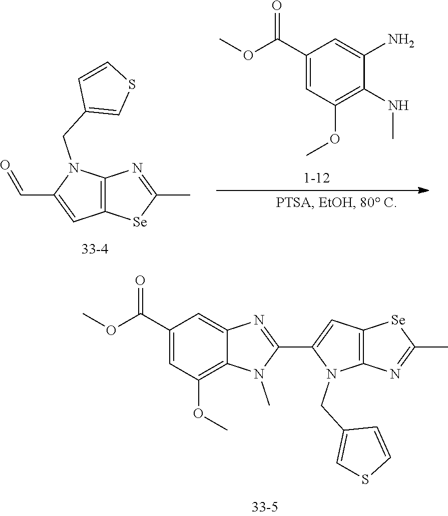

Compound 33-5: To a solution of compound 33-4 (0.2 g, 0.65 mmol) in EtOH (10 mL) was added compound 1-12 (135.9 mg, 0.65 mmol) and PTSA (11.1 mg, 0.06 mmol), The reaction mixture was stirred at 80° C. for 12 h. The mixture was concentrated under vacuum to give the crude product, which was purified by silica gel chromatography (elution gradient: petroleum ether/EtOAc, 5/1, v/v) to afford compound 33-5 (0.3 g) as a yellow oil, yield: 92.8%. LCMS: Rt=1.428 min, MS (ESI) m/z=500.9 [M+H]⁺.

### Step 5

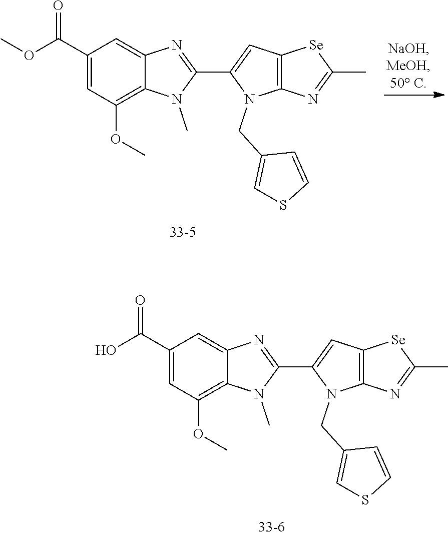

Compound 33-6: To a solution of compound 33-5 (300 mg, 0.60 mmol) in MeOH (10 mL) was added sodium hydroxide (2 N, 0.6 mL). The mixture was stirred at 50° C. for 3 h. The mixture was concentrated and adjusted pH to 5-6 with hydrochloric acid aqueous solution (2N), then extracted with ethyl acetate (50 mL×3). The organic phase was washed with water (50 mL), brine (50 mL), and concentrated under vacuum to give compound 33-6 (200 mg) as a yellow solid, yield: 68.6%. LCMS: Rt=1.297 min, MS (ESI) m/z=486.9 [M+H]⁺.

### Step 6

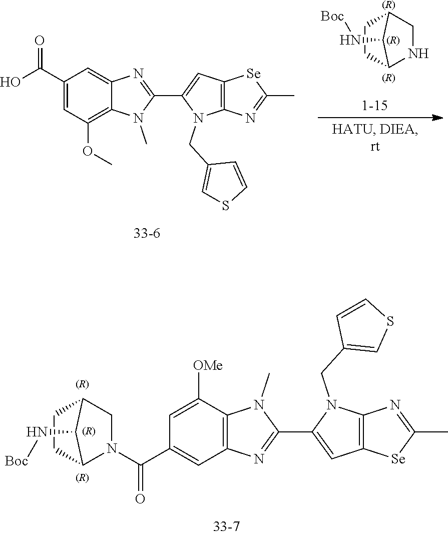

Compound 33-7: To a solution of compound 33-6 (100 mg, 0.21 mmol) in DMF (5 mL) was added compound 1-15 (48.6 mg, 0.23 mmol), HATU (94.0 mg, 0.25 mmol) and DIEA (79.9 mg, 0.62 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/99, v/v) to afford compound 33-7 (100 mg) as a brown solid, yield: 71.2%. LCMS: Rt=1.402 min, MS (ESI) m/z=683.0 [M+H]⁺.

### Step 7

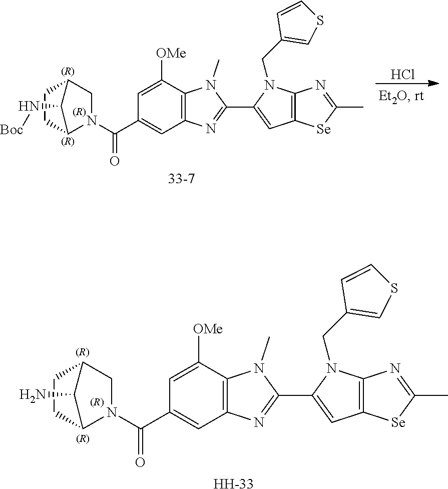

HH-33: To a solution of compound 33-7 (100 mg, 0.15 mmol) in Et₂O (10 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-33 (22 mg, hydrochloric acid salt form) as a yellow solid. yield: 66.6%. ¹H NMR (400 MHz, DMSO-d₆) δ ppm 8.26 (s, 3H), 7.38-7.33 (m, 2H), 7.07 (s, 1H), 7.01 (s, 1H), 6.91 (s, 1H), 6.76 (d, J=4.8 Hz, 1H), 5.80 (s, 2H), 4.03 (s, 3H), 3.99 (s, 3H), 3.23-3.09 (m, 2H), 3.07-3.02 (m, 1H), 2.82 (s, 3H), 1.92-1.75 (m, 3H), 1.62-1.52 (m, 1H), 1.22-1.19 (m, 3H). LCMS: Rt=1.139 min, MS (ESI) m/z=583.1 [M+H]⁺. HPLC: Rt=5.436 min, Purity: 100% (214 nm) and 100% (254 nm).

### Example 34. Synthesis of HH-34

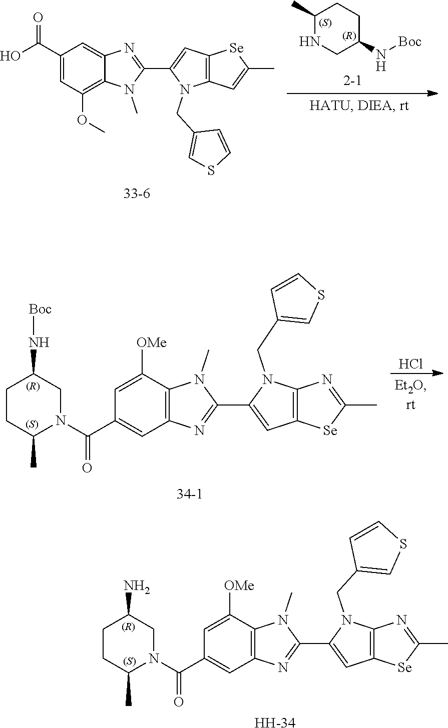

### Step 1

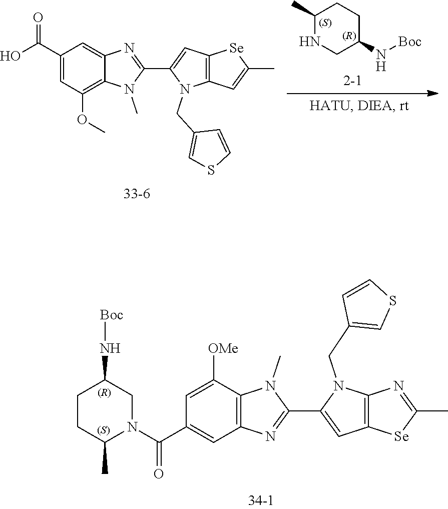

Compound 34-1: To a solution of compound 33-6 (100 mg, 0.21 mmol) in DMF (10 mL) was added compound 2-1 (48.1 mg, 0.23 mmol), HATU (94.0 mg, 0.25 mmol) and DIEA (79.9 mg, 0.62 mmol). The reaction mixture was stirred at 25° C. for 3 h. The mixture was diluted with water (20 mL), extracted with ethyl acetate (50 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/10, v/v) to afford compound 34-1 (100 mg) as a brown solid, yield: 71.4%. LCMS: Rt=1.359 min, MS (ESI) m/z=681.1 [M+H]⁺.

### Step 2

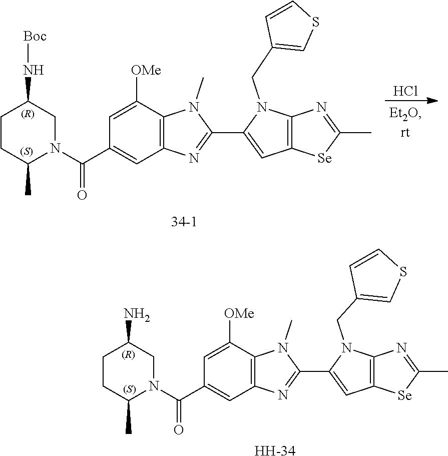

HH-34: To a solution of compound 34-1 (100 mg, 0.15 mmol) in Et₂O (10 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-34 (50 mg, hydrochloric acid salt form) as a yellow solid. yield: 58.5. ¹H NMR (400 MHz, DMSO-d₆) δ ppm 8.38 (s, 3H), 7.43 (s, 1H), 7.31 (dd, J=4.8, 3.2 Hz, 1H), 6.99 (s, 2H), 6.96 (s, 1H), 6.76 (d, J=5.2 Hz, 1H), 5.78 (s, 2H), 3.99 (s, 6H), 3.63 (s, 1H), 3.50 (s, 1H), 3.17 (d, J=10.0 Hz, 1H), 2.81 (s, 3H), 2.04-1.87 (m, 4H), 1.65-1.60 (m, 1H), 1.25 (s, 3H). LCMS: Rt=1.134 min, MS (ESI) m/z=580.9 [M+H]⁺. HPLC: Rt=4.735 min, Purity: 98.65% (214 nm) and 98.67% (254 nm).

### Example 35. Synthesis of HH-35

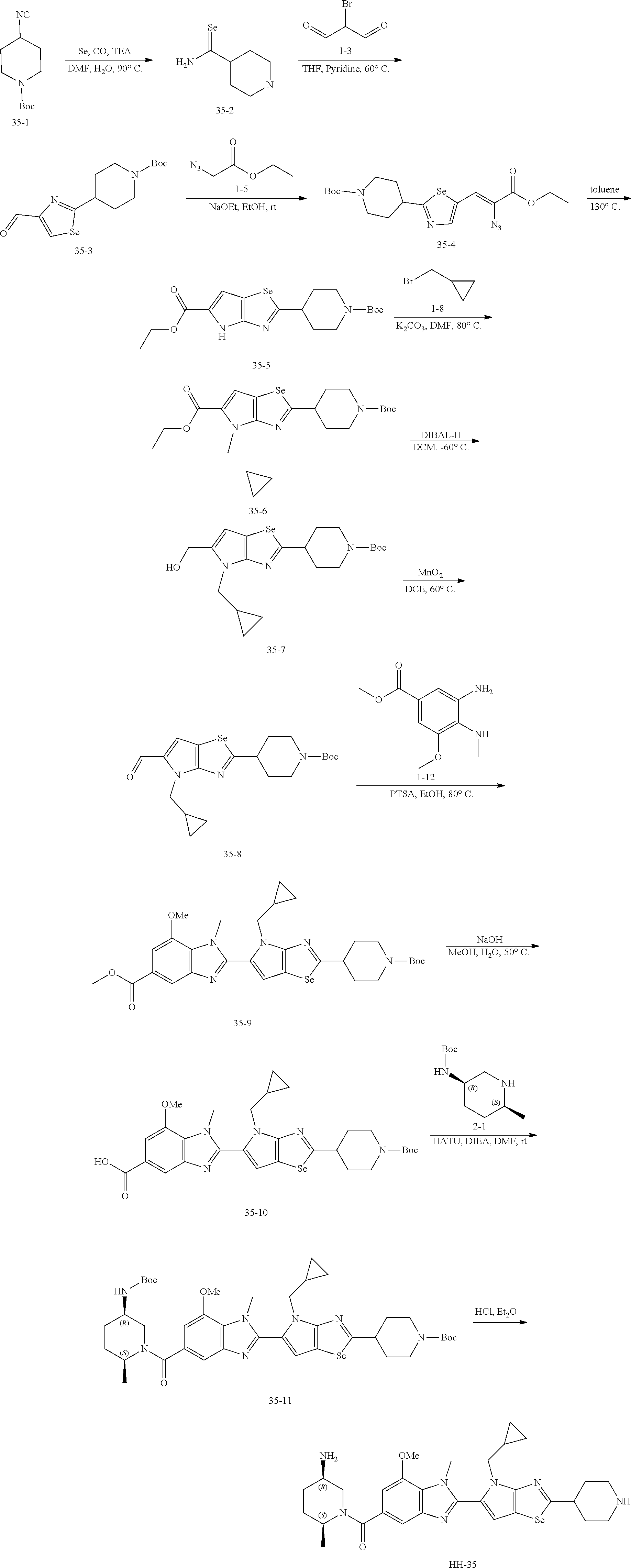

### Step 1

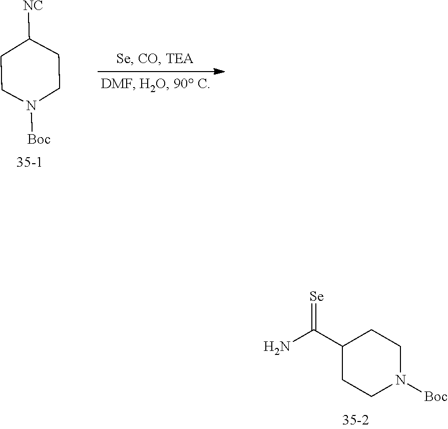

Compound 35-2: To a solution of compound 35-1 (6 g, 28.6 mmol) in DMF (50 mL)/H₂O (5 mL) was added Se (2.7 g, 33.8 mmol). The mixture was stirred at 90° C. under CO for 5 h. The mixture was filtered and diluted with ethyl acetate (200 mL) and washed with H₂O (50 mL), brine (50 mL×2). The organic layer was dried over anhydrous sodium sulfate, filtered and concentrated under vacuum to afford crude product, which was purified by flash chromatography (elution gradient: petroleum ether/ethyl acetate, 2/1, v/v) to afford compound 35-2 (7.1 g) as a yellow oil, yield: 85.5%. LCMS: Rt=1.16 min, MS (ESI) m/z=237.0 [M−56]⁺.

### Step 2

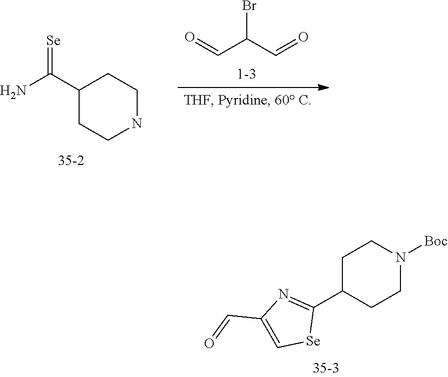 [SMILES: NC(=[Se])C1CCNCC1 ; O=CC(Br)C=O ; CC(C)(C)OC(=O)N1CCC(c2nc(C=O)c[se]2)CC1]

Compound 35-3: To a solution of compound 35-2 (7.1 g, 24.3 mmol) in THE (200 mL) was added compound 1-3 (3.7 g, 24.7 mmol) and pyridine (3.8 g, 48.1 mmol). The mixture was stirred at 60° C. for 16 h. The mixture was filtered and concentrated under reduced pressure to afford residue. The residue was diluted with water (100 mL), extracted with ethyl acetate (200 mL×2). The combined organic layers were dried over anhydrous sodium sulfate and concentrated under vacuum to afford crude product, which was purified by flash chromatography (elution gradient: petroleum ether/ethyl acetate, 5/1, v/v) to afford compound 35-3 (4.4 g) as yellow oil, yield: 52.6%. ¹H NMR (400 MHz, CDCl₃): δ ppm 9.91 (s, 1H), 8.34 (s, 1H), 4.25-4.22 (m, 2H), 3.20-3.12 (m, 1H), 2.89 (t, J=12.4 Hz, 2H), 2.17-2.13 (m, 2H), 1.77-1.67 (m, 2H), 1.47 (s, 9H). LCMS: Rt=1.31 min, MS (ESI) m/z=288.9 [M−56]⁺.

### Step 3

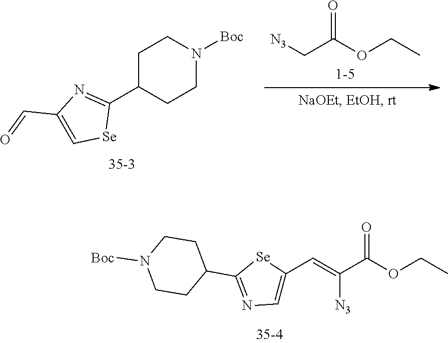

Compound 35-4: To a solution of sodium ethoxide (13.0 g, 38.2 mmol, 20% wt) in EtOH (50 mL) was added compound 35-3 (4.4 g, 12.8 mmol) and compound 1-5 (4.9 g, 38.0 mmol) at −10° C. under N₂. The mixture was stirred at −10° C. under N₂ for 1 h and at 10° C. for further 1 h. The mixture was diluted with ethyl acetate (200 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 35-4 (2.7 g) as a yellow solid, yield: 46.6%. ¹H NMR (400 MHz, CDCl₃): δ ppm 7.86 (s, 1H), 7.18 (s, 1H), 4.37 (q, J=7.2 Hz, 2H), 4.20 (br s, 2H), 3.22-3.12 (m, 1H), 2.93-2.88 (m, 2H), 2.15-2.13 (m, 2H), 1.78-1.68 (m, 2H), 1.48 (s, 9H), 1.40 (t, J=7.0 Hz, 3H). LCMS: Rt=1.51 min, MS (ESI) m/z=455.9 [M+H]⁺.

### Step 4

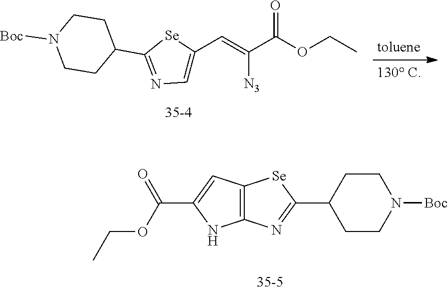

Compound 35-5: A solution of compound 35-4 (2.7 g, 5.9 mmol) in toluene (50 mL) was stirred at 130° C. for 5 h. The mixture was concentrated under vacuum to afford crude product, which was purified by flash chromatography (elution gradient: petroleum ether/ethyl acetate, 5/1, v/v) to afford compound 35-5 (2.1 g) as yellow solid, yield: 84.0%. LCMS: Rt=1.41 min, MS (ESI) m/z=371.9 [M−56]⁺.

### Step 5

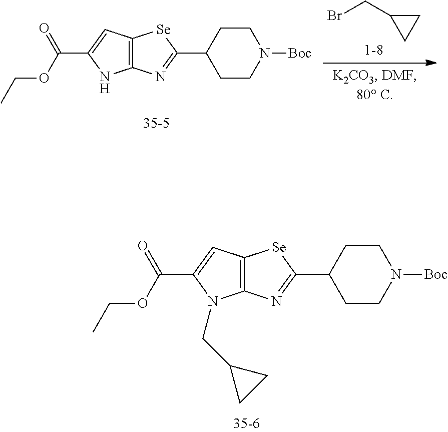 [SMILES: CCOC(=O)c1cc2[se]c(C3CCN(C(=O)OC(C)(C)C)CC3)nc2[nH]1 ; BrCC1CC1 ; CCOC(=O)c1cc2[se]c(C3CCN(C(=O)OC(C)(C)C)CC3)nc2n1CC1CC1]

Compound 35-6: To a solution of compound 35-5 (2.1 g, 4.9 mmol) in DMF (20 mL) was added compound 1-8 (996 mg, 7.4 mmol) and K₂CO₃ (1.4 g, 10.2 mmol). The mixture was stirred at 80° C. for 5 h. The mixture was diluted with ethyl acetate (100 mL), washed with water (50 mL×2) and brine (50 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 35-6 (1.6 g) as a yellow solid, yield: 67.5. LCMS: Rt=1.66 min, MS (ESI) m/z=426.1 [M−56]⁺.

### Step 6

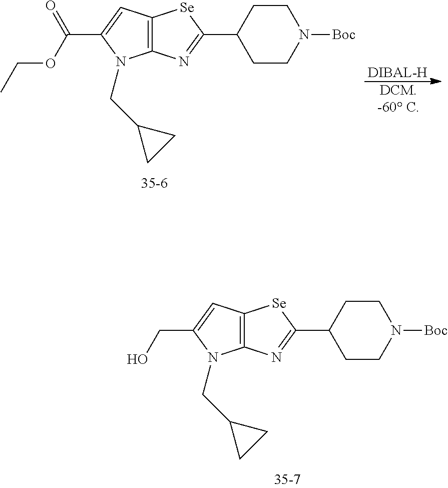 [SMILES: CCOC(=O)c1cc2[se]c(C3CCN(C(=O)OC(C)(C)C)CC3)nc2n1CC1CC1 ; "DIBAL-H" ; "DCM. -60° C." ; CC(C)(C)OC(=O)N1CCC(c2nc3c(cc(CO)n3CC3CC3)[se]2)CC1]

Compound 35-7: To a solution of compound 35-6 (1 g, 2.1 mmol) in DCM (10 mL) was added 1N DIBAL-H (3.1 mL, 3.1 mmol) under N₂ at −60° C. The mixture was stirred at −60° C. for 4 h. The mixture was quenched with saturated ammonium chloride aqueous solution (10 mL), filtered and extracted with ethyl acetate (50 mL×2). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 35-7 (800 mg) as a yellow solid, yield: 87.7%. LCMS: Rt=1.39 min, MS (ESI) m/z=462.2 [M+Na]⁺.

### Step 7

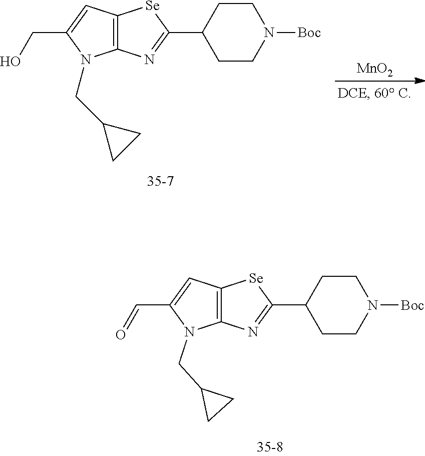

Compound 35-8: To a solution of compound 35-7 (800 mg, 1.82 mmol) in DCE (20 mL) was added MnO₂ (1.6 g, 1.84 mmol). The mixture was stirred at 60° C. for 16 h. The mixture was filtered and concentrated under reduced pressure to afford crude product, which was purified by flash chromatography (elution gradient: petroleum ether/ethyl acetate, 5/1, v/v) to afford compound 35-8 (600 mg) as a red solid, yield: 75.4%. LCMS: Rt=1.50 min, MS (ESI) m/z=460.1 [M+Na]⁺.

### Step 8

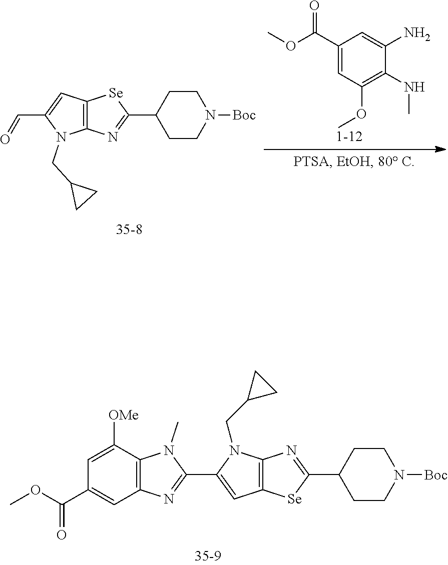

Compound 35-9: To a solution of compound 35-8 (600 mg, 1.37 mmol) in EtOH (10 mL) was added compound 1-12 (317 mg, 1.51 mmol) and PTSA (24 mg, 0.14 mmol). The mixture was stirred at 80° C. for 2 h. The mixture was filtered and concentrated under reduced pressure to afford crude product, which was purified by flash chromatography (elution gradient: petroleum ether/ethyl acetate, 3/1, v/v) to afford compound 35-9 (640 mg) as a red solid, yield: 74.3%. LCMS: Rt=1.59 min, MS (ESI) m/z=628.2 [M+H]⁺.

### Step 9

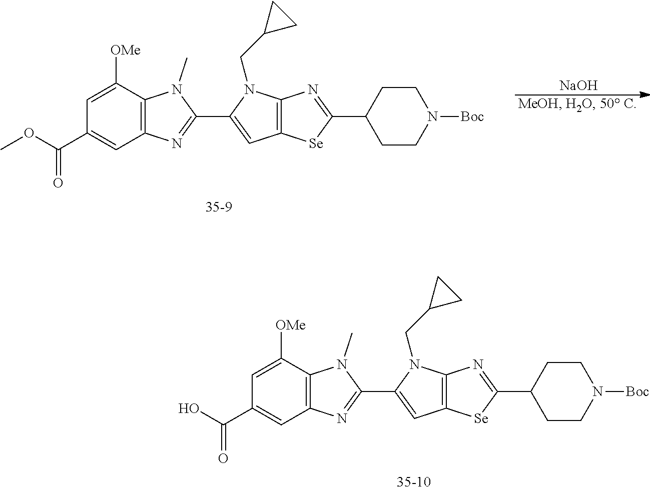 [SMILES: COC(=O)c1cc(OC)c2c(c1)nc(-c1cc3[se]c(C4CCN(C(=O)OC(C)(C)C)CC4)nc3n1CC1CC1)n2C ; "NaOH" ; "MeOH, H2O, 50° C." ; COc1cc(C(=O)O)cc2nc(-c3cc4[se]c(C5CCN(C(=O)OC(C)(C)C)CC5)nc4n3CC3CC3)n(C)c12]

Compound 35-10: To a solution of compound 35-9 (640 mg, 1.02 mmol) in MeOH (5 mL)/THF (5 mL) was added 2N NaOH (2 mL, 4 mmol). The mixture was stirred at 50° C. for 5 h. The mixture was concentrated and adjusted pH to 5-6 with 1N hydrochloric acid aqueous solution, then extracted with dichloromethane (50 mL×3). The organic phases were combined, dried over Na₂SO₄, filtered and concentrated under vacuum to give compound 35-10 (560 mg) as a yellow solid, yield: 89.5%. LCMS: Rt=1.42 min, MS (ESI) m/z=614.1 [M+H]⁺.

### Step 10

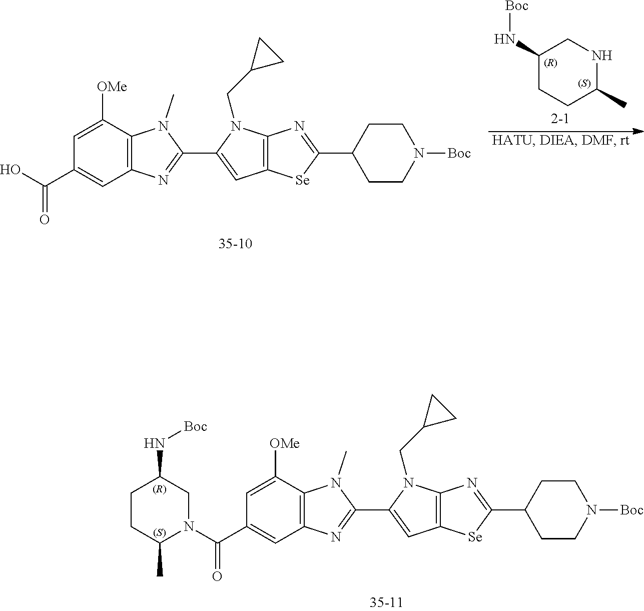

Compound 35-11: To a solution of compound 35-10 (80 mg, 0.13 mmol), DIEA (51 mg, 0.39 mol), HATU (65 mg, 0.17 mmol) in DMF (5 mL) was added compound 2-1 (31 mg, 0.14 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 35-11 (100 mg) as a yellow solid, yield: 94.7%. LCMS: Rt=1.50 min, MS (ESI) m/z=810.1 [M+H]⁺.

### Step 11

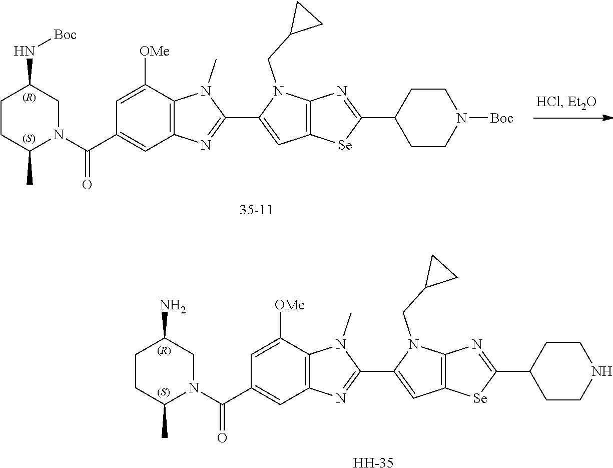

HH-35: To a solution of compound 35-11 (100 mg, 0.16 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-40%) to afford HH-35 (47.2 mg, hydrochloric acid salt form) as a yellow solid. yield: 56.1%. ¹H NMR (400 MHz, DMSO-d₆): δ ppm 9.31-9.29 (m, 1H), 9.05-9.02 (m, 1H), 8.37 (s, 3H), 7.35 (s, 1H), 7.09 (s, 1H), 6.96 (s, 1H), 4.38 (d, J=6.8 Hz, 2H), 4.13 (s, 3H), 4.04 (s, 3H), 3.48-3.42 (m, 1H), 3.37-3.34 (m, 2H), 3.17-3.00 (m, 4H), 2.27-2.24 (m, 2H), 2.08-1.74 (m, 5H), 1.59-1.56 (m, 1H), 1.23-1.15 (m, 4H), 0.37-0.35 (m, 2H), 0.22-0.21 (m, 2H). LCMS: Rt=1.04 min, MS (ESI) m/z=610.1 [M+H]⁺. HPLC: Rt=3.54 min, Purity: 99.79% (214 nm) and 100% (254 nm).

### Example 36. Synthesis of HH-36

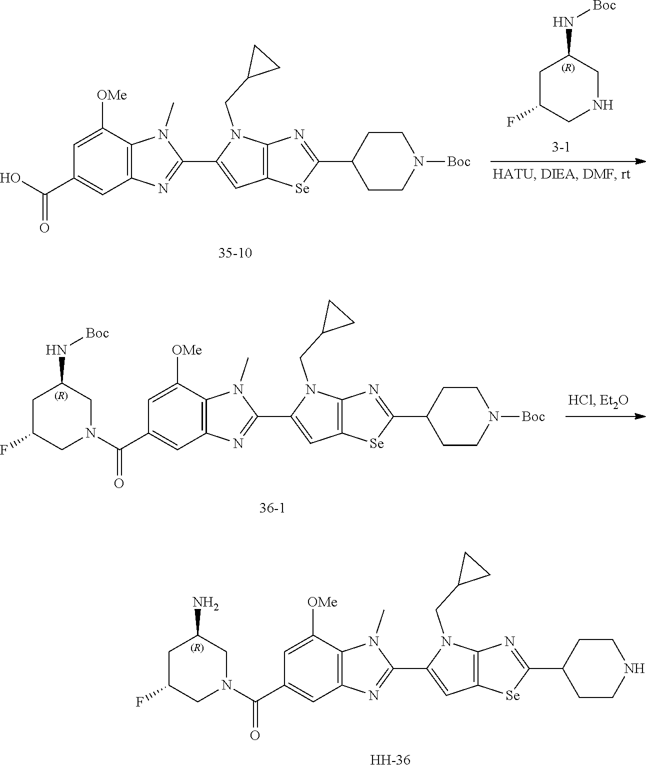

### Step 1

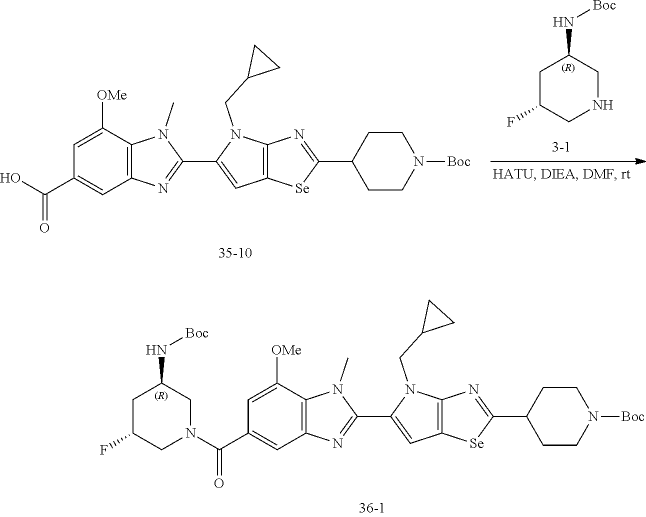

Compound 36-1: To a solution of compound 35-10 (80 mg, 0.13 mmol), DIEA (51 mg, 0.39 mol), HATU (65 mg, 0.17 mmol) in DMF (5 mL) was added compound 3-1 (31 mg, 0.14 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 36-1 (90 mg) as a yellow solid, yield: 84.9%. LCMS: Rt=1.47 min, MS (ESI) m/z=814.0 [M+H]⁺.

### Step 2

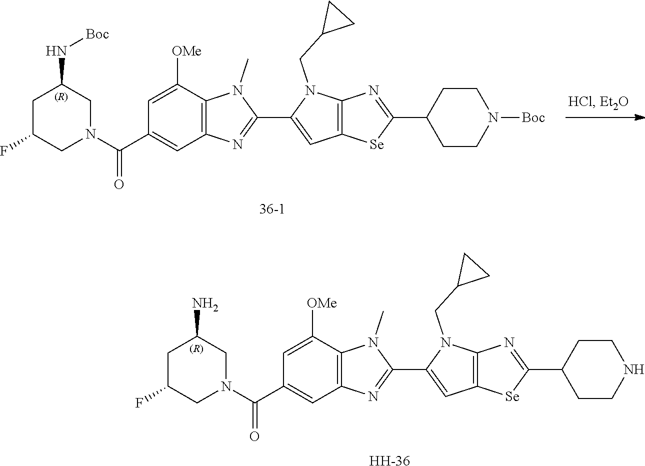

HH-36: To a solution of compound 36-1 (90 mg, 0.11 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-30%) to afford HH-36 (56.7 mg, hydrochloric acid salt form) as a yellow solid. yield: 74.8%. ¹H NMR (400 MHz, DMSO-d₆): δ ppm 9.27-9.24 (m, 1H), 9.02-8.98 (m, 1H), 8.47 (s, 3H), 7.37 (s, 1H), 7.08 (s, 1H), 6.96 (s, 1H), 5.06-4.95 (m, 2H), 4.38 (d, J=6.8 Hz, 2H), 4.13 (s, 3H), 4.00 (s, 3H), 4.13-4.00 (m, 1H), 3.49-3.35 (m, 5H), 3.09-3.00 (m, 3H), 2.43-2.41 (m, 1H), 2.27-2.24 (m, 2H), 2.06-1.87 (m, 3H), 1.23-1.13 (m, 1H), 0.37-0.35 (m, 2H), 0.23-0.21 (m, 2H). LCMS: Rt=0.93 min, MS (ESI) m/z=614.1 [M+H]⁺. HPLC: Rt=4.22 min, Purity: 99.01% (214 nm) and 100% (254 nm).

### Example 37. Synthesis of HH-37

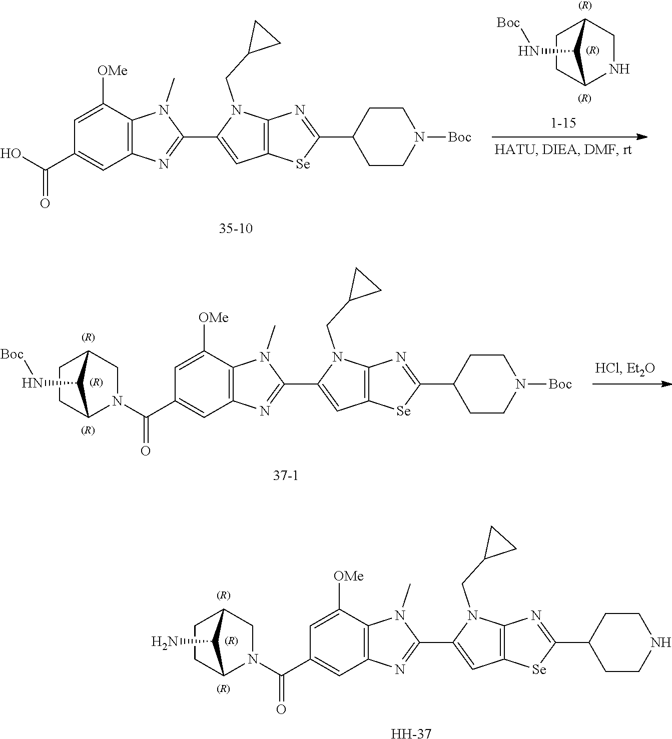

### Step 1

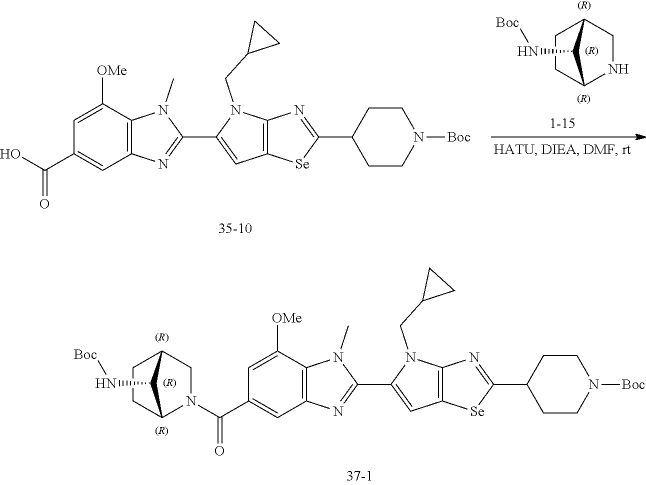

Compound 37-1: To a solution of compound 35-10 (80 mg, 0.13 mmol), DIEA (51 mg, 0.39 mol), HATU (65 mg, 0.17 mmol) in DMF (5 mL) was added compound 1-15 (31 mg, 0.15 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 37-1 (92 mg) as a yellow solid, yield: 87.4%. LCMS: Rt=1.49 min, MS (ESI) m/z=808.2 [M+H]⁺.

### Step 4

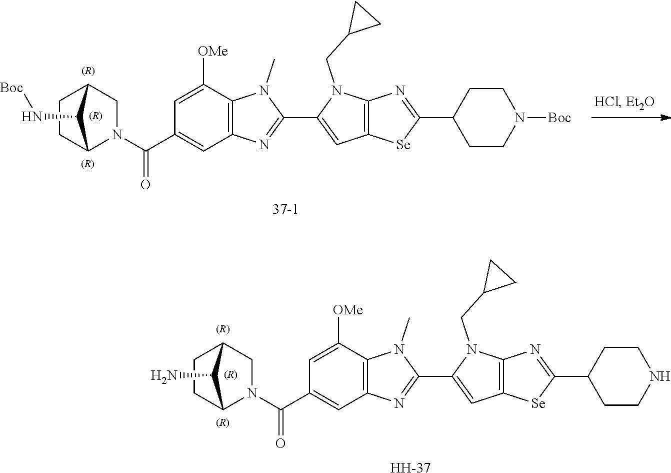

HH-37: To a solution of compound 37-1 (92 mg, 0.16 mmol) in ethyl ether (2 mL) was added hydrochloric acid solution (2 mL, 2M in ethyl ether). The mixture was stirred at room temperature for 2 h. The mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-30%) to afford HH-37 (46.8 mg, hydrochloric acid salt form) as a yellow solid. yield: 60.5%. ¹H NMR (400 MHz, DMSO-d₆): δ ppm 9.29 (s, 1H), 9.06 (s, 1H), 8.47 (s, 3H), 7.42 (s, 1H), 7.00 (s, 2H), 4.40 (d, J=6.8 Hz, 2H), 4.10 (s, 3H), 4.01 (s, 3H), 3.65-3.64 (m, 1H), 3.50-3.41 (m, 2H), 3.37-3.34 (m, 2H), 3.18-3.16 (m, 1H), 3.10-3.02 (m, 2H), 2.66 (br. s, 1H), 2.29-2.26 (m, 2H), 2.12-1.89 (m, 6H), 1.64-1.60 (m, 1H), 1.25-1.16 (m, 1H), 0.38-0.33 (m, 2H), 0.23-0.19 (m, 2H). LCMS: Rt=1.01 min, MS (ESI) m/z=608.0 [M+H]⁺. HPLC: Rt=3.34 min, Purity: 96.88% (214 nm) and 99.70% (254 nm).

### Example 38. Synthesis of HH-38

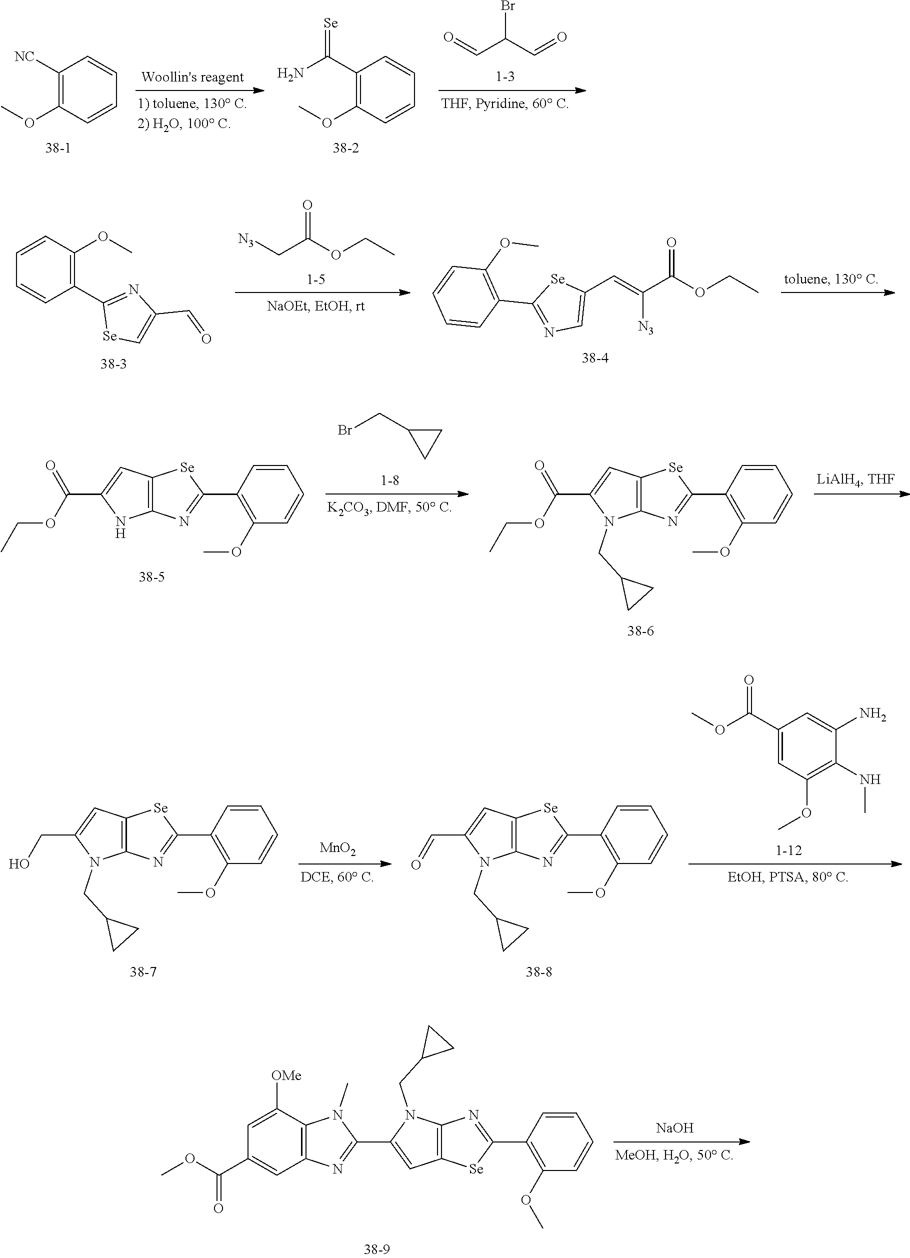

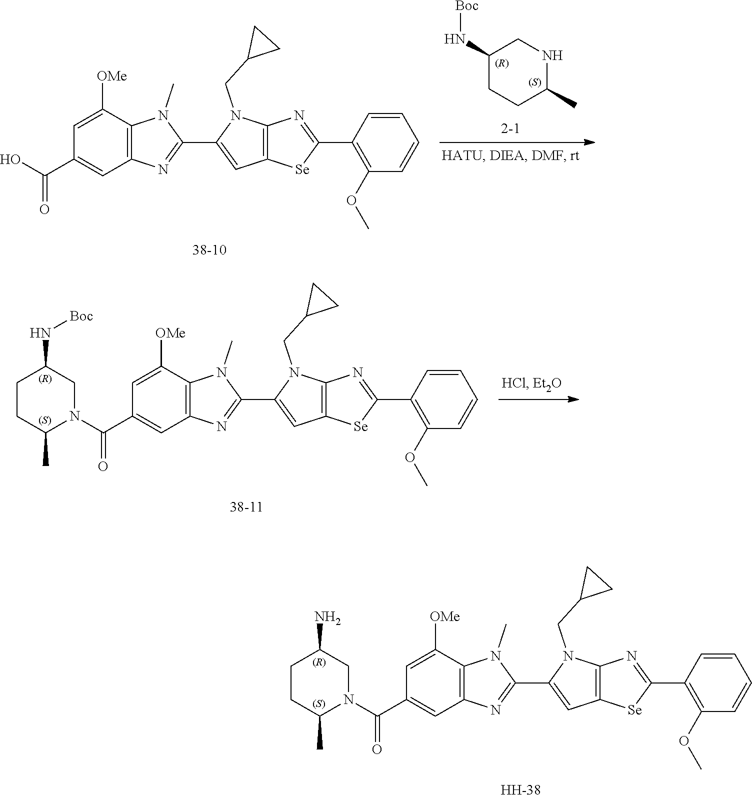

### Step 1

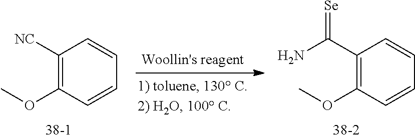

Compound 38-2: To a solution of compound 38-1 (1 g, 7.51 mmol) in toluene (20 mL) was added Woollin's reagent (2.0 g, 3.75 mmol). The mixture was stirred at 130° C. for 5 h. Water (1 mL) was added into the mixture and stirred at 100° C. for 1 h. The residue was dissolved with EtOAc (50 mL) and washed with H₂O (20 mL), brine (20 mL). The organic layer was dried over Na₂SO₄, filtered and concentrated under reduced pressure to afford crude product, which was purified by flash chromatography (elution gradient: petroleum ether/ethyl acetate, 5/1, v/v) to afford compound 38-2 (820 mg) as a yellow solid, yield: 50.8%. ¹H NMR (400 MHz, DMSO-d₆): δ ppm 10.91 (s, 1H), 10.06 (s, 1H), 7.72 (d, J=7.6 Hz, 1H), 7.39 (t, J=7.6 Hz, 1H), 7.04 (d, J=8.4 Hz, 1H), 6.95 (t, J=7.6 Hz, 1H), 3.80 (s, 3H). LCMS: Rt=1.09 min, MS (ESI) m/z=216.0 [M+H]⁺.

### Step 2

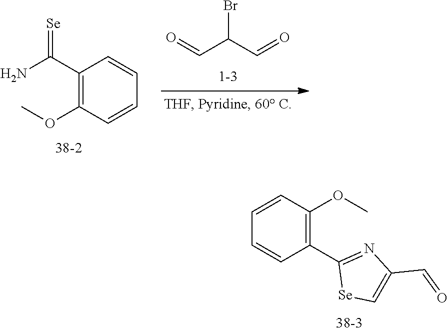

Compound 38-3: To a solution of compound 38-2 (820 mg, 3.81 mmol) in THF (30 mL) was added compound 1-3 (578 mg, 3.86 mmol) and pyridine (606 mg, 7.67 mmol). The mixture was stirred at 60° C. for 16 h. The mixture was filtered and concentrated under reduced pressure to afford residue. The residue was diluted with water (30 mL), extracted with ethyl acetate (50 mL×3). The combined organic layers were dried over anhydrous sodium sulfate and concentrated under vacuum to afford crude product, which was purified by flash chromatography (elution gradient: petroleum ether/ethyl acetate, 5/1, v/v) to afford compound 38-3 (480 mg) as yellow solid, yield: 47.2%. LCMS: Rt=1.27 min, MS (ESI) m/z=268.0 [M+H]⁺.

### Step 3

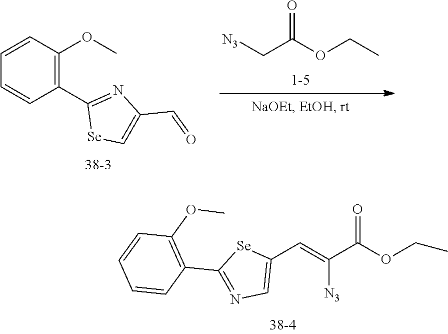

Compound 38-4: To a solution of sodium ethoxide (1.84 g, 5.41 mmol, 20% wt) in EtOH (20 mL) was added compound 38-3 (480 mg, 1.80 mmol) and compound 5 (699 mg, 5.41 mmol) at −10° C. under N₂. The mixture was stirred at −10° C. under N₂ for 1 h and at 10° C. for further 1 h. The mixture was quenched with saturated ammonium chloride aqueous solution (10 mL), extracted with ethyl acetate (50 mL×3). The organic layers were combined, dried over anhydrous sodium sulfate, filtered and concentrated under vacuum to afford crude product, which was purified by flash chromatography (elution gradient: petroleum ether/ethyl acetate, 5/1, v/v) to afford compound 38-4 (370 mg) as yellow solid, yield: 54.4%. LCMS: Rt=1.51 min, MS (ESI) m/z=378.9 [M+H]⁺.

### Step 4

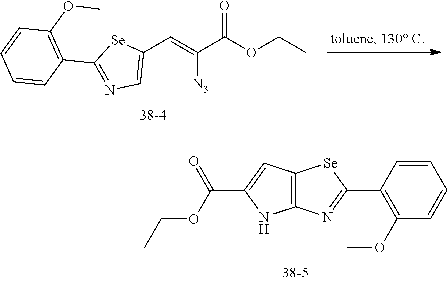

Compound 38-5: A solution of compound 38-4 (370 mg, 0.98 mmol) in toluene (10 mL) was stirred at 130° C. for 5 h. The mixture was concentrated under vacuum to afford crude product, which was purified by flash chromatography (elution gradient: petroleum ether/ethyl acetate, 5/1, v/v) to afford compound 38-5 (180 mg) as yellow solid, yield: 52.5%. LCMS: Rt=1.37 min, MS (ESI) m/z=351.0 [M+H]⁺.

### Step 5

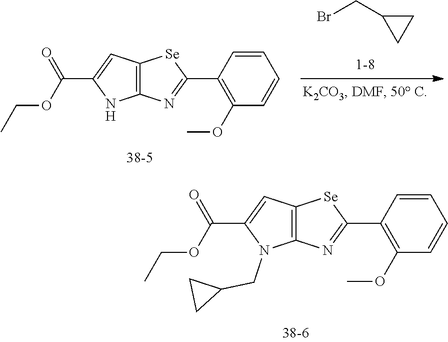 [SMILES: BrCC1CC1 ; CCOC(=O)c1cc2[se]c(-c3ccccc3OC)nc2[nH]1 ; CCOC(=O)c1cc2[se]c(-c3ccccc3OC)nc2n1CC1CC1]

Compound 38-6: To a solution of compound 38-5 (180 g, 0.51 mmol) in DMF (5 mL) was added compound 1-8 (104 mg, 0.78 mmol) and K₂CO₃ (214 mg, 1.55 mmol). The mixture was stirred at 80° C. for 5 h. The mixture was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 38-6 (90 mg) as a yellow oil, yield: 43.3%. LCMS: Rt=1.65 min, MS (ESI) m/z=405.0 [M+H]⁺.

### Step 6

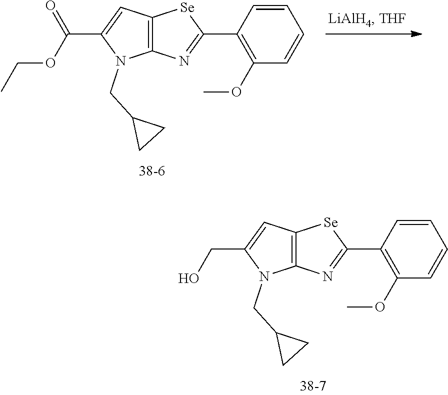

Compound 38-7: To a solution of compound 38-6 (90 mg, 0.22 mmol) in THE (5 mL) was added LiAlH₄ (1M in THF, 0.7 mL, 0.7 mmol). The mixture was stirred at room temperature for 2 h. The mixture was quenched with 2N sodium hydroxide aqueous solution (5 mL), filtered and extracted with ethyl acetate (30 mL×2). The organic layers were combined, dried over anhydrous sodium sulfate, filtered and concentrated under vacuum to afford compound 38-7 (50 mg) as a yellow oil, yield: 62.0%. LCMS: Rt=1.37 min, MS (ESI) m/z=363.1 [M+H]⁺.

### Step 7

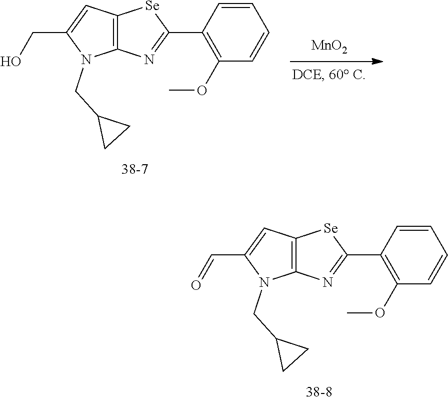

Compound 38-8: To a solution of compound 38-7 (50 mg, 0.14 mmol) in DCE (5 mL) was added MnO₂ (120 mg, 1.38 mmol). The mixture was stirred at 60° C. for 16 h. The mixture was filtered and concentrated under reduced pressure to afford compound 38-8 (45 mg) as a yellow oil, yield: 90.5%. LCMS: Rt=1.52 min, MS (ESI) m/z=360.9 [M+H]⁺.

### Step 8

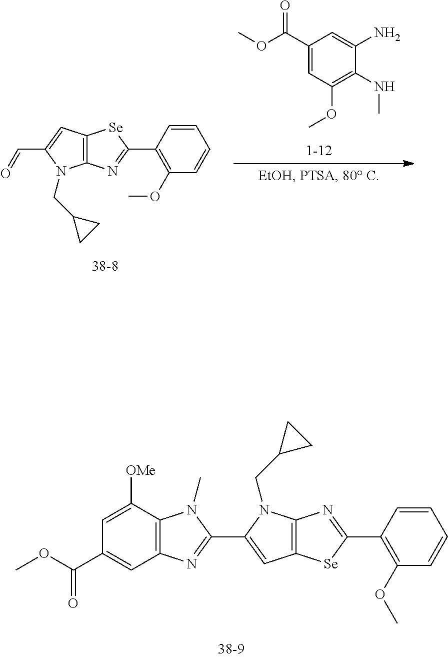

Compound 38-9: To a solution of compound 38-8 (45 mg, 0.125 mmol) in EtOH (5 mL) was added compound 1-12 (29 mg, 0.14 mmol) and PTSA (4.3 mg, 0.25 mmol). The mixture was stirred at 80° C. for 4 h. The mixture was concentrated under reduced pressure to compound 38-9 (100 mg, crude) as yellow solid. The crude product was used into next step without further purification. LCMS: Rt=1.59 min, MS (ESI) m/z=551.0 [M+H]⁺.

### Step 9

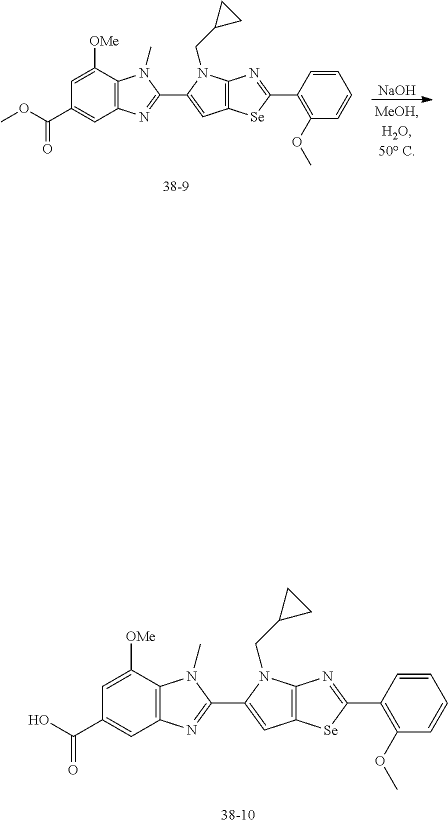

Compound 38-10: To a solution of compound 38-9 (100 mg, crude) in MeOH (5 mL) was added 2N NaOH (0.5 mL, 1 mmol). The mixture was stirred at 50° C. for 5 h. The mixture was concentrated and adjusted pH to 5-6 with 1N hydrochloric acid aqueous solution, then extracted with dichloromethane (20 mL×3). The organic phases were combined, dried over Na₂SO₄, filtered and concentrated under vacuum to give compound 38-10 (40 mg) as a yellow oil, yield: 59.7% (2 steps). LCMS: Rt=1.45 min, MS (ESI) m/z=536.9 [M+H]⁺.

### Step 10

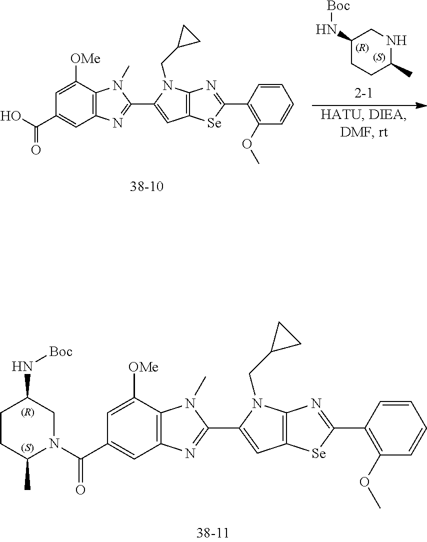

Compound 38-11: To a solution of compound 38-10 (40 mg, 0.075 mmol), DIEA (29 mg, 0.22 mol), HATU (37 mg, 0.097 mmol) in DMF (5 mL) was added compound 2-1 (18 mg, 0.084 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 16 (36 mg) as a yellow oil, yield: 65.9%. LCMS: Rt=1.55 min, MS (ESI) m/z=733.0 [M+H]⁺.

### Step 11

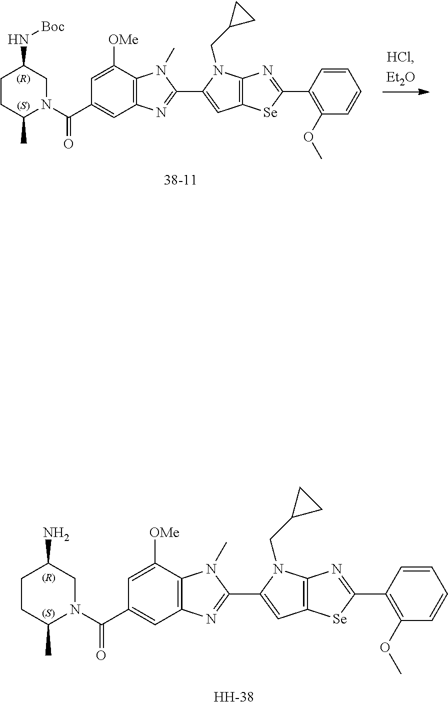

HHBP-9459: To a solution of compound 38-11 (36 mg, 0.057 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-38 (16.3 mg, hydrochloric acid salt form) as a yellow solid. yield: 49.7%. ¹H NMR (400 MHz, DMSO-d₆): δ ppm 8.37-8.33 (m, 4H), 7.54-7.49 (m, 1H), 7.37 (s, 1H), 7.32-7.30 (m, 1H), 7.19-7.15 (m, 2H), 6.97 (s, 1H), 4.50 (d, J=7.2 Hz, 2H), 4.18 (s, 3H), 4.10 (s, 3H), 4.03 (s, 3H), 3.17-3.09 (m, 2H), 1.91-1.78 (m, 3H), 1.59-1.51 (m, 1H), 1.23-1.21 (m, 5H), 0.38-0.37 (m, 2H), 0.30-0.26 (m, 2H). LCMS: Rt=1.17 min, MS (ESI) m/z=633.0 [M+H]⁺. HPLC: Rt=6.07 min, Purity: 100% (214 nm and 254 nm)

### Example 39. Synthesis of HH-39

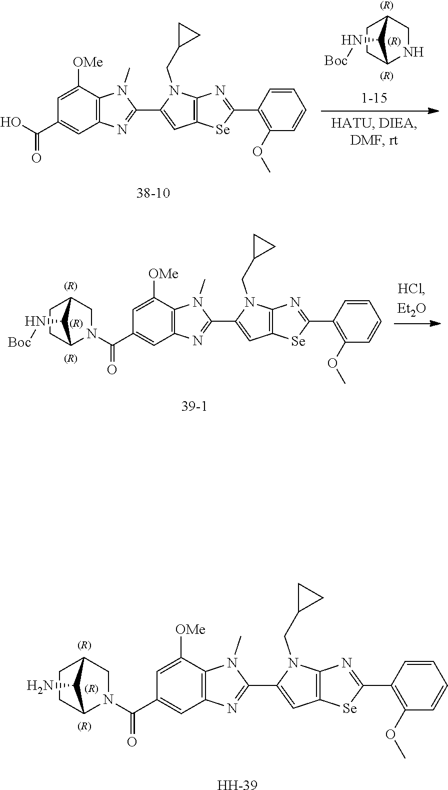

### Step 1

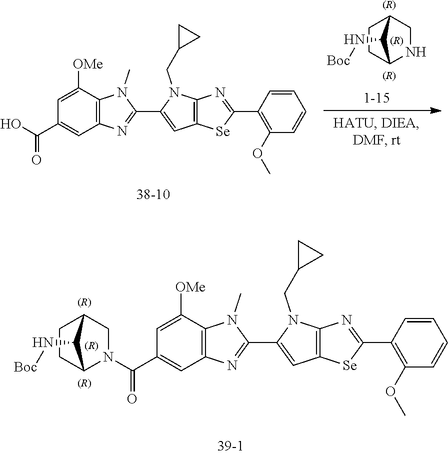

Compound 39-1: To a solution of compound 38-10 (55 mg, 0.10 mmol), DIEA (40 mg, 0.31 mol), HATU (51 mg, 0.13 mmol) in DMF (5 mL) was added compound 1-15 (24 mg, 0.11 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 39-1 (68 mg) as a yellow solid, yield: 90.7%. LCMS: Rt=1.51 min, MS (ESI) m/z=731.0 [M+H]⁺.

### Step 2

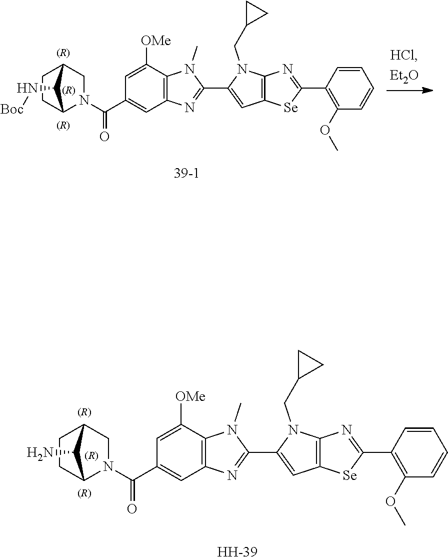

HH-39: To a solution of compound 39-1 (68 mg, 0.093 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-39 (41.6 mg, hydrochloric acid salt form) as a brown solid. yield: 67.1%. ¹H NMR (400 MHz, DMSO-d₆): δ ppm 8.51-8.33 (m, 4H), 7.49-7.44 (m, 2H), 7.29-7.27 (m, 1H), 7.14 (t, J=7.6 Hz, 1H), 7.06 (s, 1H), 7.00 (s, 1H), 4.51 (d, J=6.8 Hz, 2H), 4.15 (s, 3H), 4.09 (s, 3H), 4.02 (s, 3H), 3.66-3.64 (m, 1H), 3.52 (m, 1H), 3.19 (d, J=10.0 Hz, 1H), 2.67 (s, 1H), 2.02-1.90 (m, 3H), 1.63-1.61 (m, 1H), 1.26-1.25 (m, 1H), 0.38-0.36 (m, 2H), 0.28-0.27 (m, 2H). LCMS: Rt=1.27 min, MS (ESI) m/z=631.0 [M+H]⁺. HPLC: Rt=5.94 min, Purity: 100% (214 nm and 254 nm)

### Example 40. Synthesis of HH-40

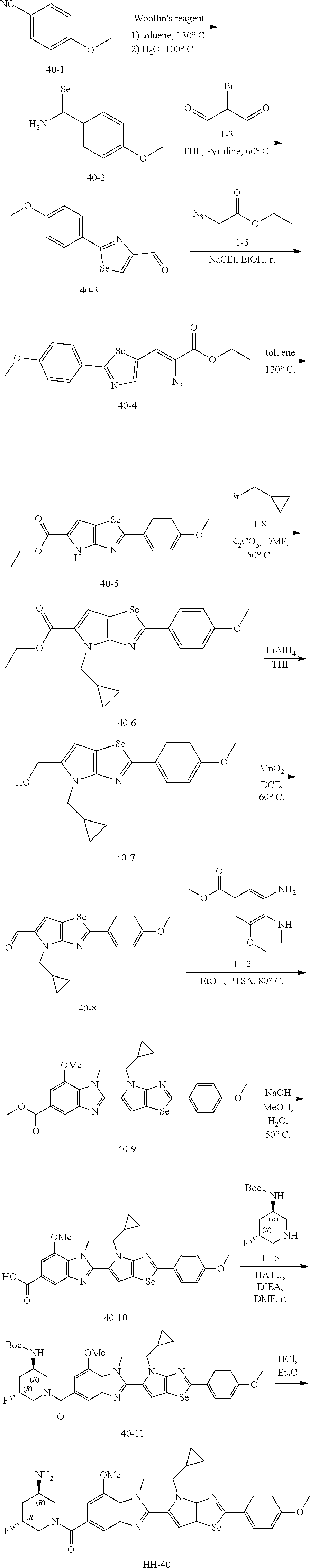

### Step 1

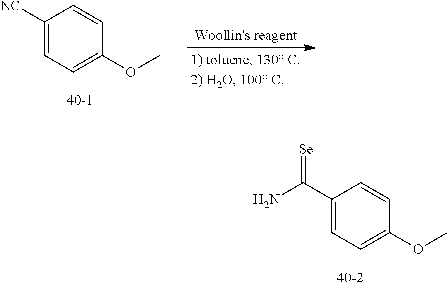

Compound 40-2: To a solution of 4-methoxybenzonitrile (800 mg, 6.02 mmol) in Toluene (20 mL) was added Woollin's reagent (1.6 g, 3.01 mmol). The reaction mixture was stirred at 130° C. for 3 h. To this H₂O (1 mL) was added at 90° C. The resulting mixture was stirred at 90° C. for further 1 h. The mixture was concentrated under vacuum to afford crude product, which was purified by silica gel chromatography (elution gradient: petroleum ether/EtOAc, 1/1, v/v) to afford compound 40-2 (650 mg) as a brown solid, yield: 50.3%. LCMS: Rt=1.065 min, MS (ESI) m/z=216.0 [M+H]⁺.

### Step 2

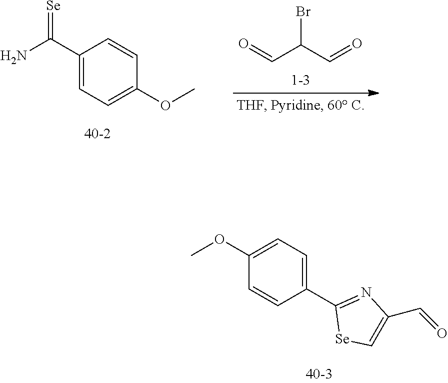

Compound 40-3: To a solution of compound 40-2 (650 mg, 3.04 mmol) in THF (10 mL) was added 2-bromopropanedial (458 mg, 3.04 mmol) and pyridine (480 mg, 6.07 mmol). The reaction mixture was stirred at 60° C. for 16 h. The solvent was evaporated and diluted with water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 40-3 (520 mg) as a yellow solid, yield: 64.4%. LCMS: Rt=1.261 min, MS (ESI) m/z=267.9 [M+H]⁺.

### Step 3

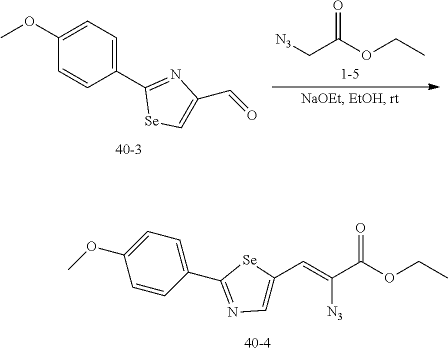

Compound 40-4: To a solution of sodium ethoxide (20% wt. in EtOH) (2.0 g, 5.84 mmol) in EtOH (20 mL) was added compound 40-3 (520 mg, 1.95 mmol) and compound 1-5 (753 mg, 5.84 mmol) in EtOH (50 mL) at 0° C. under N₂. The reaction mixture was stirred at 0° C. for 1 h and 25° C. for 16 h. The solvent was evaporated and diluted with ice water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 6 (300 mg) as a yellow solid, yield: 40.8%. LCMS: Rt=1.509 min, MS (ESI) m/z=378.9 [M+H]⁺.

### Step 4

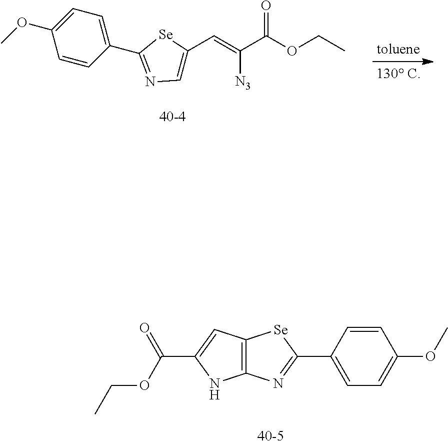

Compound 40-5: A solution of compound 40-4 (300 mg, 0.80 mmol) in toluene (10 mL) was stirred at 130° C. for 2 h. The mixture was concentrated under vacuum to give the crude product, which was purified by silica gel chromatography (elution gradient: petroleum ether/EtOAc, 5/1, v/v) to afford compound 40-5 (100 mg) as a yellow solid, yield: 36.1%. LCMS: Rt=1.379 min, MS (ESI) m/z=350.9 [M+H]⁺.

### Step 5

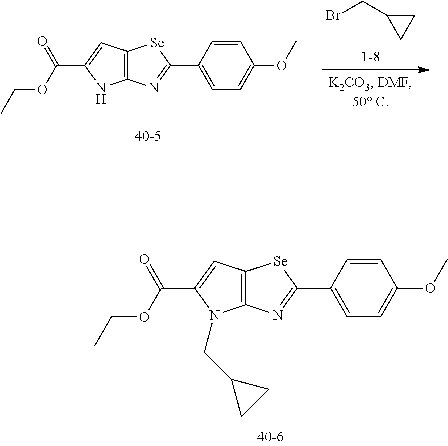

Compound 40-6: To a solution of compound 40-5 (100 mg, 0.29 mmol) in DMF (5 mL) was added (bromomethyl)cyclopropane (58 mg, 0.43 mmol) and K₂CO₃ (79 mg, 0.57 mmol). The reaction mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 40-6 (80 mg) as a yellow solid, yield: 69.6%. LCMS: Rt=1.647 min, MS (ESI) m/z=405.1 [M+H]⁺.

### Step 6

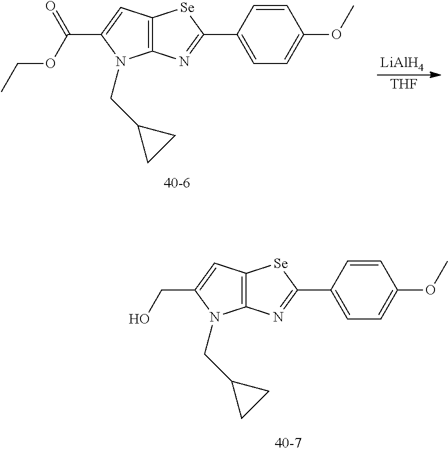 [SMILES: CCOC(=O)c1cc2[se]c(-c3ccc(OC)cc3)nc2n1CC1CC1 ; "LiAlH4" ; COc1ccc(-c2nc3c(cc(CO)n3CC3CC3)[se]2)cc1 ; "THF"]

Compound 40-7: To a solution of compound 40-6 (80 mg, 0.20 mmol) in THF (5 mL), then was added LiAlH₄ (1M in THF, 0.6 mL, 0.59 mmol) at 0° C. The reaction mixture was stirred at 25° C. for 2 h. The mixture was quenched with ice water (20 mL) and extracted with ethyl acetate (20 mL×3). The organic layers were dried over anhydrous Na₂SO₄ and concentrated under vacuum to afford crude product (70 mg) as a yellow solid. LCMS: Rt=1.376 min, MS (ESI) m/z=362.9 [M+H]⁺.

### Step 7

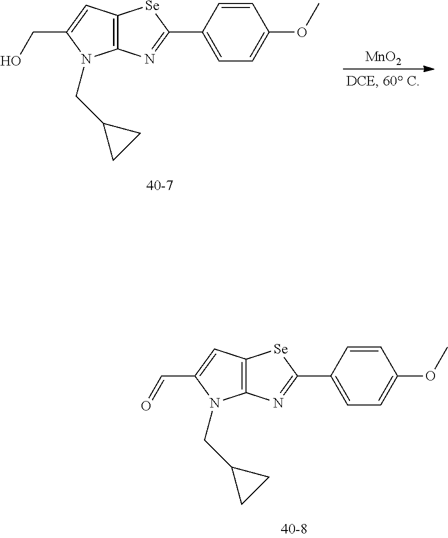

Compound 40-8: To a solution of compound 40-7 (70 mg, 0.19 mmol) in DCE (5 mL), then was added MnO₂ (168 mg, 1.93 mmol). The reaction mixture was stirred at 60° C. for 16 h. The mixture was filtered and the filtrate was concentrated under vacuum to afford crude product (70 mg) as a brown solid. LCMS: Rt=1.512 min, MS (ESI) m/z=361.0 [M+H]⁺.

### Step 8

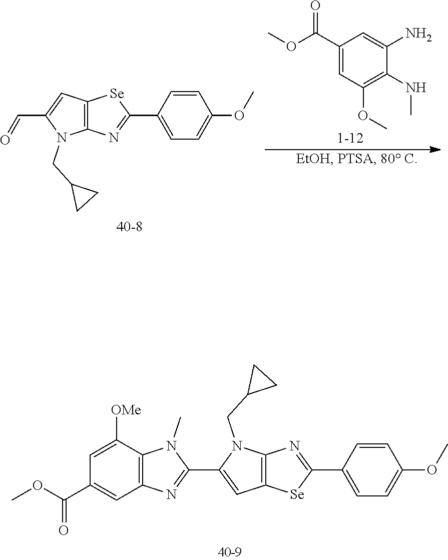

Compound 40-9: To a solution of compound 40-8 (70 mg, 0.19 mmol) in EtOH (10 mL) was added compound 1-12 (41 mg, 0.19 mmol) and PTSA (3.4 mg, 0.019 mmol). The reaction mixture was stirred at 80° C. for 16 h. The mixture was concentrated under vacuum to give the crude product, which was purified by silica gel chromatography (elution gradient: petroleum ether/EtOAc, 3/1, v/v) to afford compound 40-9 (30 mg) as a yellow gum, yield: 28.6%. LCMS: Rt=1.589 min, MS (ESI) m/z=551.0 [M+H]⁺.

### Step 9

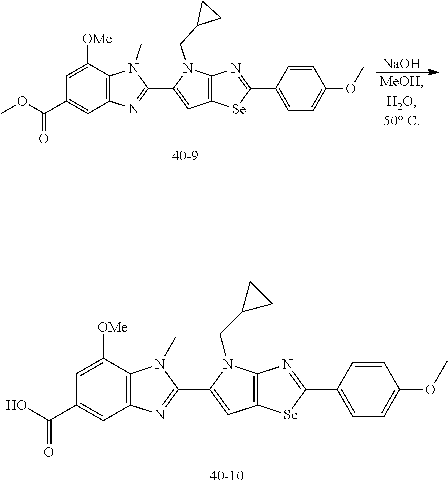

Compound 40-10: To a solution of compound 40-9 (30 mg, 0.055 mmol) in MeOH/H₂O (5/1 mL) was added NaOH (6.55 mg, 0.16 mmol). The reaction mixture was stirred at 50° C. for 16 h. The mixture was concentrated and adjusted pH to 5-6 with hydrochloric acid aqueous solution (2 N), then extracted with ethyl acetate (20 mL×3). The organic phase was washed with water (20 mL), brine (20 mL), dried over anhydrous Na₂SO₄ and concentrated under vacuum to give crude product (30 mg) as a yellow solid. LCMS: Rt=1.448 min, MS (ESI) m/z=536.9 [M+H]⁺.

### Step 10

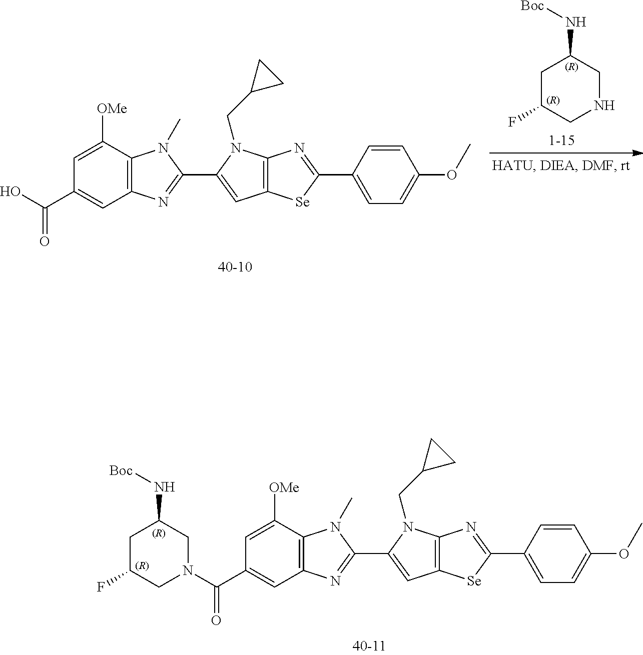

Compound 40-11: To a solution of compound 40-10 (30 mg, 0.056 mmol) in DMF (5 mL) was compound 1-15 (12.3 mg, 0.056 mmol), HATU (21.3 mg, 0.056 mmol) and DIEA (14.2 mg, 0.11 mmol), The reaction mixture was stirred at room temperature for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 40-11 (30 mg) as a yellow solid, yield: 75.0%. LCMS: Rt=1.489 min, MS (ESI) m/z=737.2 [M+H]⁺.

### Step 11

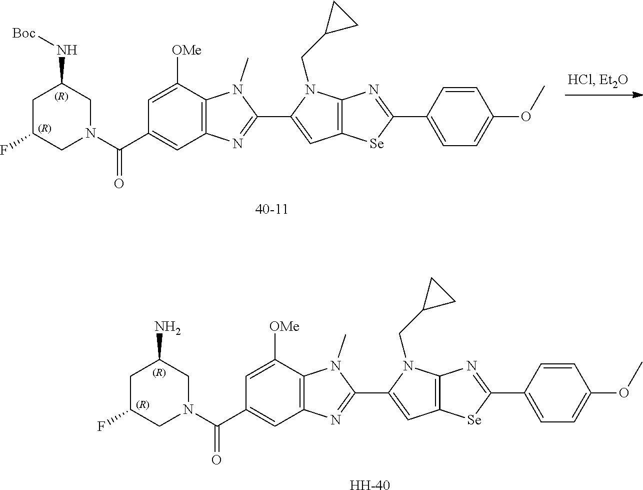

HH-40: To a solution of compound 40-11 (30 mg, 0.041 mmol) in Et₂O (5 mL) was added 2M HCl in Et₂O (6 mL, 12 mmol). The reaction mixture was stirred at 25° C. for 2 h. The mixture was concentrated under vacuum to afford crude product, which was purified by preparative HPLC [(Gemini-C18 column, 5μ silica, 21 mm diameter, 150 mm length), using decreasingly polar mixtures of water (containing 0.1% FA) and MeCN as eluents (30-70%)] to afford HH-40 (3 mg, hydrochloric acid salt form) as an off-white solid, yield: 11.5%. ¹H NMR (400 MHz, DMSO-d₆) δ ppm 8.28 (br. s, 3H), 7.89 (d, J=8.6 Hz, 2H), 7.35 (s, 1H), 7.10-7.05 (m, 3H), 6.89 (s, 1H), 5.11-4.75 (m, 2H), 4.48 (d, J=6.8 Hz, 2H), 4.15 (s, 3H), 3.99 (s, 4H), 3.84 (s, 4H), 3.12-3.00 (m, 1H), 2.43-2.31 (m, 1H), 1.99-1.81 (m, 1H), 0.88-0.80 (m, 1H), 0.41-0.37 (m, 2H), 0.32-0.28 (m, 2H). LCMS: Rt=1.271 min, MS (ESI) m/z=637.2 [M+H]⁺. HPLC: Rt=6.39 min, Purity: 100% (214 nm) and 100% (254 nm).

### Example 41. Synthesis of HH-41

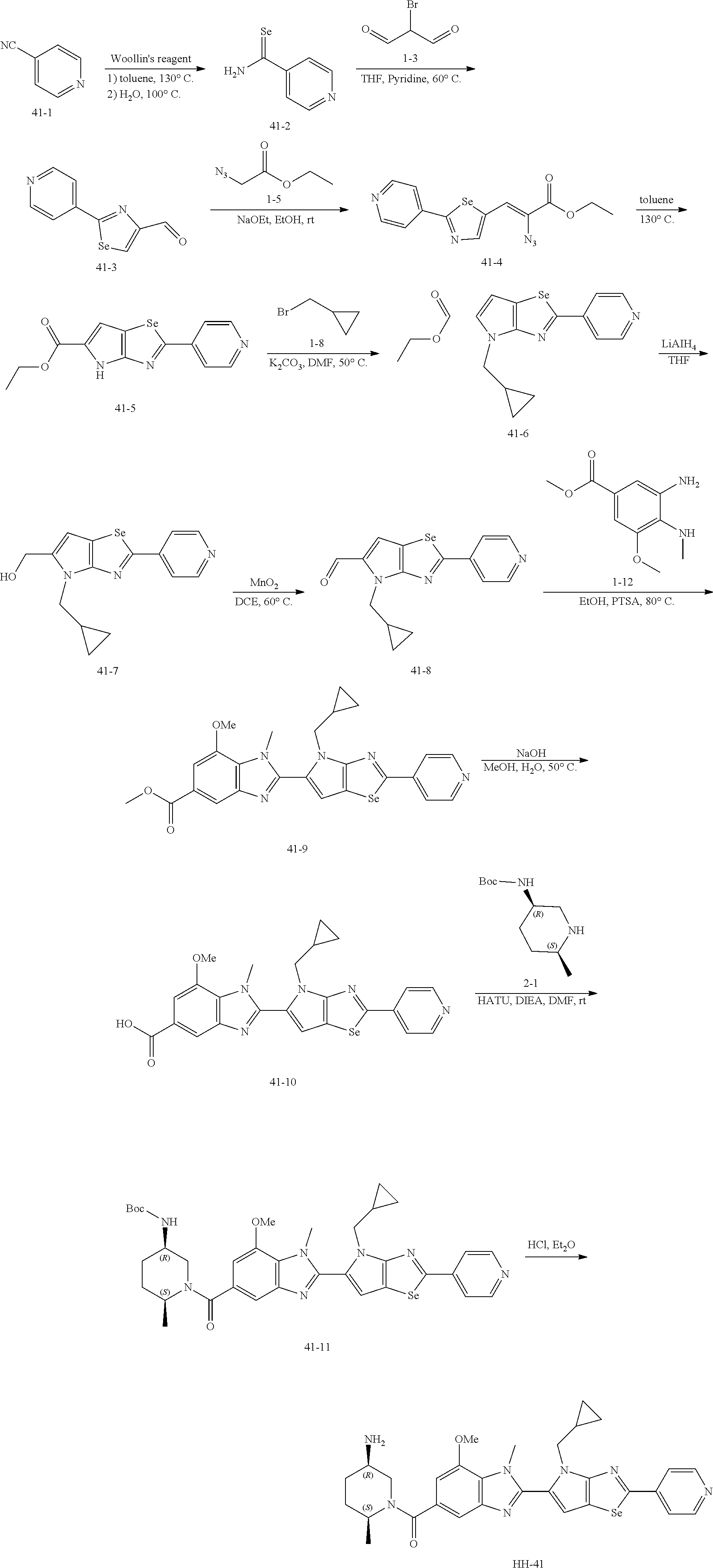

### Step 1

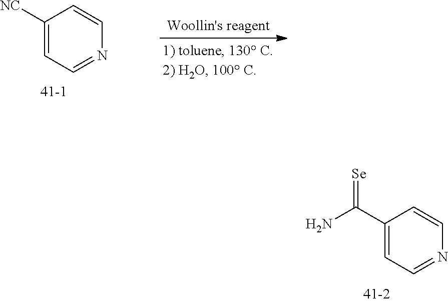 [SMILES: N#Cc1ccncc1 ; NC(=[Se])c1ccncc1]

Compound 41-2: To a solution of pyridine-4-carbonitrile (6.0 g, 57.6 mmol) in DMF/H₂O (50/5 mL) was added Se powder (5.5 g, 69.2 mmol). The reaction mixture was stirred at 90° C. under CO atmosphere for 5 h. The mixture was diluted with water (100 mL), extracted with EtOAc (3×100 mL). The organic layers were washed with brine (100 mL), dried over anhydrous Na₂SO₄ and concentrated under vacuum to afford crude product, which was purified by silica gel chromatography (elution gradient: EtOAc) to afford compound 41-2 (3.5 g) as a brown solid, yield: 32.7%. LCMS: Rt=0.343 min, MS (ESI) m/z=187.1 [M+H]⁺.

### Step 2

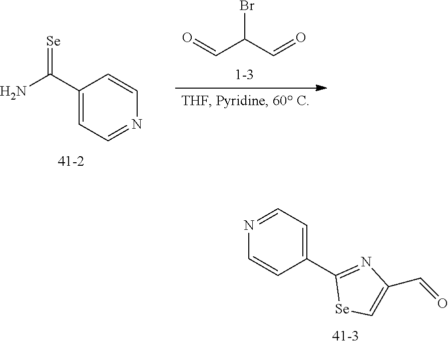 [SMILES: NC(=[Se])c1ccncc1 ; O=CC(Br)C=O ; O=Cc1c[se]c(-c2ccncc2)n1]

Compound 41-3: To a solution of compound 41-2 (3 g, 16.1 mmol) in THF (20 mL) was added 2-bromopropanedial (2.4 g, 16.3 mmol) and pyridine (2.5 g, 32.3 mmol). The reaction mixture was stirred at 60° C. for 16 h. The solvent was evaporated and diluted with water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: EtOAc) to afford compound 41-3 (1 g) as a yellow solid, yield: 26.3%. LCMS: Rt=0.923 min, MS (ESI) m/z=238.9 [M+H]⁺.

### Step 3

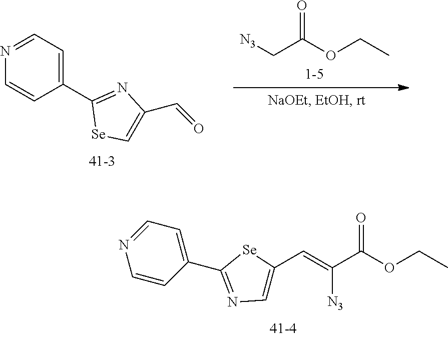

Compound 41-4: To a solution of sodium ethoxide (20% wt. in EtOH) (4.3 g, 12.65 mmol) in EtOH (50 mL) was slowly added compound 41-3 (1.0 g, 4.22 mmol) and compound 1-5 (1.6 g, 12.65 mmol) in EtOH (50 mL) at −15° C. under N₂. The reaction mixture was stirred at −15° C. for 1 h and 0° C. for further 1 h. The mixture was quenched with ice water (50 mL) and extracted with ethyl acetate (50 mL×3). The organic layers were washed with water (100 mL), brine (100 mL), dried over anhydrous Na₂SO₄ and concentrated under vacuum to afford crude product, which was purified by silica gel chromatography (EtOAc) to afford compound 41-4 (400 mg) as a yellow oil, yield: 27.2%. LCMS: Rt=1.215 min, MS (ESI) m/z=350.0 [M+H]⁺.

### Step 4

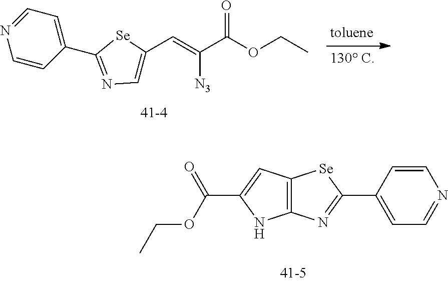

Compound 41-5: A solution of compound 41-4 (400 mg, 1.15 mmol) in toluene (20 mL) mixture was stirred at 130° C. for 2 h. The reaction mixture was concentrated under vacuum to give the crude product, which was purified by silica gel chromatography (elution gradient: petroleum ether/EtOAc, 1/1, v/v) to afford compound 41-5 (300 mg) as yellow oil, yield: 81.5%. LCMS: Rt=1.190 min, MS (ESI) m/z=322.0 [M+H]⁺.

### Step 5

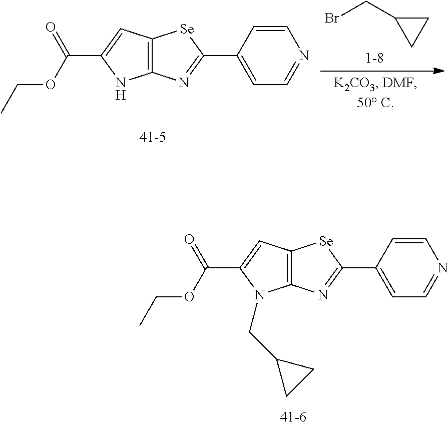 [SMILES: CCOC(=O)c1cc2[se]c(-c3ccncc3)nc2[nH]1 ; BrCC1CC1 ; CCOC(=O)c1cc2[se]c(-c3ccncc3)nc2n1CC1CC1]

Compound 41-6: To a solution of compound 41-5 (300 mg, 0.93 mmol) and compound 1-8 (150.28 mg, 1.12 mmol) in DMF (10 mL) was added potassium carbonate (385.02 mg, 2.79 mmol). The mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 1/1, v/v) to afford compound 41-6 (300 mg) as a yellow oil, yield: 85.7%. LCMS: Rt=1.461 min, MS (ESI) m/z=375.9 [M+H]⁺.

### Step 6

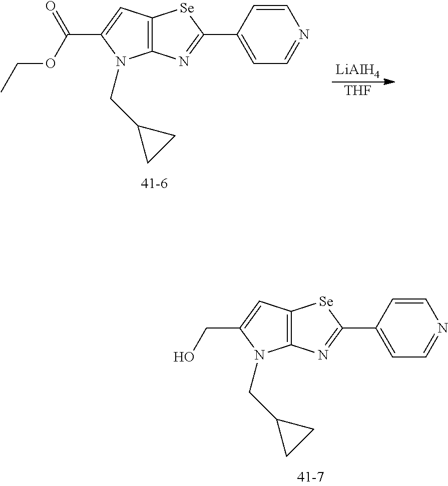

Compound 41-7: To a solution of compound 41-6 (300 mg, 0.80 mmol) in THF (5 mL), then was added LiAlH₄ (1M in THF, 2.54 mL, 2.40 mmol) at 0° C., The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product (200 mg) as a yellow oil. LCMS: Rt=0.993 min, MS (ESI) m/z=334.1 [M+H]⁺.

### Step 7

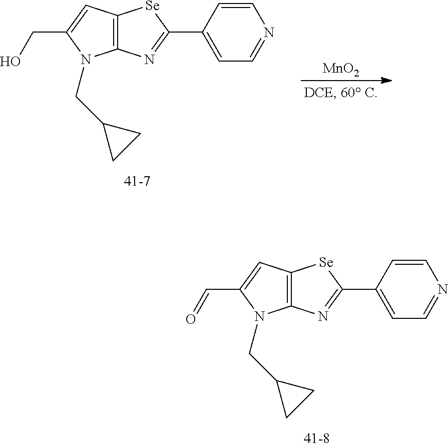

Compound 41-8: To a solution of compound 41-7 (200 mg, 0.60 mmol) in DCE (10 mL) was added MnO₂ (262 mg, 3.01 mmol). The reaction mixture was stirred at 60° C. for 16 h. The mixture was filtered and the filtrate was concentrated under vacuum to afford crude product (180 mg) as a brown oil. LCMS: Rt=1.248 min, MS (ESI) m/z=331.9 [M+H]⁺

### Step 8

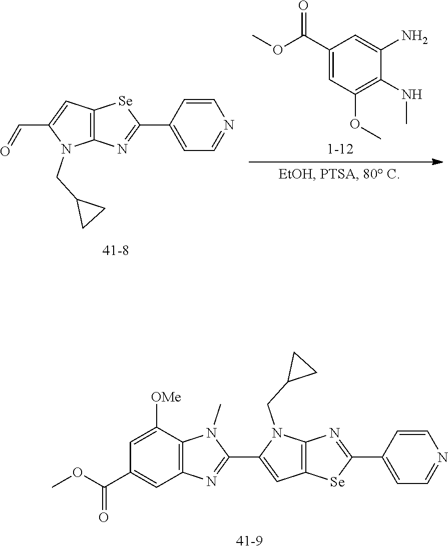

Compound 41-9: To a solution of compound 41-8 (170 mg, 0.51 mmol) in EtOH (10 mL) was added compound 1-12 (108 mg, 0.51 mmol) and PTSA (8.8 mg, 0.05 mmol), The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 41-9 (150 mg) as a yellow oil, yield: 53.0%. LCMS: Rt=1.352 min, MS (ESI) m/z=522.0 [M+H]⁺.

### Step 9

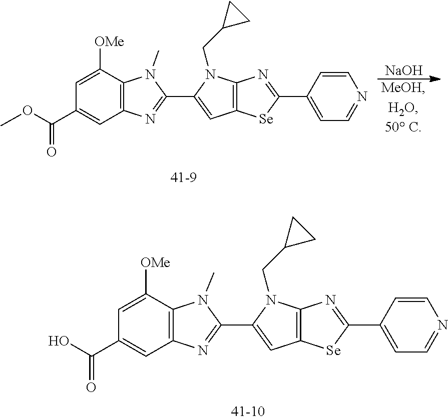

Compound 41-10: To a solution of compound 41-9 (150 mg, 0.29 mmol) in MeOH/H₂O (5/1 mL) was added NaOH (58 mg, 1.44 mmol), The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 41-10 (100 mg) as a yellow solid, yield: 68.5%. LCMS: Rt=1.187 min, MS (ESI) m/z=507.9 [M+H]⁺.

### Step 10

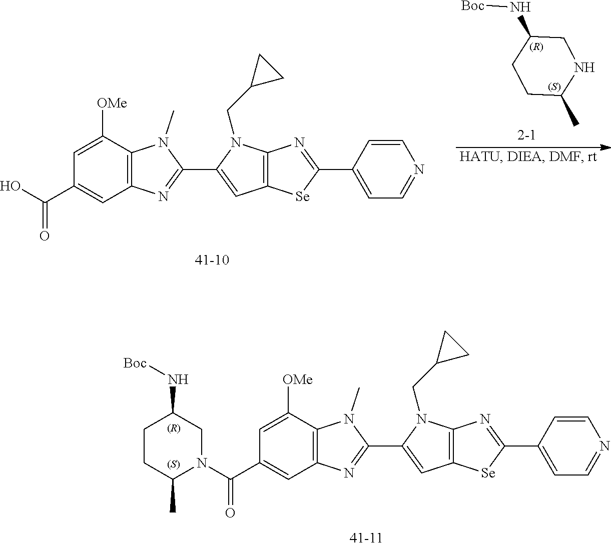

Compound 41-11: To a solution of compound 41-10 (50 mg, 0.099 mmol) in DMF (5 mL) was added compound 2-1 (25.5 mg, 0.12 mmol), HATU (37.5 mg, 0.099 mmol) and DIEA (25.5 mg, 0.20 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1, v/v) to afford compound 41-11 (30 mg) as a brown solid, yield: 43.5%. LCMS: Rt=1.341 min, MS (ESI) m/z=704.0 [M+H]⁺.

### Step 11

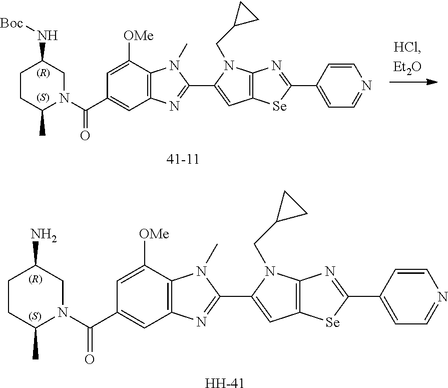

HH-41: To a solution of compound 41-11 (30 mg, 0.043 mmol) in Et₂O (5 mL) was added 2M HCl in Et₂O (6 mL, 12 mmol). The reaction mixture was stirred at 25° C. for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 10-30%) to afford HH-41 (11.2 mg, hydrochloric acid salt form) as a yellow solid. yield: 43.6%. ¹H NMR (400 MHz, DMSO-d₆) δ ppm 8.79-8.71 (m, 2H), 8.17-8.05 (m, 2H), 8.04-7.80 (m, 3H), 7.31-7.28 (m, 1H), 7.14-7.08 (m, 1H), 6.84 (s, 1H), 4.51-4.41 (m, 3H), 4.12 (s, 3H), 3.92 (s, 3H), 3.20-3.08 (m, 1H), 3.02-2.84 (m, 1H), 2.51-2.47 (m, 1H), 1.88-1.82 (m, 1H), 1.79-1.64 (m, 2H), 1.59-1.49 (m, 1H), 1.32-1.22 (m, 1H), 1.26 (t, J=5.8 Hz, 3H), 0.42-0.34 (m, 2H), 0.32-0.22 (m, 2H). LCMS: Rt=1.137 min, MS (ESI) m/z=604.0 [M+H]⁺. HPLC: Rt=4.67 min, Purity: 100% (214 nm) and 100% (254 nm).

### Example 42. Synthesis of HH-42

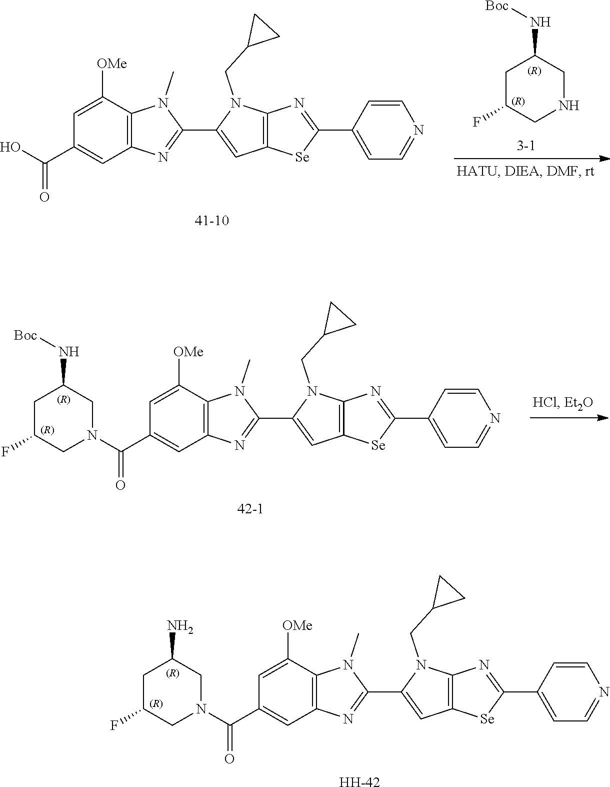

### Step 1

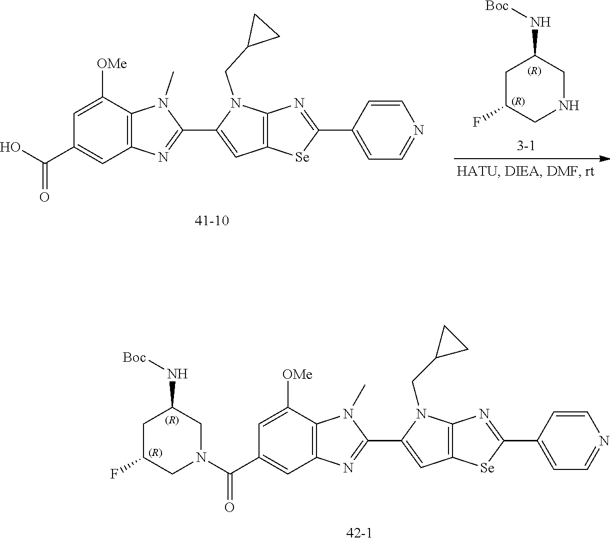

Compound 42-1: To a solution of compound 41-10 (50 mg, 0.099 mmol) in DMF (5 mL) was compound 3-1 (21.6 mg, 0.099 mmol), HATU (37.5 mg, 0.099 mmol) and DIEA (25.5 mg, 0.20 mmol), The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1, v/v) to afford compound 42-1 (30 mg) as a brown solid, yield: 47.5%. LCMS: Rt=1.287 min, MS (ESI) m/z=708.0 [M+H]⁺.

### Step 2

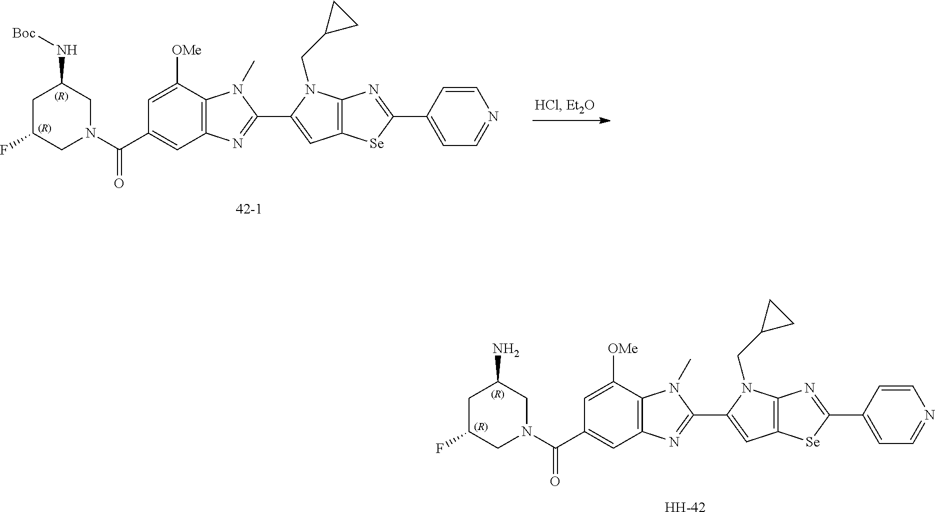

HH-42: To a solution of compound 42-1 (30 mg, 0.042 mmol) in Et₂O (5 mL) was added 2M HCl in Et₂O (6 mL, 12 mmol). The reaction mixture was stirred at 25° C. for 2 h. The mixture was concentrated under vacuum to afford crude product, which was purified by preparative HPLC (Gemini-C18 column, 5μ silica, 21 mm diameter, 150 mm length), using decreasingly polar mixtures of water (containing 0.05% FA) and MeCN as eluents 20-40%) to afford HH-42 (7.9 mg, hydrochloric acid salt form) as a yellow solid. yield: 30.7%. ¹H NMR (400 MHz, DMSO-d₆) δ ppm 8.87 (br. s, 2H), 8.63-8.47 (m, 3H), 8.42-8.34 (m, 2H), 7.38 (s, 1H), 7.20 (s, 1H), 6.92 (s, 1H), 4.52 (d, J=7.0 Hz, 2H), 4.16 (s, 3H), 4.00 (s, 3H), 3.36-3.34 (m, 2H), 3.22-3.09 (m, 4H), 2.54 (s, 1H), 2.18-2.12 (m, 1H), 2.04-1.99 (m, 1H), 1.35-1.29 (m, 1H), 0.44-0.39 (m, 2H), 0.34-0.30 (m, 2H). LCMS: Rt=1.081 min, MS (ESI) m/z=608.0 [M+H]⁺. HPLC: Rt=4.561 min, Purity: 96.4% (214 nm) and 96.3% (254 nm).

### Example 43. Synthesis of HH-43

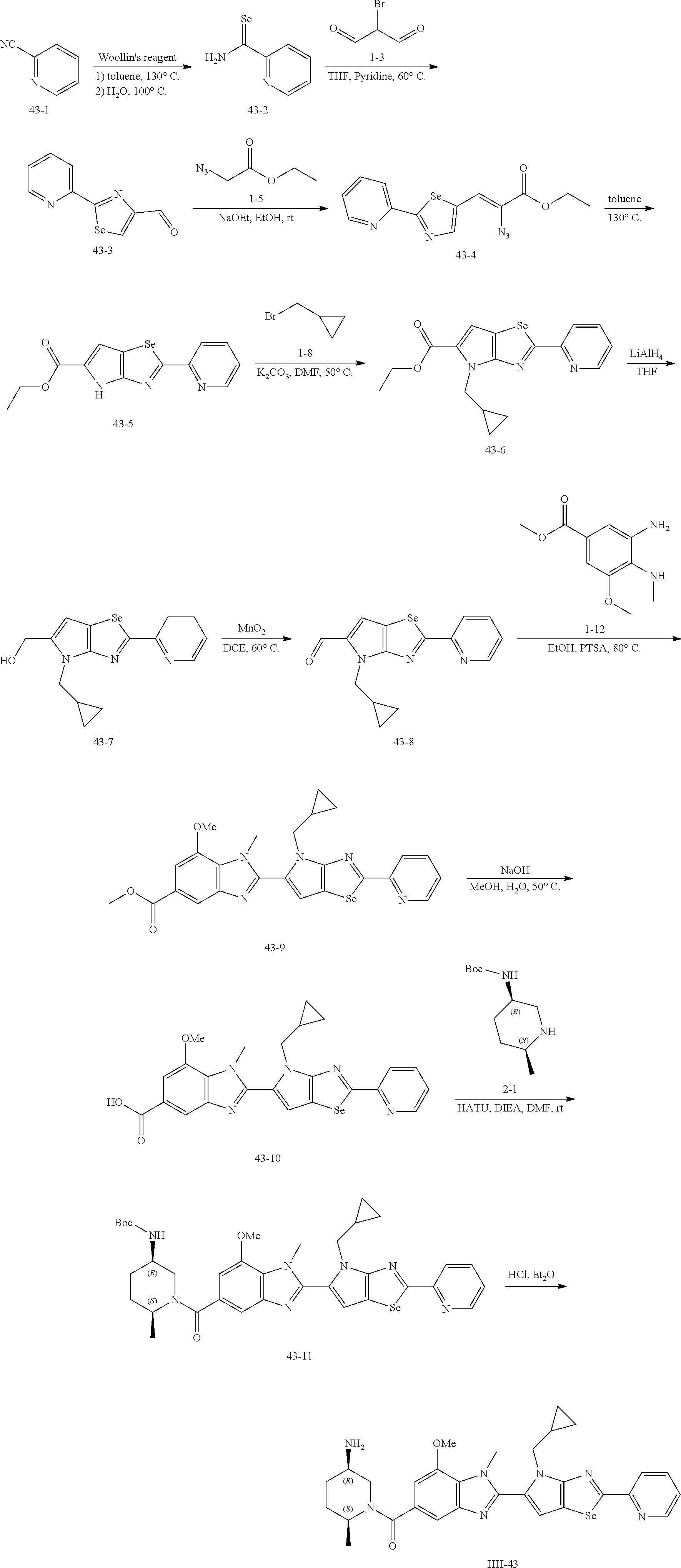

### Step 1

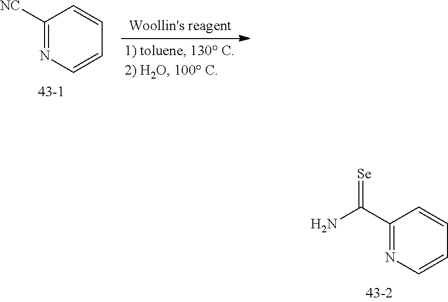 [SMILES: N#Cc1ccccn1 ; NC(=[Se])c1ccccn1]

Compound 43-2: To a solution of compound 43-1 (6 g, 57.6 mmol) in DMF (60 mL) and H₂O (60 mL) was added Selenium (5.46 g, 69.1 mmol). The mixture was stirred at 90° C. under CO for 12 h. The mixture was filtered, the filtrate was diluted with water (200 mL) and extracted with ethyl acetate (200 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 43-2 (9 g) as a yellow solid, yield: 76.4%. LCMS: Rt=1.00 min, MS (ESI) m/z=186.9 [M+H]⁺.

### Step 2

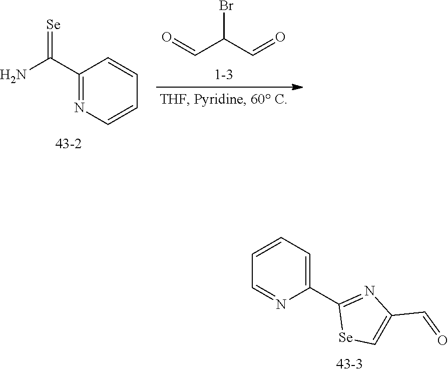

Compound 3: To a solution of compound 43-2 (8 g, 43.2 mmol) and compound 1-3 (6.52 g, 43.2 mmol) in THF (100 mL) was added pyridine (6.84 g, 86.4 mmol). The mixture was stirred at 60° C. for 12 h. The mixture was filtered, the filtrate was diluted with water (100 mL) and extracted with dichloromethane (200 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 43-3 (7 g) as a yellow solid, yield: 61.5%. LCMS: Rt=1.18 min, MS (ESI) m/z=238.9 [M+H]⁺.

### Step 3

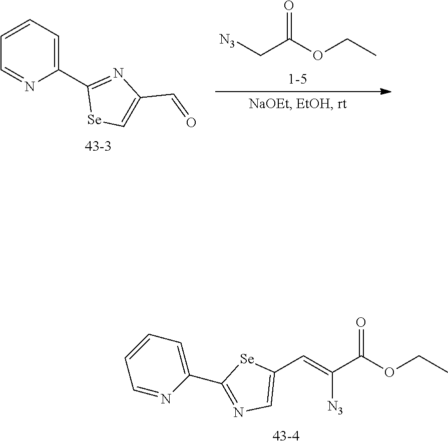

Compound 43-4: To a solution of sodium ethoxide (20% wt. in EtOH) (32.8 g, 94.8 mmol) in EtOH (100 mL) was slowly added the mixture of compound 43-3 (7.5 g, 31.6 mmol) and compound 1-5 (12.2 g, 94.8 mmol) in EtOH (100 mL) at −10° C. The mixture was stirred at −10° C. under N₂ for 1 h and at room temperature for further 2 h. The mixture was quenched with saturated NH₄Cl aqueous solution (100 mL) and extracted with ethyl acetate (150 mL×3). The organic layers were washed with water (100 mL), brine (100 mL), dried over anhydrous Na₂SO₄ and concentrated under vacuum to afford crude product, which was purified by silica gel chromatography (elution gradient: petroleum ether/EtOAc, 10/1, v/v) to afford compound 43-4 (4 g) as a yellow solid, yield: 32.7%. LCMS: Rt=1.46 min, MS (ESI) m/z=349.9 [M+H]⁺.

### Step 4

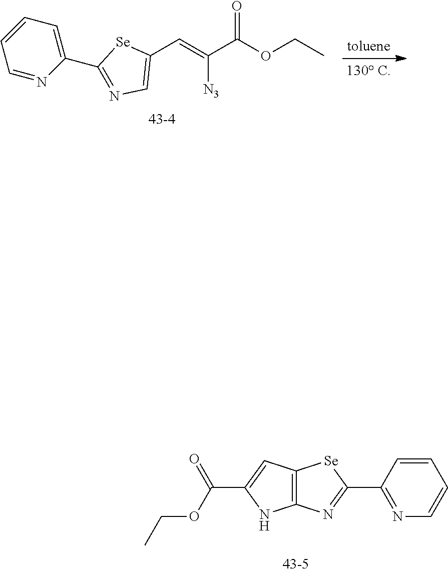

Compound 43-5: A solution of compound 43-4 (4 g, 11.5 mmol) in toluene (100 mL) was stirred at 130° C. under N₂ for 16 h. The reaction mixture was cooled to room temperature and concentrated under vacuum to give the crude product, which was purified by silica gel chromatography (elution gradient: petroleum ether/EtOAc, 10/1, v/v) to afford compound 43-5 (3.2 g) as yellow oil, yield: 78.3%. LCMS: Rt=1.31 min, MS (ESI) m/z=322.0 [M+H]⁺.

### Step 5

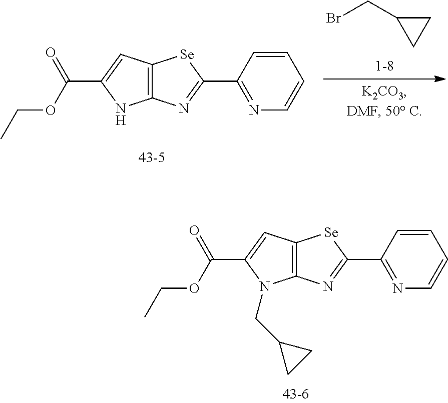 [SMILES: BrCC1CC1 ; CCOC(=O)c1cc2[se]c(-c3ccccn3)nc2[nH]1 ; CCOC(=O)c1cc2[se]c(-c3ccccn3)nc2n1CC1CC1]

Compound 43-6: To a solution of compound 43-5 (3.2 g, 9.99 mmol) and compound 1-8 (2.02 g, 14.9 mmol) in DMF (50 mL) was added potassium carbonate (2.76 g, 19.9 mmol). The mixture was stirred at 80° C. for 3 h. The mixture was diluted with water (100 mL) and extracted with ethyl acetate (100 mL×3). The organic layers were washed with water (100 mL) and brine (100 mL), dried over anhydrous Na₂SO₄ and concentrated under vacuum to afford crude product, which was purified by silica gel chromatography (elution gradient: petroleum ether/EtOAc, 4/1, v/v) to afford compound 43-6 (2 g) as a yellow solid, yield: 48.1%. LCMS: Rt=1.61 min, MS (ESI) m/z=376.1 [M+H]⁺.

### Step 6

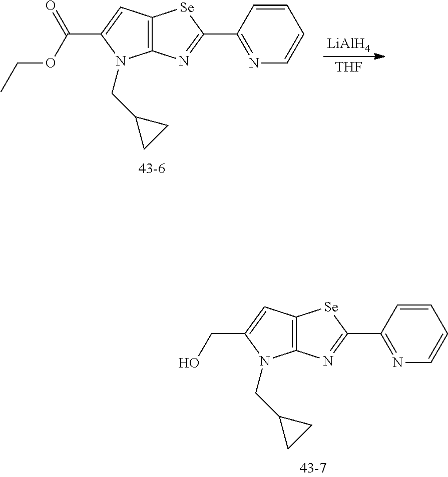

Compound 43-7: To a solution of compound 43-6 (2 g, 5.34 mmol) in THF (40 mL) was added LiAlH₄ (1 M in THF, 16 mL, 16.0 mmol) dropwise at 0° C. The mixture was stirred at 25° C. for 2 h. The mixture was quenched with 2N sodium hydroxide aqueous solution (50 mL) The mixture was extracted with ethyl acetate (50 mL×3). The organic layers were dried over anhydrous Na₂SO₄ and concentrated under vacuum to afford compound 43-7 (1.6 g) as a brown solid, yield: 81.1%. LCMS: Rt=1.29 min, MS (ESI) m/z=334.0 [M+H]⁺.

### Step 7

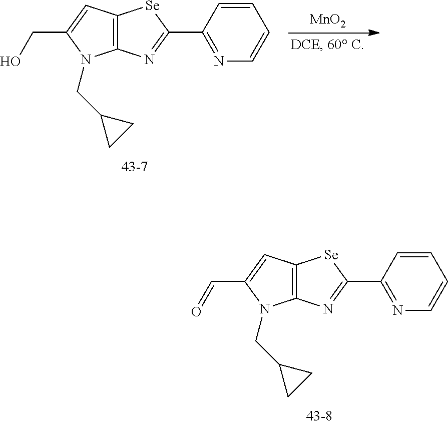

Compound 43-8: To a solution of compound 43-7 (1.6 g, 4.82 mmol) in DCE (50 mL) was added manganese dioxide (4.19 g, 48.1 mmol). The mixture was stirred at 60° C. for 16 h. The mixture was filtered and the filtrate was concentrated under vacuum to afford crude product, which was purified by silica gel chromatography (elution gradient: petroleum ether/EtOAc, 5/1, v/v) to afford compound 43-8 (0.7 g) as a brown solid, yield: 39.6%. LCMS: Rt=1.47 min, MS (ESI) m/z=331.9 [M+H]⁺.

### Step 8

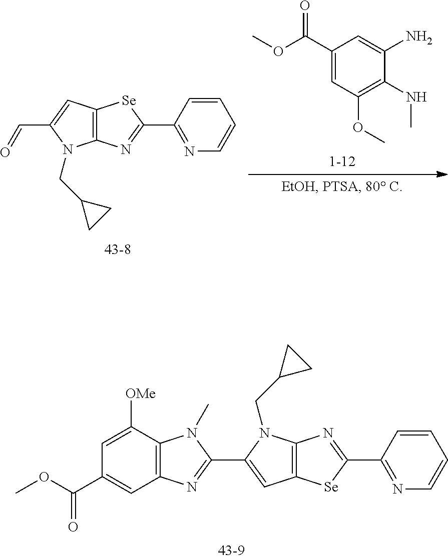 [SMILES: O=Cc1cc2[se]c(-c3ccccn3)nc2n1CC1CC1 ; CNc1c(N)cc(C(=O)OC)cc1OC ; COC(=O)c1cc(OC)c2c(c1)nc(-c1cc3[se]c(-c4ccccn4)nc3n1CC1CC1)n2C]

Compound 43-9: To a solution of compound 43-8 (350 mg, 1.06 mmol) and compound 1-12 (222.8 mg, 1.06 mmol) in EtOH (40 mL) was added p-toluenesulfonic acid (18.2 mg, 0.02 mmol). The mixture was stirred at 80° C. for 3 h. The solvent was evaporated and diluted with water (30 mL), extracted with ethyl acetate (30 mL×3). The organic layers were washed with water (15 mL), brine (15 mL), dried over anhydrous Na₂SO₄ and concentrated under vacuum to afford crude product, which was purified by silica gel chromatography (elution gradient: petroleum ether/EtOAc, 10/1, v/v) to afford compound 43-9 (300 mg) as a yellow solid, yield: 57.1%. LCMS: Rt=1.55 min, MS (ESI) m/z=521.9 [M+H]⁺.

### Step 9

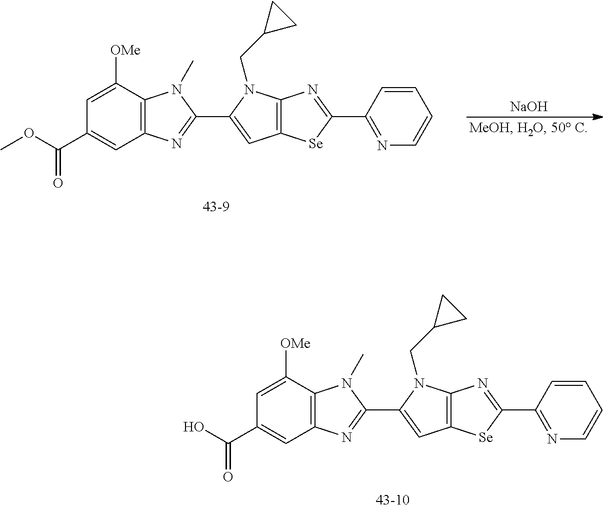 [SMILES: COC(=O)c1cc(OC)c2c(c1)nc(-c1cc3[se]c(-c4ccccn4)nc3n1CC1CC1)n2C ; "NaOH" ; "MeOH, H2O, 50° C." ; COc1cc(C(=O)O)cc2nc(-c3cc4[se]c(-c5ccccn5)nc4n3CC3CC3)n(C)c12]

Compound 43-10: To a suspension of compound 43-9 (300 mg, 0.58 mmol) in MeOH (20 mL) was added 2N sodium hydroxide aqueous solution (0.60 mL, 1.15 mmol). The mixture was stirred at 50° C. for 3 h. The mixture was concentrated and adjusted pH to 5-6 with 1N hydrochloric acid aqueous solution, then extracted with ethyl acetate (30 mL×3). The organic phase was washed with water (20 mL), brine (20 mL), dried over anhydrous Na₂SO₄ and concentrated under vacuum to give compound 43-10 (200 mg) as a yellow solid, yield: 67.8%. LCMS: Rt=1.40 min, MS (ESI) m/z=508.0 [M+H]⁺.

### Step 10

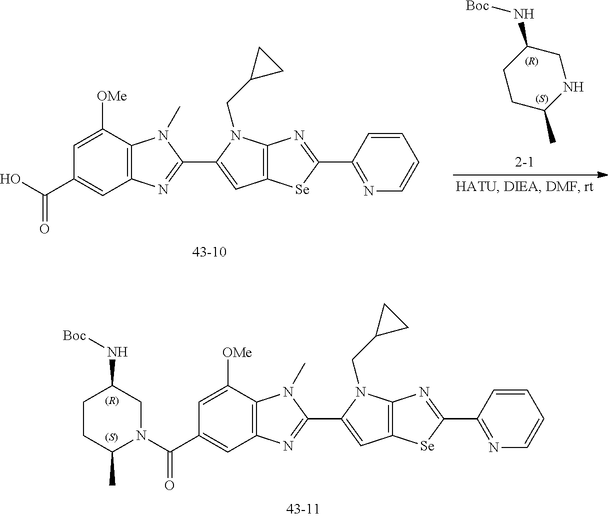

Compound 43-10: To a solution of compound 43-9 (100 mg, 0.19 mmol), DIEA (76.6 mg, 0.59 mmol) and HATU (90.1 mg, 0.24 mmol) in DMF (10 mL) was added compound 2-1 (46.1 mg, 0.22 mmol). The mixture was stirred at room temperature for 3 h. The mixture was diluted with water (60 mL), extracted with ethyl acetate (40 mL×3). The organic layers were washed with water (20 mL), brine (20 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 43-11 (100 mg) as a brown solid, yield: 64.8%. LCMS: Rt=1.50 min, MS (ESI) m/z=704.0 [M+H]⁺.

### Step 11

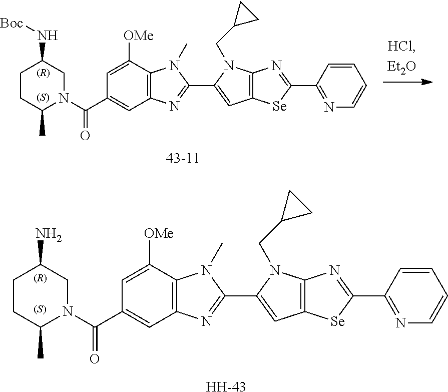

HH-43: To a solution of compound 43-11 (100 mg, 0.14 mmol) in ethyl ether (6 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-43 (59.3 mg, hydrochloric acid salt form) as a yellow solid. yield: 69.1%. ¹H NMR (400 MHz, DMSO-d₆): δ ppm 8.64 (d, J=4.4 Hz, 1H), 8.33 (s, 3H), 8.14 (d, J=7.6 Hz, 1H), 7.99 (t, J=7.6 Hz, 1H), 7.56-7.50 (m, 1H), 7.38 (s, 1H), 7.18 (s, 1H), 6.99 (s, 1H), 4.48 (d, J=6.8 Hz, 2H), 4.17 (s, 3H), 4.03 (s, 3H), 3.18 (s, 2H), 1.95-1.75 (m, 3H), 1.60 (s, 1H), 1.22 (d, J=6.8 Hz, 4H), 0.45-0.33 (m, 2H), 0.32-0.23 (m, 2H). LCMS: Rt=1.20 min, MS (ESI) m/z=604.1 [M+H]⁺. HPLC: Rt=5.64 min, Purity: 100% (214 nm) and 100% (254 nm).

### Example 44. Synthesis of HH-44

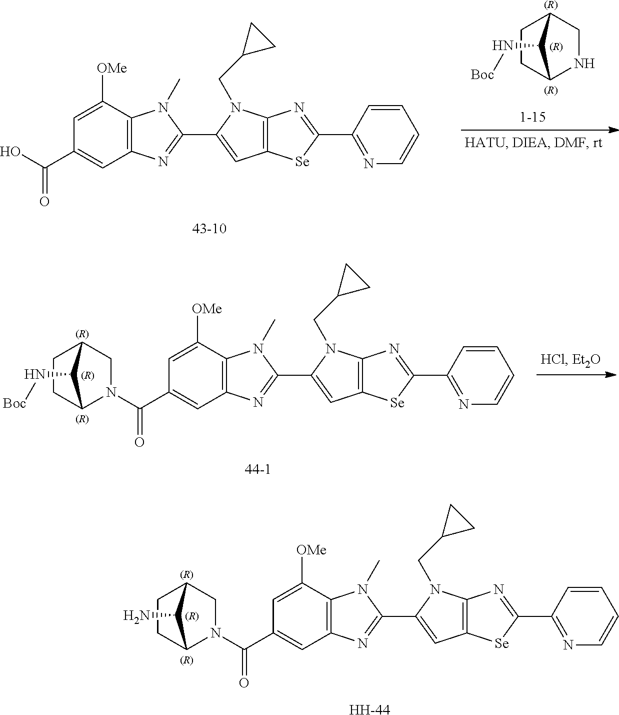

### Step 1

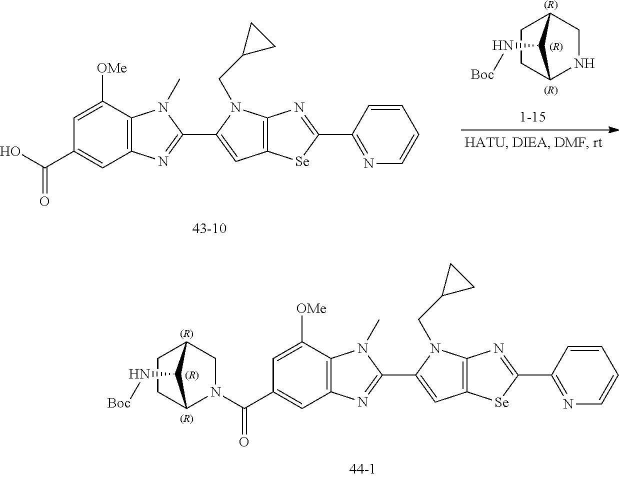

Compound 44-1: To a solution of compound 43-10 (100 mg, 0.20 mmol), DIEA (76.6 mg, 0.41 mmol) and HATU (90.1 mg, 0.24 mmol) in DMF (10 mL) was added compound 1-15 (46.1 mg, 0.22 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 44-1 (100 mg) as a yellow oil, yield: 72.3%. LCMS: Rt=1.48 min, MS (ESI) m/z=702.0 [M+H]⁺.

### Step 2

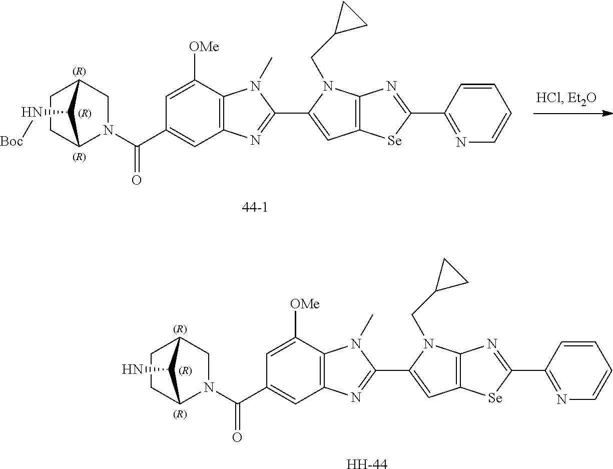

HH-44: To a solution of compound 44-1 (100 mg, 0.14 mmol) in ethyl ether (6 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-44 (49.8 mg, hydrochloric acid salt form) as a yellow solid. yield: 58.1%. ¹H NMR (400 MHz, DMSO-d₆): δ ppm 8.62 (d, J=4.4 Hz, 1H), 8.50 (s, 3H), 8.16-8.10 (m, 1H), 7.96 (td, J=7.6, 1.2 Hz, 1H), 7.48 (dd, J=7.6, 5.2 Hz, 2H), 7.13 (s, 1H), 7.05 (s, 1H), 4.49 (d, J=7.2 Hz, 2H), 4.15 (s, 3H), 4.04 (s, 3H), 3.65 (s, 1H), 3.52 (s, 1H), 3.18 (d, J=10 Hz, 1H), 2.68 (s, 1H), 2.08-1.88 (m, 3H), 1.63 (s, 1H), 1.24 (td, J=7.6, 2.8 Hz, 1H), 0.42-0.34 (m, 2H), 0.30-0.20 (m, 2H). LCMS: Rt=1.16 min, MS (ESI) m/z=602.1 [M+H]⁺. HPLC: Rt=5.41 min, Purity: 99.01% (214 nm) and 98.88% (254 nm).

### Example 45. Synthesis of HH-45

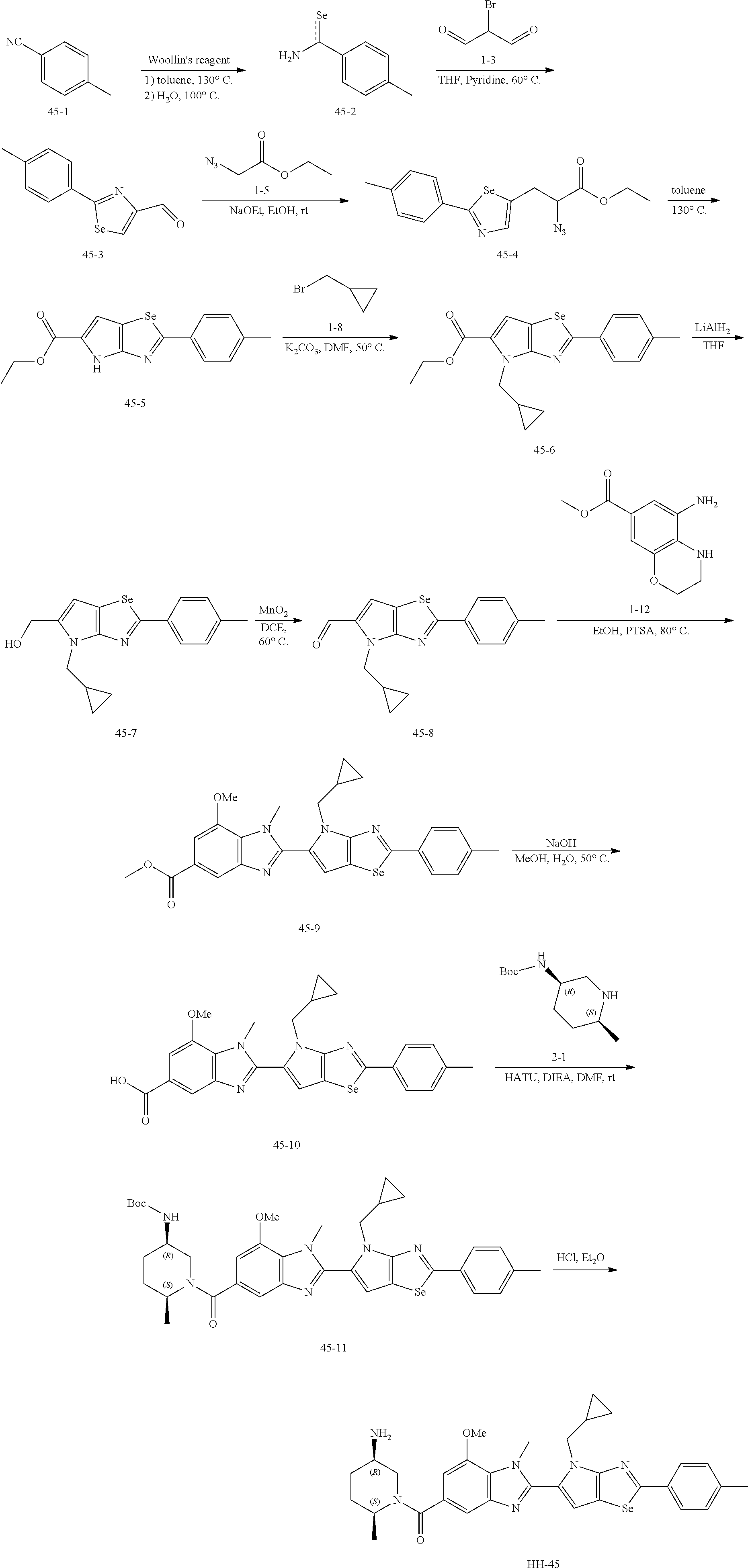

### Step 1

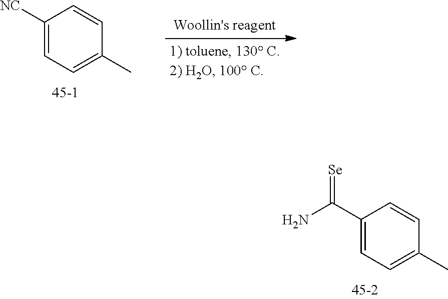

Compound 45-2: To a solution of 4-methylbenzonitrile (1.5 g, 12.82 mmol) in Toluene (20 mL) was added Woollin's reagent (3.4 g, 6.41 mmol), The reaction mixture was stirred at 130° C. for 3 h. To this H₂O (2 mL) was added at 90° C. The resulting mixture was stirred at 90° C. for further 1 h. The product was concentrated under vacuum to afford crude product, which was purified by silica gel chromatography (elution gradient: petroleum ether/EtOAc, 5/1, v/v) to afford compound 45-2 (2.0 g) as a brown solid, yield: 78.4%. LCMS: Rt=1.124 min, MS (ESI) m/z=199.9 [M+H]⁺.

### Step 2

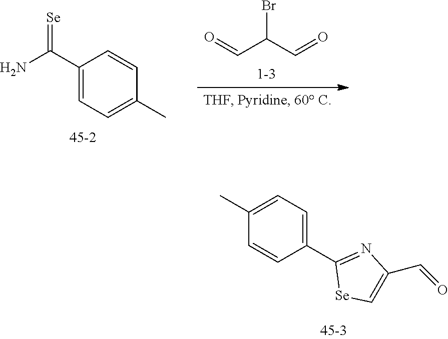

Compound 45-3: To a solution of compound 45-2 (2.0 g, 10.05 mmol) in THF (20 mL) was added 2-bromopropanedial (1.51 g, 10.05 mmol) and pyridine (1.59 g, 20.10 mmol). The reaction mixture was stirred at 60° C. for 16 h. The solvent was evaporated and diluted with water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 45-3 (1.0 g) as a yellow solid, yield: 40.0%. LCMS: Rt=1.323 min, MS (ESI) m/z=252.1[M+H]⁺.

### Step 3

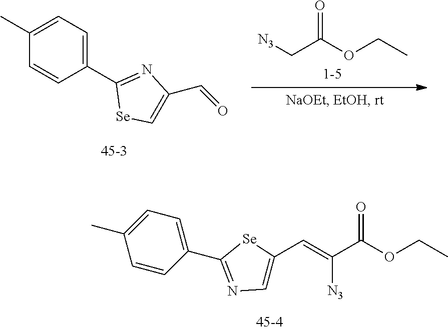

Compound 45-4: To a solution of sodium ethoxide (20% wt. in EtOH) (4.06 g, 11.95 mmol) in EtOH (50 mL) was slowly added compound 45-3 (1 g, 3.98 mmol) and compound 5 (1.54 g, 11.95 mmol) in EtOH (50 mL) at 0° C. under N₂. The reaction mixture was stirred at 0° C. for 1 h and 25° C. for 16 h. The mixture was quenched with ice water (50 mL) and extracted with ethyl acetate (50 mL×3). The organic layers were washed with water (200 mL), brine (200 mL), dried over anhydrous Na₂SO₄ and concentrated under vacuum to afford crude product, which was purified by silica gel chromatography (elution gradient: petroleum ether/EtOAc, 5/1, v/v) to afford compound 45-4 (500 mg) as a yellow oil, yield: 34.7%. LCMS: Rt=1.601 min, MS (ESI) m/z=362.9 [M+H]⁺.

### Step 4

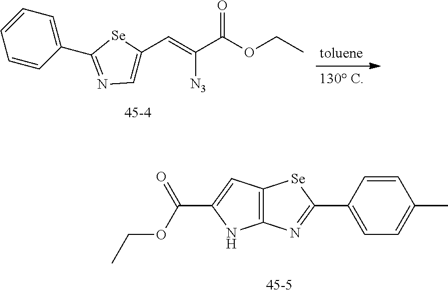

Compound 45-5: A solution of compound 45-4 (500 mg, 1.38 mmol) in toluene (20 mL) was stirred at 130° C. for 2 h. The reaction mixture was concentrated under vacuum to give the crude product, which was purified by silica gel chromatography (elution gradient: petroleum ether/EtOAc, 1/1, v/v) to afford compound 45-5 (300 mg) as yellow oil, yield: 65.1%. LCMS: Rt=1.449 min, MS (ESI) m/z=334.9 [M+H]⁺.

### Step 5

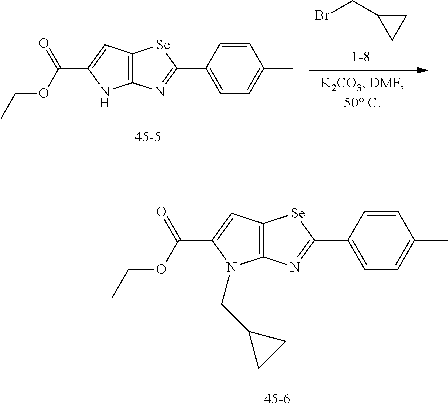

Compound 45-6: To a solution of compound 45-5 (300 mg, 0.90 mmol) and compound 1-8 (120 mg, 0.90 mmol) in DMF (10 mL) was added potassium carbonate (372.6 mg, 2.70 mmol). The mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 45-6 (280 mg) as a yellow oil, yield: 80.5%. LCMS: Rt=1.771 min, MS (ESI) m/z=389.0 [M+H]⁺.

### Step 6

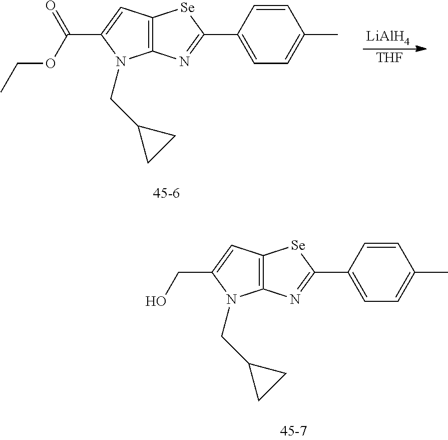

Compound 45-7: To a solution of compound 45-6 (280 mg, 0.72 mmol) in THE (5 mL) was added LiAlH₄ (1M in THF, 2.16 mL, 2.16 mmol) at 0° C. The reaction mixture was stirred at 25° C. for 2 h. The mixture was quenched with water (10 mL) and filtered. The filtrate was extracted with ethyl acetate (10 mL×3). The organic layers were dried over anhydrous Na₂SO₄ and concentrated under vacuum to afford crude product (230 mg) as a yellow oil. LCMS: Rt=1.532 min, MS (ESI) m/z=347.0 [M+H]⁺.

### Step 7

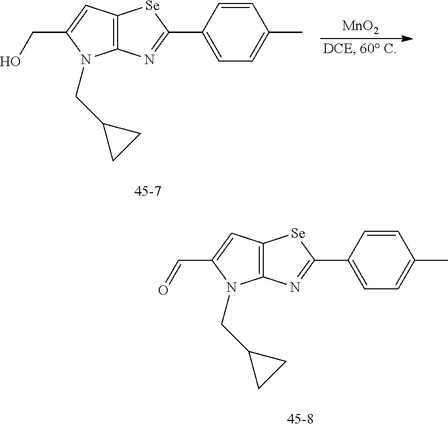

Compound 45-8: To a solution of compound 45-7 (230 mg, 0.66 mmol) in DCE (10 mL) was added MnO₂ (578 mg, 6.65 mmol). The reaction mixture was stirred at 60° C. for 16 h. The mixture was filtered and the filtrate was concentrated under vacuum to afford crude product (200 mg) as a brown oil. LCMS: Rt=1.643 min, MS (ESI) m/z=345.0 [M+H]⁺.

### Step 8

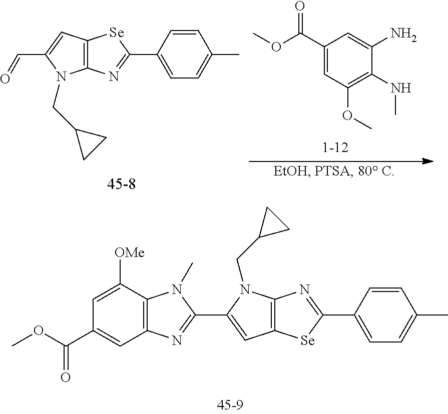

Compound 45-9: To a solution of compound 45-8 (200 mg, 0.58 mmol) in EtOH (10 mL) was added compound 1-12 (122 mg, 0.58 mmol) and PTSA (10 mg, 0.058 mmol). The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 45-9 (150 mg) as a yellow oil, yield: 48.4%. LCMS: Rt=1.743 min, MS (ESI) m/z=535.2 [M+H]⁺.

### Step 9

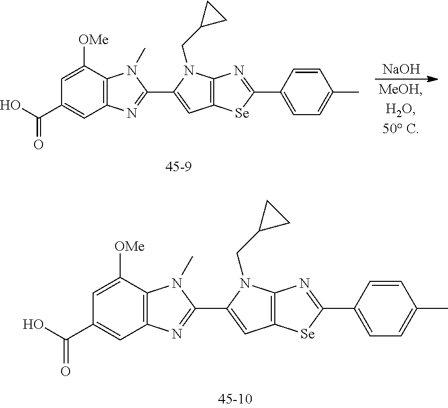

Compound 45-10: To a solution of compound 45-9 (150 mg, 0.28 mmol) in MeOH/H₂O (5/1 mL) was added NaOH (34 mg, 0.84 mmol). The reaction mixture was stirred at 50° C. for 16 h. The mixture was concentrated and adjusted pH to 5-6 with 1N hydrochloric acid aqueous solution, then extracted with ethyl acetate (10 mL×3). The organic phase was washed with water (10 mL), brine (10 mL), dried over anhydrous Na₂SO₄ and concentrated under vacuum to give compound 45-10 (140 mg) as a yellow solid, yield: 95.9%. LCMS: Rt=1.486 min, MS (ESI) m/z=521.1[M+H]⁺.

### Step 10

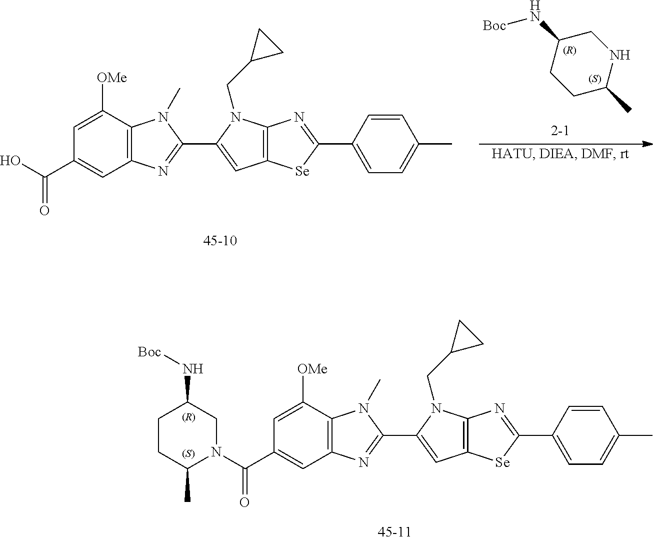

Compound 45-11: To a solution of compound 45-10 (140 mg, 0.27 mmol) in DMF (5 mL) was added compound 2-1 (58 mg, 0.27 mmol), HATU (103 mg, 0.27 mmol), DIEA (104 mg, 0.81 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1, v/v) to afford compound 45-11 (160 mg) as a brown solid, yield: 82.9%. LCMS: Rt=1.601 min, MS (ESI) m/z=717.2 [M+H]⁺.

### Step 11

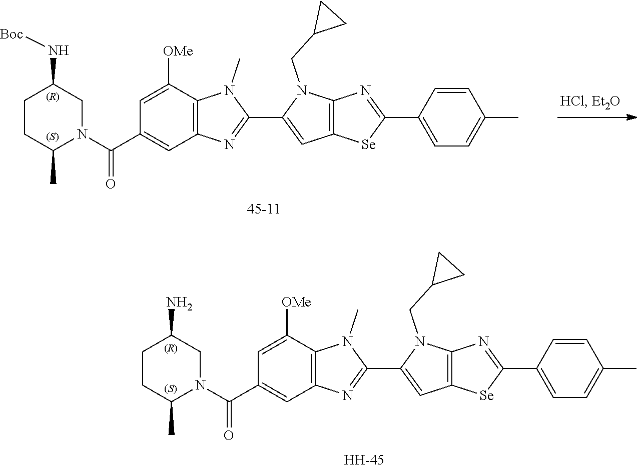

HH-45: To a solution of compound 45-11 (160 mg, 0.22 mmol) in Et₂O (5 mL) was added 2M HCl in Et₂O (6 mL, 12 mmol). The mixture was stirred at 25° C. for 2 h. The mixture was concentrated under vacuum to afford crude product, which was purified by preparative HPLC (Gemini-C18 column, 5μ silica, 21 mm diameter, 150 mm length), using decreasingly polar mixtures of water (containing 0.05% FA) and MeCN as eluents 30-70%) to afford HH-45 (98 mg, hydrochloric acid salt form) as a yellow solid. yield: 71.0%. ¹H NMR (400 MHz, DMSO-d₆) δ ppm 8.14 (br. s, 3H), 7.85 (d, J=8.2 Hz, 2H), 7.35-7.31 (m, 3H), 7.08-7.06 (m, 1H), 6.89-6.85 (m, 1H), 4.49 (d, J=7.2 Hz, 2H), 4.15 (s, 3H), 4.00 (s, 3H), 3.51-3.45 (m, 1H), 3.20-3.03 (s, 3H), 2.37 (s, 3H), 1.92-1.75 (m, 3H), 1.61-1.54 (m, 1H), 1.32-1.19 (m, 4H), 0.41-0.37 (m, 2H), 0.32-0.28 (m, 2H). LCMS: Rt=1.358 min, MS (ESI) m/z=617.0 [M+H]⁺. HPLC: Rt=6.377 min, Purity: 100% (214 nm) and 100% (254 nm).

### Example 46. Synthesis of HH-46

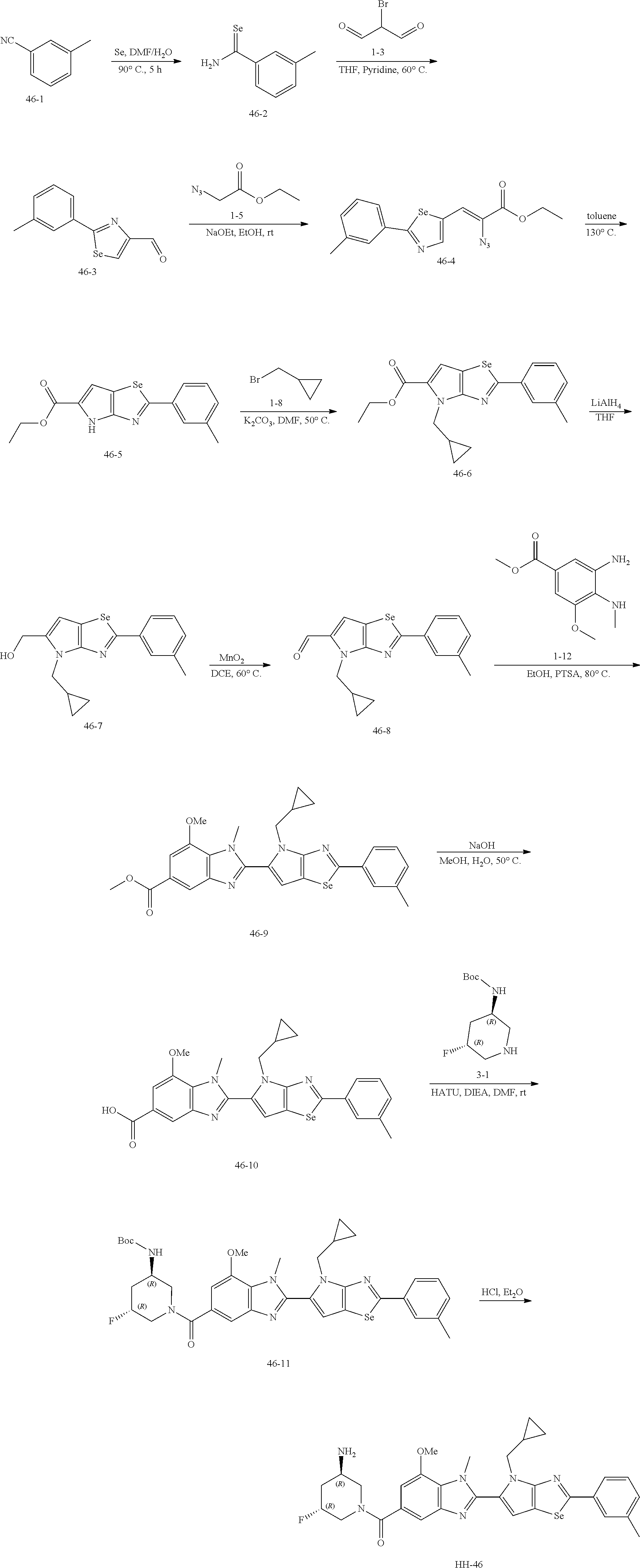

### Step 1

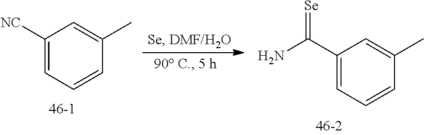

Compound 46-2: To a solution of 2-methylbenzonitrile (5 g, 0.043 mol) in DMF/H₂O (50/5 mL) was added Se powder (3.4 g, 0.043 mol). The reaction mixture was stirred at 90° C. under CO atmosphere for 5 h. The mixture was diluted with water (100 mL), extracted with EtOAc (3×100 mL). The organic layers were washed with brine (100 mL), dried over anhydrous Na₂SO₄ and concentrated under vacuum to afford crude product, which was purified by silica gel chromatography (elution gradient: petroleum ether/EtOAc, 5/1, v/v) to afford compound 46-2 (8 g) as a brown solid, yield: 94.1%. LCMS: Rt=1.128 min, MS (ESI) m/z=200.1 [M+H]⁺.

### Step 2

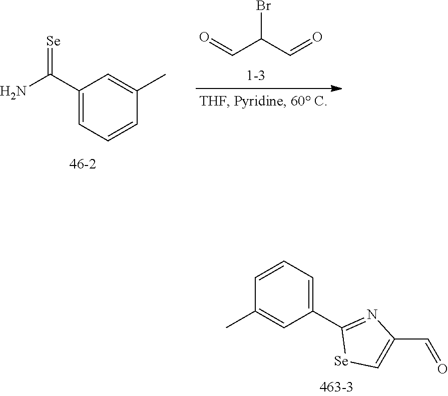

Compound 46-3: To a solution of compound 46-2 (8 g, 40.38 mmol) in THF (50 mL) was added 2-bromopropanedial (6.10 g, 40.38 mmol) and pyridine (6.38 g, 80.76 mmol). The reaction mixture was stirred at 60° C. for 16 h. The reaction mixture was stirred at 60° C. for 16 h. The solvent was evaporated and diluted with water (20 mL). The mixture was extracted with ethyl acetate (20 mL×3). The organic layers were dried over anhydrous Na₂SO₄ and concentrated under vacuum to afford crude product, which was purified by silica gel chromatography (elution gradient: petroleum ether/EtOAc, 5/1, v/v) to afford compound 46-3 (6.0 g) as a yellow solid, yield: 60.0%. LCMS: Rt=1.331 min, MS (ESI) m/z=252.0 [M+H]⁺.

### Step 3

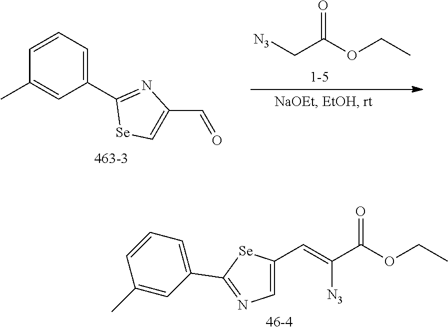

Compound 46-4: To a solution of sodium ethoxide (20% wt. in EtOH) (16.31 g, 47.97 mmol) in EtOH (100 mL) was added compound 46-3 (4 g, 15.99 mmol) and ethyl 2-azidoacetate (6.19 g, 47.99 mmol) at 0° C. The reaction mixture was stirred at 0° C. for 1 h and 25° C. for 16 h. The mixture was quenched with ice water (100 mL) and extracted with ethyl acetate (100 mL×3). The organic layers were washed with water (200 mL), brine (200 mL), dried over anhydrous Na₂SO₄ and concentrated under vacuum to afford crude product, which was purified by silica gel chromatography (elution gradient: petroleum ether/EtOAc, 5/1, v/v) to afford compound 46-4 (3 g) as a yellow oil, yield: 51.7%. LCMS: Rt=1.603 min, MS (ESI) m/z=362.9 [M+H]⁺.

### Step 4

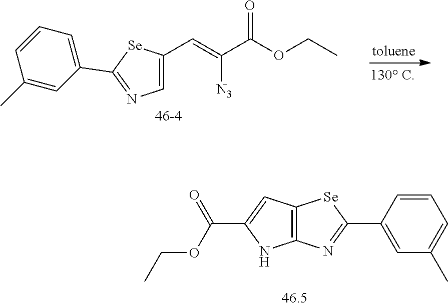

Compound 46-5: A solution of compound 46-4 (3 g, 8.30 mmol) in toluene (20 mL) was stirred at 130° C. for 2 h. The reaction mixture was concentrated under vacuum to give the crude product, which was purified by silica gel chromatography (elution gradient: petroleum ether/EtOAc, 1/1, v/v) to afford compound 46-5 (1.2 g) as yellow oil, yield: 44.4%. LCMS: Rt=1.450 min, MS (ESI) m/z=334.9 [M+H]⁺.

### Step 5

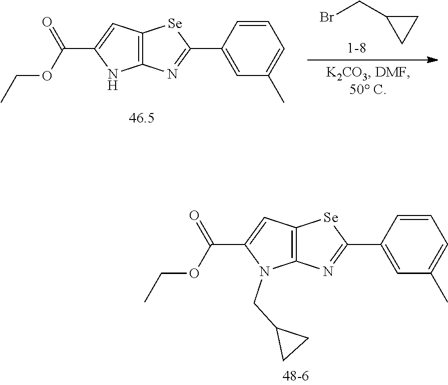

Compound 46-6: To a solution of compound 46-5 (1.2 g, 3.60 mmol) in DMF (10 mL) was added compound 1-8 (583 mg, 4.32 mmol) and K₂CO₃ (994 mg, 7.20 mmol). The reaction mixture was stirred at 50° C. for 16 h. The mixture was diluted with water (20 mL) and extracted with ethyl acetate (20 mL×3). The organic layers were washed with brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 46-6 (750 mg) as a yellow oil, yield: 53.6%. LCMS: Rt=1.769 min, MS (ESI) m/z=389.0 [M+H]⁺.

### Step 6

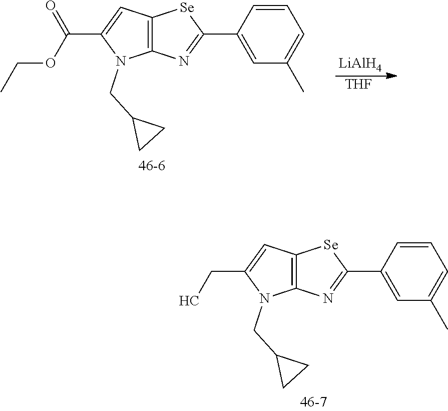

Compound 46-7: To a solution of compound 46-6 (750 mg, 1.94 mmol) in THE (20 mL) was added LiAlH₄ (1M in THF, 5.8 mL, 5.81 mmol) at 0° C. The reaction mixture was stirred at 25° C. for 16 h. The mixture was quenched with water (10 mL) and filtered. The filtrate was extracted with ethyl acetate (10 mL×3). The organic layers were dried over anhydrous Na₂SO₄ and concentrated under vacuum to afford crude product (500 mg) as a yellow oil. LCMS: Rt=1.460 min, MS (ESI) m/z=347.0 [M+H]⁺.

### Step 7

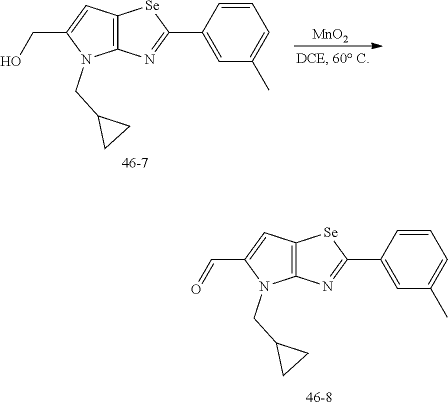

Compound 46-8: To a solution of compound 46-7 (500 mg, 1.44 mmol) in DCE (10 mL) was added MnO₂ (1.25 g, 14.41 mmol), The reaction mixture was stirred at 60° C. for 16 h. The mixture was filtered and the filtrate was concentrated under vacuum to afford crude product (480 mg) as a brown oil, yield: 97.8%. LCMS: Rt=1.566 min, MS (ESI) m/z=345.0 [M+H]⁺.

### Step 8

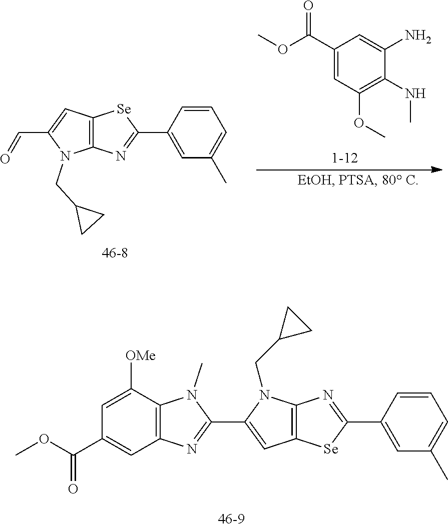

Compound 46-9: To a solution of compound 46-8 (400 mg, 1.16 mmol) in EtOH (10 mL) was added compound 1-12 (243 mg, 1.16 mmol) and PTSA (21 mg, 0.12 mmol). The reaction mixture was stirred at 80° C. for 16 h. The mixture was concentrated under vacuum to give the crude product, which was purified by silica gel chromatography (elution gradient: petroleum ether/EtOAc, 1/1, v/v) to afford compound 46-9 (400 mg) as a yellow oil, yield: 53.7%. LCMS: Rt=1.664 min, MS (ESI) m/z=535.1 [M+H]⁺.

### Step 9

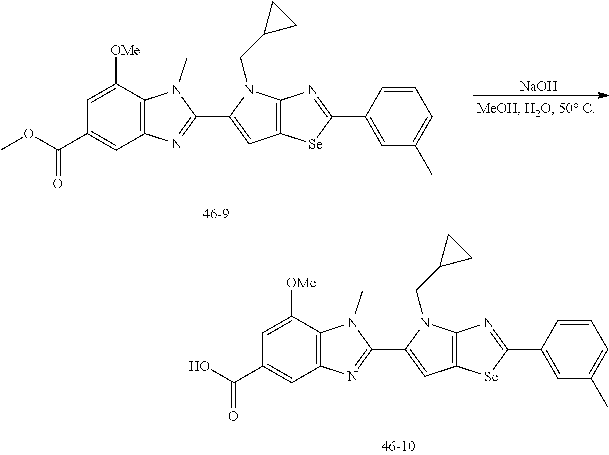

Compound 46-10: To a solution of compound 46-9 (400 mg, 0.75 mmol) in MeOH/H₂O (10/2 mL) was added NaOH (90 mg, 2.25 mmol). The reaction mixture was stirred at 50° C. for 16 h. The mixture was concentrated and adjusted pH to 5-6 with 1N hydrochloric acid aqueous solution, then extracted with ethyl acetate (10 mL×3). The organic phases were washed with water (10 mL), brine (10 mL), dried over anhydrous Na₂SO₄ and concentrated under vacuum to give compound 46-10 (320 mg) as a yellow solid, yield: 82.1%. LCMS: Rt=1.494 min, MS (ESI) m/z=521.1[M+H]⁺.

### Step 10

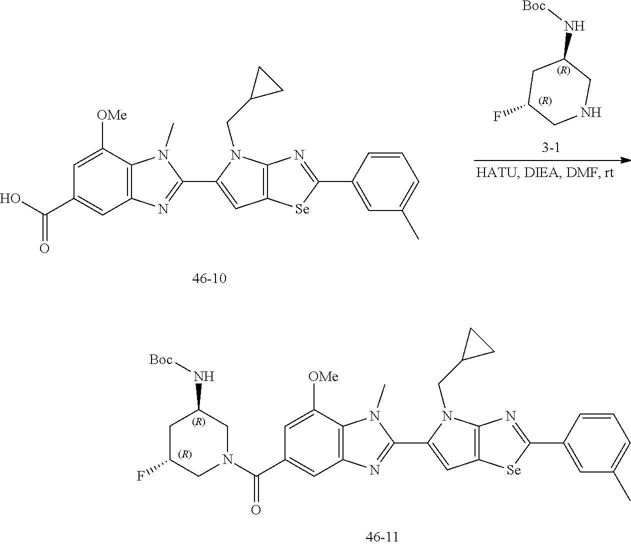

Compound 46-11: To a solution of compound 46-10 (100 mg, 0.19 mmol) in DMF (10 mL) was added compound 3-1 (42.2 mg, 0.19 mmol), HATU (73.1 mg, 0.19 mmol) and DIEA (74.5 mg, 0.58 mmol). The reaction mixture was stirred at 25° C. for 16 h. The mixture was diluted with water (10 mL), extracted with ethyl acetate (15 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1, v/v) to afford compound 46-11 (100 mg) as a brown solid, yield: 72.5%. LCMS: Rt=1.555 min, MS (ESI) m/z=721.2 [M+H]⁺.

### Step 11

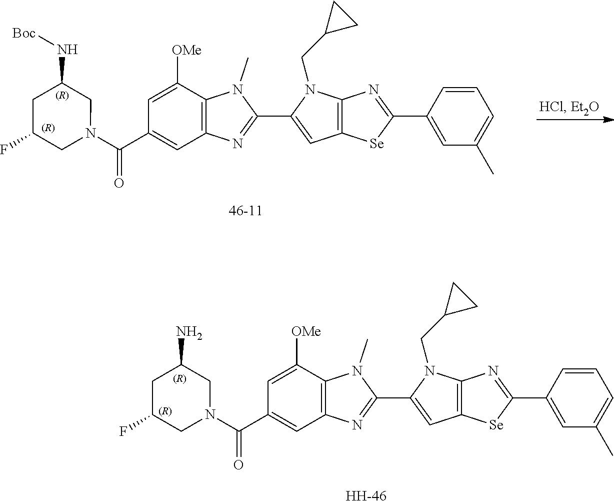

HH-46: To a solution of compound 46-11 (100 mg, 0.14 mmol) in Et₂O (5 mL) was added 2M HCl in Et₂O (6 mL, 12 mmol). The reaction mixture was stirred at 25° C. for 2 h. The mixture was concentrated under vacuum to afford crude product, which was purified by preparative HPLC (Gemini-C18 column, 5μ silica, 21 mm diameter, 150 mm length), using decreasingly polar mixtures of water (containing 0.05% FA) and MeCN as eluents 13-25%) to afford HH-46 (45.6 mg, hydrochloric acid salt form) as a yellow solid. yield: 53.0%. ¹H NMR (400 MHz, DMSO-d6) δ ppm 8.48 (br. s, 3H), 7.79-7.77 (m, 2H), 7.43-7.39 (m, 2H), 7.35-7.32 (m, 1H), 7.19-7.16 (m, 1H), 7.02-6.96 (m, 1H), 5.06-4.70 (m, 3H), 4.47 (d, J=6.8 Hz, 2H), 4.17 (s, 3H), 4.02 (s, 3H), 3.39-3.34 (m, 2H), 3.15-3.00 (m, 1H), 2.05-1.87 (m, 1H), 1.28-1.20 (m, 1H), 0.41-0.36 (m, 2H), 0.28-0.24 (m, 2H). LCMS: Rt=1.173 min, MS (ESI) m/z=621.2 [M+H]⁺. HPLC: Rt=6.478 min, Purity: 99.7% (214 nm) and 99.9% (254 nm).

### Example 47. Synthesis of HH-47

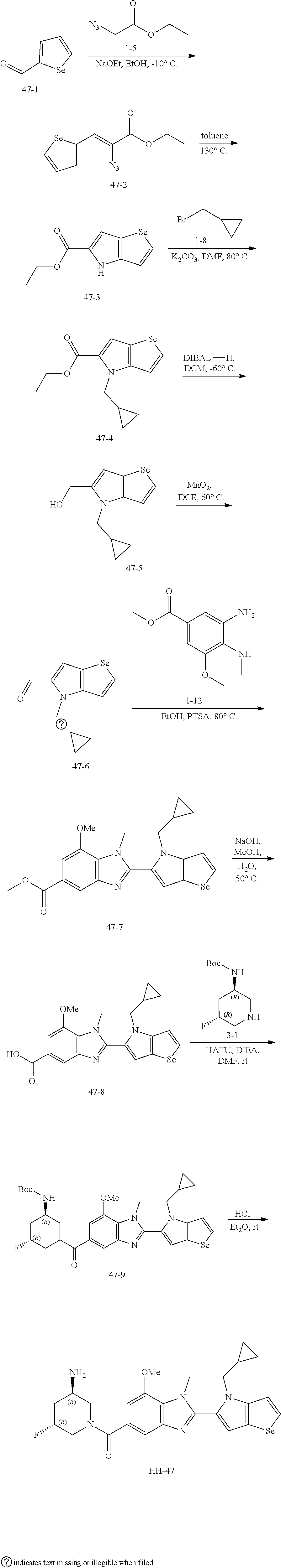

### Step 1

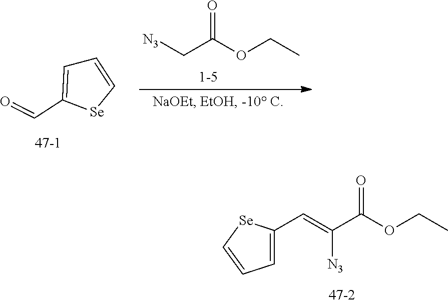

Compound 47-2: To a solution of sodium ethoxide (20% wt. in EtOH) (6.42 g, 18.86 mmol) in EtOH (20 mL) was slowly added compound 47-1 (1.0 g, 6.29 mmol) and compound 1-5 (2.43 g, 18.86 mmol) in EtOH (50 mL) at −15° C. under N₂. The reaction mixture was stirred at −15° C. for 1 h and 0° C. for further 1 h. The mixture was quenched with ice water (100 mL) and extracted with ethyl acetate (50 mL×3). The organic layers were washed with water (50 mL), brine (50 mL), dried over anhydrous Na₂SO₄ and concentrated under vacuum to afford crude product, which was purified by silica gel chromatography (PE/EtOAc: 20/1, v/v) to afford compound 47-2 (0.8 g) as a yellow oil, yield: 47.1%. LCMS: Rt=1.32 min, MS (ESI) m/z=272.0 [M+H]⁺.

### Step 2

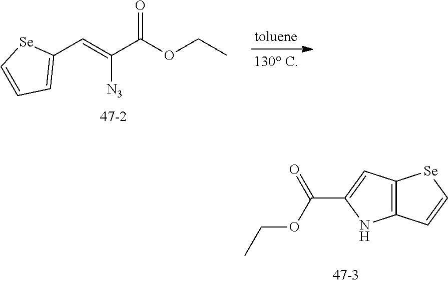

Compound 47-3: A solution of compound 47-2 (0.8 g, 2.96 mmol) in toluene (20 mL) was stirred at 130° C. under N₂ for 16 h. The mixture was concentrated under vacuum to give the crude product, which was purified by silica gel chromatography (elution gradient: petroleum ether/EtOAc, 5/1, v/v) to afford compound 47-3 (0.7 g) as yellow solid, yield: 97.6%. LCMS: Rt=1.24 min, MS (ESI) m/z=244.1 [M+H]⁺.

### Step 3

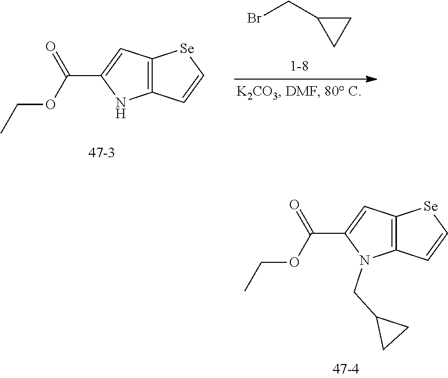 [SMILES: CCOC(=O)c1cc2[se]ccc2[nH]1 ; BrCC1CC1 ; CCOC(=O)c1cc2[se]ccc2n1CC1CC1]

Compound 47-4: To a solution of compound 47-3 (0.7 g, 2.89 mmol) and compound 1-8 (0.58 g, 4.34 mmol) in DMF (10 mL) was added potassium carbonate (0.8 g, 5.78 mmol). The mixture was stirred at 80° C. for 3 h. The solvent was evaporated under vacuum and water (50 mL) was added. The mixture was extracted with ethyl acetate (50 mL×3). The organic phase was washed with water (50 mL), brine (50 mL), dried over anhydrous Na₂SO₄ and concentrated under vacuum to afford crude product, which was purified by silica gel chromatography (elution gradient: petroleum ether/EtOAc, 10/1, v/v) to afford compound 47-4 (0.65 g) as a yellow oil, yield: 75.9%. LCMS: Rt=1.44 min, MS (ESI) m/z=298.0 [M+H]⁺.

### Step 4

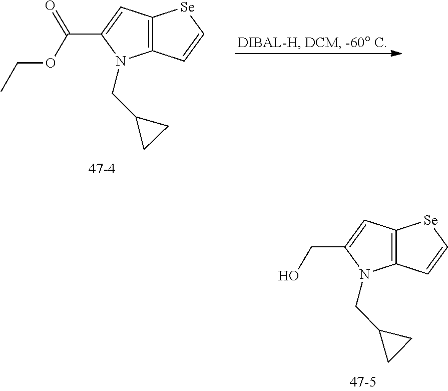

Compound 47-5: To a solution of compound 47-4 (0.6 g, 2.02 mmol) in DCM (10 mL) cooled at −60° C. under N₂ was slowly added DIBAL-H (1 M in THF, 6.1 mL, 6.1 mmol). The mixture was stirred at −60° C. under N₂ for 2 h. The mixture was quenched with water (30 mL) and filtered. The filtrate was extracted with dichloromethane (50 mL×3). The organic layers were dried over anhydrous Na₂SO₄ and concentrated under vacuum to afford compound 47-5 (0.5 g) as a yellow oil, yield: 97.1%. LCMS: Rt=1.23 min, MS (ESI) m/z=255.9 [M+H]⁺.

### Step 5

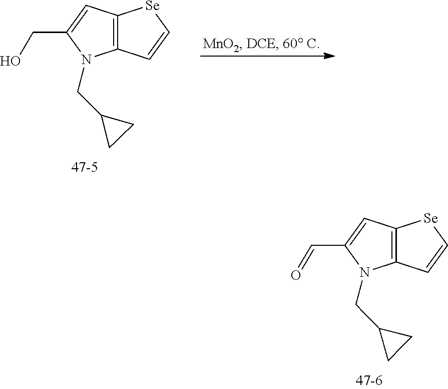

Compound 47-6: To a solution of compound 47-5 (0.5 g, 1.97 mmol) in DCE (30 mL) was added manganese dioxide (1.71 g, 19.67 mmol). The mixture was stirred at 60° C. for 16 h. The mixture was filtered and the filtrate was concentrated under vacuum to afford crude product, which was purified by silica gel chromatography (elution gradient: petroleum ether/EtOAc, 5/1, v/v) to afford compound 47-6 (260 mg) as a brown oil, yield: 52.4%. LCMS: Rt=1.32 min, MS (ESI) m/z=254.0 [M+H]⁺.

### Step 6

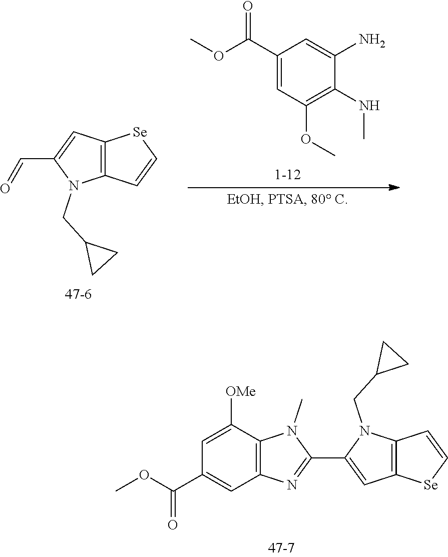 [SMILES: O=Cc1cc2[se]ccc2n1CC1CC1 ; CNc1c(N)cc(C(=O)OC)cc1OC ; COC(=O)c1cc(OC)c2c(c1)nc(-c1cc3[se]ccc3n1CC1CC1)n2C]

Compound 47-7: To a solution of compound 47-6 (260 mg, 1.03 mmol) and compound 1-12 (238.4 mg, 1.13 mmol) in EtOH (20 mL) was added p-toluenesulfonic acid (17.76 mg, 0.10 mmol). The mixture was stirred at 80° C. for 3 h. The solvent was evaporated and diluted with water (20 mL), extracted with ethyl acetate (20 mL×3). The organic layers were washed with water (10 mL), brine (10 mL), dried over anhydrous Na₂SO₄ and concentrated under vacuum to afford crude product, which was purified by silica gel chromatography (elution gradient: DCM/MeOH, 10/1, v/v) to afford compound 47-7 (200 mg) as a yellow oil, yield: 43.8%. LCMS: Rt=1.43 min, MS (ESI) m/z=443.9 [M+H]⁺.

### Step 7

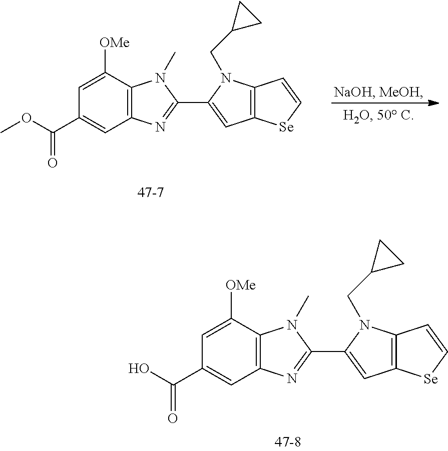

Compound 47-8: To a solution of compound 47-7 (200 mg, 0.45 mmol) in MeOH (10 mL) was added sodium hydroxide (2 N, 0.5 mL). The mixture was stirred at 50° C. for 3 h. The mixture was concentrated and adjusted pH to 5-6 with hydrochloric acid aqueous solution (2N), then extracted with ethyl acetate (30 mL×3). The organic phase was washed with water (30 mL), brine (30 mL), and concentrated under vacuum to give compound 11 (140 mg) as a yellow solid, yield: 72.3%. LCMS: Rt=1.29 min, MS (ESI) m/z=429.9 [M+H]⁺.

### Step 8

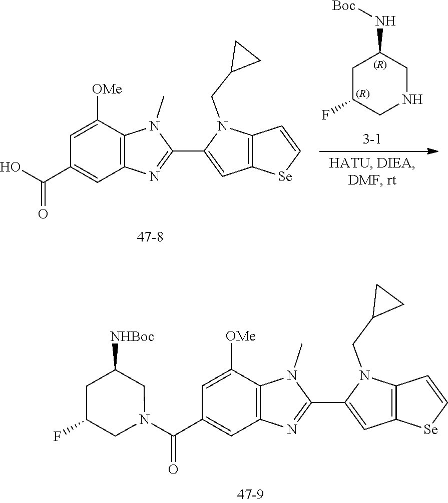

Compound 47-9: To a solution of compound 47-8 (70 mg, 0.16 mmol), DIEA (63.3 mg, 0.49 mmol) and HATU (74.6 mg, 0.19 mmol) in DMF (5 mL) was added compound 3-1 (39.4 mg, 0.18 mmol). The mixture was stirred at room temperature for 3 h. The mixture was diluted with water (20 mL), extracted with ethyl acetate (30 mL×3). The organic phase was washed with water (20 mL), brine (20 mL) and concentrated under vacuum to give the crude product, which was purified by silica gel chromatography (elution gradient: petroleum ether/EtOAc, 1/1 to 0/100, v/v) to afford product (80 mg) as a yellow oil, yield: 77.8%. LCMS: Rt=1.36 min, MS (ESI) m/z=630.0 [M+H]⁺.

### Step 9

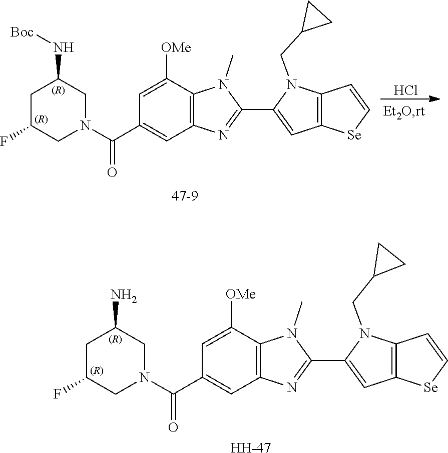

HH-47: To a solution of compound 47-9 (80 mg, 0.13 mmol) in ethyl ether (5 mL) was added hydrochloric acid solution (5 mL, 2N in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-47 (36 mg) as a yellow solid. yield: 52.8%. ¹H NMR (400 MHz, MeOD) ppm δ 8.06 (d, J=5.9 Hz, 1H), 7.50 (d, J=5.9 Hz, 1H), 7.40 (s, 1H), 7.24 (s, 1H), 7.17 (s, 1H), 4.96 (s, 1H), 4.22 (s, 3H), 4.20-4.15 (m, 3H), 4.06 (s, 4H), 3.60-3.35 (m, 2H), 3.05-2.95 (m, 1H), 2.55-2.42 (m, 1H), 2.01-1.85 (m, 1H), 0.91 (s, 1H), 0.36 (d, J=7.7 Hz, 2H), 0.03-0.01 (m, 2H). LCMS: Rt=1.10 min, MS (ESI) m/z=530.1 [M+H]⁺. HPLC: Rt=5.25 min, Purity: 99.48% (214 nm) and 99.09% (254 nm).

### Example 48. Synthesis of HH-48

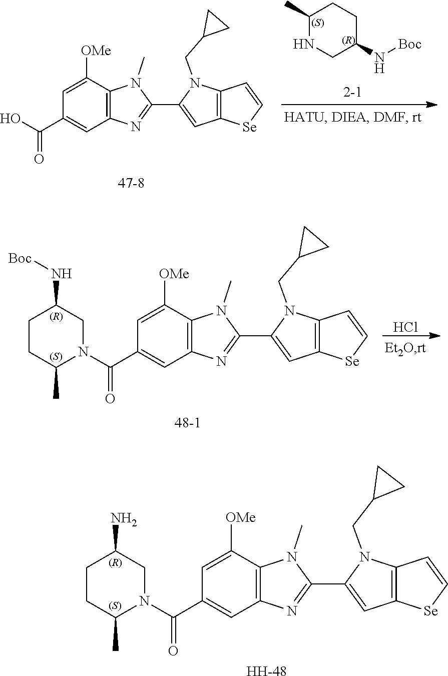

### Step 1

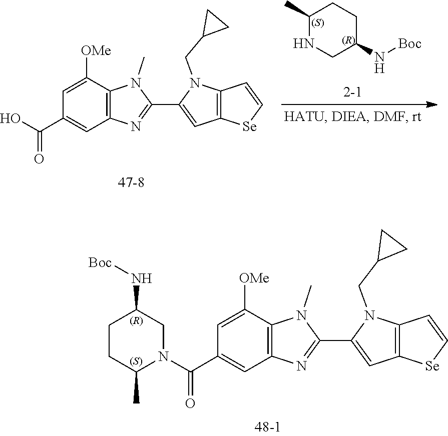

Compound 48-1: To a solution of compound 47-8 (70 mg, 0.16 mmol), DIEA (63.3 mg, 0.49 mmol) and HATU (74.6 mg, 0.19 mmol) in DMF (5 mL) was added compound 2-1 (38.7 mg, 0.18 mmol). The mixture was stirred at room temperature for 3 h. The mixture was diluted with water (20 mL), extracted with ethyl acetate (30 mL×3). The organic phase was washed with water (20 mL), brine (20 mL) and concentrated under vacuum to give the crude product, which was purified by silica gel chromatography (elution gradient: petroleum ether/EtOAc, 1/1 to 0/100, v/v) to afford compound 48-1 (80 mg) as a yellow oil, yield: 77.9%. LCMS: Rt=1.40 min, MS (ESI) m/z=626.0 [M+H]⁺.

### Step 2

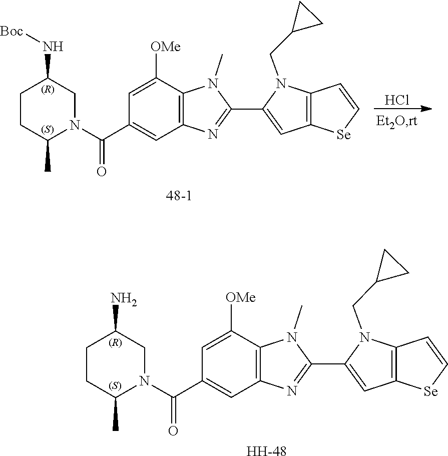

HH-48: To a solution of compound 48-1 (80 mg, 0.13 mmol) in ethyl ether (5 mL) was added hydrochloric acid solution (5 mL, 2N in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-48 (38 mg) as a yellow solid. yield: 56.4%. ¹H NMR (400 MHz, MeOD) ppm δ 8.06 (d, J=5.9 Hz, 1H), 7.50 (d, J=5.9 Hz, 1H), 7.37 (s, 1H), 7.23 (s, 1H), 7.15 (s, 1H), 4.22-4.16 (m, 6H), 4.05 (s, 4H), 3.33-3.25 (m, 1H), 3.11-3.01 (m, 1H), 2.01-1.95 (m, 1H), 1.88-1.82 (m, 2H), 1.73-1.62 (m, 1H), 1.29-1.27 (m, 3H), 0.94-0.87 (m, 1H), 0.38-0.33 (m, 2H), 0.03-0.01 (m, 2H). LCMS: Rt=1.11 min, MS (ESI) m/z=526.1 [M+H]⁺. HPLC: Rt=5.26 min, Purity: 99.39% (214 nm) and 99.56% (254 nm).

### Example 49. Synthesis of HH-49

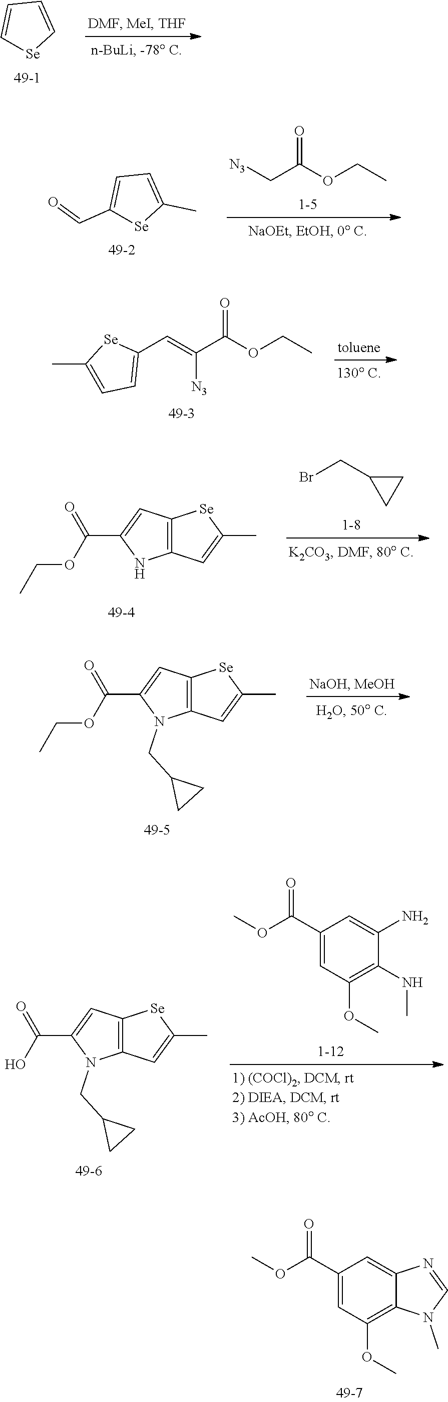

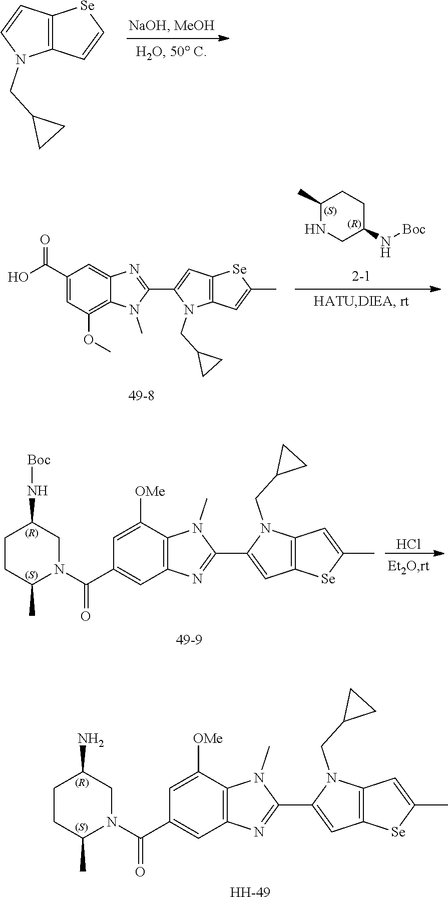

### Step 1

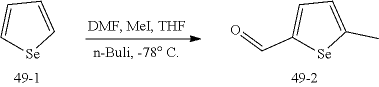

Compound 49-2: To a solution of compound 49-1 (5 g, 38.1 mmol) in THE (60 mL) cooled to −78° C. under N₂ was slowly added n-butyllithium (2.4 M in hexane, 15.9 mL). The mixture was warmed up to −20° C. and stirred for 1 h. The reaction mixture was cooled down to −78° C., Mel (2.19 mL, 38.1 mmol) was added dropwise at −78° C. The cooling bath was removed and the mixture was stirred at room temperature for 2 h. Then n-butyllithium (2.4 M in hexane, 15.9 mL) was added dropwise to the mixture at −20° C., the mixture was stirred at this temperature for 30 min. The reaction mixture was cooling down to −78° C. again, N,N-dimethylformamide (4.19 g, 57 mmol) was added dropwise at −78° C. The mixture was warmed up to room temperature and stirred for 1 h. The solvent was quenched with NH₄Cl (50 mL) and extracted with ethyl acetate (100 mL×4). The organic layers were dried over anhydrous Na₂SO₄ and concentrated under vacuum to afford crude product, which was purified by silica gel chromatography (elution gradient: petroleum ether/EtOAc, 10/1, v/v) to afford compound 2 (3.5 g) as a yellow oil, yield: 47.6%. ¹H NMR (400 MHz, CDCl3) δ ppm 9.70 (s, 1H), 7.82 (d, J=4.0 Hz, 1H), 7.10-7.04 (m, 1H), 2.65 (s, 3H).

### Step 2

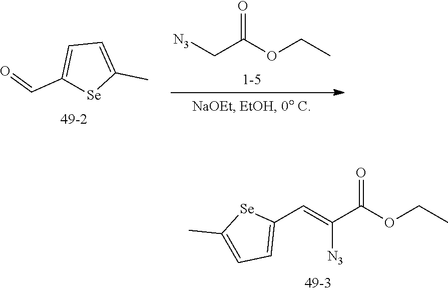

Compound 49-3: To a solution of sodium ethoxide (20% wt. in EtOH) (20.64 g, 60.66 mmol) in EtOH (100 mL) was slowly added compound 49-2 (3.5 g, 20.22 mmol) and compound 1-5 (7.83 g, 60.66 mmol) in EtOH (100 mL) at −15° C. under N₂. The reaction mixture was stirred at −15° C. for 1 h and 0° C. for further 1 h. The mixture was quenched with ice water (100 mL) and extracted with ethyl acetate (100 mL×3). The organic layers were washed with water (200 mL), brine (200 mL), dried over anhydrous Na₂SO₄ and concentrated under vacuum to afford crude product, which was purified by silica gel chromatography (EtOAc) to afford compound 49-3 (2.2 g) as a yellow oil, yield: 38.3%. LCMS: Rt=1.253 min, MS (ESI) m/z=286.01 [M+H]⁺.

### Step 3

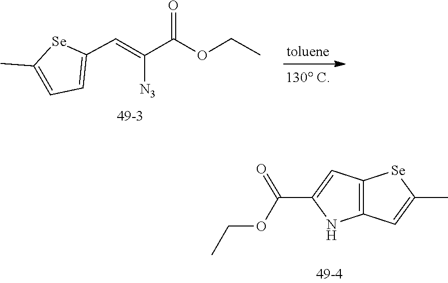 [SMILES: CCOC(=O)/C(=C/c1ccc(C)[se]1)N=[N+]=[N-] ; CCOC(=O)c1cc2[se]c(C)cc2[nH]1]

Compound 49-4: A solution of compound 49-3 (2.2 g, 7.74 mmol) in toluene (50 mL) was stirred at 130° C. under N₂ for 16 h. The mixture was concentrated under vacuum to give the crude product, which was purified by silica gel chromatography (elution gradient: petroleum ether/EtOAc, 10/1, v/v) to afford compound 49-4 (1.9 g) as yellow oil, yield: 95.8%. LCMS: Rt=1.296 min, MS (ESI) m/z=258.0 [M+H]⁺.

### Step 4

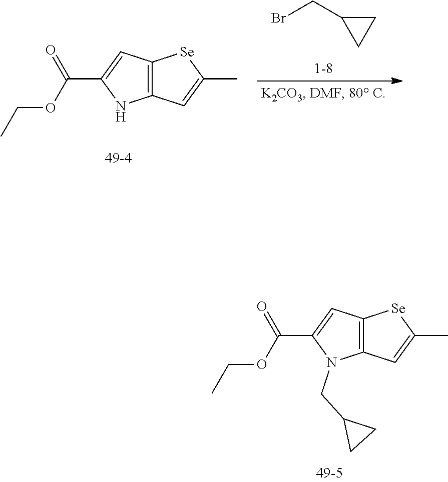 [SMILES: BrCC1CC1 ; CCOC(=O)c1cc2[se]c(C)cc2[nH]1 ; CCOC(=O)c1cc2[se]c(C)cc2n1CC1CC1]

Compound 49-5: To a solution of compound 49-4 (1.9 g, 7.42 mmol) and compound 1-8 (1.50 g, 11.1 mmol) in DMF (40 mL) was added potassium carbonate (2.05 g, 14.8 mmol). The mixture was stirred at 80° C. for 3 h. The solvent was evaporated and water (200 mL) was added. The mixture was extracted with ethyl acetate (100 mL×3). The organic phase was washed with water (100 mL), brine (100 mL), The organic layers were dried over anhydrous Na₂SO₄ and concentrated under vacuum to afford crude product, which was purified by silica gel chromatography (elution gradient: petroleum ether/EtOAc, 5/1, v/v) to afford compound 49-5 (2.2 g) as a yellow oil, yield: 95.6%. LCMS: Rt=1.523 min, MS (ESI) m/z=312.0 [M+H]⁺.

### Step 5

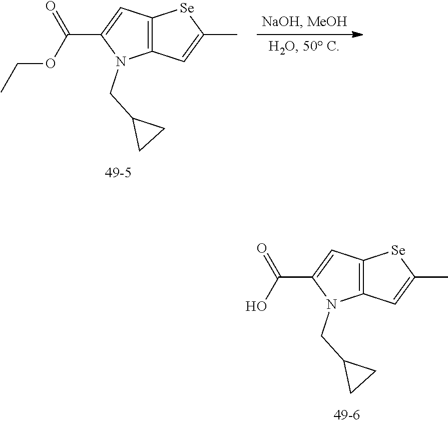 [SMILES: CCOC(=O)c1cc2[se]c(C)cc2n1CC1CC1 ; Cc1cc2c(cc(C(=O)O)n2CC2CC2)[se]1]

Compound 49-6: To a solution of compound 49-5 (2.2 g, 7.09 mmol) in MeOH (30 mL) was added sodium hydroxide (2 N, 7.1 mL). The mixture was stirred at 50° C. for 3 h. The mixture was concentrated and adjusted pH to 5-6 with hydrochloric acid aqueous solution (2N), then extracted with ethyl acetate (100 mL×3). The organic phase was washed with water (50 mL), brine (50 mL), and concentrated under vacuum to give compound 49-6 (1.7 g) as a yellow solid, yield: 92.3%. LCMS: Rt=1.304 min, MS (ESI) m/z=284.0 [M+H]⁺.

### Step 6

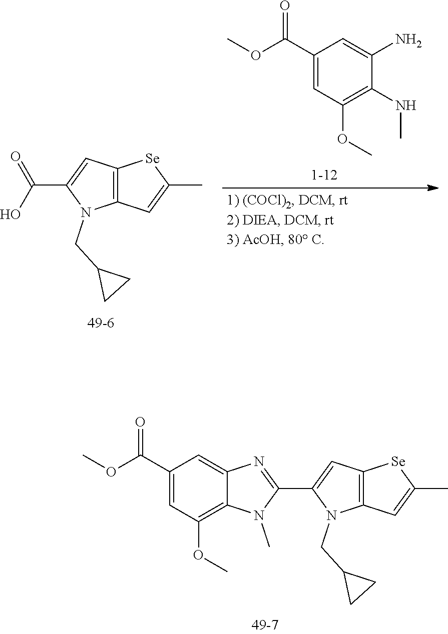

Compound 49-7: To a solution of compound 49-6 (1.7 g, 6.02 mmol) in DCM (40 mL) was added oxalyl chloride (1.53 g, 12.1 mmol) and DMF (0.05 mL). The mixture was stirred at room temperature for 1 h. The mixture was concentrated under vacuum to afford residue. The residue was dissolved in DCM (40 mL), DIEA (2.19 g, 16.9 mmol) and compound 1-12 (1.43 g, 6.79 mmol) was added. The mixture was stirred at room temperature for 2 h. The mixture was concentrated under vacuum to afford residue. The residue was dissolved in AcOH (50 mL) and stirred at 80° C. for 2 h. The mixture was concentrated under reduced pressure to afford residue. The residue was diluted with water (50 mL), extracted with ethyl acetate (50 mL×3). The combined organic layers were dried over anhydrous sodium sulfate and concentrated under vacuum to afford crude product, which was purified by flash chromatography (elution gradient: petroleum ether/ethyl acetate, 1/1, v/v) to afford compound 49-7 (2.0 g) as yellow solid, yield: 74.1%. LCMS: Rt=1.461 min, MS (ESI) m/z=458.1 [M+H]⁺.

### Step 7

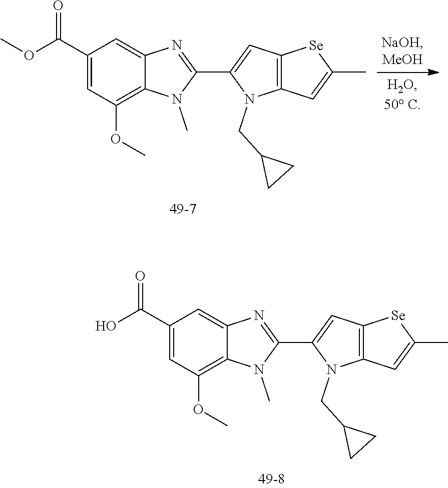

Compound 49-8: To a solution of compound 49-7 (2.0 g, 4.38 mmol) in MeOH (30 mL) was added sodium hydroxide (2 N, 4.4 mL). The mixture was stirred at 50° C. for 3 h. The mixture was concentrated and adjusted pH to 5-6 with hydrochloric acid aqueous solution (2N), then extracted with ethyl acetate (100 mL×3). The organic phase was washed with water (50 mL), brine (50 mL), and concentrated under vacuum to give compound 49-8 (1.9 g) as a yellow solid, yield: 98.0%. LCMS: Rt=1.334 min, MS (ESI) m/z=443.9 [M+H]⁺.

### Step 8

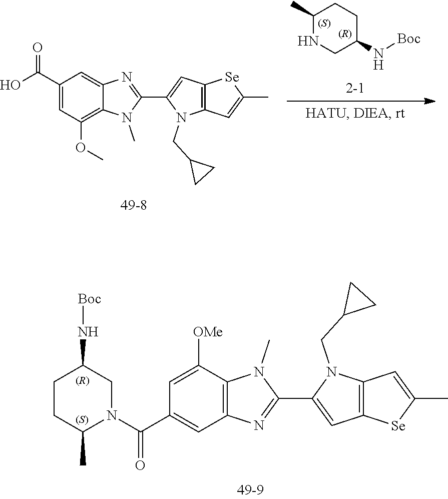

Compound 49-9: To a solution of compound 49-8 (1.0 g, 2.26 mmol), DIEA (876.5 mg, 6.78 mmol) and HATU (1.03 g, 2.71 mmol) in DMF (20 mL) was added compound 2-1 (532.9 mg, 2.49 mmol). The mixture was stirred at room temperature for 3 h. The mixture was diluted with water (100 mL), extracted with ethyl acetate (100 mL×3). The organic phase was washed with water (100 mL), brine (100 mL) and concentrated under vacuum to give the crude product, which was purified by silica gel chromatography (elution gradient: petroleum ether/EtOAc, 1/1 to 0/100, v/v) to afford compound 49-9 (1.2 g) as a brown solid, yield: 83.1%. LCMS: Rt=1.446 min, MS (ESI) m/z=640.1 [M+H]⁺.

### Step 9

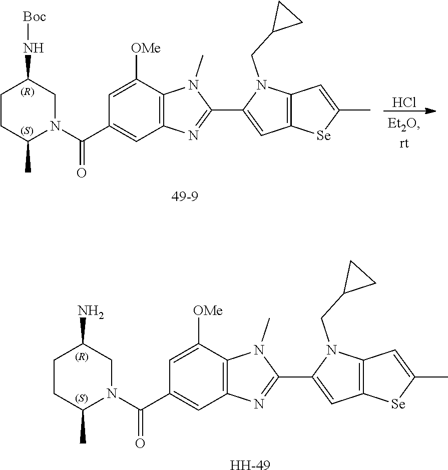

HH-49: To a solution of compound 49-9 (1.2 g, 1.88 mmol) in ethyl ether (10 mL) was added hydrochloric acid solution (10 mL, 2N in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-49 (740 mg, hydrochloric acid salt form) as a yellow solid. yield: 41.6%. ¹H NMR (400 MHz, DMSO-d₆): δ ppm 8.39 (s, 3H), 7.36 (s, 1H), 7.34 (s, 1H), 7.11 (s, 1H), 7.03 (s, 1H), 4.28 (d, J=7.2 Hz, 2H), 4.13 (s, 3H), 4.03 (s, 3H), 3.21-3.08 (m, 2H), 2.63 (s, 3H), 1.95-1.50 (m, 4H), 1.22 (d, J=6.8 Hz, 3H), 1.03-0.96 (m, 1H), 0.36-0.30 (m, 2H), 0.13-0.05 (m, 2H). LCMS: Rt=1.243 min, MS (ESI) m/z=540.0 [M+H]⁺. HPLC: Rt=4.96 min, Purity: 97.55% (214 nm) and 99.13% (254 nm).

### Example 50. Synthesis of HH-50

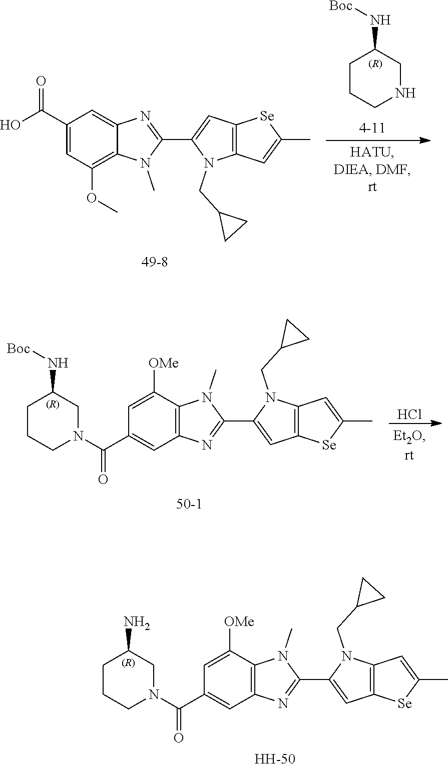

### Step 1

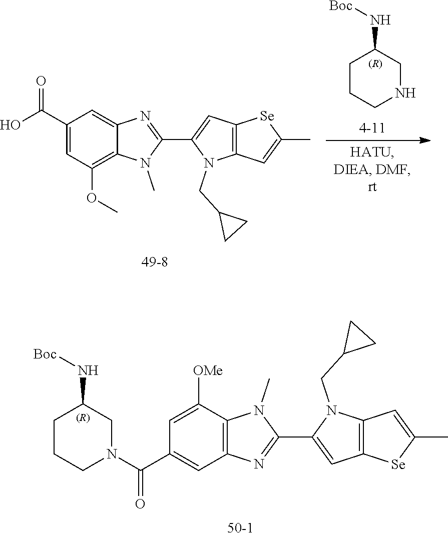

Compound 50-1: To a solution of compound 49-8 (500 mg, 1.13 mmol), DIEA (438.2 mg, 3.39 mmol) and HATU (515.7 mg, 1.36 mmol) in DMF (20 mL) was added compound 4-11 (249 mg, 1.24 mmol). The mixture was stirred at room temperature for 3 h. The mixture was diluted with water (100 mL), extracted with ethyl acetate (50 mL×4). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 3 (400 mg) as a yellow oil, yield: 56.7%. LCMS: Rt=1.381 min, MS (ESI) m/z=626.0 [M+H]⁺.

### Step 2

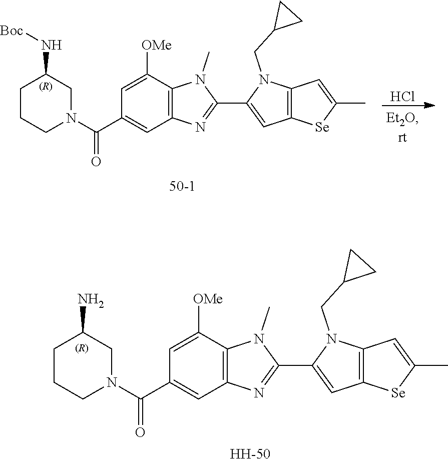

HH-50: To a solution of compound 50-1 (400 mg, 0.64 mmol) in ethyl ether (10 mL) was added hydrochloric acid solution (10 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-50 (307 mg, hydrochloric acid salt form) as a yellow solid. yield: 91.4%. ¹H NMR (400 MHz, DMSO-d₆): δ ppm 8.25 (s, 3H), 7.38 (s, 1H), 7.31 (s, 1H), 7.03 (s, 1H), 7.01 (s, 1H), 4.29 (d, J=6.8 Hz, 2H), 4.11 (s, 3H), 4.01 (s, 3H), 3.34-3.06 (m, 3H), 2.63 (s, 3H), 2.10-1.98 (m, 1H), 1.80-1.50 (m, 3H), 1.08-0.98 (m, 1H), 0.33-0.29 (m, 2H), 0.13-0.06 (m, 2H). LCMS: Rt=1.095 min, MS (ESI) m/z=526.2 [M+H]⁺. HPLC: Rt=4.784 min, Purity: 98.07% (214 nm) and 99.32% (254 nm).

### Example 51. Synthesis of HH-51

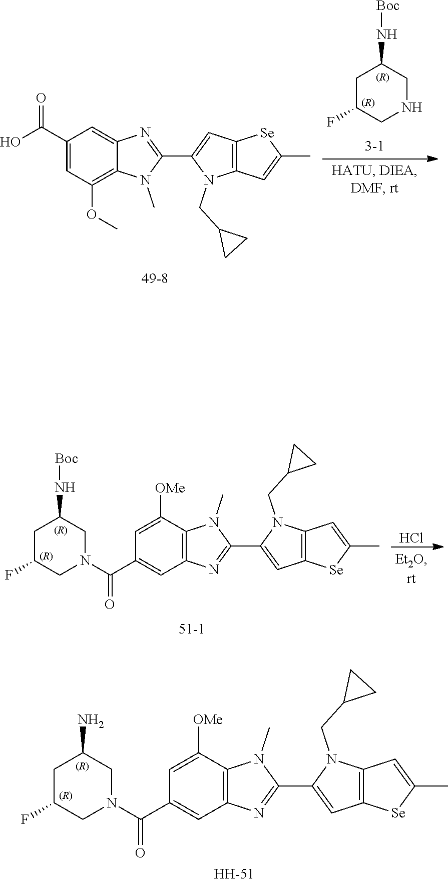

### Step 1

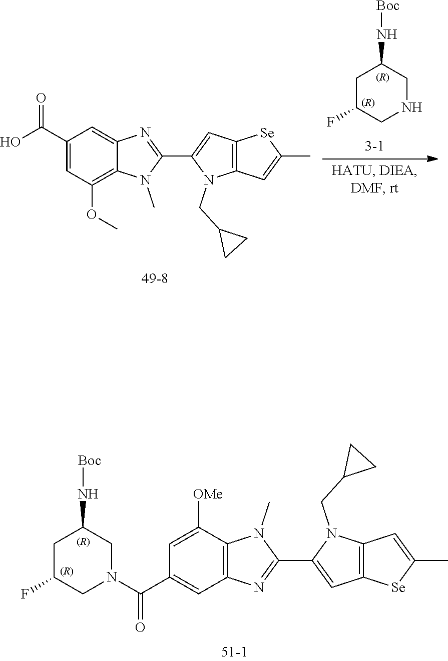

Compound 51-1: To a solution of compound 49-8 (1000 mg, 2.26 mmol), DIEA (876.5 mg, 6.78 mmol) and HATU (1030 mg, 2.71 mmol) in DMF (30 mL) was added compound 3-1 (493 mg, 2.26 mmol). The mixture was stirred at room temperature for 3 h. The mixture was diluted with water (150 mL), extracted with ethyl acetate (100 mL×4). The organic layers were washed with water (100 mL), brine (100 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 3 (1089 mg) as a yellow oil, yield: 75%. LCMS: Rt=1.370 min, MS (ESI) m/z=644.1 [M+H]⁺.

### Step 2

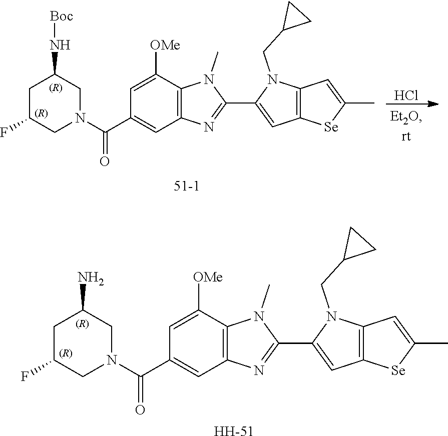

HH-51: To a solution of compound 51-1 (900 mg, 1.40 mmol) in ethyl ether (20 mL) was added hydrochloric acid solution (15 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-51 (335 mg, hydrochloric acid salt form) as a yellow solid. yield: 44.1%. ¹H NMR (400 MHz, DMSO-d₆): δ ppm 8.48 (s, 3H), 7.37 (s, 1H), 7.33 (s, 1H), 7.09 (s, 1H), 7.01 (s, 1H), 5.05-4.70 (m, 2H), 4.29 (d, J=6.8 Hz, 2H), 4.13 (s, 3H), 4.02 (s, 4H), 3.44-3.33 (m, 2H), 3.11-2.95 (m, 1H), 2.63 (s, 3H), 2.40-2.32 (m, 1H), 2.05-1.85 (m, 1H), 1.06-0.96 (m, 1H), 0.36-0.30 (m, 2H), 0.12-0.06 (m, 2H). LCMS: Rt=1.168 min, MS (ESI) m/z=544.0 [M+H]⁺. HPLC: Rt=4.828 min, Purity: 99.21% (214 nm) and 99.52% (254 nm).

### Example 52. Synthesis of HH-52

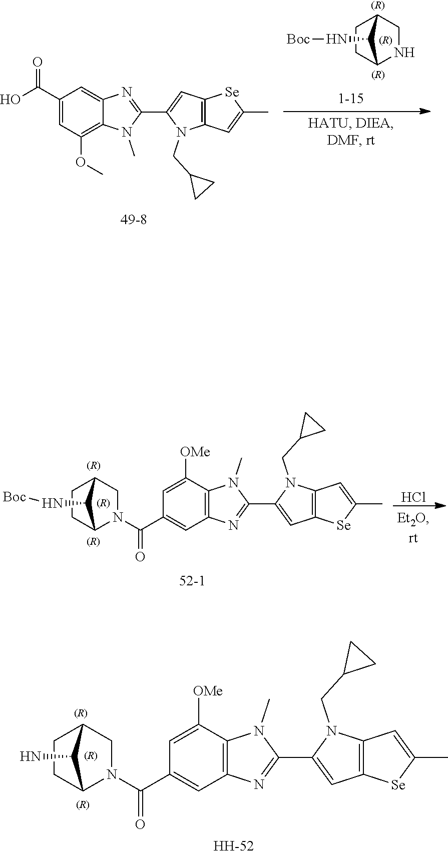

### Step 1

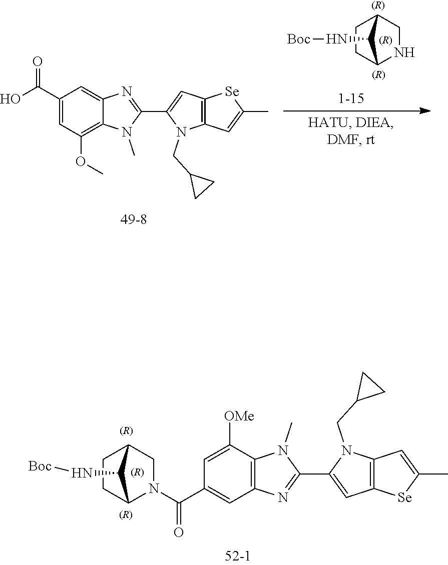

Compound 52-1: To a solution of compound 49-8 (1000 mg, 2.26 mmol), DIEA (876.5 mg, 6.78 mmol) and HATU (1030 mg, 2.71 mmol) in DMF (30 mL) was added compound 1-15 (527.9 mg, 2.49 mmol). The mixture was stirred at room temperature for 3 h. The mixture was diluted with water (150 mL), extracted with ethyl acetate (100 mL×4). The organic layers were washed with water (100 mL), brine (100 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 52-1 (1100 mg) as a yellow oil, yield: 76.4%. LCMS: Rt=1.401 min, MS (ESI) m/z=638.0 [M+H]⁺.

### Step 2

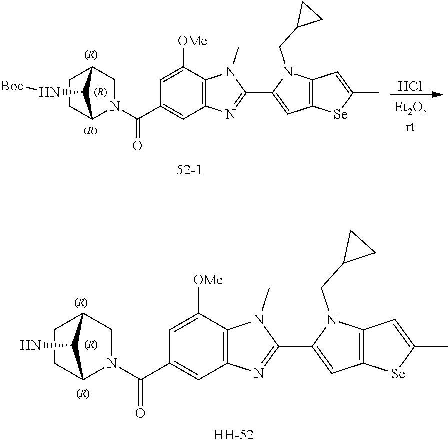

HH-52: To a solution of compound 52-1 (1100 mg, 1.73 mmol) in ethyl ether (20 mL) was added hydrochloric acid solution (15 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-52 (430 mg, hydrochloric acid salt form) as a yellow solid. yield: 46.4%. ¹H NMR (400 MHz, DMSO-d6): δ ppm 8.31 (s, 1H), 7.40 (s, 1H), 7.23 (s, 1H), 6.98 (s, 1H), 6.93 (s, 1H), 4.29 (d, J=6.8 Hz, 1H), 4.08 (s, 3H), 4.00 (s, 3H), 3.69-3.60 (m, 1H), 3.55-3.47 (m, 1H), 3.17 (d, J=10.0 Hz, 1H), 2.66 (s, 1H), 2.62 (s, 3H), 2.04-1.88 (m, 3H), 1.66-1.60 (m, 1H), 1.11-1.03 (m, 1H), 0.37-0.31 (m, 2H), 0.13-0.08 (m, 2H). LCMS: Rt=1.118 min, MS (ESI) m/z=538.1 [M+H]⁺. HPLC: Rt=5.488 min, Purity: 98.99% (214 nm) and 99.33% (254 nm).

### Example 53. Synthesis of HH-53

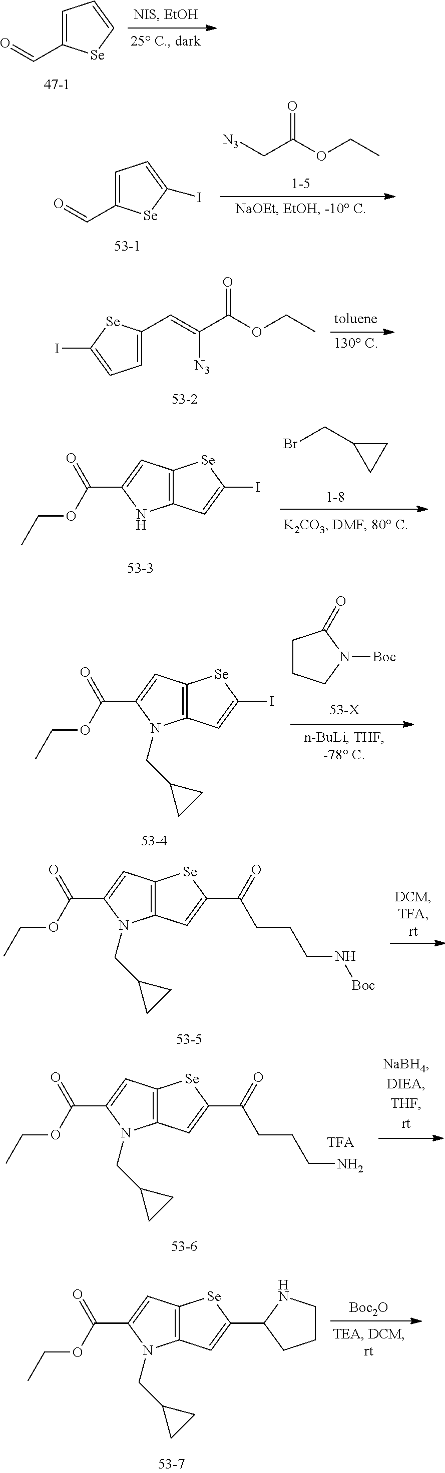

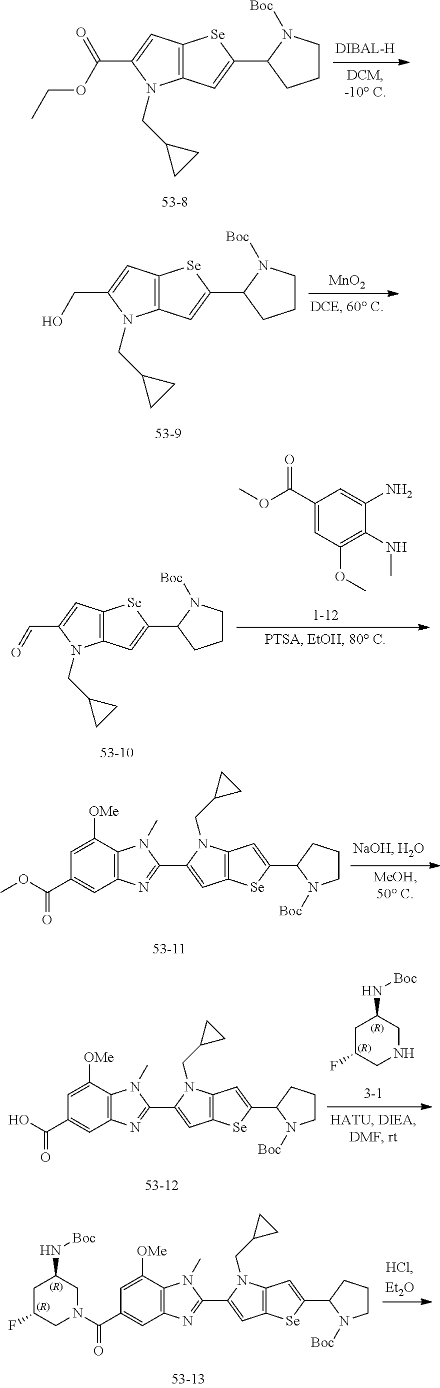

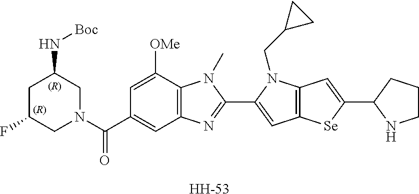

### Step 1

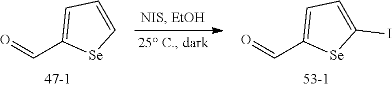 [SMILES: O=Cc1ccc[se]1 ; O=Cc1ccc(I)[se]1]

Compound 53-1: To a solution of compound 47-1 (11 g, 69.2 mmol) and NIS (17.1 g, 76.1 mmol in EtOH (200 mL) was added p-toluenesulfonic acid (1.19 g, 6.92 mmol). The mixture was stirred in the dark at 25° C. for 3 h. The reaction mixture was quenched with 1N hydrochloric acid (100 mL). EtOH was evaporated under vacuum. The residue was diluted with water (100 mL) and extracted with ethyl acetate (100 mL×3). The organic layers were dried over anhydrous Na₂SO₄ and concentrated under vacuum to afford crude product, which was purified by silica gel chromatography (elution gradient: petroleum ether/EtOAc, 10/1, v/v) to afford compound 53-1 (19 g) as a yellow solid, yield: 86.7%. ¹H NMR (400 MHz, CDCl₃) δ ppm 9.74 (s, 1H), 7.70 (d, J=4.1 Hz, 1H), 7.56 (d, J=4.1 Hz, 1H).

### Step 2

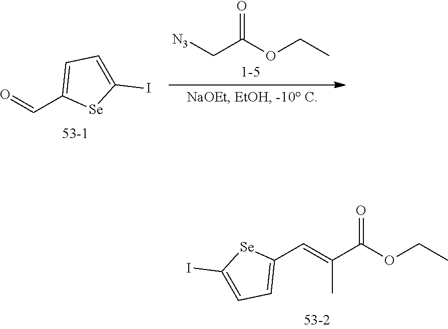

Compound 53-2: To a solution of sodium ethoxide (20% wt. in EtOH) (68.1 g, 200 mmol) in EtOH (200 mL) was slowly added the mixture of compound 3 (19 g, 66.7 mmol) and compound 53-1 (25.8 g, 200 mmol) in EtOH (200 mL) at −10° C. The mixture was stirred at −10° C. under N₂ for 1 h and at room temperature for further 2 h. The mixture was quenched with saturated NH₄Cl aqueous solution (200 mL) and extracted with ethyl acetate (200 mL×3). The organic layers were washed with water (200 mL), brine (200 mL), dried over anhydrous Na₂SO₄ and concentrated under vacuum to afford crude product, which was purified by silica gel chromatography (elution gradient: petroleum ether/EtOAc, 10/1, v/v) to afford compound 53-2 (14 g) as a yellow oil, yield: 47.7%. ¹H NMR (400 MHz, CDCl₃) δ ppm 7.50 (d, J=4.1 Hz, 1H), 7.20 (d, J=0.6 Hz, 1H), 7.03 (dd, J=4.2, 0.7 Hz, 1H), 4.35 (q, J=7.1 Hz, 2H), 1.39 (t, J=7.1 Hz, 3H).

### Step 3

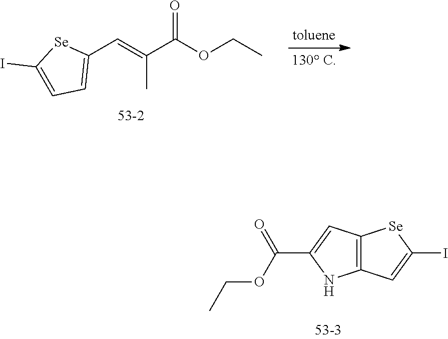

Compound 53-3: A solution of compound 53-2 (14 g, 35.5 mmol) in toluene (300 mL) was stirred at 130° C. under N₂ for 16 h. The reaction mixture was cooled to room temperature and concentrated under vacuum to give the crude product, which was purified by silica gel chromatography (elution gradient: petroleum ether/EtOAc, 10/1, v/v) to afford compound 53-3 (10.4 g) as yellow oil, yield: 71.9%. ¹H NMR (400 MHz, CDCl₃) δ ppm 9.16 (s, 1H), 7.50 (s, 1H), 7.08 (d, J=1.5 Hz, 1H), 4.36 (q, J=7.1 Hz, 2H), 1.38 (t, J=7.1 Hz, 3H).

### Step 4

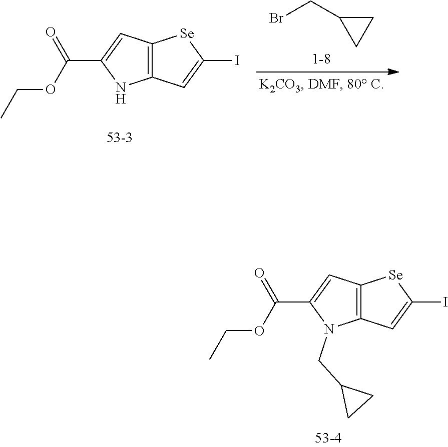 [SMILES: CCOC(=O)c1cc2[se]c(I)cc2[nH]1 ; BrCC1CC1 ; CCOC(=O)c1cc2[se]c(I)cc2n1CC1CC1]

Compound 53-4: To a solution of compound 53-3 (10.4 g, 28.3 mmol) and compound 1-8 (5.73 g, 42.4 mmol) in DMF (200 mL) was added potassium carbonate (7.82 g, 56.6 mmol). The mixture was stirred at 80° C. for 3 h. The mixture was diluted with water (200 mL) and extracted with ethyl acetate (200 mL×3). The organic layers were washed with water (200 mL) and brine (200 mL), dried over anhydrous Na₂SO₄ and concentrated under vacuum to afford crude product, which was purified by silica gel chromatography (elution gradient: petroleum ether/EtOAc, 5/1, v/v) to afford compound 53-4 (9 g) as a yellow oil, yield: 67.8%. ¹H NMR (400 MHz, CDCl₃) δ ppm 7.48 (s, 1H), 7.14 (s, 1H), 4.36 (d, J=6.9 Hz, 2H), 4.31 (q, J=7.1 Hz, 2H), 1.37 (t, J=7.1 Hz, 3H), 1.34-1.28 (m, 1H), 0.54-0.48 (m, 2H), 0.39-0.35 (m, 2H).

### Step 5

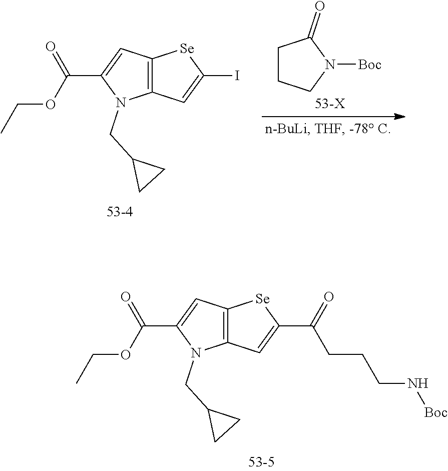

Compound 53-5: To a solution of compound 53-4 (3 g, 7.09 mmol) in dry THF (40 mL) was added n-BuLi (3 mL, 7.2 mmol) under N₂ at −78° C. The mixture was stirred at −78° C. for 1 h. To the mixture compound 53-X (1.5 g, 8.10 mmol) was added dropwise at −78° C. The reaction mixture was allowed to warm to room temperature and stirred for 15 h. The mixture was quenched with saturated ammonium chloride aqueous solution (20 mL) and extracted with ethyl acetate (50 mL×2). The organic layer was dried over anhydrous sodium sulfate, filtered and concentrated under vacuum to afford crude product, which was purified by flash chromatography (elution gradient: petroleum ether/ethyl acetate, 5/1, v/v) to afford compound 53-5 (1.4 g) as a brown oil, yield: 41.2%. ¹H NMR (400 MHz, CDCl₃): δ ppm 7.85 (s, 1H), 7.23 (s, 1H), 4.75-4.58 (m, 1H), 4.45 (d, J=6.8 Hz, 2H), 4.35 (q, J=7.2 Hz, 2H), 3.24 (m, 2H), 2.99 (t, J=7.2 Hz, 2H), 2.00-1.93 (m, 2H), 1.42 (s, 9H), 1.38 (t, J=7.2 Hz, 3H), 1.35-1.30 (m, 1H), 0.56-0.51 (m, 2H), 0.43-0.39 (m, 2H). LCMS: Rt=1.47 min, MS (ESI) m/z=427.0 [M−56]⁺.

### Step 6

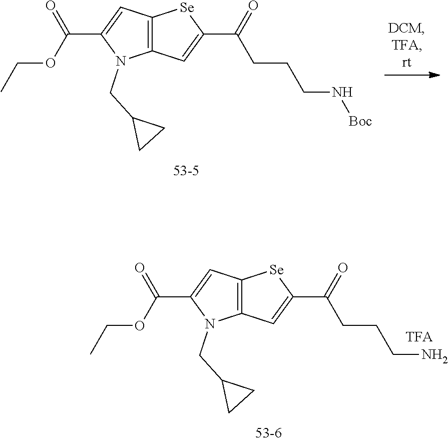 [SMILES: CCOC(=O)c1cc2[se]c(C(=O)CCCNC(=O)OC(C)(C)C)cc2n1CC1CC1 ; CCOC(=O)c1cc2[se]c(C(=O)CCCN)cc2n1CC1CC1]

Compound 53-6: To a solution of compound 53-5 (1.4 g, 2.90 mmol) in DCM (20 mL) was added TFA (4 mL). The mixture was stirred at room temperature for 3 h. The mixture was concentrated under vacuum to afford compound 53-6 (1.2 g, crude product) as a black oil, and used into next step without further purification. LCMS: Rt=1.12 min, MS (ESI) m/z=382.9 [M+H]⁺.

### Step 7

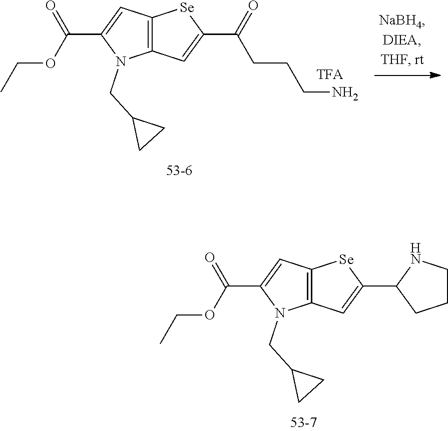 [SMILES: CCOC(=O)c1cc2[se]c(C(=O)CCCN)cc2n1CC1CC1 ; CCOC(=O)c1cc2[se]c(C3CCCN3)cc2n1CC1CC1]

Compound 53-7: To a solution of compound 53-6 (1.2 g, crude) in THF (30 mL) was added DIEA (365 mg, 2.83 mmol) and AcOH (0.5 mL). The mixture was stirred at room temperature for 1 h. Then NaBH₄ (160 mg, 4.24 mmol) was added into the mixture and stirred at room temperature for 1 h. The mixture was concentrated under reduced pressure to afford residue. The residue was diluted with water (50 mL), extracted with ethyl acetate (50 mL×2). The combined organic layers were dried over anhydrous sodium sulfate, filtered and concentrated under vacuum to afford crude product, which was purified by flash chromatography (elution gradient: dichloromethane/e methanol, 10/1, v/v) to afford compound 53-7 (600 mg) as a black oil, yield: 56.4% (2 steps). LCMS: Rt=1.05 min, MS (ESI) m/z=367.1 [M+H]⁺.

### Step 8

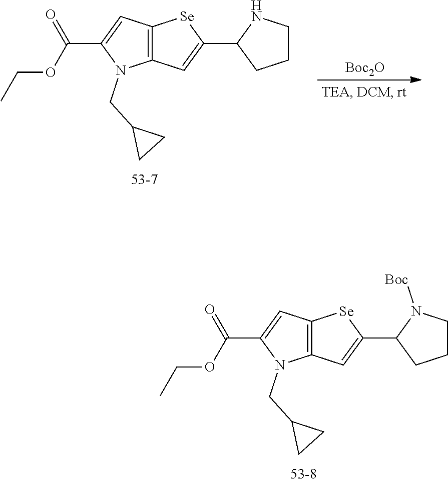 [SMILES: "Boc2O" ; CCOC(=O)c1cc2[se]c(C3CCCN3)cc2n1CC1CC1 ; CCOC(=O)c1cc2[se]c(C3CCCN3C(=O)OC(C)(C)C)cc2n1CC1CC1]

Compound 53-8: To a solution of compound 53-7 (0.6 g, 1.64 mmol) in DCM (20 mL) was added Boc₂O (538 mg, 2.47 mmol) and triethylamine (499 mg, 4.94 mmol). The mixture was stirred at room temperature for 2 h. The mixture was concentrated under vacuum to afford crude product, which was purified by flash chromatography (elution gradient: petroleum ether/ethyl acetate, 5/1, v/v) to afford compound 53-8 (520 mg) as a yellow solid, yield: 68.1%. LCMS: Rt=1.57 min, MS (ESI) m/z=489.1 [M+Na]⁺.

### Step 9

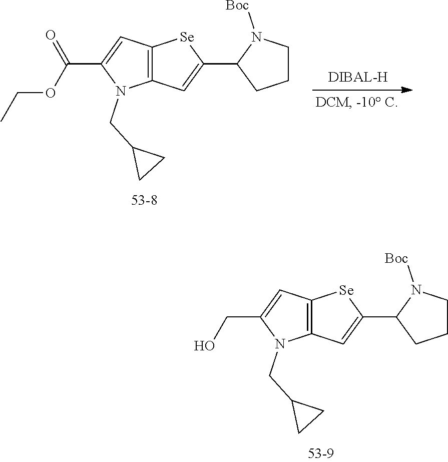 [SMILES: CCOC(=O)c1cc2[se]c(C3CCCN3C(=O)OC(C)(C)C)cc2n1CC1CC1 ; CC(C)(C)OC(=O)N1CCCC1c1cc2c(cc(CO)n2CC2CC2)[se]1]

Compound 53-9: To a solution of compound 53-8 (520 mg, 1.12 mmol) in DCM (10 mL) was added DIBAL-H (1M in THF, 2.2 mL, 2.2 mmol) under N₂ at −10° C. The mixture was stirred at −10° C. for 5 h. The mixture was quenched with saturated ammonium chloride aqueous solution (10 mL), filtered and extracted with ethyl acetate (50 mL×2). The organic layer was dried over anhydrous sodium sulfate, filtered and concentrated under vacuum to afford compound 53-9 (240 mg) as a yellow solid, yield: 50.7%. LCMS: Rt=1.36 min, MS (ESI) m/z=425.0 [M+H]⁺.

### Step 10

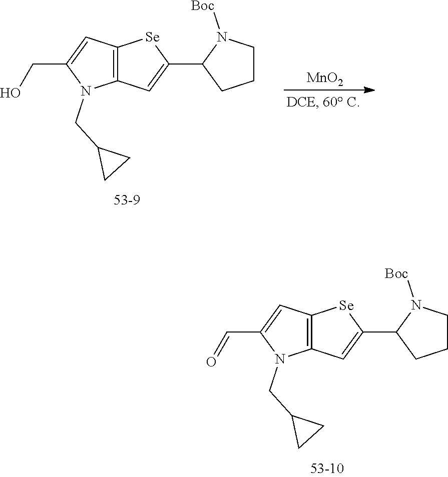

Compound 53-10: To a solution of compound 53-9 (240 mg, 0.57 mmol) in DCE (10 mL) was added MnO₂ (492 mg, 5.66 mmol). The mixture was stirred at 60° C. for 16 h. The mixture was filtered and concentrated under reduced pressure to afford crude product, which was purified by flash chromatography (elution gradient: petroleum ether/ethyl acetate, 2/1, v/v) to afford compound 53-10 (188 mg) as a yellow solid, yield: 78.7%. ¹H NMR (400 MHz, CDCl₃): δ ppm 9.55 (s, 1H), 7.09 (s, 1H), 7.00-6.96 (m, 1H), 5.19-5.08 (m, 1H), 4.44-4.29 (m, 2H), 3.56-3.46 (m, 2H), 2.36-2.30 (m, 1H), 1.99-1.91 (m, 1H), 1.47-1.24 (m, 11H), 0.90-0.84 (m, 1H), 0.52-0.47 (m, 2H), 0.40-0.36 (m, 2H).

### Step 11

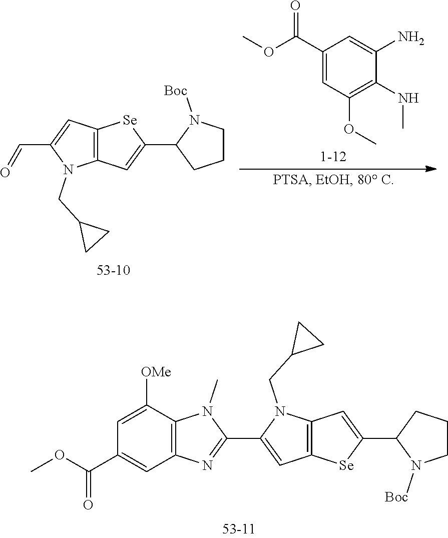

Compound 53-11: To a solution of compound 53-10 (188 mg, 0.45 mmol) in EtOH (10 mL) was added compound 1-12 (94 mg, 0.45 mmol) and PTSA (8 mg, 0.047 mmol). The mixture was stirred at 80° C. for 2 h. The mixture was filtered and concentrated under reduced pressure to afford compound 53-11 (250 mg, crude) as a yellow oil and used into next step without further purification. LCMS: Rt=1.33 min, MS (ESI) m/z=613.2 [M+H]⁺.

### Step 12

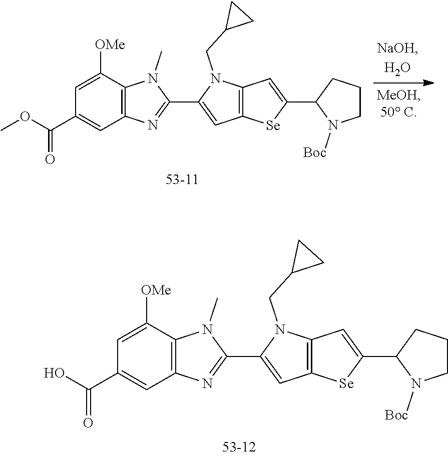 [SMILES: COC(=O)c1cc(OC)c2c(c1)nc(-c1cc3[se]c(C4CCCN4C(=O)OC(C)(C)C)cc3n1CC1CC1)n2C ; COc1cc(C(=O)O)cc2nc(-c3cc4[se]c(C5CCCN5C(=O)OC(C)(C)C)cc4n3CC3CC3)n(C)c12]

Compound 53-12: To a solution of compound 53-11 (250 mg, crude) in MeOH (5 mL)/THF (5 mL) was added 2N NaOH (1 mL, 2 mmol). The mixture was stirred at 50° C. for 2 h. The mixture was concentrated and adjusted pH to 5-6 with 1N hydrochloric acid aqueous solution, then extracted with dichloromethane (50 mL×3). The organic phases were combined, dried over Na₂SO₄, filtered and concentrated under vacuum to give compound 53-12 (220 mg) as a yellow oil, yield: 82.7%. (2 steps). LCMS: Rt=1.40 min, MS (ESI) m/z=599.0 [M+H]⁺.

### Step 13

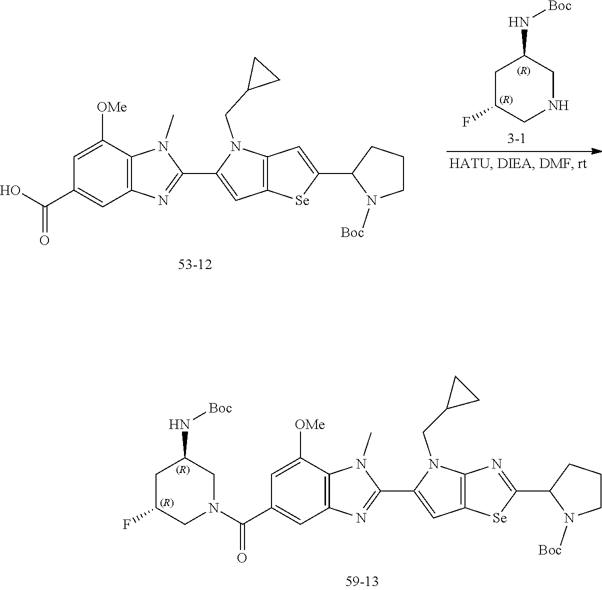

Compound 53-13: To a solution of compound 53-12 (110 mg, 0.18 mmol), DIEA (48 mg, 0.37 mol), HATU (91 mg, 0.24 mmol) in DMF (5 mL) was added compound 3-1 (44 mg, 0.20 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 53-13 (105 mg) as a yellow solid, yield: 71.5%. LCMS: Rt=1.45 min, MS (ESI) m/z=799.0 [M+H]⁺.

### Step 14

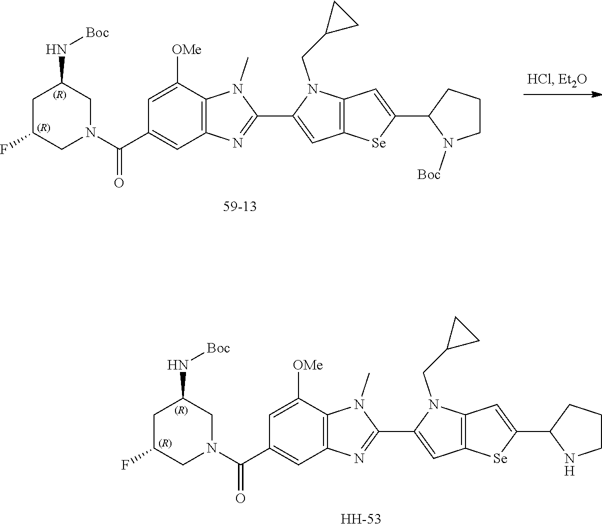

HH-53: To a solution of compound 53-13 (105 mg, 0.13 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-30%) to afford HH-53 (63 mg, hydrochloric acid salt form) as a brown solid. yield: 71.4%. ¹H NMR (400 MHz, DMSO-d₆): δ ppm 10.30 (s, 1H), 9.17 (s, 1H), 8.49 (s, 3H), 7.86 (s, 1H), 7.36 (s, 1H), 7.12 (s, 1H), 6.97 (s, 1H), 5.05 (m, 1H), 4.91-4.84 (m, 1H), 4.33 (d, J=6.8 Hz, 2H), 4.11 (s, 3H), 4.06 (s, 3H), 4.11-4.06 (m, 1H), 3.37-3.25 (m, 4H), 3.04 (br. s, 1H), 2.51-2.33 (m, 2H), 2.19-1.87 (m, 4H), 1.12-1.02 (m, 1H), 0.35-0.33 (m, 2H), 0.14 (m, 2H). LCMS: Rt=1.00 min, MS (ESI) m/z=599.0 [M+H]⁺. HPLC: Rt=4.43 min, Purity: 100% (214 nm and 254 nm)

### Example 54. Synthesis of HH-54

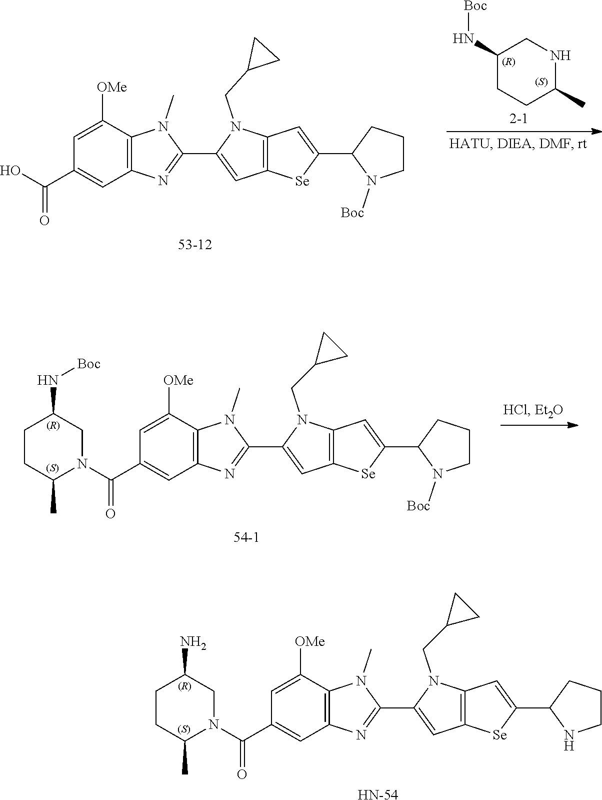

### Step 1

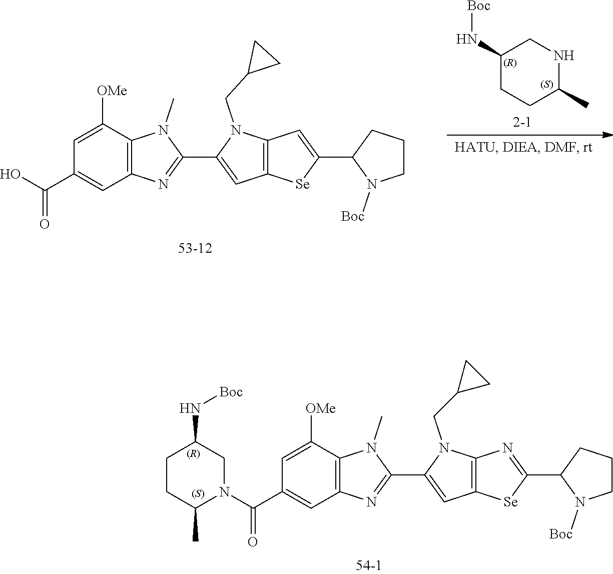

Compound 54-1: To a solution of compound 53-12 (55 mg, 0.092 mmol), DIEA (36 mg, 0.28 mol), HATU (45 mg, 0.12 mmol) in DMF (5 mL) was added compound 2-1 (22 mg, 0.10 mmol). The mixture was stirred at room temperature for 2 h. The residue was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 54-1 (42 mg) as a yellow oil, yield: 57.5%. LCMS: Rt=1.59 min, MS (ESI) m/z=795.3 [M+H]⁺.

### Step 2

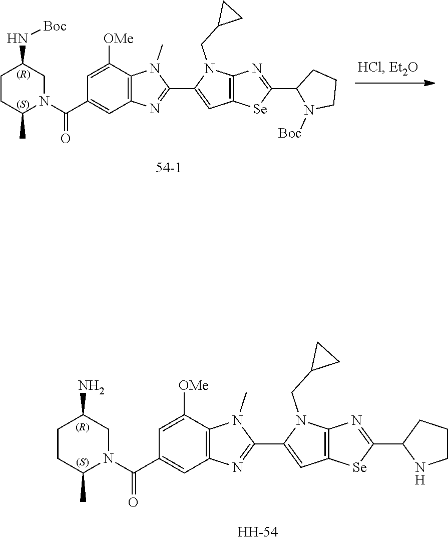

HH-54: To a solution of compound 54-1 (42 mg, 0.053 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-30%) to afford HH-54 (18 mg, hydrochloric acid salt form) as a brown solid. yield: 51.3%. ¹H NMR (400 MHz, DMSO-d₆): δ ppm 10.18 (s, 1H), 9.11 (s, 1H), 8.28 (s, 3H), 7.84 (s, 1H), 7.32 (s, 1H), 7.08 (s, 1H), 6.92 (s, 1H), 4.89-4.84 (m, 1H), 4.33 (d, J=6.8 Hz, 2H), 4.22-4.18 (m, 2H), 4.10 (s, 3H), 4.00 (s, 3H), 3.39-3.24 (m, 2H), 3.17-3.07 (m, 2H), 2.46-2.44 (m, 1H), 2.19-2.04 (m, 3H), 1.89-1.73 (m, 3H), 1.59-1.56 (m, 1H), 1.21 (d, J=6.8 Hz, 3H), 1.11-1.05 (m, 1H), 0.37-0.33 (m, 2H), 0.16-0.12 (m, 2H). LCMS: Rt=1.06 min, MS (ESI) m/z=595.0 [M+H]⁺. HPLC: Rt=3.99 min, Purity: 99.68% (214 nm) and 99.63% (254 nm).

### Example 55. Synthesis of HH-55

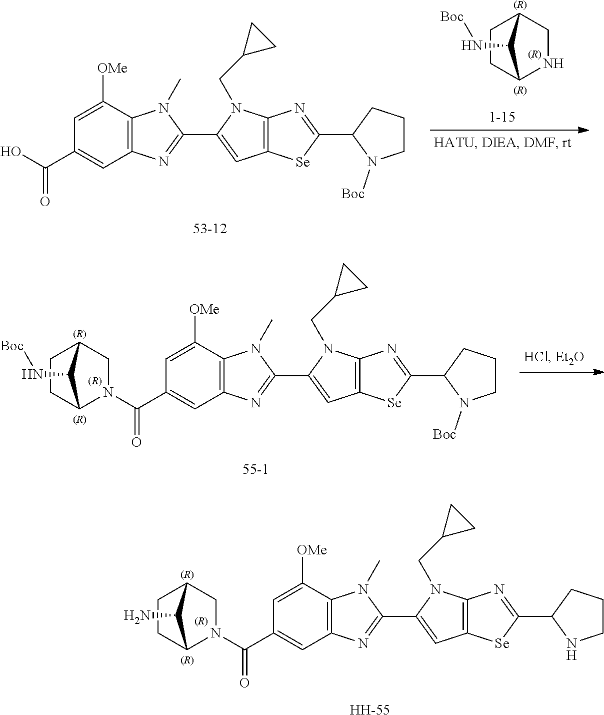

### Step 1

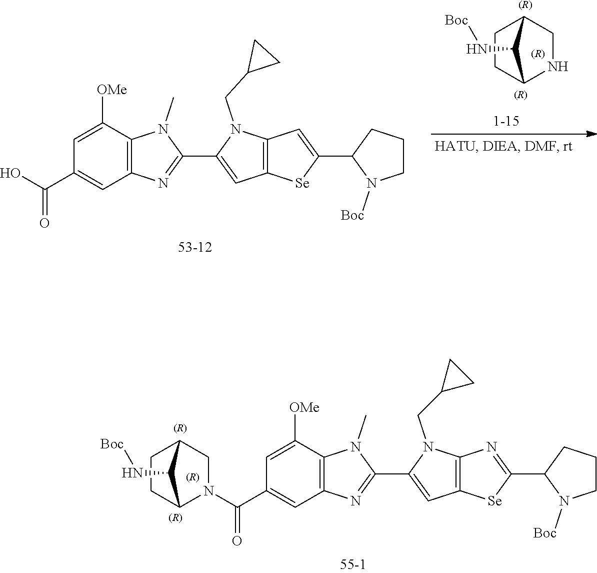

Compound 55-1: To a solution of compound 53-12 (110 mg, 0.18 mmol), DIEA (48 mg, 0.37 mol), HATU (91 mg, 0.24 mmol) in DMF (5 mL) was added compound 1-15 (43 mg, 0.20 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 55-1 (100 mg) as a yellow solid, yield: 68.6%. LCMS: Rt=1.46 min, MS (ESI) m/z=793.0 [M+H]⁺.

### Step 2

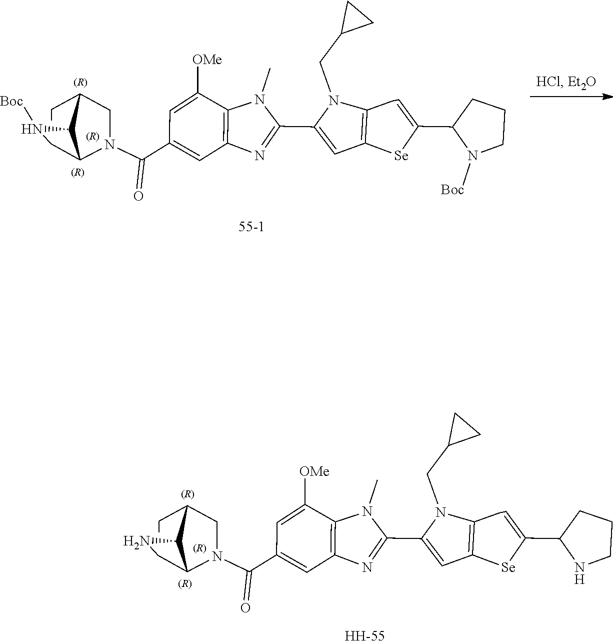

HH-55: To a solution of compound 55-1 (100 mg, 0.13 mmol) in ethyl ether (2 mL) was added hydrochloric acid solution (2 mL, 2M in ethyl ether). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 10-30%) to afford HH-55 (49.9 mg, hydrochloric acid salt form) as a brown solid. yield: 59.5%. ¹H NMR (400 MHz, DMSO-d₆): δ ppm 10.33 (s, 1H), 9.10 (s, 1H), 8.45 (s, 3H), 7.81 (s, 1H), 7.41 (s, 1H), 7.03 (s, 1H), 6.99 (s, 1H), 4.89 (br. s, 2H), 4.32 (d, J=6.8 Hz, 2H), 4.33-4.25 (m, 1H), 4.08 (s, 3H), 4.01 (s, 3H), 3.65-3.63 (m, 1H), 3.50 (m, 1H), 3.41-3.29 (m, 2H), 3.18-3.16 (m, 1H), 2.70-2.66 (m, 1H), 2.20-1.89 (m, 6H), 1.64-1.60 (m, 1H), 1.14-1.08 (m, 1H), 0.37-0.35 (m, 2H), 0.17-0.13 (m, 2H). LCMS: Rt=0.99 min, MS (ESI) m/z=592.9 [M+H]⁺. HPLC: Rt=3.84 min, Purity: 99.19% (214 nm) and 100% (254 nm).

### Example 56. Synthesis of HH-56

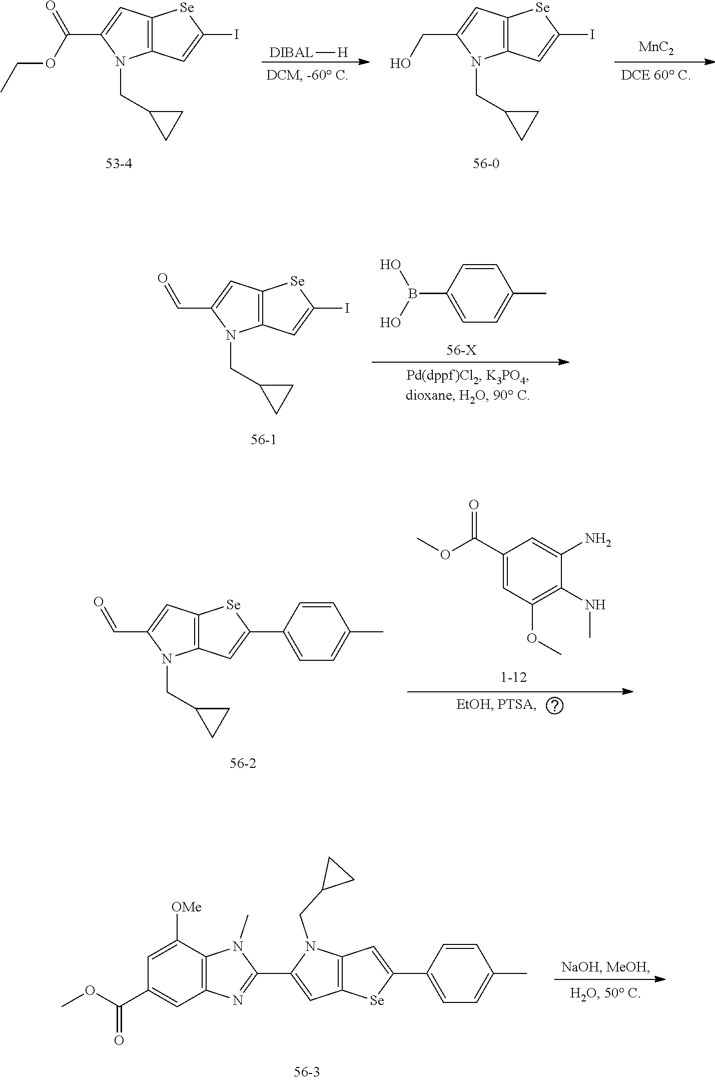

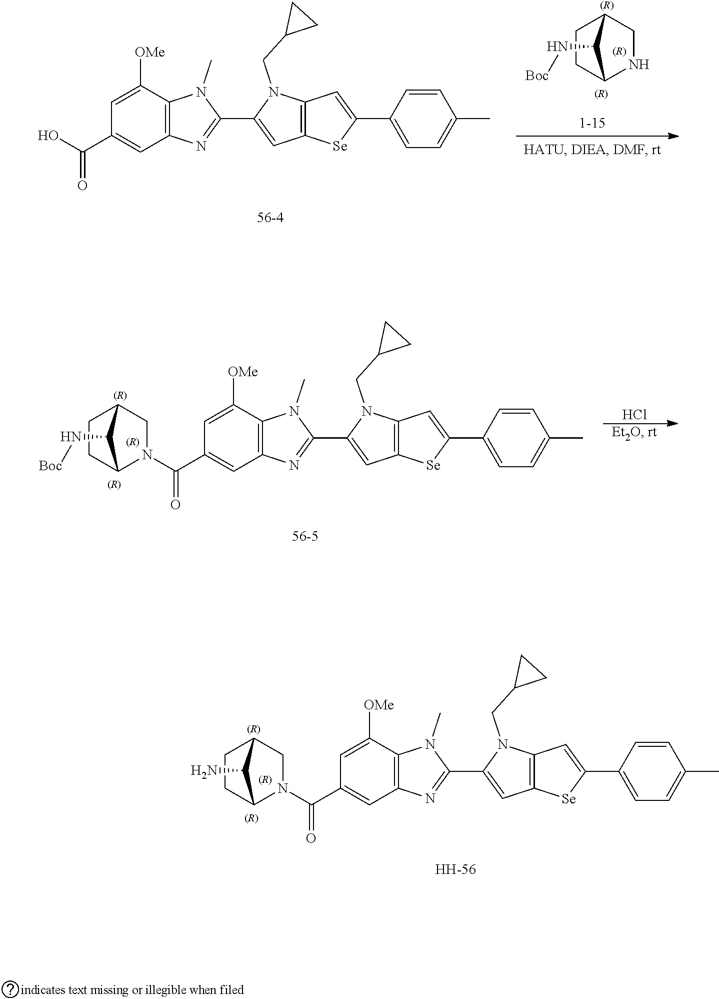

### Step 1

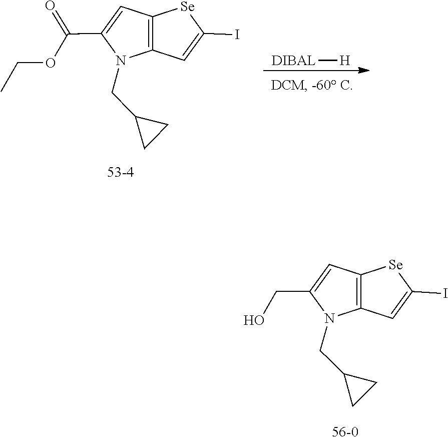

Compound 56-0: To a solution of compound 53-4 (2 g, 4.74 mmol) in DCM (200 mL) cooled at −60° C. under N₂ was slowly added DIBAL-H (1 M in THF, 14.2 mL, 14.2 mmol). The mixture was stirred at −60° C. under N₂ for 2 h. The mixture was quenched with water (50 mL) and filtered. The filtrate was extracted with dichloromethane (100 mL×3). The organic layers were dried over anhydrous Na₂SO₄ and concentrated under vacuum to afford compound 56-0 (1.8 g) as a yellow oil, yield: 89.9%. LCMS: Rt=1.33 min, MS (ESI) m/z=381.8 [M+H]⁺.

### Step 2

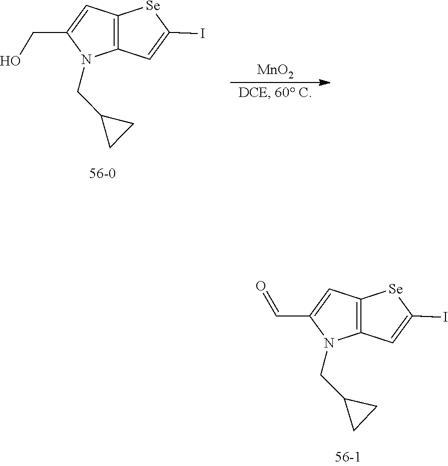 [SMILES: OCc1cc2[se]c(I)cc2n1CC1CC1 ; "MnO2" ; "DCE, 60° C." ; O=Cc1cc2[se]c(I)cc2n1CC1CC1]

Compound 56-1: To a solution of compound 56-0 (1.8 g, 4.73 mmol) in DCE (100 mL) was added manganese dioxide (4.12 g, 47.3 mmol). The mixture was stirred at 60° C. for 16 h. The mixture was filtered and the filtrate was concentrated under vacuum to afford crude product, which was purified by silica gel chromatography (elution gradient: petroleum ether/EtOAc, 5/1, v/v) to afford compound 56-1 (0.9 g) as a yellow oil, yield: 45.2%. LCMS: Rt=1.43 min, MS (ESI) m/z=379.8 [M+H]⁺.

### Step 3

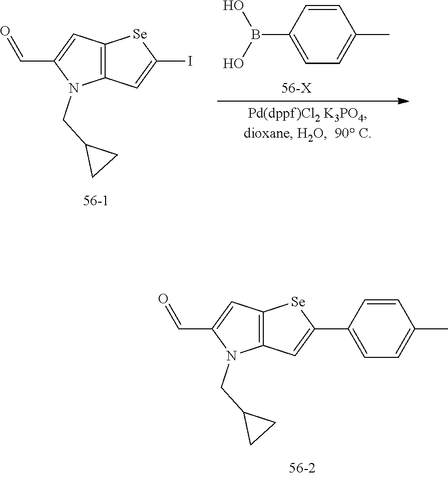

Compound 56-2: To a solution of compound 56-1 (300 mg, 0.78 mmol), compound 56-X (162 mg, 1.2 mmol), 1,1′-bis(diphenylphosphino)ferrocene-palladium(II) dichloride dichloromethane complex (32.4 mg, 0.04 mmol) in dioxane (20 mL) and water (2 mL) was added potassium phosphate tribasic (505.4 mg, 2.38 mmol). The mixture was stirred at 90° C. under N₂ for 3 h. The mixture was diluted with water (20 mL) and extracted with ethyl acetate (30 mL×4). The organic phase was washed with brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 56-2 (180 mg) as a yellow oil, yield: 59.6%. LCMS: Rt=1.49 min, MS (ESI) m/z=344.1 [M+H]⁺.

### Step 4

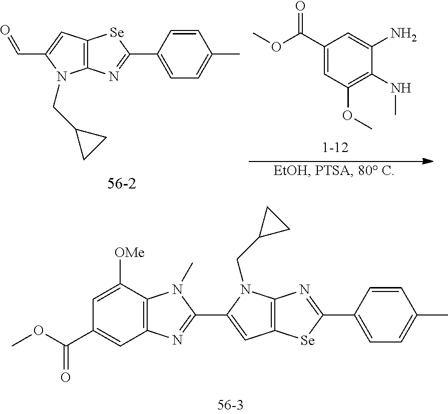

Compound 56-3: To a solution of compound 56-2 (180 mg, 0.52 mmol) and compound 1-12 (110.6 mg, 0.52 mmol) in EtOH (20 mL) was added p-toluenesulfonic acid (9 mg, 0.06 mmol). The mixture was stirred at 80° C. for 3 h. The solvent was evaporated and diluted with water (20 mL), extracted with ethyl acetate (20 mL×3). The organic layers were washed with water (10 mL), brine (10 mL), dried over anhydrous Na₂SO₄ and concentrated under vacuum to afford crude product, which was purified by silica gel chromatography (elution gradient: petroleum ether/EtOAc, 5/1, v/v) to afford compound 56-3 (160 mg) as a yellow oil, yield: 51.4%. LCMS: Rt=1.60 min, MS (ESI) m/z=533.9 [M+H]⁺.

### Step 5

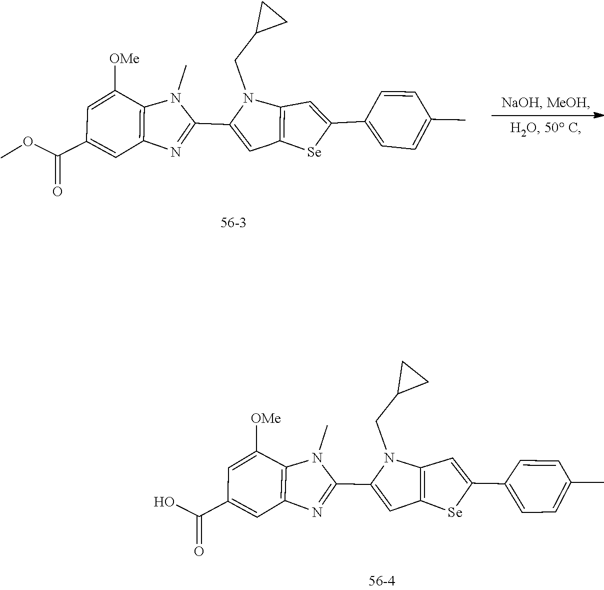

Compound 56-4: To a suspension of compound 56-3 (160 mg, 0.3 mmol) in MeOH (10 mL) was added 2N sodium hydroxide aqueous solution (0.4 mL, 0.60 mmol). The mixture was stirred at 50° C. for 3 h. The mixture was concentrated and adjusted pH to 5-6 with 1N hydrochloric acid aqueous solution, then extracted with ethyl acetate (20 mL×3). The organic phase was washed with water (15 mL), brine (15 mL), dried over anhydrous Na₂SO₄ and concentrated under vacuum to give compound 56-4 (120 mg) as a yellow solid, yield: 77.0%. LCMS: Rt=1.49 min, MS (ESI) m/z=519.9 [M+H]⁺.

### Step 6

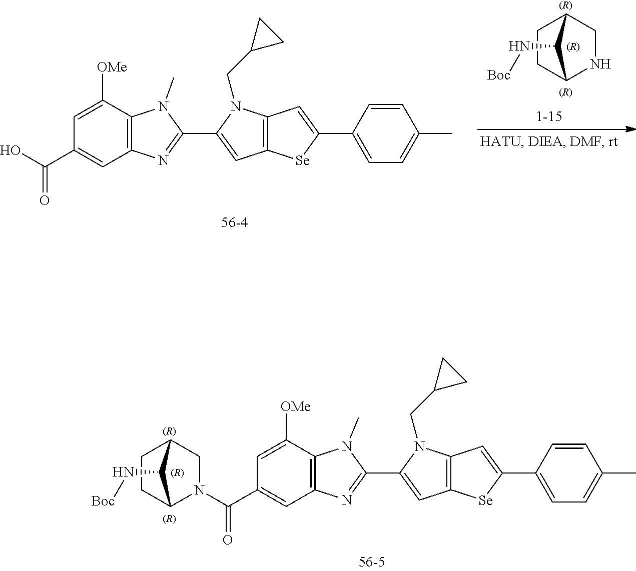

Compound 56-5: To a solution of compound 56-4 (120 mg, 0.22 mmol), DIEA (90 mg, 0.7 mmol) and HATU (105.6 mg, 0.28 mmol) in DMF (8 mL) was added compound 7 (27.0 mg, 0.13 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 56-5 (140 mg) as a brown solid, yield: 84.9%. LCMS: Rt=1.55 min, MS (ESI) m/z=714.2 [M+H]⁺.

### Step 7

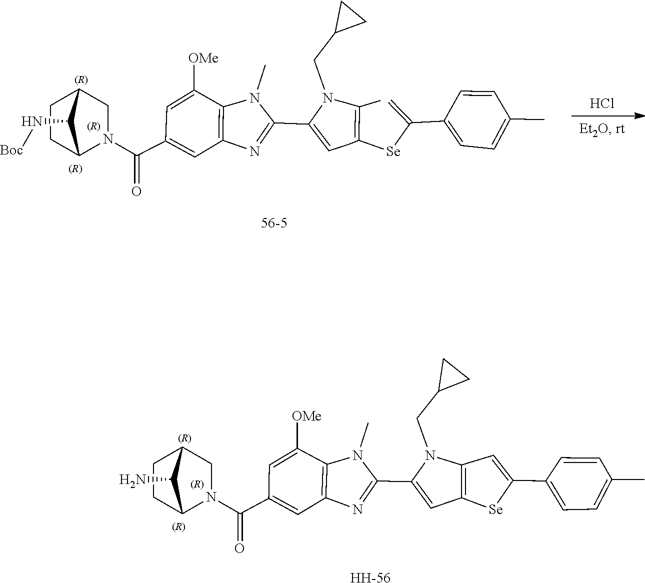

HH-56: To a solution of compound 56-5 (140 mg, 0.20 mmol) in ethyl ether (5 mL) was added hydrochloric acid solution (8 mL, 2M in ethyl ether). The mixture was stirred at room temperature for 5 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-56 (80 mg, hydrochloric acid salt form) as a yellow solid. yield: 66.4%. ¹H NMR (400 MHz, DMSO-d₆): δ ppm 8.45 (s, 3H), 7.90 (s, 1H), 7.54 (d, J=8.0 Hz, 2H), 7.43 (s, 1H), 7.23 (d, J=8.0 Hz, 2H), 7.03 (d, J=14.0 Hz, 2H), 4.39 (d, J=6.8 Hz, 3H), 4.11 (s, 3H), 4.02 (s, 3H), 3.70-3.45 (m, 2H), 3.18 (d, J=10.4 Hz, 1H), 2.67 (s, 1H), 2.33 (s, 3H), 2.07-1.85 (m, 3H), 1.68-1.55 (m, 1H), 1.17-1.07 (m, 1H), 0.40-0.30 (m, 2H), 0.22-0.12 (m, 2H). LCMS: Rt=1.31 min, MS (ESI) m/z=614.1 [M+H]⁺. HPLC: Rt=6.08 min, Purity: 98.95% (214 nm) and 98.82% (254 nm).

### Example 57. Synthesis of HH-57

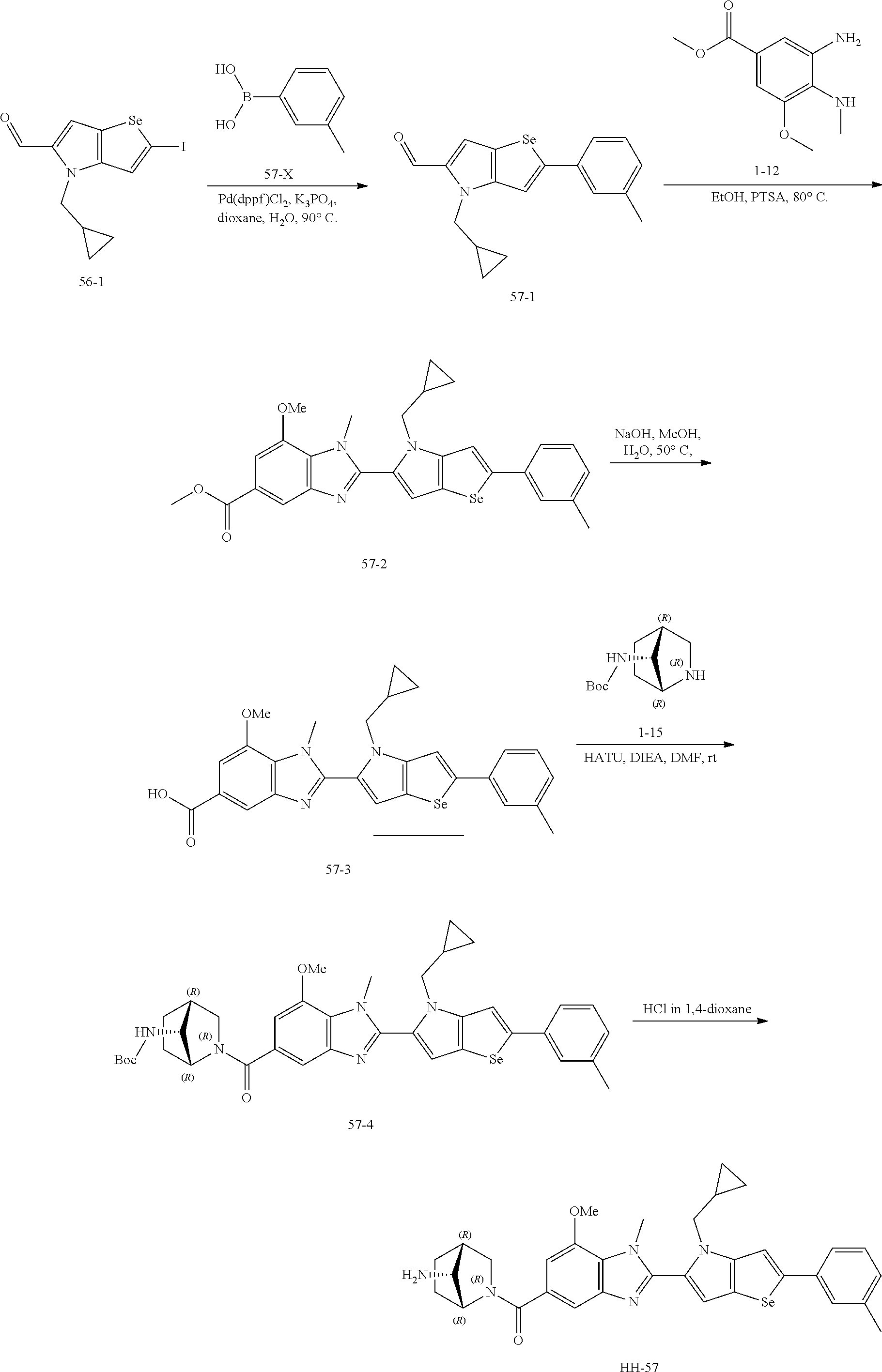

### Step 1

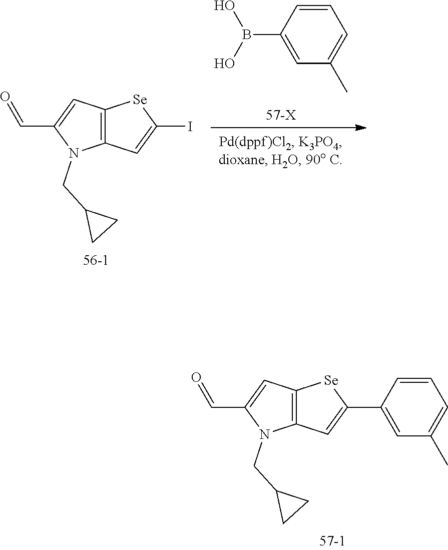

Compound 57-1: To a solution of compound 56-1 (200 mg, 0.53 mmol), compound 57-X (107.9 mg, 0.79 mmol), 1,1′-bis(diphenylphosphino)ferrocene-palladium(II) dichloride dichloromethane complex (43.2 mg, 0.05 mmol) in dioxane (10 mL) and water (2 mL) was added potassium phosphate tribasic (336.9 mg, 1.59 mmol). The mixture was stirred at 90° C. under N₂ for 3 h. The mixture was diluted with water (20 mL) and extracted with ethyl acetate (30 mL×4). The organic phase was washed with brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 57-1 (120 mg) as a yellow oil, yield: 59.6%. LCMS: Rt=1.55 min, MS (ESI) m/z=343.9 [M+H]⁺.

### Step 2

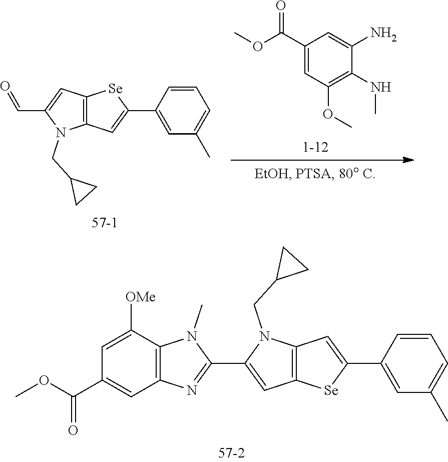

Compound 57-2: To a solution of compound 57-1 (120 mg, 0.35 mmol) and compound 1-12 (73.7 mg, 0.35 mmol) in EtOH (20 mL) was added p-toluenesulfonic acid (6.0 mg, 0.03 mmol). The mixture was stirred at 80° C. for 3 h. The solvent was evaporated and diluted with water (20 mL), extracted with ethyl acetate (20 mL×3). The organic layers were washed with water (10 mL), brine (10 mL), dried over anhydrous Na₂SO₄ and concentrated under vacuum to afford crude product, which was purified by silica gel chromatography (elution gradient: petroleum ether/EtOAc, 2/1, v/v) to afford compound 57-2 (100 mg) as a yellow oil, yield: 48.2%. LCMS: Rt=1.68 min, MS (ESI) m/z=534.2 [M+H]⁺.

### Step 3

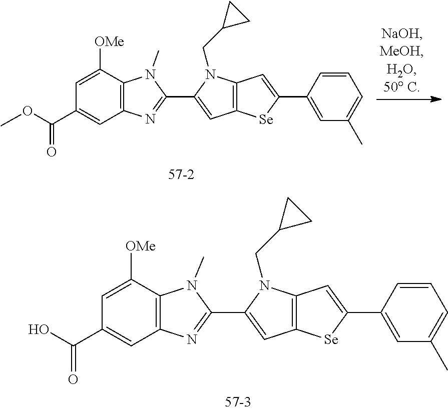

Compound 57-3: To a suspension of compound 57-2 (100 mg, 0.15 mmol) in MeOH (10 mL) was added 2N sodium hydroxide aqueous solution (0.2 mL, 0.38 mmol). The mixture was stirred at 50° C. for 3 h. The mixture was concentrated and adjusted pH to 5-6 with 1N hydrochloric acid aqueous solution, then extracted with ethyl acetate (20 mL×3). The organic phase was washed with water (15 mL), brine (15 mL), dried over anhydrous Na₂SO₄ and concentrated under vacuum to give compound 57-3 (80 mg) as a yellow solid, yield: 82.2%. LCMS: Rt=1.49 min, MS (ESI) m/z=519.9 [M+H]⁺.

### Step 4

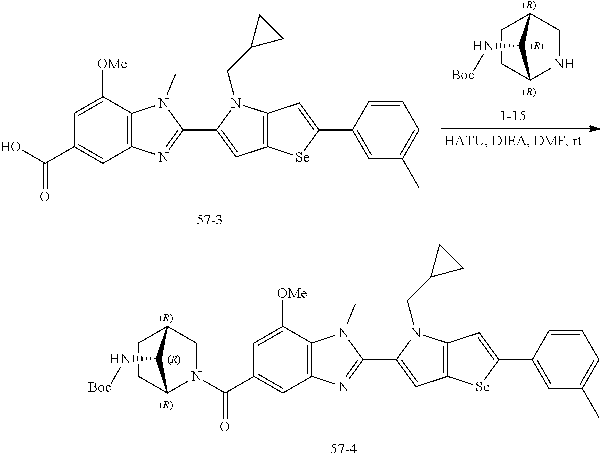

Compound 57-4: To a solution of compound 57-3 (80 mg, 0.15 mmol), DIEA (59.8 mg, 0.46 mmol) and HATU (70.4 mg, 0.18 mmol) in DMF (5 mL) was added compound 1-15 (36.2 mg, 0.17 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 57-4 (80 mg) as a brown solid, yield: 65.4%. LCMS: Rt=1.56 min, MS (ESI) m/z=714.1 [M+H]⁺.

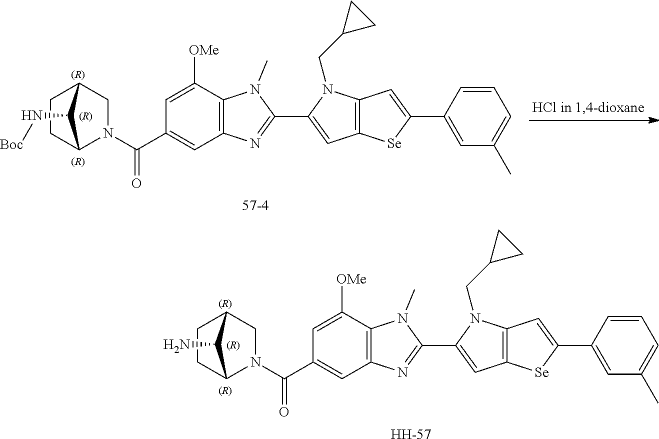

HH-57: To a solution of compound 57-4 (80 mg, 0.11 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-57 (42 mg, hydrochloric acid salt form) as a yellow solid. yield: 61%. ¹H NMR (400 MHz, DMSO-d₆): δ ppm 8.40 (s, 3H), 7.94 (s, 1H), 7.48 (s, 1H), 7.43 (s, 2H), 7.29 (s, 1H), 7.12 (d, J=7.2 Hz, 1H), 7.05 (s, 1H), 7.00 (s, 1H), 4.39 (d, J=6.8 Hz, 3H), 4.12 (s, 3H), 4.02 (s, 3H), 3.70-3.45 (m, 2H), 3.18 (d, J=10.0 Hz, 1H), 2.66 (s, 1H), 2.37 (s, 3H), 2.04-1.88 (m, 3H), 1.65-1.57 (m, 1H), 1.15-1.07 (m, 1H), 0.40-0.33 (m, 2H), 0.20-0.13 (m, 2H). LCMS: Rt=1.36 min, MS (ESI) m/z=614.1 [M+H]⁺. HPLC: Rt=6.74 min, Purity: 98.82% (214 nm) and 98.82% (254 nm).

### Example 58. Synthesis of HH-58

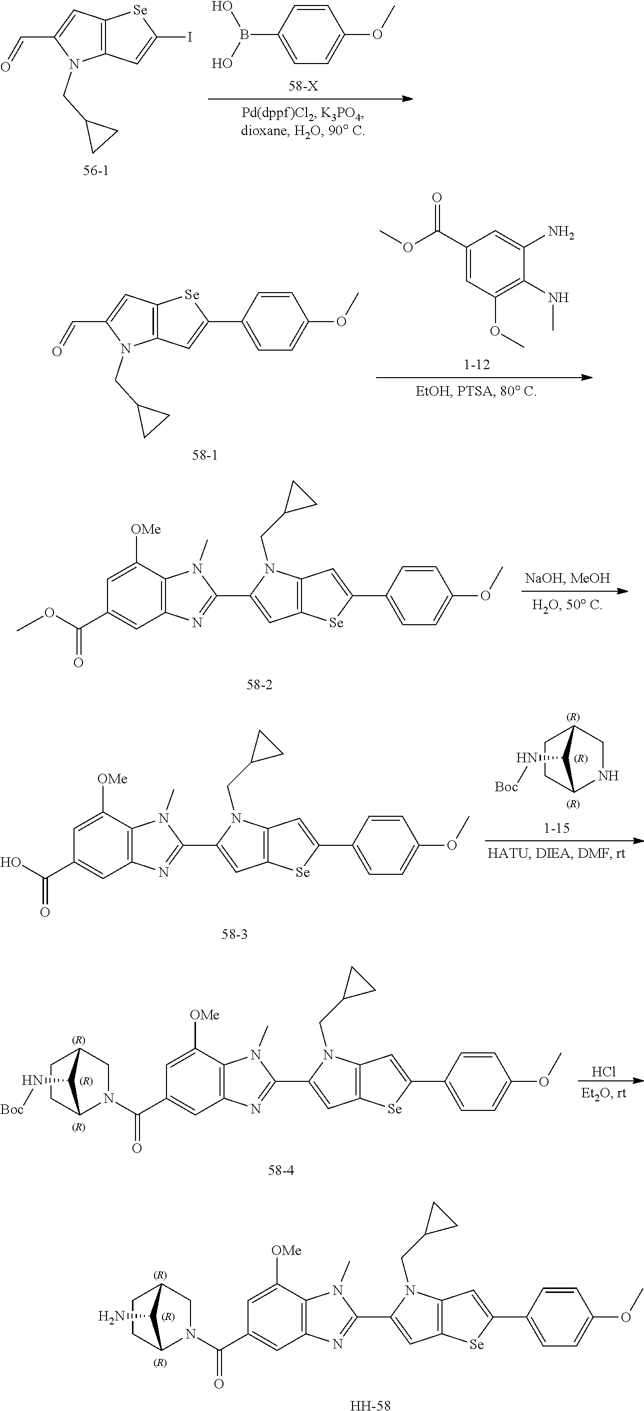

### Step 1

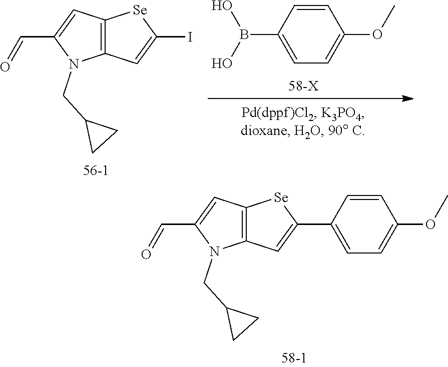

Compound 58-1: To a solution of compound 56-1 (0.45 g, 1.17 mmol), compound 58-X (0.272 g, 1.77 mmol), 1,1′-bis(diphenylphosphino)ferrocene-palladium(II) dichloride dichloromethane complex (291 mg, 0.36 mmol) in dioxane (25 mL) and water (6 mL) was added potassium phosphate tribasic (758 mg, 3.57 mmol). The mixture was stirred at 90° C. under N₂ for 3 h. The mixture was diluted with water (40 mL) and extracted with ethyl acetate (50 mL×4). The organic phase was washed with brine (40 mL), dried over anhydrous Na₂SO₄ and concentrated under vacuum to afford crude product, which was purified by silica gel chromatography (elution gradient: petroleum ether/EtOAc, 3/1, v/v) to afford compound 58-1 (240 mg) as a yellow solid, yield: 56.2%. LCMS: Rt=1.45 min, MS (ESI) m/z=360.1 [M+H]⁺.

### Step 2

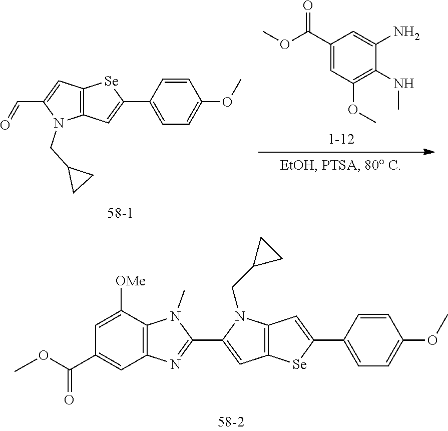

Compound 58-2: To a solution of compound 58-1 (240 mg, 0.66 mmol) and compound 1-12 (141 mg, 0.66 mmol) in EtOH (50 mL) was added p-toluenesulfonic acid (11.6 mg, 0.06 mmol). The mixture was stirred at 80° C. for 5 h. The solvent was evaporated and diluted with water (40 mL), extracted with ethyl acetate (40 mL×3). The organic layers were washed with water (20 mL), brine (20 mL), dried over anhydrous Na₂SO₄ and concentrated under vacuum to afford crude product, which was purified by silica gel chromatography (elution gradient: petroleum ether/EtOAc, 5/1, v/v) to afford compound 58-2 (240 mg) as a yellow oil, yield: 58.8%. LCMS: Rt=1.54 min, MS (ESI) m/z=549.9 [M+H]⁺.

### Step 3

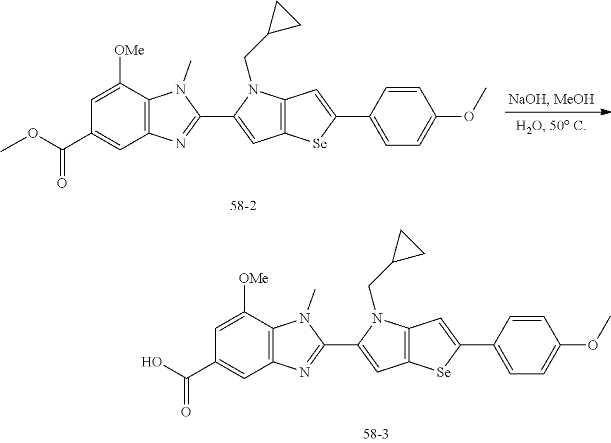

Compound 58-3: To a suspension of compound 58-2 (240 mg, 0.44 mmol) in MeOH (25 mL) was added 2N sodium hydroxide aqueous solution (0.42 mL, 0.84 mmol). The mixture was stirred at 80° C. for 6 h. The mixture was concentrated and adjusted pH to 5-6 with 1N hydrochloric acid aqueous solution, then extracted with ethyl acetate (30 mL×3). The organic phase was washed with water (15 mL), brine (15 mL), dried over anhydrous Na₂SO₄ and concentrated under vacuum to give compound 58-3 (217 mg) as a yellow solid, yield: 92.3%. LCMS: Rt=1.41 min, MS (ESI) m/z=535.9 [M+H]⁺.

### Step 4

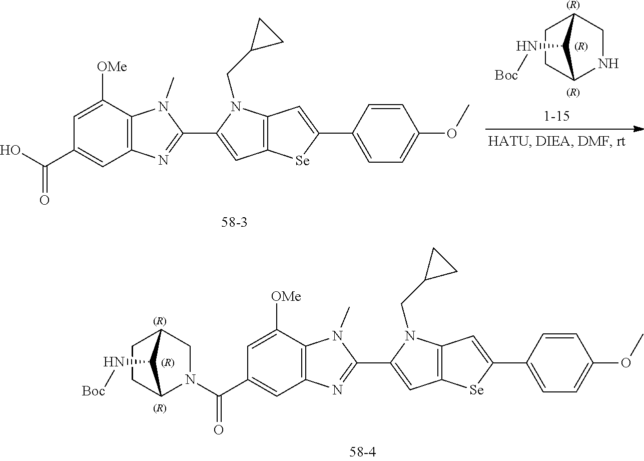

Compound 58-4: To a solution of compound 58-3 (160 mg, 0.3 mmol), DIEA (116 mg, 0.9 mmol) and HATU (135 mg, 0.35 mmol) in DMF (8 mL) was added compound 1-15 (70 mg, 0.32 mmol). The mixture was stirred at room temperature for 6 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 58-4 (200 mg) as a brown solid, yield: 82.4%. LCMS: Rt=1.45 min, MS (ESI) m/z=730.2 [M+H]⁺.

### Step 5

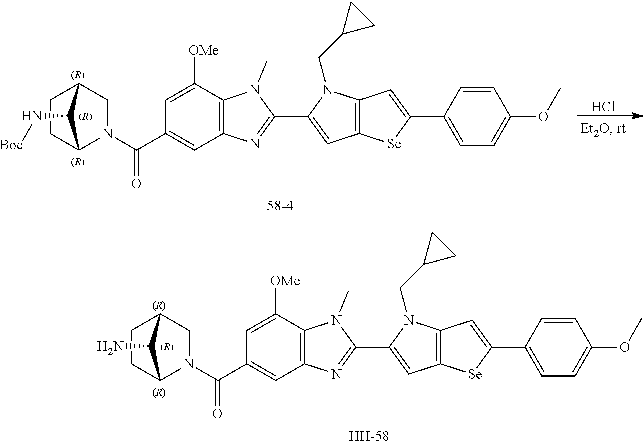

HH-58: To a solution of compound 58-4 (100 mg, 0.14 mmol) in ethyl ether (8 mL) was added hydrochloric acid solution (8 mL, 2M in ethyl ether). The mixture was stirred at room temperature for 4 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HHBP-9462 (60 mg, hydrochloric acid salt form) as a yellow solid. yield: 62.4%. 1H NMR (400 MHz, DMSO-d₆): δ ppm 8.34 (s, 3H), 7.80 (s, 1H), 7.58 (d, J=8.4 Hz, 2H), 7.41 (s, 1H), 7.02-6.97 (m, 4H), 4.38 (d, J=6.7 Hz, 2H), 4.11 (s, 3H), 4.01 (s, 3H), 3.81 (s, 3H), 3.70-3.45 (m, 2H), 3.18 (d, J=10.0 Hz, 1H), 2.66 (s, 1H), 2.03-1.92 (m, 3H), 1.68-1.58 (m, 1H), 1.18-1.13 (m, 1H), 0.38-0.32 (m, 2H), 0.19-0.16 (m, 2H). LCMS: Rt=1.29 min, MS (ESI) m/z=630.0 [M+H]⁺. HPLC: Rt=6.02 min, Purity: 99.83% (214 nm) and 99.67% (254 nm).

### Example 59. Synthesis of HH-59

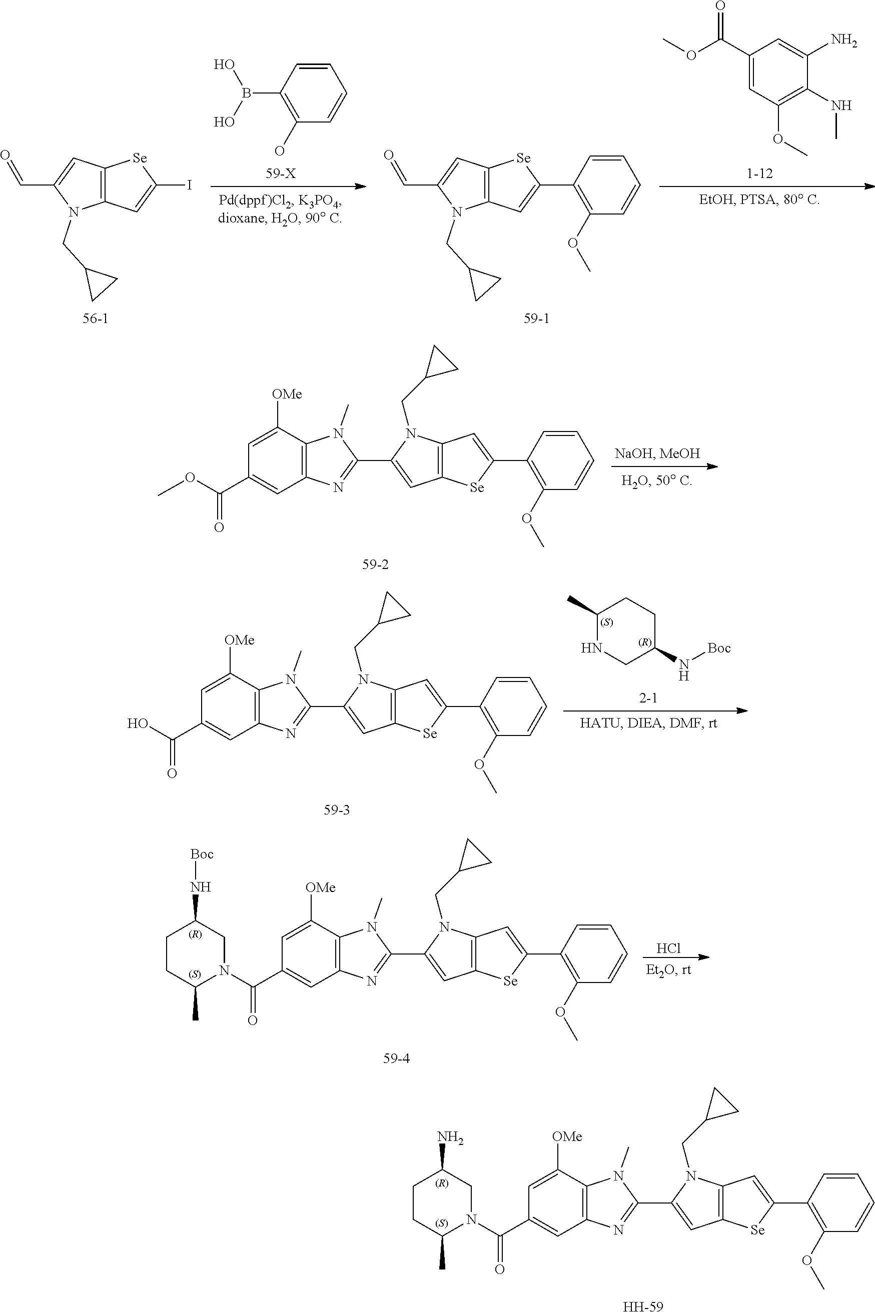

### Step 1

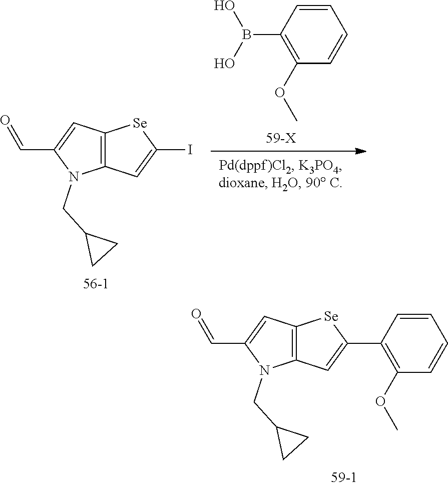 [SMILES: O=Cc1cc2[se]c(I)cc2n1CC1CC1 ; COc1ccccc1B(O)O ; COc1ccccc1-c1cc2c(cc(C=O)n2CC2CC2)[se]1]

Compound 59-1: To a solution of compound 56-1 (500 mg, 1.32 mmol), compound 59-X (301.4 mg, 1.98 mmol), 1,1′-bis(diphenylphosphino)ferrocene-palladium(II) dichloride dichloromethane complex (108 mg, 0.13 mmol) in dioxane (20 mL) and water (4 mL) was added potassium phosphate tribasic (842.2 mg, 3.97 mmol). The mixture was stirred at 90° C. under N₂ for 3 h. The mixture was diluted with water (25 mL) and extracted with ethyl acetate (35 mL×4). The organic phase was washed with brine (25 mL), dried over anhydrous Na₂SO₄ and concentrated under vacuum to afford crude product, which was purified by silica gel chromatography (elution gradient: petroleum ether/EtOAc, 5/1, v/v) to afford compound 59-1 (400 mg) as a yellow oil, yield: 75.9%. LCMS: Rt=1.47 min, MS (ESI) m/z=359.9 [M+H]⁺.

### Step 2

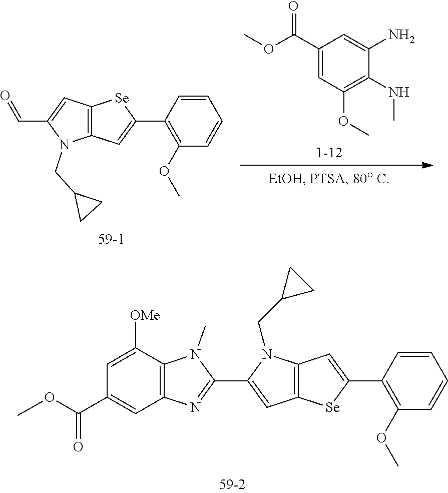

Compound 59-2: To a solution of compound 59-1 (400 mg, 1.12 mmol) and compound 1-12 (234.7 mg, 1.12 mmol) in EtOH (50 mL) was added p-toluenesulfonic acid (19.2 mg, 0.11 mmol). The mixture was stirred at 80° C. for 3 h. The solvent was evaporated and diluted with water (50 mL), extracted with ethyl acetate (50 mL×3). The organic layers were washed with water (30 mL), brine (30 mL), dried over anhydrous Na₂SO₄ and concentrated under vacuum to afford crude product, which was purified by silica gel chromatography (elution gradient: petroleum ether/EtOAc, 2/1, v/v) to afford compound 59-2 (350 mg) as a yellow oil, yield: 57.2%. LCMS: Rt=1.52 min, MS (ESI) m/z=550.0 [M+H]⁺.

### Step 3

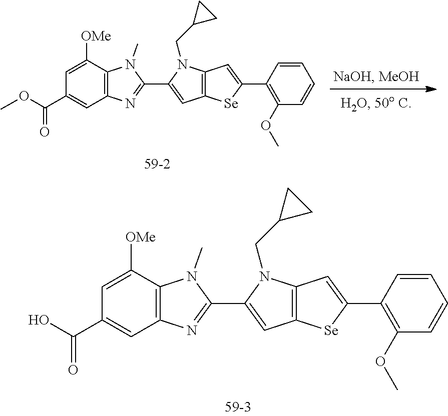 [SMILES: COC(=O)c1cc(OC)c2c(c1)nc(-c1cc3[se]c(-c4ccccc4OC)cc3n1CC1CC1)n2C ; COc1ccccc1-c1cc2c(cc(-c3nc4cc(C(=O)O)cc(OC)c4n3C)n2CC2CC2)[se]1]

Compound 59-3: To a suspension of compound 59-2 (350 mg, 0.64 mmol) in MeOH (20 mL) was added 2N sodium hydroxide aqueous solution (0.6 mL, 1.28 mmol). The mixture was stirred at 50° C. for 3 h. The mixture was concentrated and adjusted pH to 5-6 with 1N hydrochloric acid aqueous solution, then extracted with ethyl acetate (50 mL×3). The organic phase was washed with water (25 mL), brine (25 mL), dried over anhydrous Na₂SO₄ and concentrated under vacuum to give compound 59-3 (160 mg) as a yellow solid, yield: 42.2%. LCMS: Rt=1.42 min, MS (ESI) m/z=535.9 [M+H]⁺.

### Step 4

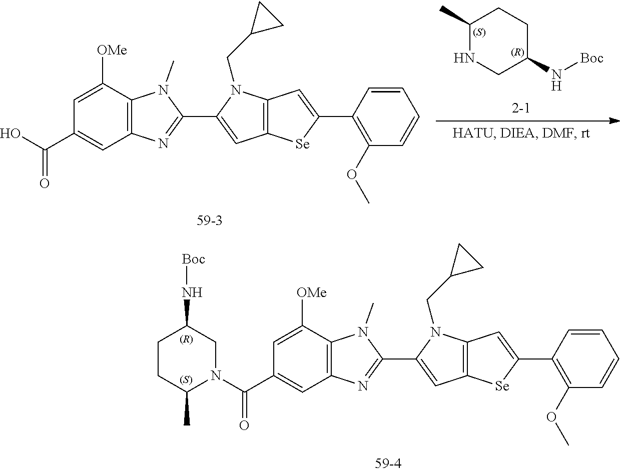

Compound 59-4: To a solution of compound 59-3 (80 mg, 0.15 mmol), DIEA (58.4 mg, 0.45 mmol) and HATU (68.3 mg, 0.18 mmol) in DMF (5 mL) was added compound 2-1 (35.5 mg, 0.16 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 59-4 (80 mg) as a brown solid, yield: 65.7%. LCMS: Rt=1.52 min, MS (ESI) m/z=732.3 [M+H]⁺.

### Step 5

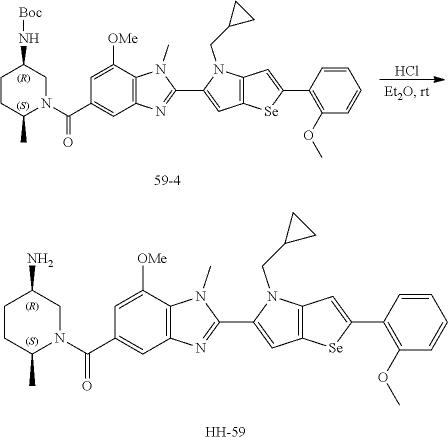

HH-59: To a solution of compound 59-4 (80 mg, 0.11 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-59 (40 mg, hydrochloric acid salt form) as a yellow solid. yield: 66.4%. ¹H NMR (400 MHz, DMSO-d₆): δ ppm 8.29 (s, 3H), 8.20 (s, 1H), 7.96 (d, J=7.6 Hz, 1H), 7.35-7.30 (m, 2H), 7.19-6.98 (m, 4H), 4.41 (d, J=7.2 Hz, 2H), 4.16 (s, 3H), 4.03 (s, 3H), 3.97 (s, 3H), 3.25-3.00 (m, 2H), 1.94-1.50 (m, 4H), 1.22 (d, J=6.8 Hz, 3H), 1.12-1.04 (m, 1H), 0.43-0.26 (m, 2H), 0.20-0.10 (m, 2H). LCMS: Rt=1.31 min, MS (ESI) m/z=632.2 [M+H]⁺. HPLC: Rt=5.78 min, Purity: 95.48% (214 nm) and 95.03% (254 nm).

### Example 60. Synthesis of HH-60

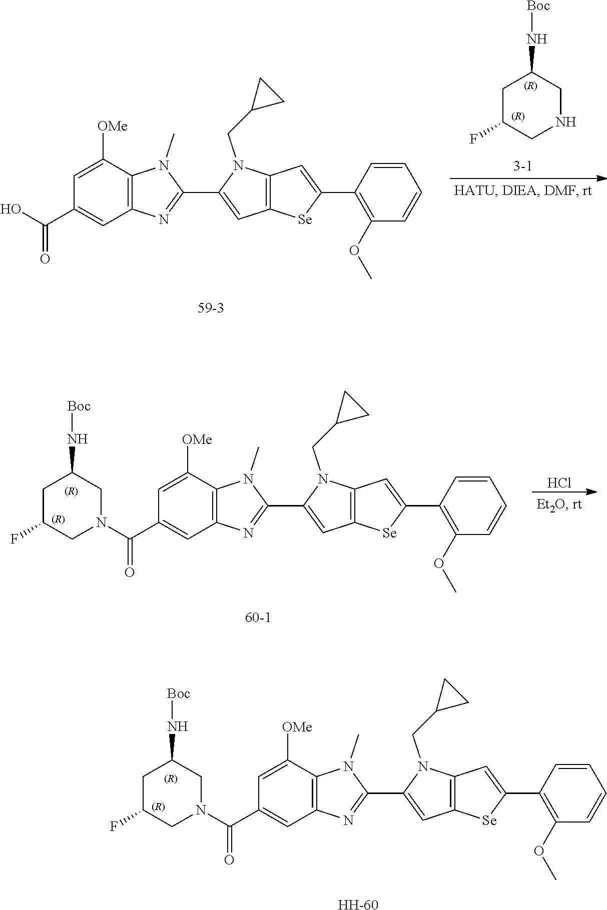

### Step 1

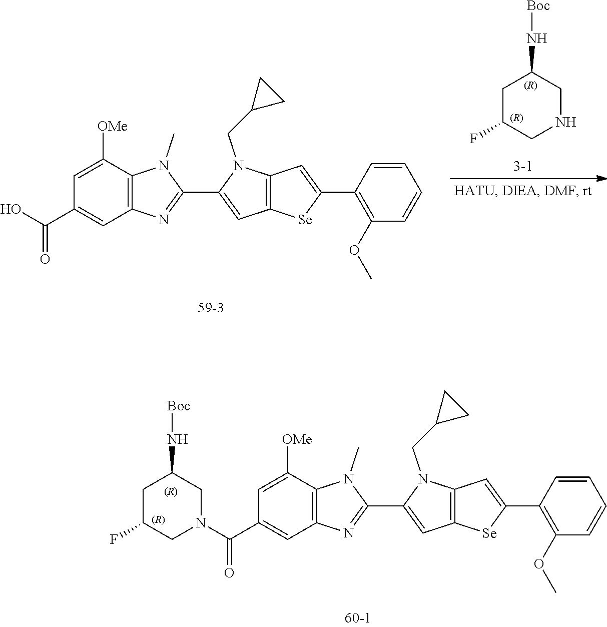

Compound 60-1: To a solution of compound 59-3 (80 mg, 0.15 mmol), DIEA (58.4 mg, 0.45 mmol) and HATU (68.3 mg, 0.18 mmol) in DMF (5 mL) was added compound 3-1 (35.9 mg, 0.16 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 60-1 (90 mg) as a yellow oil, yield: 81.8%. LCMS: Rt=1.48 min, MS (ESI) m/z=736.2 [M+H]⁺.

### Step 2

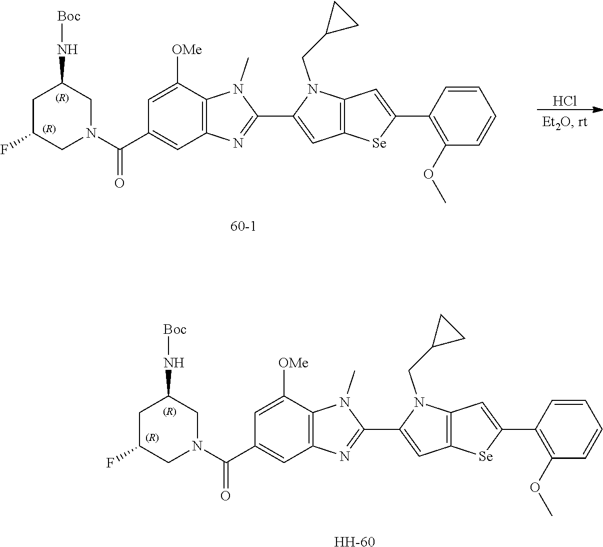

HH-60: To a solution of compound 60-1 (100 mg, 0.12 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-60 (51 mg, hydrochloric acid salt form) as a yellow solid. yield: 65.6%. ¹H NMR (400 MHz, DMSO-d₆): δ ppm 8.44 (s, 3H), 8.21 (s, 1H), 7.96 (d, J=8.0 Hz, 1H), 7.38 (s, 1H), 7.32-7.30 (m, 1H), 7.19-7.07 (m, 3H), 7.01 (s, 1H), 4.41 (d, J=6.8 Hz, 2H), 4.16 (s, 3H), 4.02 (s, 3H), 3.97 (s, 4H), 3.39 (s, 2H), 3.16-2.70 (m, 2H), 2.45-2.38 (m, 1H), 2.03-1.86 (m, 1H), 1.13-1.03 (m, 1H), 0.36-0.32 (m, 2H), 0.16-0.11 (m, 2H). LCMS: Rt=1.28 min, MS (ESI) m/z=636.2 [M+H]⁺. HPLC: Rt=5.67 min, Purity: 96.61% (214 nm) and 96.48% (254 nm).

### Example 61. Synthesis of HH-61

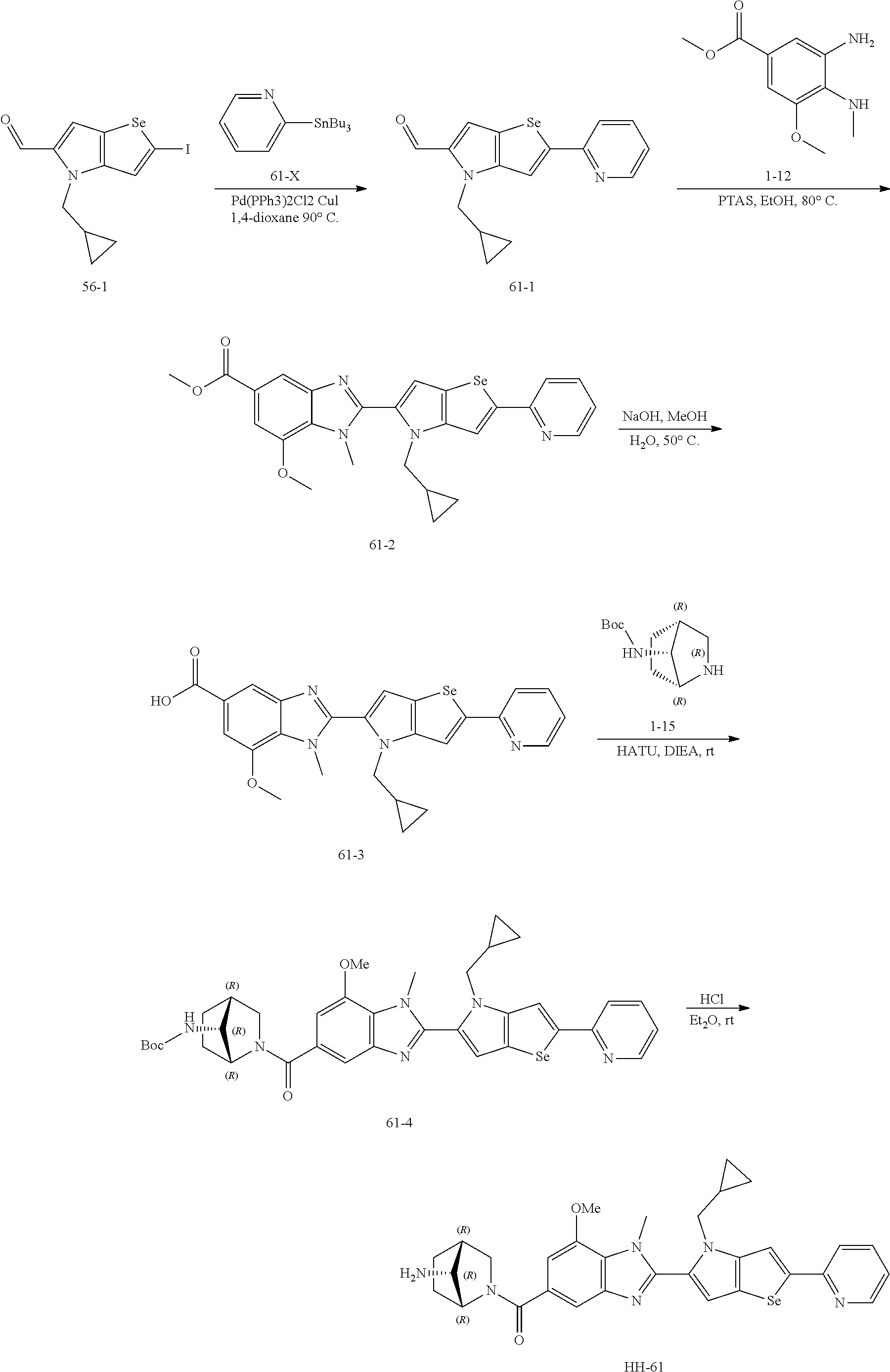

### Step 1

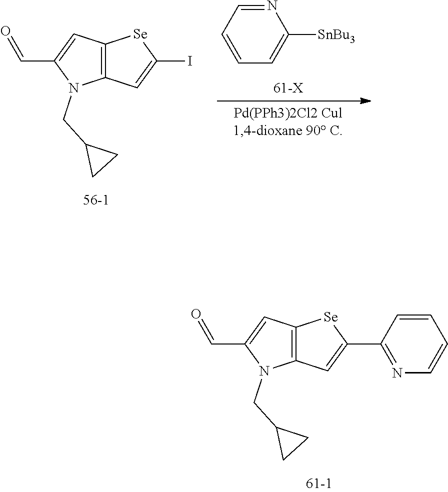

Compound 61-1: To a solution of compound 56-1 (300 mg, 0.82 mmol), compound 61-X (452.5 mg, 1.23 mmol), bis(triphenylphosphine)palladium(II) chloride (57.5 mg, 0.08 mmol) in dioxane (20 mL) was added cuprous iodide (46.8 mg, 0.24 mmol). The mixture was stirred at 90° C. under N₂ for 3 h. The mixture was diluted with water (30 mL) and extracted with ethyl acetate (35 mL×4). The organic phase was washed with brine (30 mL), dried over anhydrous Na₂SO₄ and concentrated under vacuum to afford crude product, which was purified by silica gel chromatography (elution gradient: petroleum ether/EtOAc, 5/1, v/v) to afford compound 61-1 (200 mg) as a yellow oil, yield: 66.7%. LCMS: Rt=1.37 min, MS (ESI) m/z=330.9 [M+H]⁺.

### Step 2

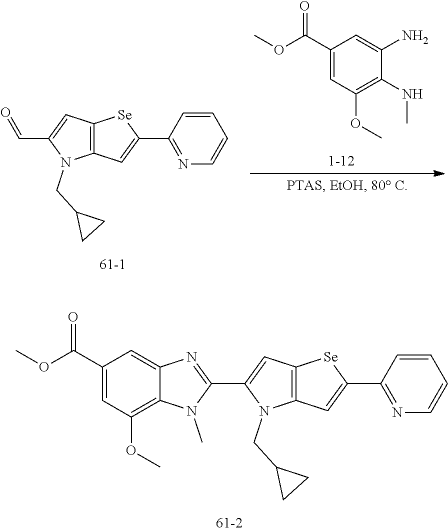

Compound 61-2: To a solution of compound 61-1 (200 mg, 0.61 mmol) and compound 1-12 (140.5 mg, 0.67 mmol) in EtOH (30 mL) was added p-toluenesulfonic acid (10.6 mg, 0.06 mmol). The mixture was stirred at 80° C. for 3 h. The solvent was evaporated and diluted with water (40 mL), extracted with ethyl acetate (30 mL×3). The organic layers were washed with water (15 mL), brine (15 mL), dried over anhydrous Na₂SO₄ and concentrated under vacuum to afford crude product, which was purified by silica gel chromatography (elution gradient: petroleum ether/EtOAc, 10/1, v/v) to afford compound 61-2 (200 mg) as a yellow oil, yield: 57.0%. LCMS: Rt=1.45 min, MS (ESI) m/z=521.1 [M+H]⁺.

### Step 3

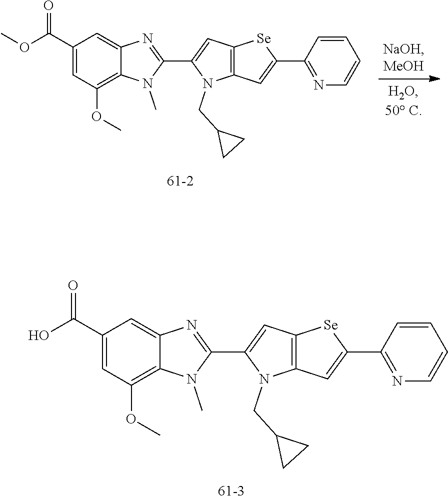 [SMILES: COC(=O)c1cc(OC)c2c(c1)nc(-c1cc3[se]c(-c4ccccn4)cc3n1CC1CC1)n2C ; COc1cc(C(=O)O)cc2nc(-c3cc4[se]c(-c5ccccn5)cc4n3CC3CC3)n(C)c12]

Compound 61-3: To a suspension of compound 61-2 (200 mg, 0.38 mmol) in MeOH (10 mL) was added 2N sodium hydroxide aqueous solution (0.4 mL, 0.77 mmol). The mixture was stirred at 50° C. for 3 h. The mixture was concentrated and adjusted pH to 5-6 with 1N hydrochloric acid aqueous solution, then extracted with ethyl acetate (30 mL×3). The organic phase was washed with water (20 mL), brine (20 mL), dried over anhydrous Na₂SO₄ and concentrated under vacuum to give compound 61-3 (120 mg) as a yellow solid, yield: 55.5%. LCMS: Rt=1.33 min, MS (ESI) m/z=506.9 [M+H]⁺.

### Step 4

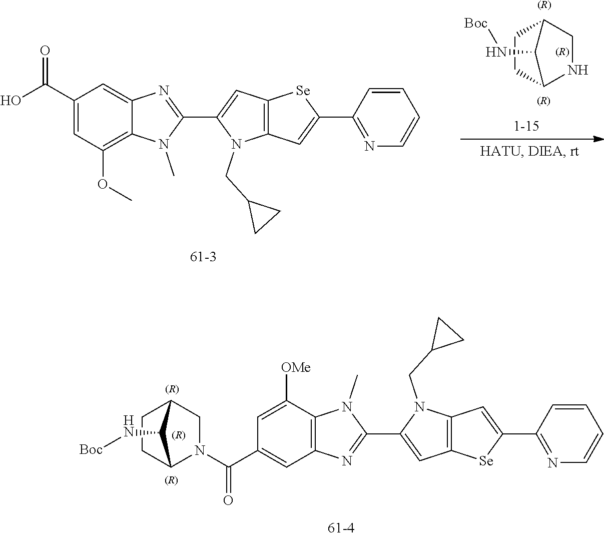

Compound 61-4: To a solution of compound 61-3 (60 mg, 0.12 mmol), DIEA (46.0 mg, 0.36 mmol) and HATU (54.2 mg, 0.14 mmol) in DMF (5 mL) was added compound 1-15 (27.8 mg, 0.13 mmol). The mixture was stirred at room temperature for 6 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 61-4 (70 mg) as a brown solid, yield: 75.7%. LCMS: Rt=1.40 min, MS (ESI) m/z=701.0 [M+H]⁺.

### Step 5

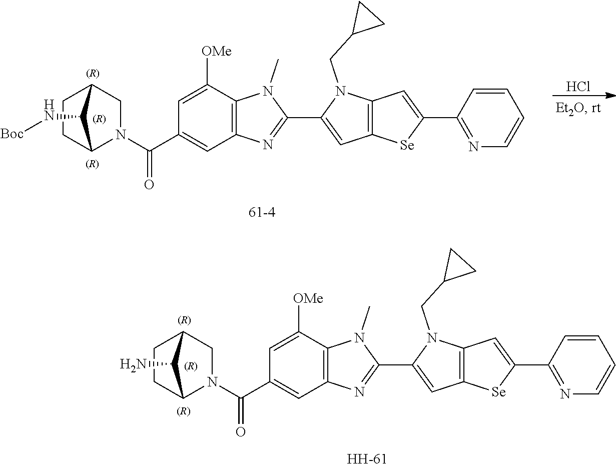

HH-61: To a solution of compound 61-4 (70 mg, 0.10 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-61 (25 mg, hydrochloric acid salt form) as a yellow solid. yield: 37.5%. ¹H NMR (400 MHz, DMSO-d₆): δ ppm 8.49 (d, J=4.8 Hz, 1H), 8.42 (s, 3H), 8.30 (s, 1H), 7.99 (d, J=7.6 Hz, 1H), 7.83 (t, J=7.2 Hz, 1H), 7.43 (s, 1H), 7.28-7.23 (m, 1H), 7.05 (s, 1H), 7.00 (s, 1H), 4.40 (d, J=6.8 Hz, 2H), 4.12 (s, 3H), 4.02 (s, 3H), 3.69-3.45 (m, 2H), 3.18 (d, J=9.6 Hz, 1H), 2.66 (s, 1H), 2.07-1.86 (m, 3H), 1.65-1.61 (m, 1H), 1.10-1.20 (s, 1H), 0.41-0.34 (m, 2H), 0.21-0.15 (m, 2H). LCMS: Rt=1.13 min, MS (ESI) m/z=601.2 [M+H]⁺. HPLC: Rt=5.03 min, Purity: 95.00% (214 nm) and 95.06% (254 nm).

### Example 62. Synthesis of HH-62

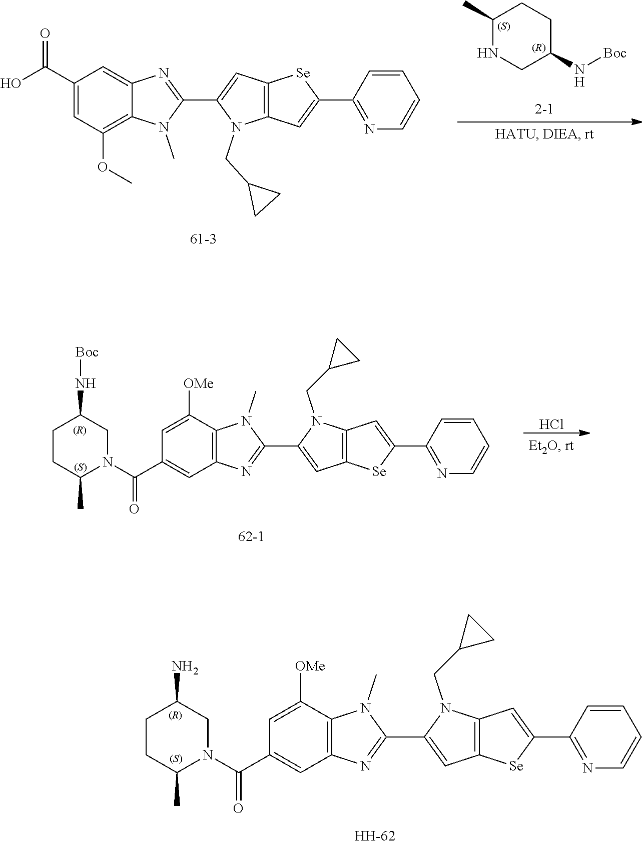

### Step 1

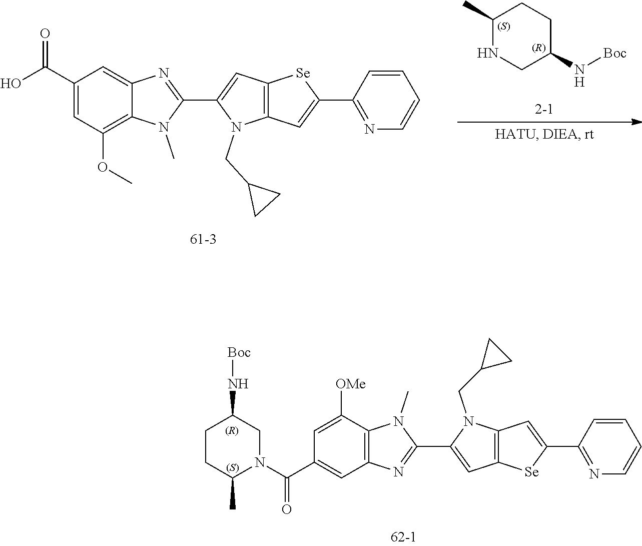

Compound 62-1: To a solution of compound 61-3 (70 mg, 0.14 mmol), DIEA (53.7 mg, 0.41 mmol) and HATU (63.2 mg, 0.17 mmol) in DMF (5 mL) was added compound 2-1 (32.6 mg, 0.15 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 62-1 (80 mg) as a yellow oil, yield: 82.3%. LCMS: Rt=1.42 min, MS (ESI) m/z=703.0 [M+H]⁺.

### Step 2

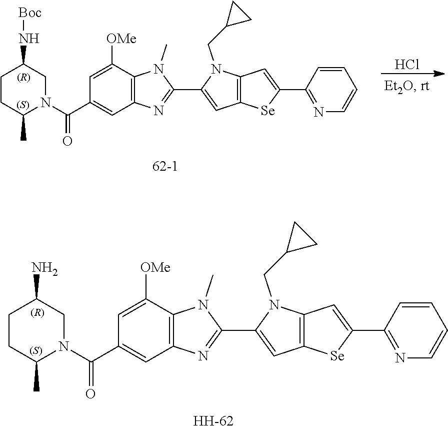

HH-62: To a solution of compound 62-1 (80 mg, 0.11 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-62 (24 mg, hydrochloric acid salt form) as a yellow solid. yield: 35%. ¹H NMR (400 MHz, DMSO-d₆): δ ppm 8.51 (d, J=4.4 Hz, 1H), 8.44 (s, 1H), 8.28 (s, 3H), 8.05 (d, J=8.0 Hz, 1H), 7.88 (t, J=7.8 Hz, 1H), 7.35 (s, 1H), 7.30 (dd, J=6.8, 5.2 Hz, 1H), 7.14 (s, 1H), 6.97 (s, 1H), 4.39 (d, J=7.2 Hz, 3H), 4.15 (s, 3H), 4.02 (s, 3H), 3.30-3.00 (m, 2H), 1.97-1.72 (m, 3H), 1.56-1.63 (m, 1H), 1.22 (d, J=6.8 Hz, 3H), 1.18-1.13 (m, 1H), 0.40-0.32 (m, 2H), 0.21-0.14 (m, 2H). LCMS: Rt=1.19 min, MS (ESI) m/z=603.0 [M+H]⁺. HPLC: Rt=5.21 min, Purity: 99.61% (214 nm) and 99.38% (254 nm).

### Example 63. Synthesis of HH-63

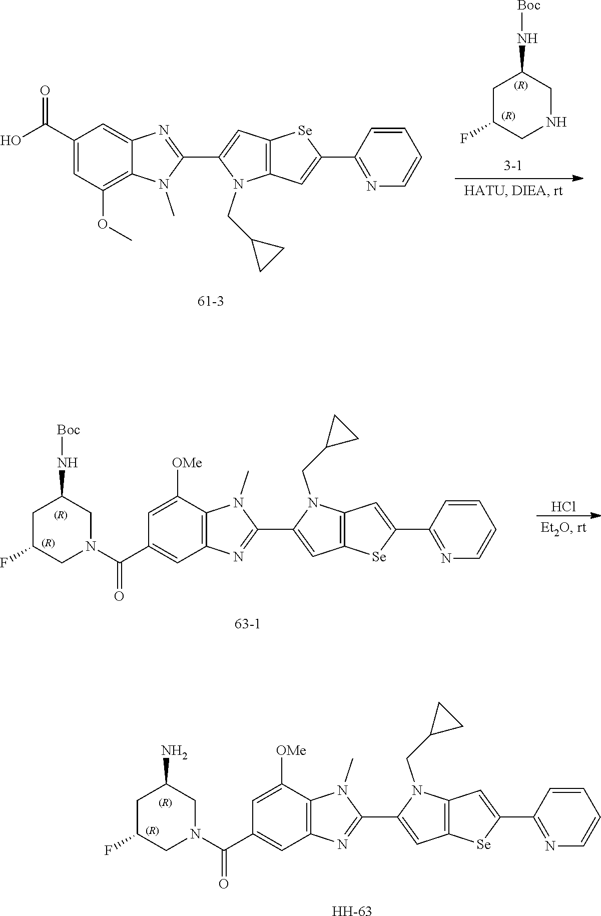

### Step 1

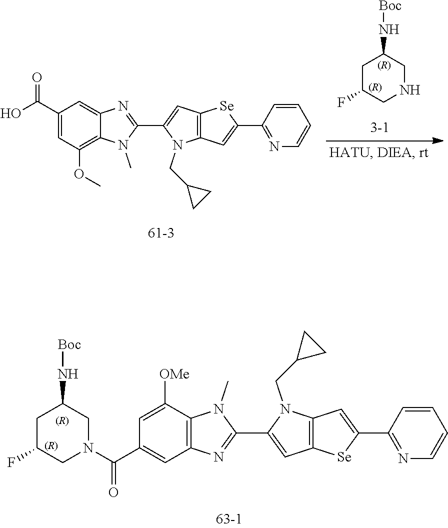

Compound 63-1: To a solution of compound 61-3 (70 mg, 0.14 mmol), DIEA (53.7 mg, 0.41 mmol) and HATU (63.2 mg, 0.17 mmol) in DMF (5 mL) was added compound 3-1 (33.2 mg, 0.15 mmol). The mixture was stirred at room temperature for 3 h. The mixture was diluted with water (50 mL), extracted with ethyl acetate (25 mL×3). The organic layers were washed with water (15 mL), brine (15 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 63-1 (80 mg) as a yellow oil, yield: 81.8%. LCMS: Rt=1.39 min, MS (ESI) m/z=707.0 [M+H]⁺.

### Step 2

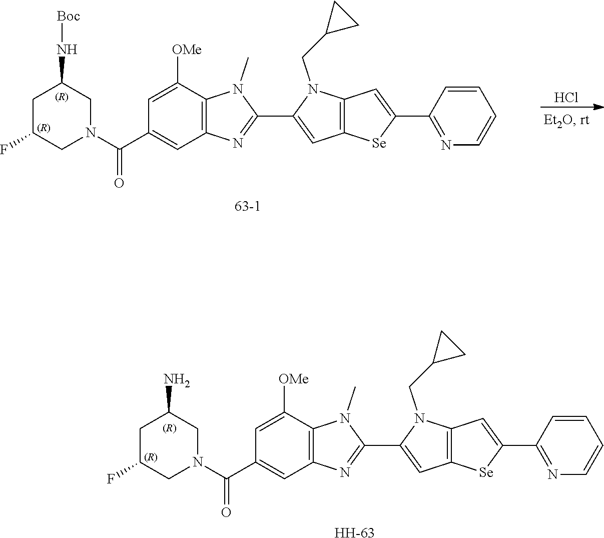

HH-63: To a solution of compound 63-1 (80 mg, 0.11 mmol) in ethyl ether (5 mL) was added hydrochloric acid solution (4 mL, 2M in ethyl ether). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% FA) and MeCN as eluents 30-40%) to afford HH-63 (29.2 mg, hydrochloric acid salt form) as a yellow solid. yield: 42.6%. ¹H NMR (400 MHz, DMSO-d₆): δ ppm 8.58 (s, 3H), 8.52 (s, 1H), 8.08 (d, J=8.0 Hz, 1H), 7.92 (t, J=7.6 Hz, 1H), 7.40 (s, 1H), 7.36-7.30 (m, 1H), 7.22 (s, 1H), 7.05 (s, 1H), 5.14-4.64 (m, 2H), 4.39 (d, J=6.8 Hz, 2H), 4.16 (s, 3H), 4.03 (s, 3H), 3.43-3.35 (m, 1H), 3.09 (s, 1H), 2.43-2.38 (m, 1H), 2.10-1.85 (m, 1H), 1.14-1.07 (m, 1H), 0.40-0.33 (m, 2H), 0.21-0.12 (m, 2H). LCMS: Rt=1.14 min, MS (ESI) m/z=607.2 [M+H]⁺. HPLC: Rt=4.72 min, Purity: 98.74% (214 nm) and 99.03% (254 nm).

### Example 64. Synthesis of HH-64

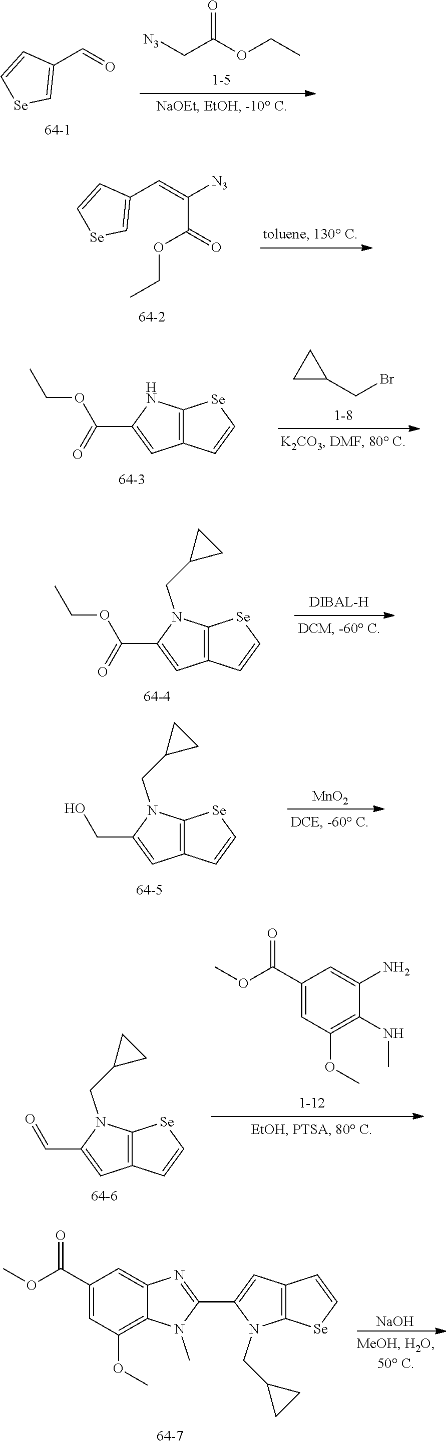

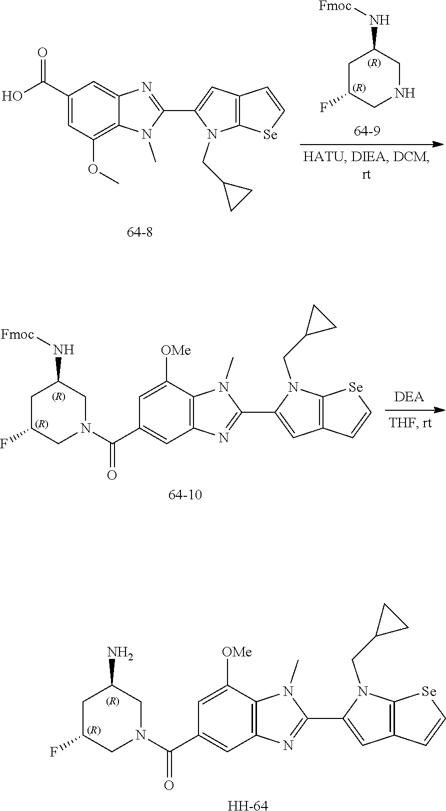

### Step 1

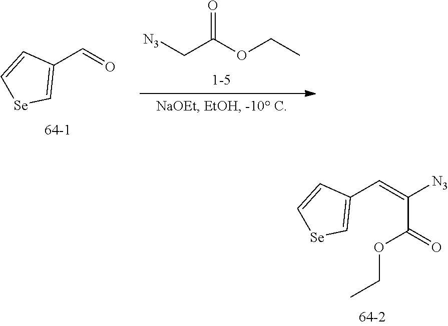

Compound 64-2: To a solution of sodium ethoxide (20% wt. in EtOH) (51.3 g, 150.9 mmol) in EtOH (100 mL) was slowly added the mixture of compound 64-1 (8 g, 50.3 mmol) and compound 1-5 (19.5 g, 150.9 mmol) in EtOH (100 mL) at −10° C. The mixture was stirred at −10° C. under N₂ for 1 h and at room temperature for further 2 h. The mixture was quenched with saturated NH₄Cl aqueous solution (200 mL) and extracted with ethyl acetate (200 mL×3). The organic layers were washed with water (200 mL), brine (200 mL), dried over anhydrous Na₂SO₄ and concentrated under vacuum to afford crude product, which was purified by silica gel chromatography (elution gradient: petroleum ether/EtOAc, 10/1, v/v) to afford compound 64-2 (6 g) as a yellow oil, yield: 44.2%. ¹H NMR (400 MHz, CDCl₃) δ ppm 8.63 (s, 1H), 8.02-7.96 (m, 1H), 7.76 (d, J=5.6 Hz, 1H), 6.93 (s, 1H), 4.36 (q, J=7.2 Hz, 2H), 1.39 (t, J=7.0 Hz, 3H).

### Step 2

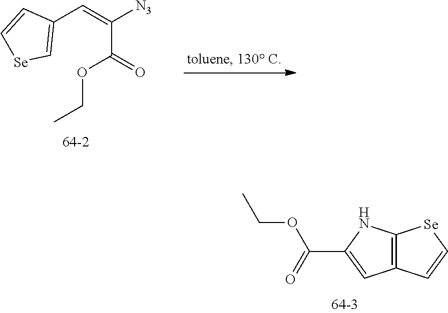

Compound 64-3: A solution of compound 64-2 (6 g, 22.2 mmol) in toluene (300 mL) was stirred at 130° C. under N₂ for 16 h. The reaction mixture was cooled to room temperature and concentrated under vacuum to give the crude product, which was purified by silica gel chromatography (elution gradient: petroleum ether/EtOAc, 4/1, v/v) to afford compound 64-3 (4.5 g) as yellow oil, yield: 83.7%. LCMS: Rt=1.257 min, MS (ESI) m/z=243.9 [M+H]⁺.

### Step 3

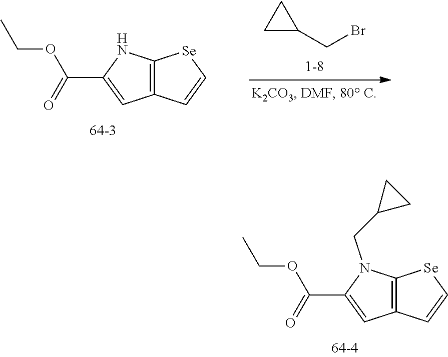 [SMILES: CCOC(=O)c1cc2cc[se]c2[nH]1 ; BrCC1CC1 ; CCOC(=O)c1cc2cc[se]c2n1CC1CC1]

Compound 64-4: To a solution of compound 64-3 (4 g, 16.5 mmol) and compound 1-8 (3.35 g, 24.8 mmol) in DMF (100 mL) was added potassium carbonate (4.57 g, 33.0 mmol). The mixture was stirred at 80° C. for 3 h. The mixture was diluted with water (100 mL) and extracted with ethyl acetate (100 mL×3). The organic layers were washed with water (100 mL) and brine (100 mL), dried over anhydrous Na₂SO₄ and concentrated under vacuum to afford crude product, which was purified by silica gel chromatography (elution gradient: petroleum ether/EtOAc, 10/1, v/v) to afford compound 64-4 (4 g) as a yellow oil, yield: 81.7%. LCMS: Rt=1.434 min, MS (ESI) m/z=298.0 [M+H]⁺.

### Step 4

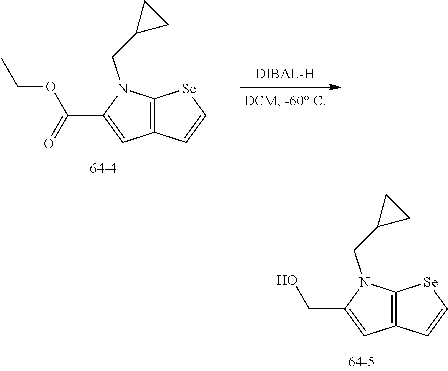 [SMILES: CCOC(=O)c1cc2cc[se]c2n1CC1CC1 ; OCc1cc2cc[se]c2n1CC1CC1]

Compound 64-5: To a solution of compound 64-4 (1 g, 3.38 mmol) in DCM (100 mL) cooled at −60° C. under N₂ was slowly added DIBAL-H (1 M in THF, 10.1 mL, 10.1 mmol). The mixture was stirred at −60° C. under N₂ for 2 h. The mixture was quenched with water (50 mL) and filtered. The filtrate was extracted with dichloromethane (100 mL×3). The organic layers were dried over anhydrous Na₂SO₄ and concentrated under vacuum to afford compound 64-5 (0.8 g) as a yellow oil, yield: 93.2%. LCMS: Rt=1.238 min, MS (ESI) m/z=256.0 [M+H]⁺.

### Step 5

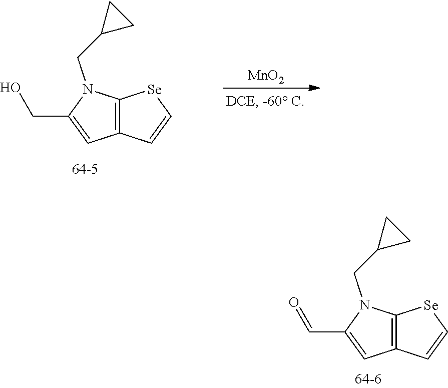 [SMILES: OCc1cc2cc[se]c2n1CC1CC1 ; "MnO2" ; O=Cc1cc2cc[se]c2n1CC1CC1]

Compound 64-6: To a solution of compound 64-5 (0.8 g, 3.15 mmol) in DCE (50 mL) was added manganese dioxide (2.74 g, 31.5 mmol). The mixture was stirred at 60° C. for 16 h. The mixture was filtered and the filtrate was concentrated under vacuum to afford crude product, which was purified by silica gel chromatography (elution gradient: petroleum ether/EtOAc, 5/1, v/v) to afford compound 64-6 (0.4 g) as a yellow oil, yield: 50.4%. LCMS: Rt=1.313 min, MS (ESI) m/z=254.0 [M+H]⁺.

### Step 6

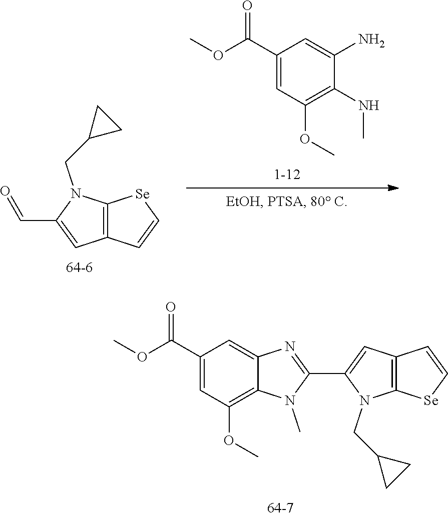 [SMILES: O=Cc1cc2cc[se]c2n1CC1CC1 ; CNc1c(N)cc(C(=O)OC)cc1OC ; COC(=O)c1cc(OC)c2c(c1)nc(-c1cc3cc[se]c3n1CC1CC1)n2C]

Compound 64-7: To a solution of compound 64-6 (400 mg, 1.59 mmol) and compound 1-12 (333.5 mg, 1.59 mmol) in EtOH (100 mL) was added p-toluenesulfonic acid (27.3 mg, 0.16 mmol). The mixture was stirred at 80° C. for 3 h. The solvent was evaporated and diluted with water (50 mL), extracted with ethyl acetate (100 mL×3). The organic layers were washed with water (50 mL), brine (50 mL), dried over anhydrous Na₂SO₄ and concentrated under vacuum to afford crude product, which was purified by silica gel chromatography (elution gradient: petroleum ether/EtOAc, 5/1, v/v) to afford compound 64-7 (500 mg) as a yellow oil, yield: 71.3%. LCMS: Rt=1.447 min, MS (ESI) m/z=443.9 [M+H]⁺.

### Step 7

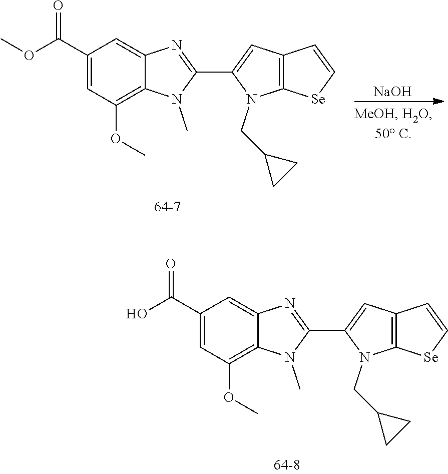

Compound 64-8: To a suspension of compound 64-7 (500 mg, 1.13 mmol) in MeOH (20 mL) was added 2N sodium hydroxide aqueous solution (1.1 mL, 2.26 mmol). The mixture was stirred at 50° C. for 3 h. The mixture was concentrated and adjusted pH to 5-6 with 1N hydrochloric acid aqueous solution, then extracted with ethyl acetate (50 mL×3). The organic phase was washed with water (100 mL), brine (100 mL), dried over anhydrous Na₂SO₄ and concentrated under vacuum to give compound 64-8 (400 mg) as a yellow solid, yield: 82.6%. LCMS: Rt=1.327 min, MS (ESI) m/z=429.9 [M+H]⁺.

### Step 8

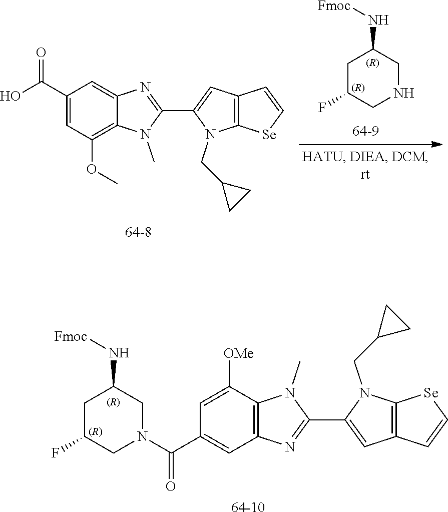

Compound 64-10: To a solution of compound 64-8 (150 mg, 0.35 mmol), DIEA (90.5 mg, 0.70 mmol) and HATU (159.8 mg, 0.42 mmol) in DMF (10 mL) was added compound 64-9 (131.1 mg, 0.38 mmol). The mixture was stirred at room temperature for 3 h. The mixture was diluted with water (50 mL), extracted with ethyl acetate (50 mL×3). The organic layers were washed with water (05 mL), brine (50 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 64-10 (150 mg) as a brown solid, yield: 57.1%. LCMS: Rt=1.423 min, MS (ESI) m/z=752.1 [M+H]⁺.

### Step 9

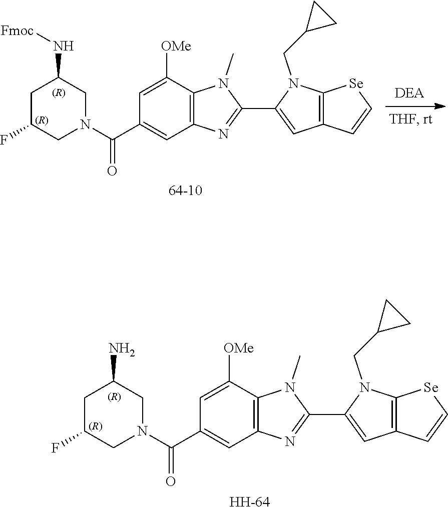

HH-64: To a solution of compound 64-10 (150 mg, 0.20 mmol) in THE (10 mL) was added diethylamine (2 mL). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% NH₃) and MeCN as eluents 30-40%) to afford HH-64 (25 mg) as a yellow solid. yield: 23.7%. ¹H NMR (400 MHz, DMSO-d₆): δ ppm 7.62 (d, J=5.6 Hz, 1H), 7.32-7.24 (m, 2H), 6.94 (s, 1H), 6.81 (s, 1H), 5.12-4.34 (m, 3H), 4.26 (d, J=6.8 Hz, 2H), 4.09 (s, 3H), 3.96 (s, 3H), 3.09-3.03 (m, 1H), 2.35-1.90 (m, 2H), 1.69-1.42 (m, 1H), 1.20-0.91 (m, 2H), 0.86-0.83 (m, 1H), 0.47-0.43 (m, 2H), 0.25-0.21 (m, 2H). LCMS: Rt=1.144 min, MS (ESI) m/z=529.9 [M+H]⁺. HPLC: Rt=5.109 min, Purity: 98.88% (214 nm) and 98.63% (254 nm).

### Example 65. Synthesis of HH-65

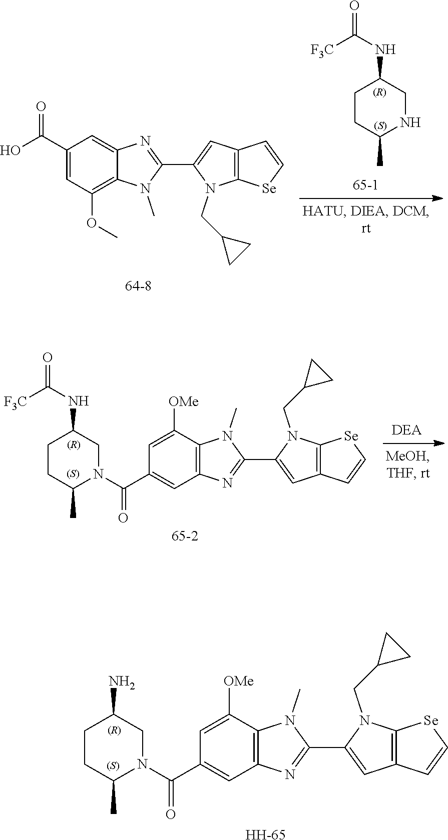

### Step 1

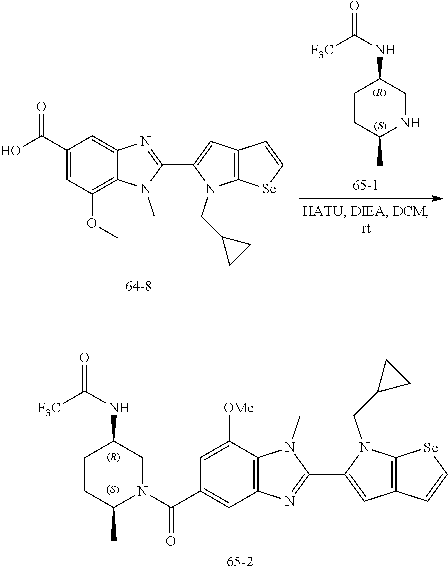

Compound 65-2: To a solution of compound 64-8 (150 mg, 0.35 mmol), DIEA (135.8 mg, 1.05 mmol) and HATU (159.8 mg, 0.42 mmol) in DMF (10 mL) was added compound 65-1 (80.9 mg, 0.38 mmol). The mixture was stirred at room temperature for 3 h. The mixture was diluted with water (50 mL), extracted with ethyl acetate (30 mL×3). The organic layers were washed with water (50 mL), brine (50 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 65-2 (120 mg) as a brown solid, yield: 55.2%. LCMS: Rt=1.337 min, MS (ESI) m/z=622.0 [M+H]⁺.

### Step 2

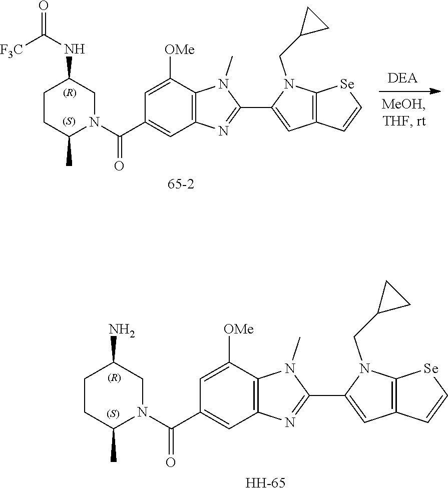

HH-65: To a solution of compound 65-2 (120 mg, 0.19 mmol) in MeOH (10 mL) and THE (2 mL) was added diethylamine (2 mL). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% NH₃) and MeCN as eluents 30-40%) to afford HH-65 (40 mg) as a yellow solid. yield: 39.4%. ¹H NMR (400 MHz, DMSO-d₆): δ ppm 7.61 (d, J=5.6 Hz, 1H), 7.29 (d, J=5.6 Hz, 1H), 7.20 (s, 1H), 6.93 (s, 1H), 6.77 (s, 1H), 4.25 (d, J=6.8 Hz, 3H), 4.08 (s, 3H), 3.96 (s, 3H), 2.72-2.55 (m, 2H), 1.75-1.62 (m, 2H), 1.61-1.34 (m, 2H), 1.33-1.21 (m, 2H), 1.19-1.16 (m, 4H), 0.47-0.43 (m, 2H), 0.26-0.22 (m, 2H). LCMS: Rt=1.092 min, MS (ESI) m/z=526.1 [M+H]⁺. HPLC: Rt=6.734 min, Purity: 97.44% (214 nm) and 98.16% (254 nm).

### Example 66. Synthesis of HH-66

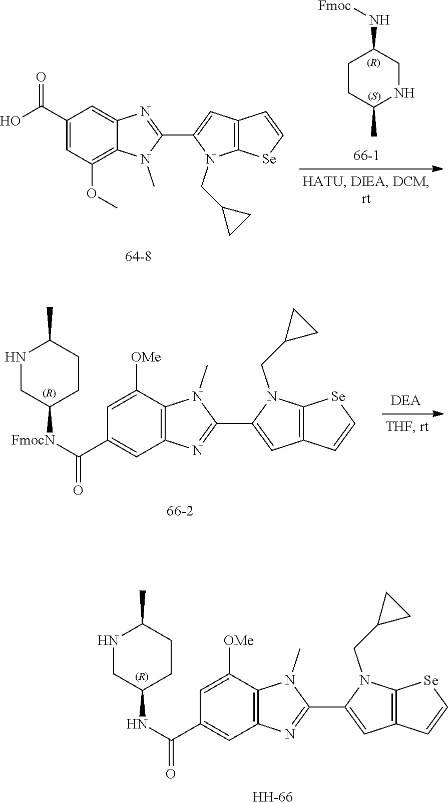

### Step 1

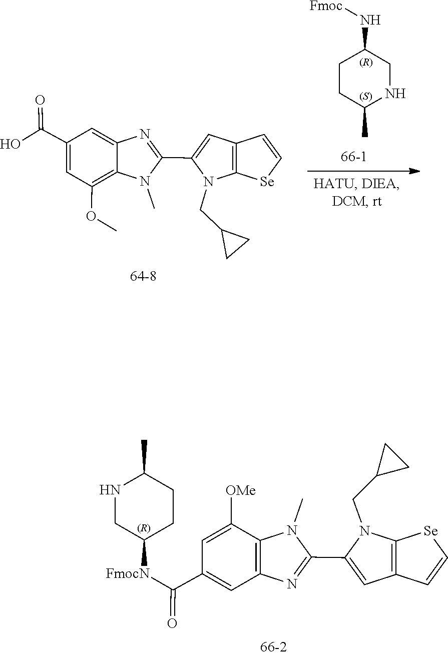

Compound 66-2: To a solution of compound 64-8 (150 mg, 0.35 mmol), DIEA (90.5 mg, 0.70 mmol) and HATU (159.8 mg, 0.42 mmol) in DMF (10 mL) was added compound 66-1 (127.8 mg, 0.38 mmol). The mixture was stirred at room temperature for 3 h. The mixture was diluted with water (50 mL), extracted with ethyl acetate (50 mL×3). The organic layers were washed with water (05 mL), brine (50 mL) over anhydrous Na₂SO₄ and concentrated under vacuum to give the crude product, which was purified by silica gel chromatography (elution gradient: petroleum ether/EtOAc, 1/1 to 0/100, v/v) to afford compound 66-2 (195.2 mg) as a brown solid, yield: 74.7%. LCMS: Rt=1.223 min, MS (ESI) m/z=748.2 [M+H]⁺.

### Step 2

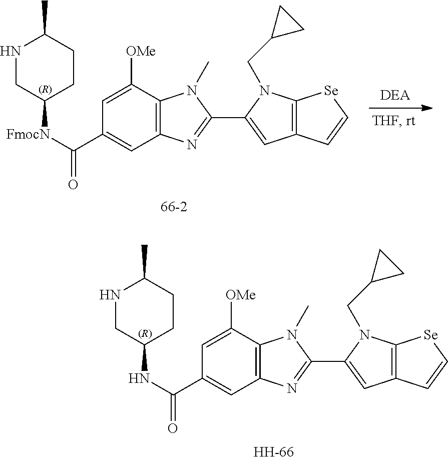

HH-66: To a solution of compound 66-2 (150 mg, 0.20 mmol) in THF (10 mL) was added diethylamine (2 mL). The mixture was stirred at room temperature for 2 h. The resulting mixture was concentrated under vacuum, which was purified by preparative HPLC (Gemini-C18 column, 5μ silica, 21 mm diameter, 150 mm length), using decreasingly polar mixtures of water (containing 0.05% NH₃) and MeCN as eluents 30-40%) to afford HH-66 (50 mg) as a yellow solid. yield: 40%. ¹H NMR (400 MHz, DMSO-d₆): δ ppm 7.61 (d, J=5.6 Hz, 1H), 7.29 (d, J=5.6 Hz, 1H), 7.20 (s, 1H), 6.93 (s, 1H), 6.77 (s, 1H), 4.25 (d, J=6.8 Hz, 3H), 4.08 (s, 3H), 3.96 (s, 3H), 2.72-2.55 (m, 2H), 1.75-1.62 (m, 2H), 1.61-1.34 (m, 2H), 1.33-1.21 (m, 2H), 1.19-1.16 (m, 4H), 0.47-0.43 (m, 2H), 0.26-0.22 (m, 2H). LCMS: Rt=1.110 min, MS (ESI) m/z=526.1 [M+H]⁺. HPLC: Rt=6.734 min, Purity: 97.81% (214 nm) and 98.84% (254 nm).

### Example 67. Synthesis of HH-67

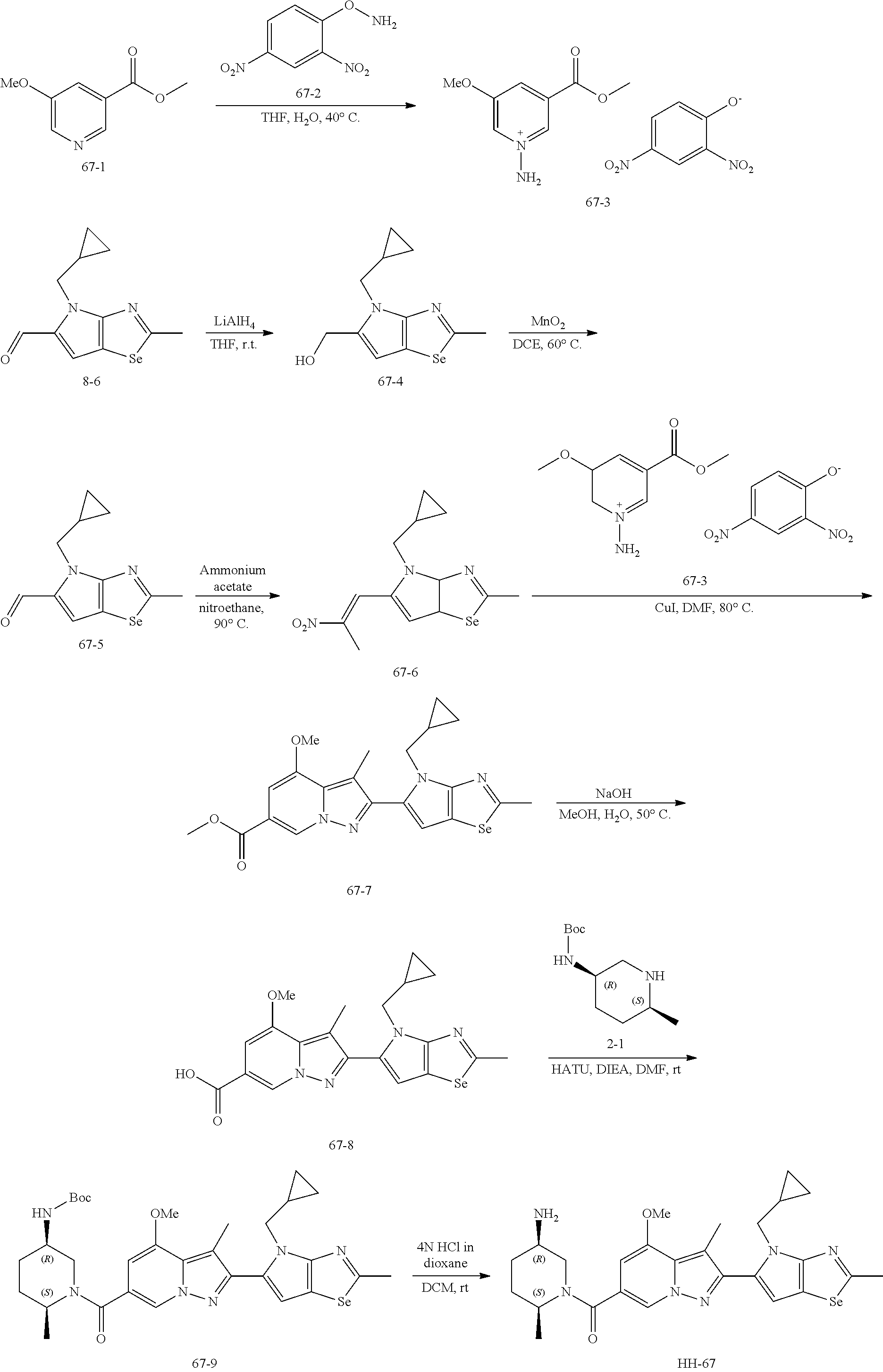

### Step 1

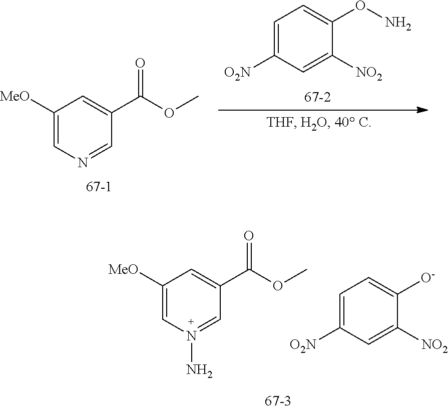

Compound 67-3: To a solution of compound 67-1 (1 g, 5.98 mmol) in THF (10 mL)/H₂O (10 mL) was added compound 67-2 (1.3 g, 6.53 mmol). The mixture was stirred at 40° C. for 16 h. The mixture was concentrated under reduced pressure to afford residue. The residue was triturate with MeCN (2 mL)/Et₂O (10 mL) to afford compound 67-3 (1.5 g) as a yellow solid, yield: 68.5%. ¹H NMR (400 MHz, CDCl₃): δ ppm 8.91 (t, J=1.2 Hz, 1H), 8.79-8.78 (m, 1H), 8.65 (s, 2H), 8.59 (d, J=3.2 Hz, 1H), 8.19-8.18 (m, 1H), 7.78 (dd, J=9.6, 3.2 Hz, 1H), 6.32 (d, J=10.0 Hz, 1H), 4.04 (s, 3H), 3.97 (s, 3H).

### Step 2

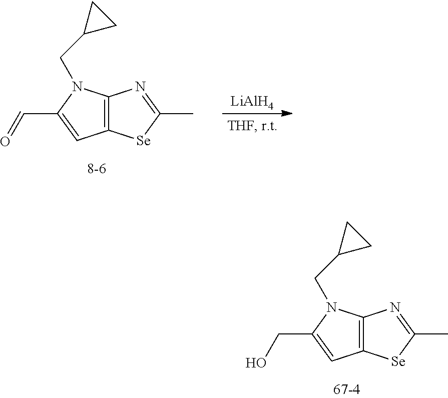

Compound 67-4: To a solution of compound 8-6 (1.47 g, 5.5 mmol) in THF (45 mL), then was added LiAlH₄ (1M in THF, 16.6 mL, 16.6 mmol) at 0° C., The reaction mixture was stirred at 25° C. for 6 h. The mixture was quenched with water (80 mL) and filtered. The filtrate was extracted with ethyl acetate (80 mL×3). The organic layers were dried over anhydrous Na₂SO₄ and concentrated under vacuum to afford crude product (1.18 g) as a yellow oil. LCMS: Rt=1.268 min, MS (ESI) m/z=271.0 [M+H]⁺.

### Step 3

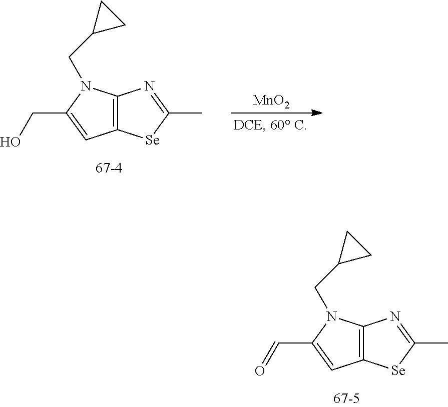

Compound 67-5: To a solution of compound 67-4 (1.18 g, 4.4 mmol) in DCE (80 mL) was added MnO₂ (3.2 g, 36.82 mmol). The reaction mixture was stirred at 60° C. for 16 h. The mixture was filtered and the filtrate was concentrated under vacuum to afford crude product (1 g) as a brown oil. LCMS: Rt=1.403 min, MS (ESI) m/z=269.0 [M+H]⁺.

### Step 4

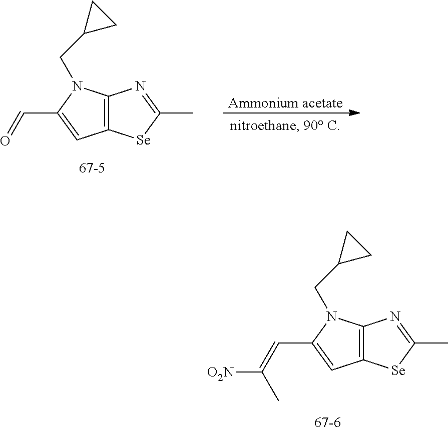

Compound 67-6: A round-bottom flask containing a mixture of compound 67-5 (1 g, 3.73 mmol), nitroethane (2.8 g, 37.4 mmol) and ammonium acetate (433 mg, 5.61 mmol) was placed in oil bath heated to 90° C. and stirred at 90° C. for 2 h. The residue was diluted with water (30 mL), extracted with ethyl acetate (50 mL×2). The combined organic layers were dried over anhydrous sodium sulfate and concentrated under vacuum to afford crude product, which was purified by flash chromatography (elution gradient: petroleum ether/ethyl acetate, 5/1, v/v) to afford compound 67-6 (1.1 g) as a yellow solid, yield: 91.7%. LCMS: Rt=1.40 min, MS (ESI) m/z=326.0 [M+H]⁺.

### Step 5

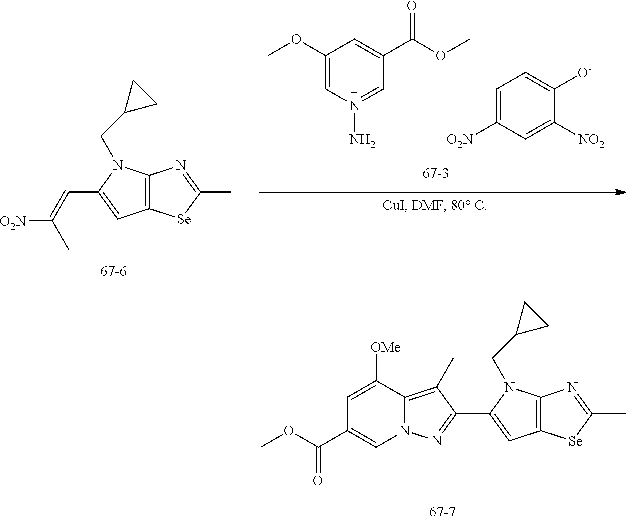

Compound 67-7: To a solution of compound 67-6 (800 mg, 2.46 mmol) in DMF (16 mL) was added compound 67-3 (2.72 g, 7.42 mmol) and CuI (240 mg, 1.26 mmol). The mixture was stirred at 80° C. for 16 h. The mixture was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/1, v/v) to afford compound 67-7 (520 mg) as a yellow solid, yield: 46.1%. LCMS: Rt=1.61 min, MS (ESI) m/z=458.9 [M+H]⁺.

### Step 6

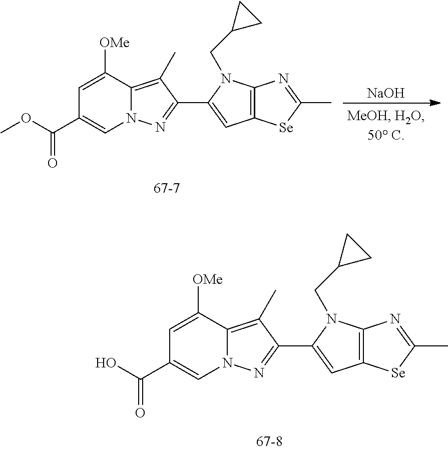

Compound 67-8: To a solution of compound 67-7 (520 mg, 1.14 mmol) in MeOH (4 mL)/THF (4 mL) was added 2N NaOH (2.2 mL, 4.4 mmol). The mixture was stirred at 50° C. for 7 h. The mixture was concentrated and adjusted pH to 5-6 with 1N hydrochloric acid aqueous solution and diluted with H₂O (20 mL), then extracted with dichloromethane (30 mL×3). The organic phases were combined, dried over Na₂SO₄, filtered and concentrated under vacuum to give compound 67-8 (400 mg) as a yellow oil, yield: 79.4%. LCMS: Rt=1.41 min, MS (ESI) m/z=444.9 [M+H]⁺.

### Step 7

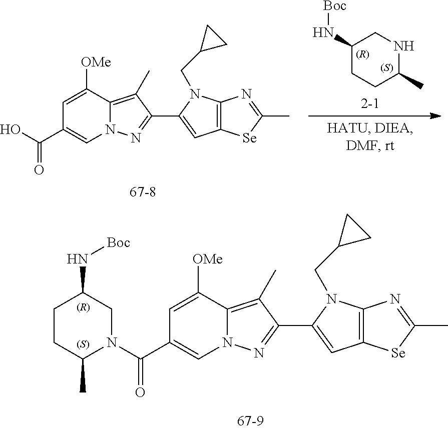

Compound 67-9: To a solution of compound 67-8 (100 mg, 0.23 mmol), DIEA (88 mg, 0.68 mol), HATU (112 mg, 0.29 mmol) in DMF (10 mL) was added compound 2-1 (54 mg, 0.25 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 67-9 (120 mg) as a yellow solid, yield: 83.3%. LCMS: Rt=1.52 min, MS (ESI) m/z=641.2[M+H]⁺.

### Step 8

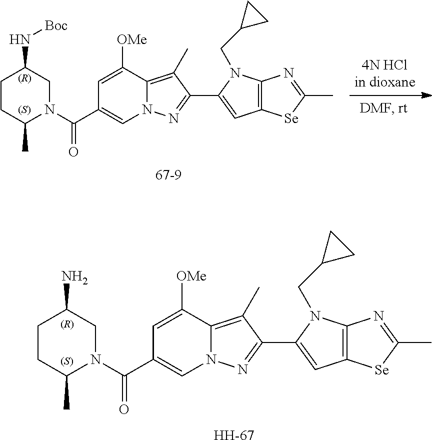

HH-67: To a solution of compound 67-9 (120 mg, 0.19 mmol) in DCM (5 mL) was added hydrochloric acid solution (5 mL, 4M in dioxane). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-60%) to afford HH-67 (83.3 mg, hydrochloric acid salt form) as a green solid. yield: 77.1%. ¹H NMR (400 MHz, DMSO-d₆): δ ppm 8.40 (s, 1H), 8.23 (s, 3H), 6.65 (s, 1H), 6.55 (s, 1H), 4.35-4.33 (m, 2H), 4.15 (m, 4H), 3.98 (s, 3H), 3.19-3.10 (m, 2H), 2.78 (s, 3H), 1.89-1.76 (m, 3H), 1.61-1.59 (m, 1H), 1.24-1.22 (m, 4H), 0.35-0.30 (m, 2H), 0.28-0.26 (m, 2H). LCMS: Rt=1.13 min, MS (ESI) m/z=541.2 [M+H]⁺. HPLC: Rt=6.17 min, Purity: 100% (214 nm and 254 nm).

### Example 68. Synthesis of HH-68

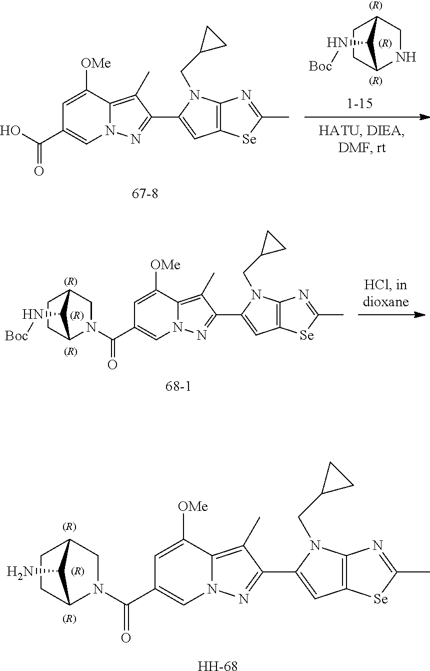

### Step 1

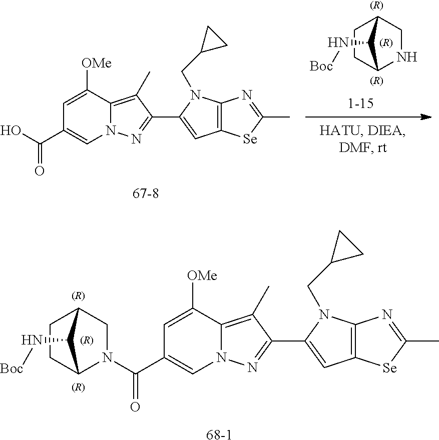

Compound 68-1: To a solution of compound 67-8 (80 mg, 0.18 mmol), DIEA (70 mg, 0.54 mol), HATU (90 mg, 0.24 mmol) in DMF (6 mL) was added compound 1-15 (44 mg, 0.20 mmol). The mixture was stirred at room temperature for 2 h. The mixture was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 68-1 (96 mg) as a yellow oil, yield: 83.5%. LCMS: Rt=1.49 min, MS (ESI) m/z=639.2 [M+H]⁺.

### Step 2

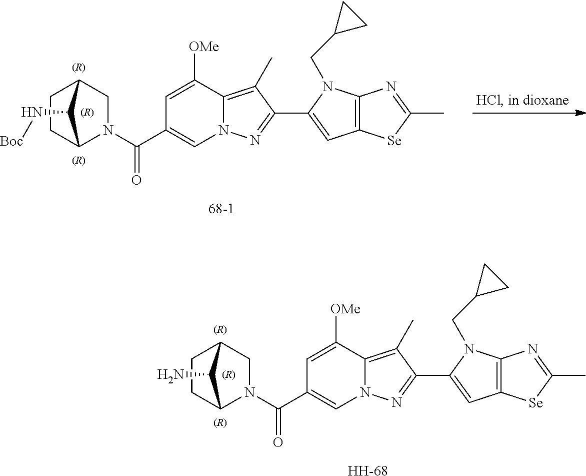

HH-68: To a solution of compound 68-1 (96 mg, 0.15 mmol) in DCM (3 mL) was added hydrochloric acid solution (3 mL, 4M in dioxane). The mixture was stirred at room temperature for 4 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-68 (44.8 mg, hydrochloric acid salt form) as a brown solid. yield: 53%. ¹H NMR (400 MHz, DMSO-d₆): δ ppm 8.39-8.36 (m, 4H), 6.62 (s, 1H), 6.59 (s, 1H), 4.46 (br s, 1H), 6.34 (d, J=7.2 Hz, 2H), 3.98 (s, 3H), 3.70 (m, 4H), 3.53 (s, 1H), 3.24 (d, J=10.0 Hz, 1H), 2.77 (s. 3H), 2.66 (m, 1H), 2.01-1.90 (m, 3H), 1.68-1.60 (m, 1H), 1.25-1.16 (m, 1H), 0.35-0.30 (m, 2H), 0.24-0.21 (m, 2H). LCMS: Rt=1.09 min, MS (ESI) m/z=539.0 [M+H]⁺. HPLC: Rt=6.16 min, Purity: 99.37% (214 nm) and 99.27% (254 nm).

### Example 69. Synthesis of HH-69

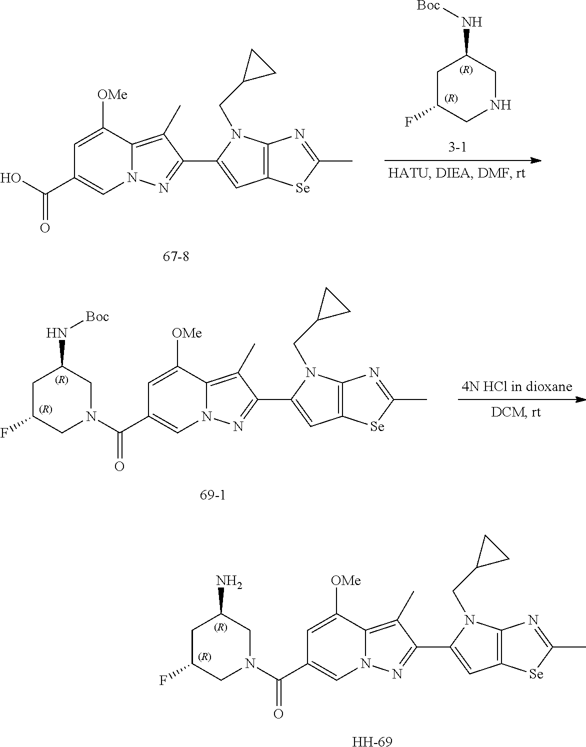

### Step 1

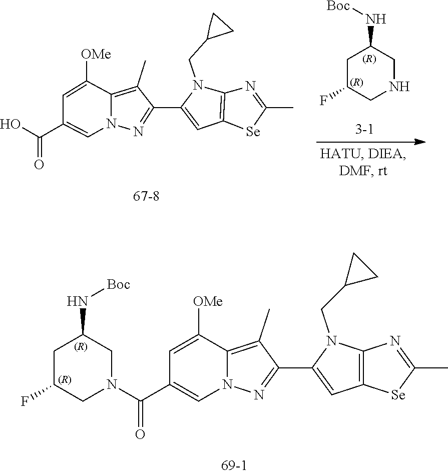

Compound 69-1: To a solution of compound 67-8 (100 mg, 0.23 mmol), DIEA (88 mg, 0.68 mol), HATU (112 mg, 0.29 mmol) in DMF (10 mL) was added compound 3-1 (55 mg, 0.25 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 69-1 (120 mg) as a yellow solid, yield: 82.8%. LCMS: Rt=1.46 min, MS (ESI) m/z=645.2[M+H]⁺.

### Step 2

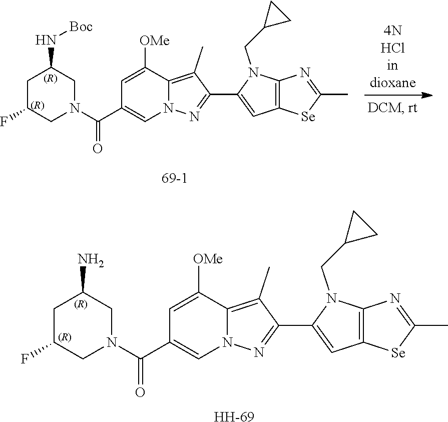

HH-69: To a solution of compound 69-1 (120 mg, 0.19 mmol) in DCM (5 mL) was added hydrochloric acid solution (5 mL, 4M in dioxane). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-60%) to afford HH-69 (59.2 mg, hydrochloric acid salt form) as a green solid. yield: 54.8%. ¹H NMR (400 MHz, DMSO-d4): δ ppm 8.39 (m, 4H), 6.66 (s, 1H), 6.55 (s, 1H), 5.09-4.92 (m, 6H), 4.34 (d, J=7.2 Hz, 2H), 3.97 (s, 3H), 3.39-3.08 (m, 3H), 2.78 (s, 3H), 2.43-2.33 (m, 1H), 2.03-1.87 (m, 1H), 1.29-1.19 (m, 1H), 0.36-0.30 (m, 2H), 0.27-0.25 (m, 2H). LCMS: Rt=1.13 min, MS (ESI) m/z=545.1 [M+H]⁺. HPLC: Rt=3.66 min, Purity: 99.87% (214 nm) and 100% (254 nm).

### Example 70. Synthesis of HH-70

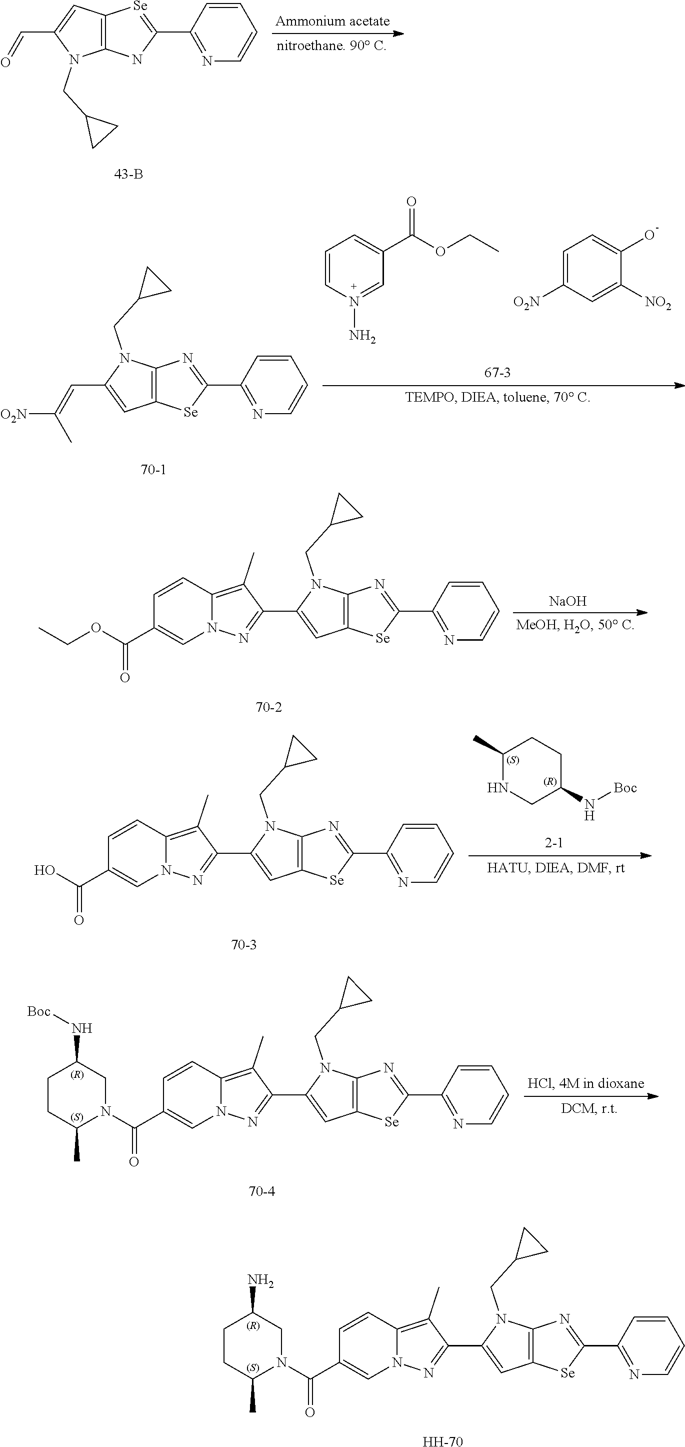

### Step 1

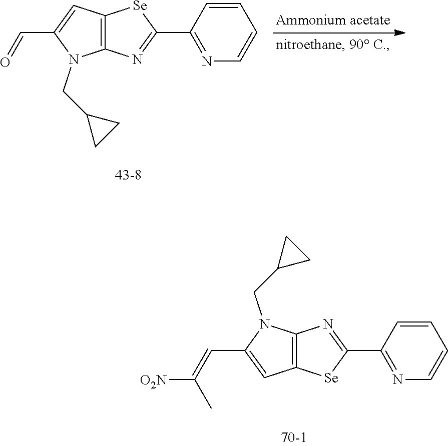 [SMILES: O=Cc1cc2[se]c(-c3ccccn3)nc2n1CC1CC1 ; C/C(=C\c1cc2[se]c(-c3ccccn3)nc2n1CC1CC1)[N+](=O)[O-]]

Compound 70-1: A round-bottom flask containing a mixture of compound 43-8 (300 mg, 0.91 mmol), nitroethane (682 mg, 9.08 mmol) and ammonium acetate (105 mg, 1.36 mmol) was placed in oil bath heated to 90° C. and stirred at 90° C. for 2 h. The residue was diluted with water (30 mL), extracted with ethyl acetate (50 mL×2). The combined organic layers were dried over anhydrous sodium sulfate and concentrated under vacuum to afford crude product, which was purified by flash chromatography (elution gradient: petroleum ether/ethyl acetate, 3/1, v/v) to afford compound 70-1 (180 mg) as a yellow solid, yield: 51.3%. LCMS: Rt=1.50 min, MS (ESI) m/z=389.0 [M+H]⁺.

### Step 2

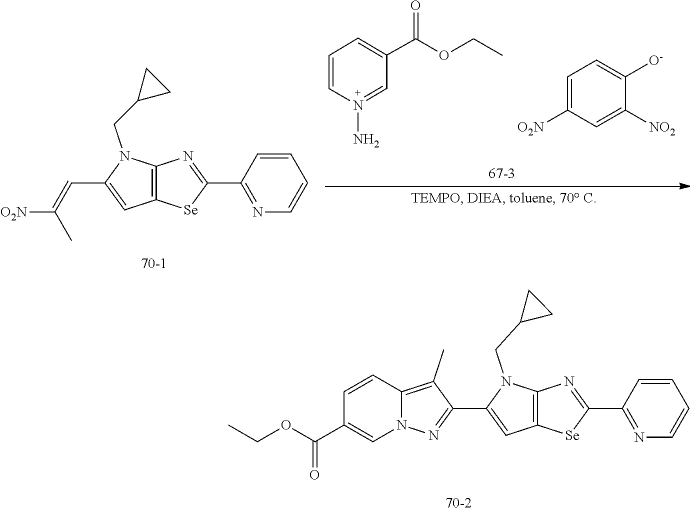

Compound 70-2: To a solution of compound 70-1 (150 mg, 0.39 mmol) in toluene (5 mL) was added compound 67-3 (204 mg, 0.58 mmol), TEMPO (73 mg, 0.46 mmol) and DIEA (100 mg, 0.77 mmol). The mixture was stirred at 70° C. for 16 h. The mixture was concentrated in vacuo to afford residue. The residue was diluted with ethyl acetate (50 mL), washed with water (20 mL×2) and brine (20 mL). The organic layer was dried over anhydrous sodium sulfate, filtered and concentrated under vacuum to afford crude product, which was purified by flash chromatography (elution gradient: petroleum ether/ethyl acetate, 1/1, v/v) to afford compound 70-2 (190 mg) as a yellow solid, yield: 97.4%. LCMS: Rt=1.75 min, MS (ESI) m/z=506.1 [M+H]⁺.

### Step 3

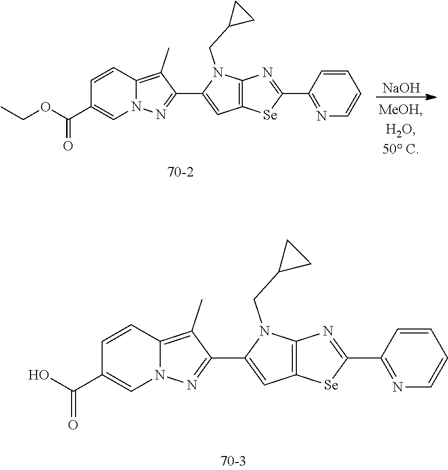

Compound 70-3: To a solution of compound 70-2 (190 mg, 0.37 mmol) in MeOH (2 mL)/THF (2 mL) was added 2N NaOH (0.8 mL, 1.6 mmol). The mixture was stirred at 50° C. for 5 h. The mixture was concentrated and adjusted pH to 5-6 with 1N hydrochloric acid aqueous solution and diluted with H₂O (20 mL), then extracted with dichloromethane (30 mL×3). The organic phases were combined, dried over Na₂SO₄, filtered and concentrated under vacuum to give compound 70-3 (174 mg) as a yellow oil, yield: 97.2%. LCMS: Rt=1.47 min, MS (ESI) m/z=478.1 [M+H]⁺.

### Step 4

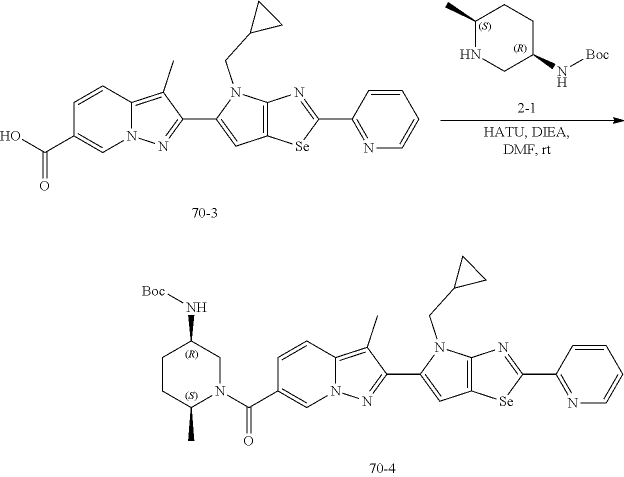

Compound 70-4: To a solution of compound 70-3 (58 mg, 0.12 mmol), DIEA (48 mg, 0.37 mol), HATU (60 mg, 0.16 mmol) in DMF (5 mL) was added compound 2-1 (29 mg, 0.14 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 70-4 (70 mg) as a yellow solid, yield: 85.6%. LCMS: Rt=1.57 min, MS (ESI) m/z=674.2 [M+H]⁺.

### Step 5

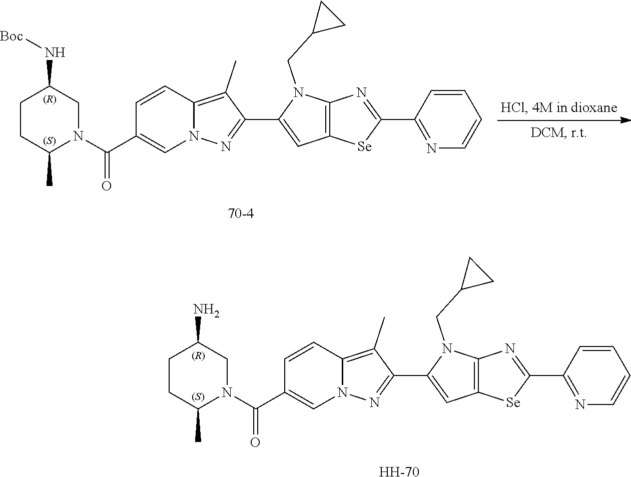

HH-70: To a solution of compound 70-4 (70 mg, 0.10 mmol) in DCM (2 mL) was added hydrochloric acid solution (2 mL, 4M in dioxane). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-70 (35.2 mg, hydrochloric acid salt form) as a red solid. yield: 55.6%. ¹H NMR (400 MHz, DMSO-d₆): δ ppm 8.85 (s, 1H), 8.61 (d, J=4.4 Hz, 1H), 8.31 (br s, 3H), 8.10-8.08 (m, 1H), 7.97-7.93 (m, 1H), 7.79 (d, J=8.8 Hz, 1H), 7.49-7.46 (m, 1H), 7.24-7.22 (m, 1H), 6.89 (s, 1H), 4.58-4.15 (m, 3H), 3.18 (s, 2H), 2.44 (s, 3H), 1.91-1.79 (m, 3H), 1.62-1.59 (m, 1H), 1.40-1.35 (m, 1H), 1.24 (d, J=6.8 Hz, 3H), 0.43-0.41 (m, 4H). LCMS: Rt=1.16 min, MS (ESI) m/z=574.2 [M+H]⁺. HPLC: Rt=6.36 min, Purity: 98.00% (214 nm) and 96.01% (254 nm).

### Example 71. Synthesis of HH-71

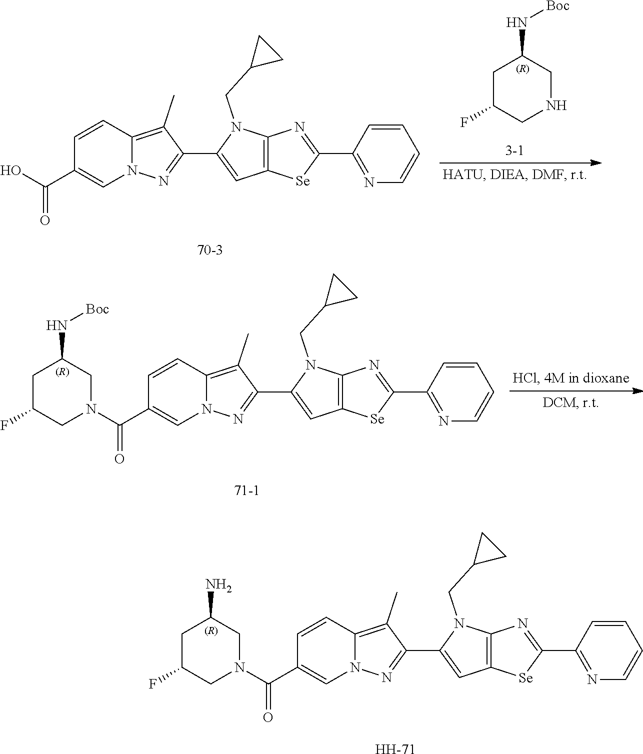

### Step 1

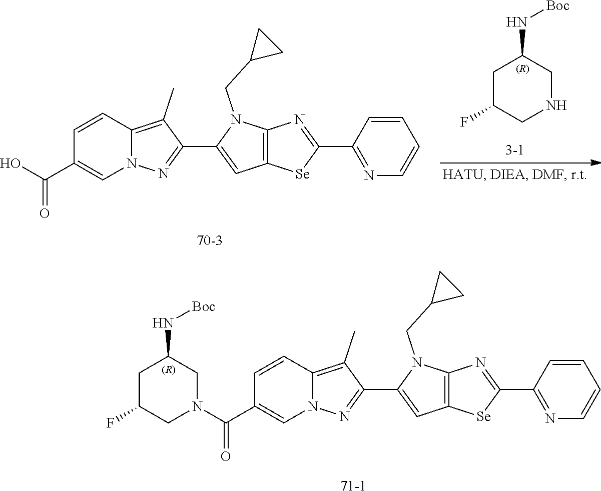

Compound 71-1: To a solution of compound 70-3 (58 mg, 0.12 mmol), DIEA (48 mg, 0.37 mol), HATU (60 mg, 0.16 mmol) in DMF (5 mL) was added compound 3-1 (29 mg, 0.13 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 71-1 (74 mg) as a yellow solid, yield: 89.9%. LCMS: Rt=1.51 min, MS (ESI) m/z=678.2 [M+H]⁺.

### Step 2

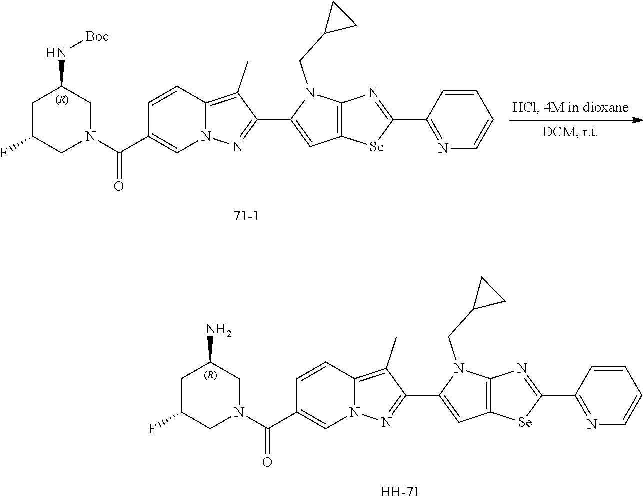

HH-71: To a solution of compound 71-1 (74 mg, 0.11 mmol) in DCM (2 mL) was added hydrochloric acid solution (2 mL, 4M in dioxane). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-71 (39.8 mg, hydrochloric acid salt form) as a red solid. yield: 59.4%. ¹H NMR (400 MHz, DMSO-d₆): δ ppm 8.83 (m, 1H), 8.61 (d, J=4.4 Hz, 1H), 8.43 (br s, 3H), 8.10-8.08 (m, 1H), 7.97-7.93 (m, 1H), 7.80 (d, J=9.2 Hz, 1H), 7.49-7.46 (m, 1H), 7.24 (d, J=9.2 Hz, 1H), 6.90 (s, 1H), 5.09-4.98 (m, 1H), 4.59-4.12 (m, 4H), 3.40-3.11 (m, 3H), 2.44 (s, 3H), 2.41-2.34 (m, 1H), 2.05-1.89 (m, 1H), 1.43-1.34 (m, 1H), 0.43-0.41 (m, 4H). LCMS: Rt=1.17 min, MS (ESI) m/z=578.1 [M+H]⁺. HPLC: Rt=6.42 min, Purity: 97.93% (214 nm) and 96.84% (254 nm).

### Example 72. Synthesis of HH-72

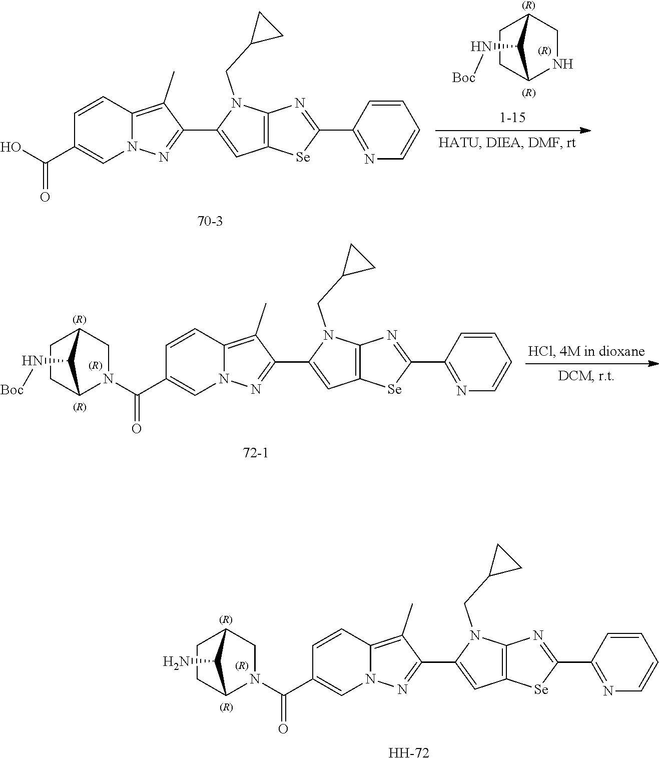

### Step 1

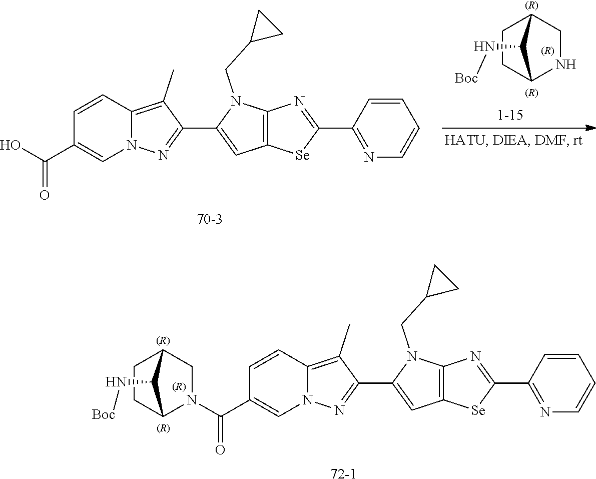

Compound 72-1: To a solution of compound 70-3 (58 mg, 0.12 mmol), DIEA (48 mg, 0.37 mol), HATU (60 mg, 0.16 mmol) in DMF (5 mL) was added compound 1-15 (29 mg, 0.14 mmol). The mixture was stirred at room temperature for 2 h. The residue was diluted with ethyl acetate (50 mL), washed with water (30 mL×2) and brine (30 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 72-1 (72 mg) as a yellow solid, yield: 88.2%. LCMS: Rt=1.54 min, MS (ESI) m/z=672.0 [M+H]⁺.

### Step 2

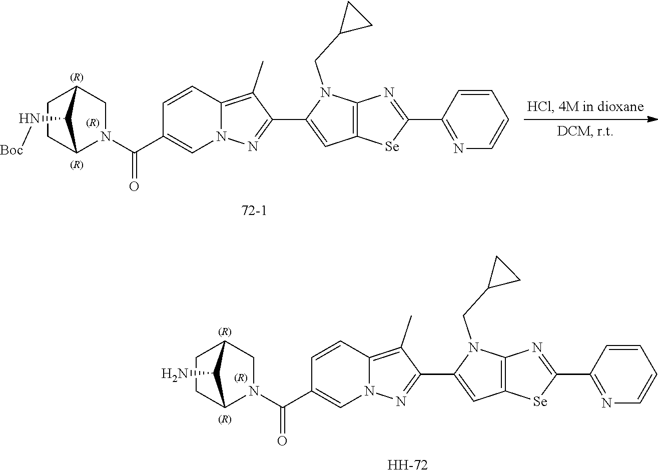

HH-72: To a solution of compound 72-1 (72 mg, 0.11 mmol) in DCM (2 mL) was added hydrochloric acid solution (2 mL, 4M in dioxane). The mixture was stirred at room temperature for 2 h. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 15-45%) to afford HH-72 (29.4 mg, hydrochloric acid salt form) as a red solid. yield: 45.2%. ¹H NMR (400 MHz, DMSO-d₆): δ ppm 8.81 (s, 1H), 8.58 (d, J=4.8 Hz, 1H), 8.43 (br s, 3H), 8.09-8.07 (m, 1H), 7.94-7.90 (m, 1H), 7.71 (d, J=9.2 Hz, 1H), 7.44-7.41 (m, 1H), 7.29 (d, J=9.2 Hz, 1H), 6.85 (s, 1H), 4.58-4.46 (m, 3H), 3.69 (br s, 1H), 3.53 (s, 1H), 3.26-3.24 (m, 1H), 2.68 (s, 1H), 2.42 (s, 3H), 2.06-1.91 (m, 3H), 1.69-1.62 (m, 1H), 1.37-1.30 (m, 1H), 0.42-0.35 (m, 4H). LCMS: Rt=1.15 min, MS (ESI) m/z=572.2 [M+H]⁺. HPLC: Rt=6.27 min, Purity: 98.90% (214 nm) and 97.19% (254 nm).

### Example 73. Synthesis of HH-73

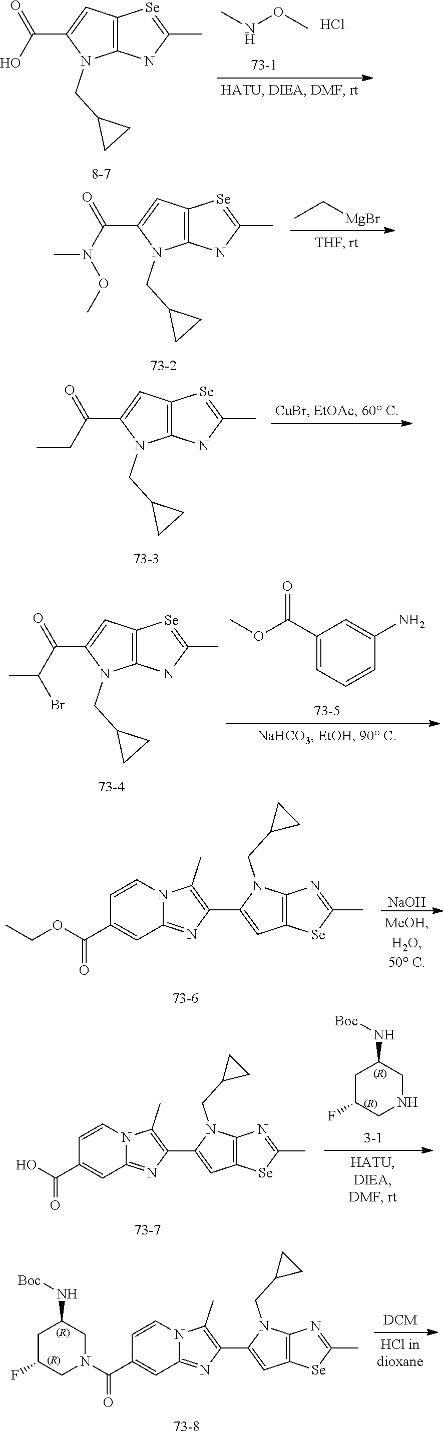

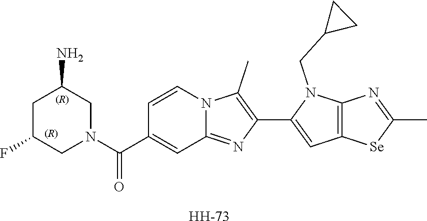

### Step 1

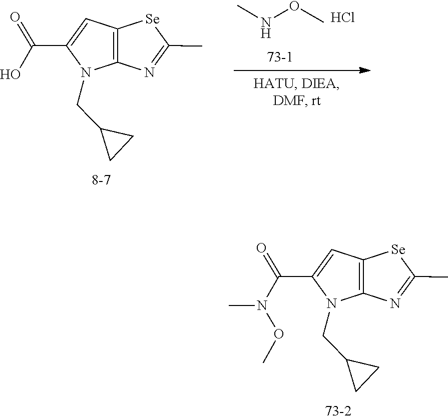

Compound 73-2: To a suspension of compound 8-7 (300 mg, 1.06 mmol), HATU (524 mg, 1.38 mmol) and DIEA (411 mg, 3.18 mmol) in DMF (5 mL) was added compound 73-1 (155 mg, 1.59 mmol). The mixture was stirred at room temperature for 2 hrs. The residue was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 10/1, v/v) to afford compound 73-2 (300 mg) as a yellow oil, yield: 87.0%. LCMS: Rt=1.29 min, MS (ESI) m/z=328.1 [M+H]⁺.

### Step 2

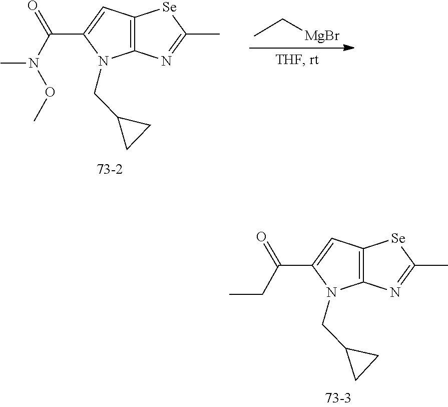

Compound 73-3: To a solution of compound 73-2 (300 mg, 0.92 mmol) in THF (10 mL) under N₂ was added ethylmagnesium bromide (0.9 mL, 2.7 mmol). The mixture was stirred at room temperature under N₂ for 2 hrs. The mixture was diluted with water (20 mL), extracted with ethyl acetate (30 mL×2). The combined organic layers were dried over anhydrous sodium sulfate and concentrated under vacuum to afford crude product, which was purified by flash chromatography (elution gradient: petroleum ether/ethyl acetate, 5/1, v/v) to compound 73-3 (220 mg) as a yellow oil, yield: 81.0%. LCMS: Rt=1.42 min, MS (ESI) m/z=297.1 [M+H]⁺.

### Step 3

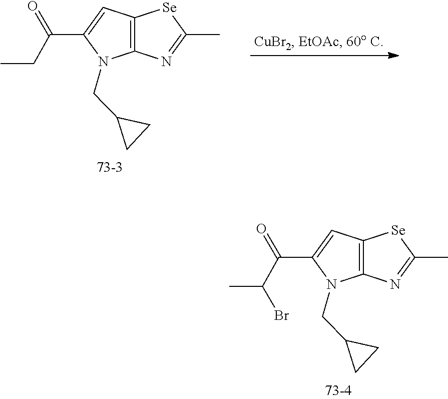 [SMILES: CCC(=O)c1cc2[se]c(C)nc2n1CC1CC1 ; Cc1nc2c(cc(C(=O)C(C)Br)n2CC2CC2)[se]1]

Compound 73-4: To a solution of compound 73-3 (180 mg, 0.61 mmol) in ethyl acetate (10 mL) was added CuBr₂ (273 mg, 1.22 mmol). The mixture was stirred at 60° C. for 48 hrs. The mixture was filtered and concentrated under vacuum to afford crude product, which was purified by flash chromatography (elution gradient: petroleum ether/dichloromethane, 1/1, v/v) to afford compound 73-4 (170 mg) as a yellow oil, yield: 74.9%. LCMS: Rt=1.44 min, MS (ESI) m/z=374.9 [M+H]⁺.

### Step 4

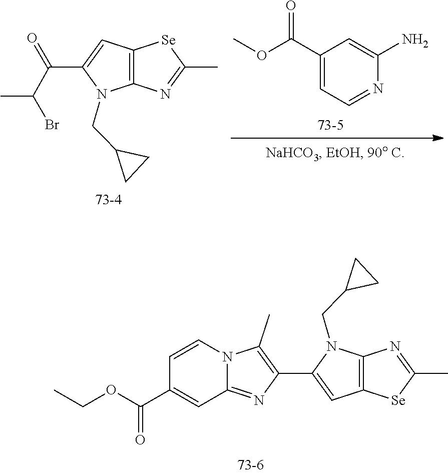

Compound 73-6: To a solution of compound 73-4 (150 mg, 0.40 mmol) in EtOH (10 mL) was added compound 73-5 (61 mg, 0.40 mmol) and NaHCO₃ (168.4 mg, 2.0 mmol). The mixture was stirred at 90° C. for 16 hrs. The mixture was concentrated under reduced pressure to afford residue. The residue was diluted with water (30 mL), extracted with ethyl acetate (30 mL×3). The combined organic layers were dried over anhydrous sodium sulfate and concentrated under vacuum to afford crude product, which was purified by flash chromatography (elution gradient: petroleum ether/ethyl acetate, 1/1, v/v) to afford compound 73-6 (70 mg) as a yellow solid, yield: 39.5%. LCMS: Rt=1.38 min, MS (ESI) m/z=443.1 [M+H]⁺.

### Step 5

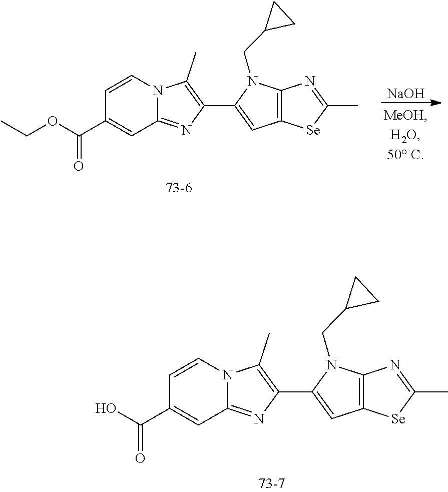

Compound 73-7: To a solution of compound 73-6 (70 mg, 0.16 mmol) in MeOH (1 mL)/H₂O (1 mL) was added 2N sodium hydroxide aqueous solution (0.3 mL, 0.6 mmol). The mixture was stirred at 50° C. for 12 hrs. The mixture was concentrated and adjusted pH to 3-4 with 1N hydrochloric acid aqueous solution and diluted with H₂O (20 mL), then extracted with dichloromethane (30 mL×3). The combined organic layers were dried over anhydrous sodium sulfate and concentrated under vacuum to afford compound 73-7 (40 mg) as a yellow solid, yield: 61.0%. LCMS: Rt=1.13 min, MS (ESI) m/z=415.1 [M+H]⁺.

### Step 6

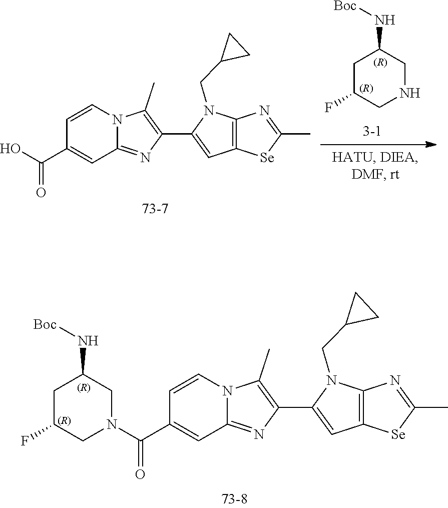

Compound 73-8: To a solution of compound 73-7 (40 mg, 0.097 mmol), DIEA (38 mg, 0.29 mmol), HATU (48 mg, 0.13 mmol) in DMF (5 mL) was added compound 3-1 (24 mg, 0.11 mmol). The mixture was stirred at room temperature for 3 hrs. The residue was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 73-8 (50 mg) as a yellow solid, yield: 84.3%. LCMS: Rt=1.20 min, MS (ESI) m/z=614.7 [M+H]⁺.

### Step 7

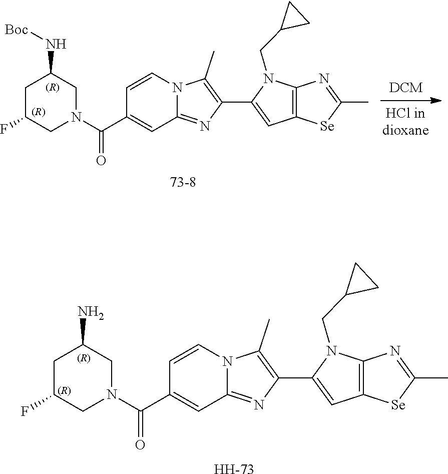

HH-73: To a solution of compound 73-8 (50 mg, 0.081 mmol) in DCM (1 mL) was added 4N HCl in dioxane (1 mL). The mixture was stirred at room temperature for 1 hr. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-73 (19.7 mg, hydrochloric acid salt form) as a yellow solid. yield: 44.0%. ¹H NMR (400 MHz, DMSO-d₆): δ ppm 8.88 (d, J=7.2 Hz, 1H), 8.61-8.53 (m, 3H), 7.94 (s, 1H), 7.48 (m, 1H), 6.74 (s, 1H), 5.20-4.93 (m, 1H), 4.78-4.56 (m, 1H), 4.19 (d, J=6.8 Hz, 2H), 4.06-3.81 (m, 1H), 3.54-3.11 (m, 3H), 2.81 (s, 3H), 2.60 (s, 3H), 2.40-2.33 (m, 1H), 2.09-1.91 (m, 1H), 1.09-1.08 (m, 1H), 0.31 (d, J=7.6 Hz, 2H), 0.17 (m, 2H). LCMS: Rt=1.00 min, MS (ESI) m/z=515.1 [M+H]⁺.

### Example 74. Synthesis of HH-74

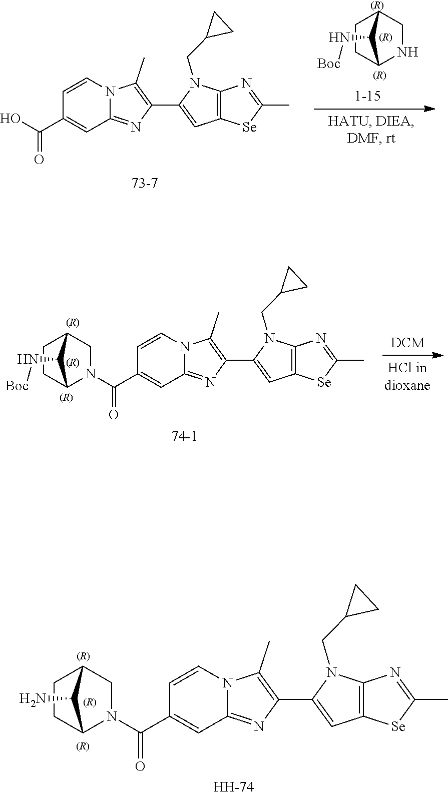

### Step 1

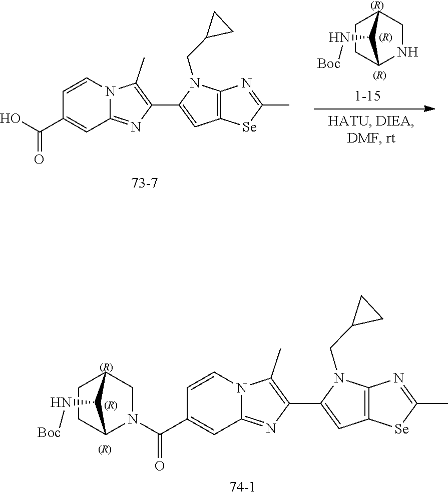

Compound 74-1: To a solution of compound 73-7 (80 mg, 0.19 mmol), DIEA (75 mg, 0.58 mmol), HATU (96 mg, 0.25 mmol) in DMF (5 mL) was added compound 1-15 (45 mg, 0.21 mmol). The mixture was stirred at room temperature for 3 hrs. The residue was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 74-1 (110 mg) as a yellow solid, yield: 94.0%. LCMS: Rt=1.21 min, MS (ESI) m/z=609.2 [M+H]⁺.

### Step 2

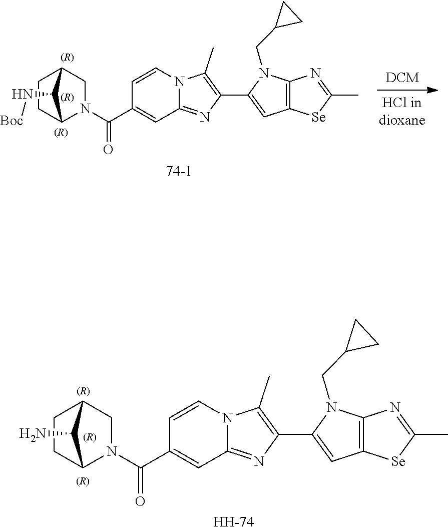

HH-74: To a solution of compound 74-1 (110 mg, 0.18 mmol) in DCM (1 mL) was added 4N HCl in dioxane (1 mL). The mixture was stirred at room temperature for 1 hr. The resulting mixture was concentrated under vacuum, which was purified by preparative HPLC (Gemini-C18 column, 5μ silica, 21.2 mm diameter, 150 mm length), using decreasingly polar mixtures of water (containing 0.05% TFA) and MeCN as eluents 20-50%) to afford HH-74 (54.6 mg, trifluoroacetic acid salt form) as a yellow solid. yield: 48.5%. ¹H NMR (400 MHz, DMSO-d₆): δ ppm 8.39 (d, J=6.8 Hz, 1H), 8.03 (br s, 3H), 7.73 (s, 1H), 7.09 (d, J=6.4 Hz, 1H), 6.57 (s, 1H), 4.43-4.42 (m, 3H), 3.67-3.58 (m, 2H), 3.25-3.22 (m, 1H), 2.78 (s, 3H), 2.66 (s, 1H), 2.61 (s, 3H), 1.94 (m, 3H), 1.71-1.65 (m, 1H), 1.25-1.20 (m, 1H), 0.32-0.29 (m, 2H), 0.25-0.23 (m, 2H). LCMS: Rt=0.99 min, MS (ESI) m/z=509.1 [M+H]⁺.

### Example 75. Synthesis of HH-75

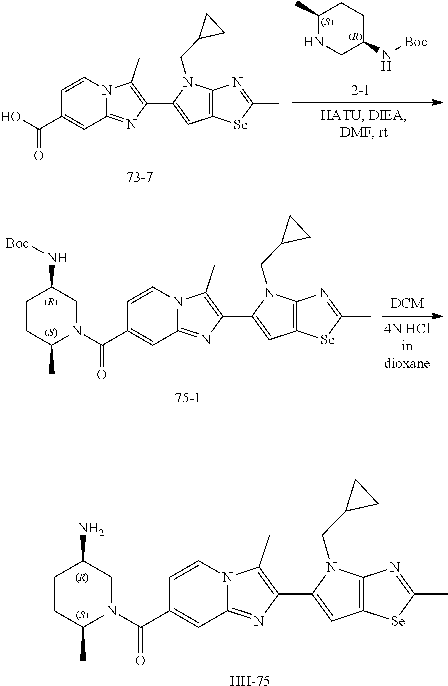

### Step 1

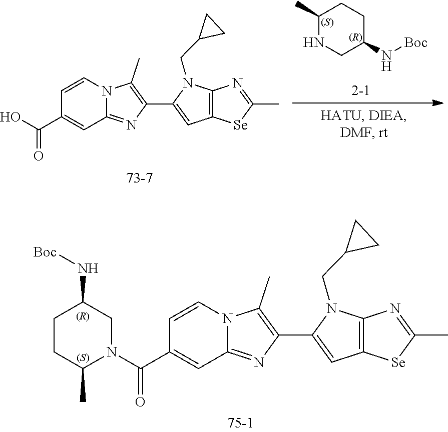

Compound 75-1: To a solution of compound 73-7 (50 mg, 0.12 mmol), DIEA (47 mg, 0.36 mmol), HATU (60 mg, 0.16 mmol) in DMF (5 mL) was added compound 2-1 (29 mg, 0.14 mmol). The mixture was stirred at room temperature for 3 hrs. The residue was diluted with ethyl acetate (30 mL), washed with water (10 mL×2) and brine (10 mL). The organic layer was dried over anhydrous sodium sulfate, filtered and concentrated under vacuum to afford crude product, which was purified by flash chromatography (elution gradient: petroleum ether/ethyl acetate, 1/9, v/v) to afford compound 75-1 (62 mg) as a yellow solid, yield: 84.1%. LCMS: Rt=1.23 min, MS (ESI) m/z=611.3 [M+H]⁺.

### Step 2

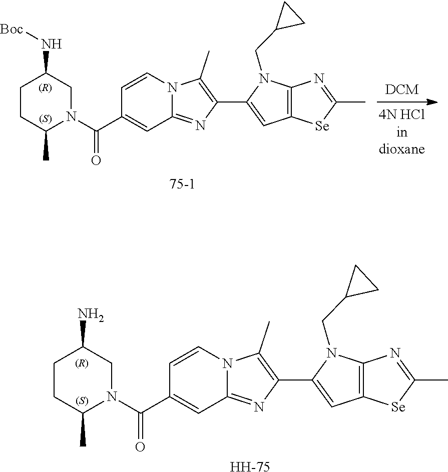

HH-75: To a solution of compound 75-1 (62 mg, 0.10 mmol) in DCM (1 mL) was added 4N HCl in dioxane (1 mL). The mixture was stirred at room temperature for 1 hr. The resulting mixture was concentrated under vacuum, which was purified by preparative HPLC (Gemini-C18 column, 5μ silica, 21.2 mm diameter, 150 mm length), using decreasingly polar mixtures of water (containing 0.05% FA) and MeCN as eluents 20-50%) to afford HH-75 (27.4 mg, hydrochloric acid salt form) as a yellow solid. yield: 49.4%. ¹H NMR (400 MHz, DMSO-d₆): δ ppm 8.86 (d, J=7.2 Hz, 1H), 8.44 (br s, 3H), 7.98 (s, 1H), 7.53 (d, J=6.4 Hz, 1H), 6.73 (s, 1H), 4.20-4.19 (m, 2H), 3.19 (m, 2H), 2.81 (s, 3H), 2.60 (s, 3H), 1.91-1.82 (m, 3H), 1.55 (m, 1H), 1.24 (d, J=6.4 Hz, 3H), 1.13-1.06 (m, 1H), 0.32-0.30 (m, 2H), 0.19-0.16 (m, 2H). LCMS: Rt=1.00 min, MS (ESI) m/z=511.2 [M+H]⁺.

The structures of the above synthesized compounds are shown as follows in Scheme 1:

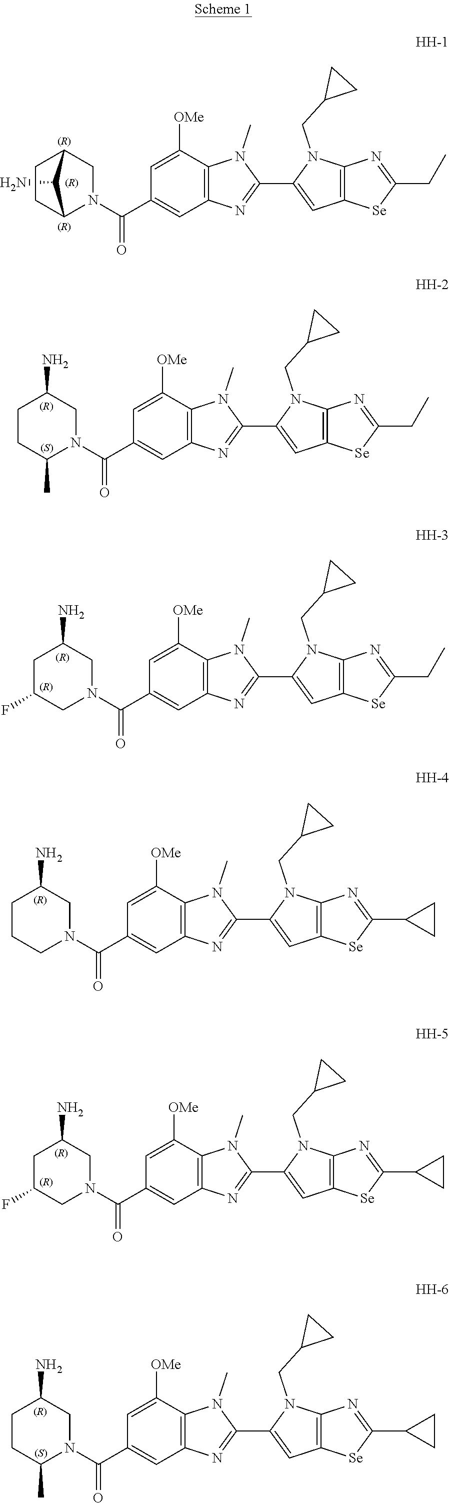

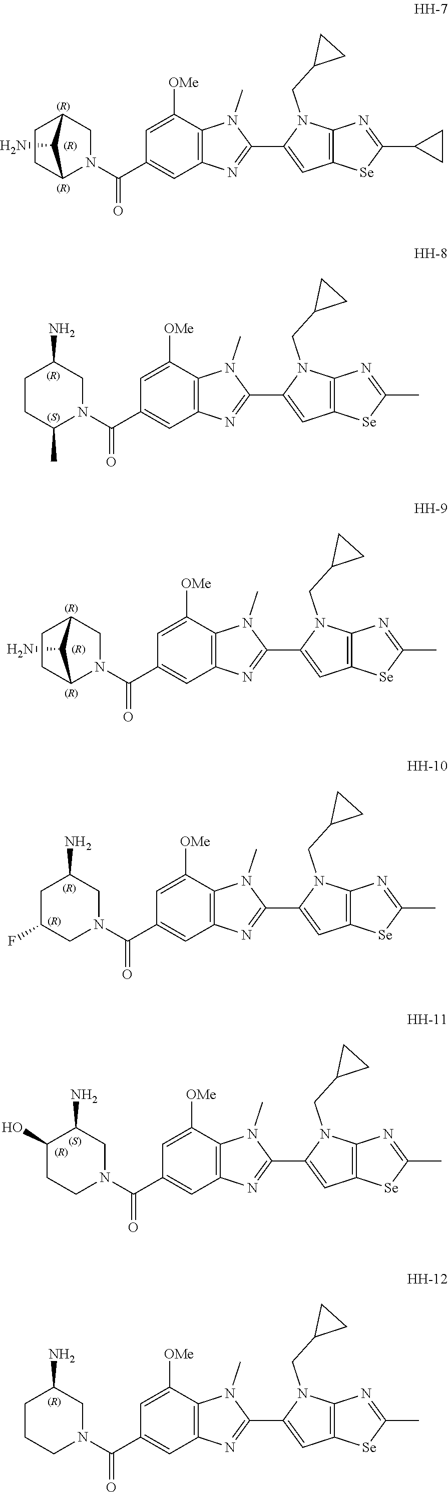

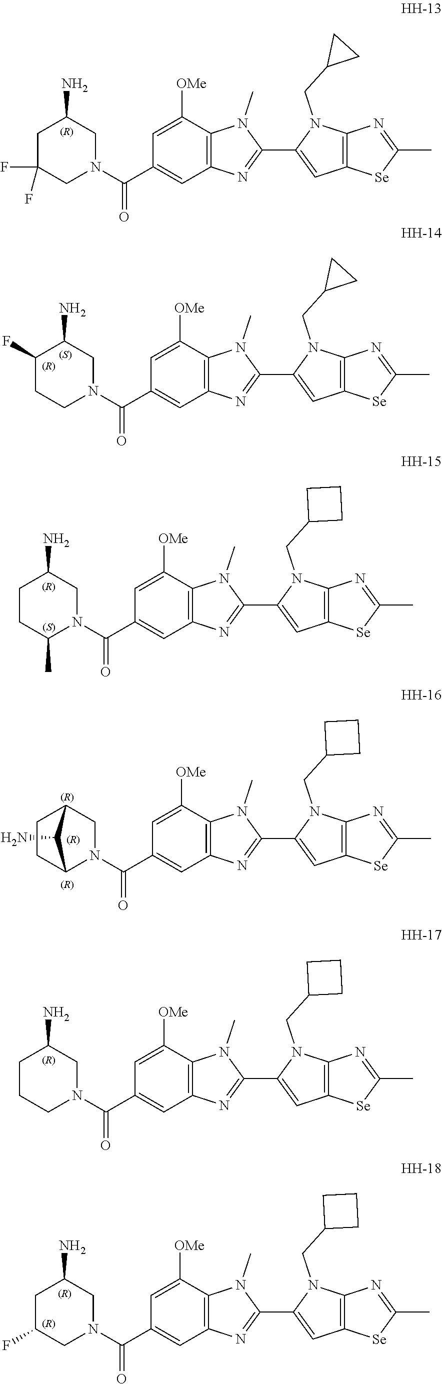

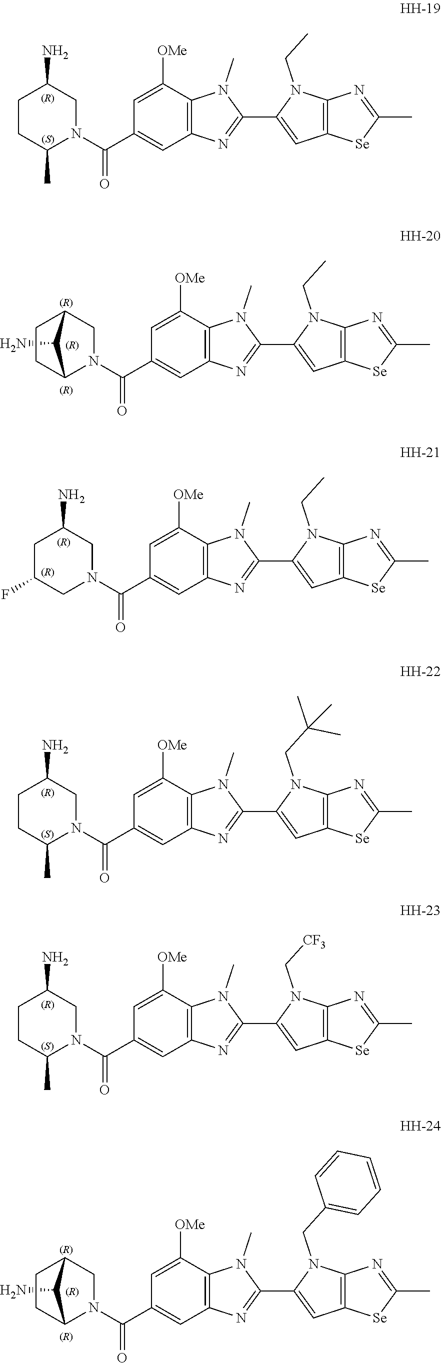

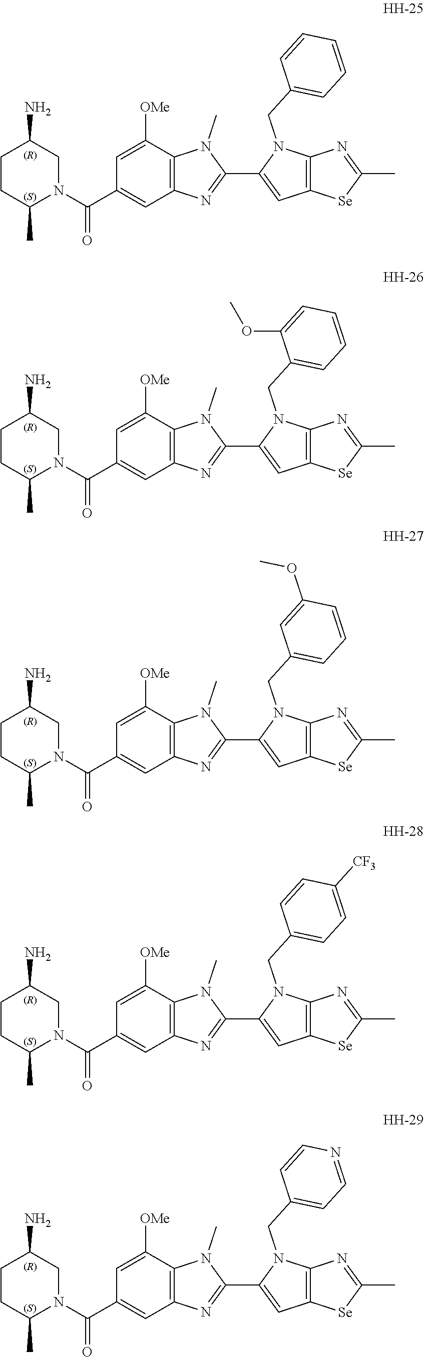

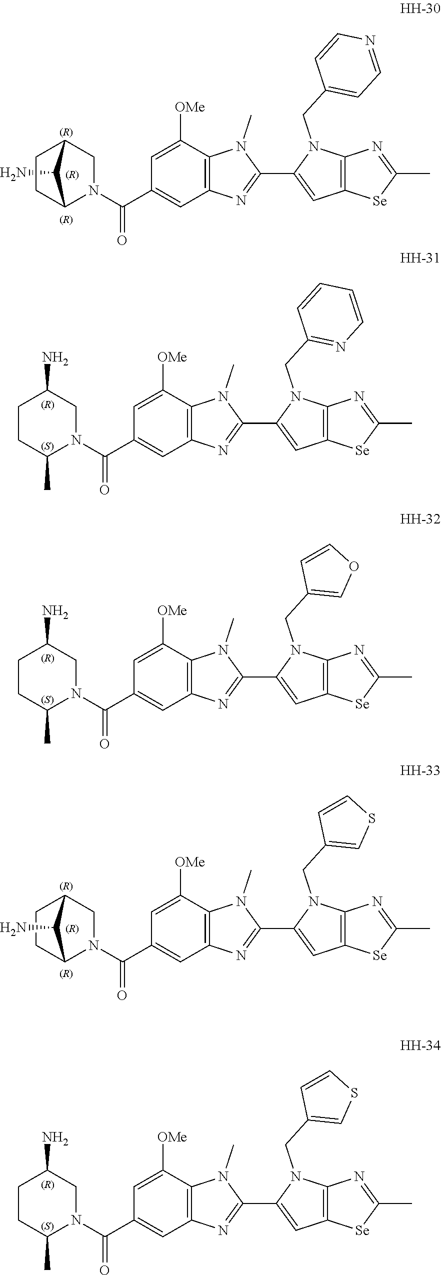

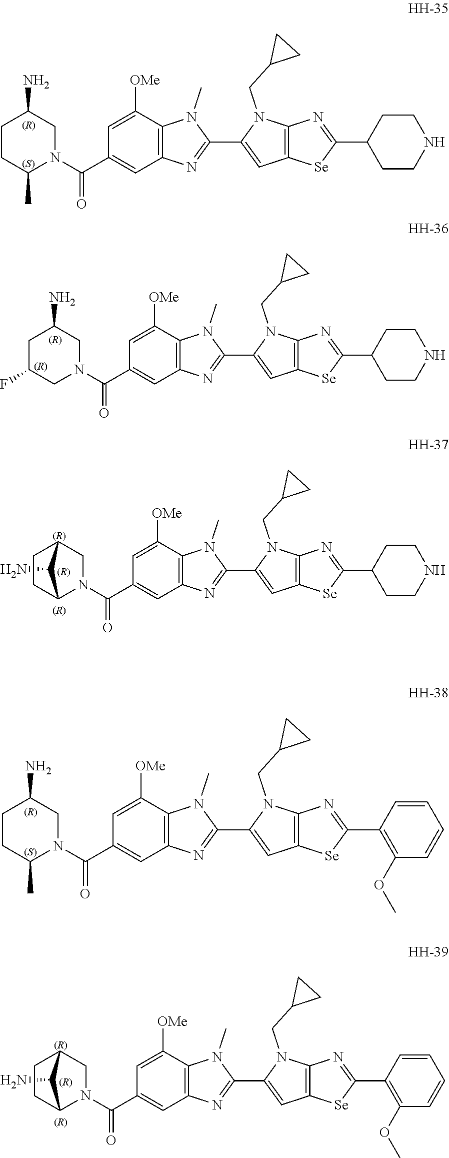

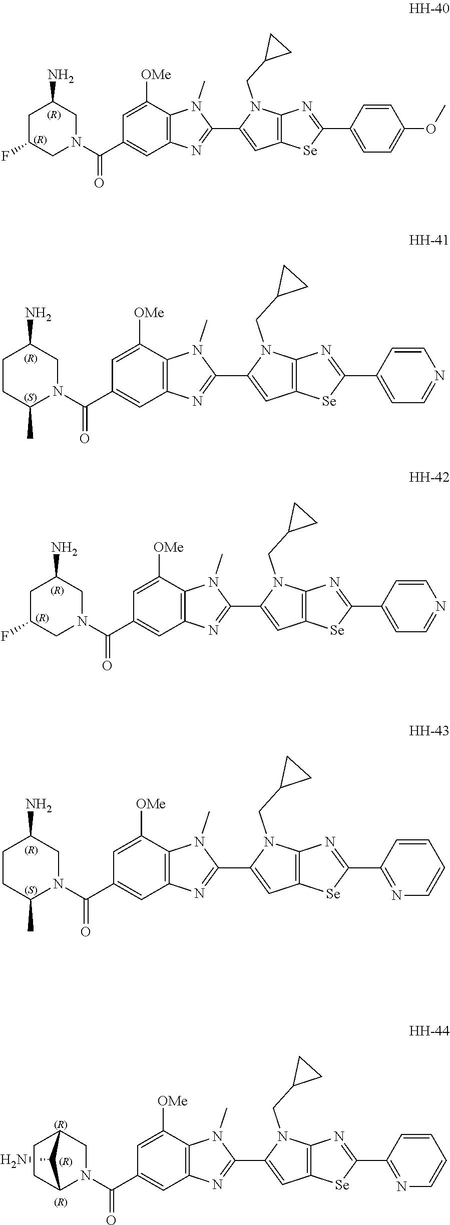

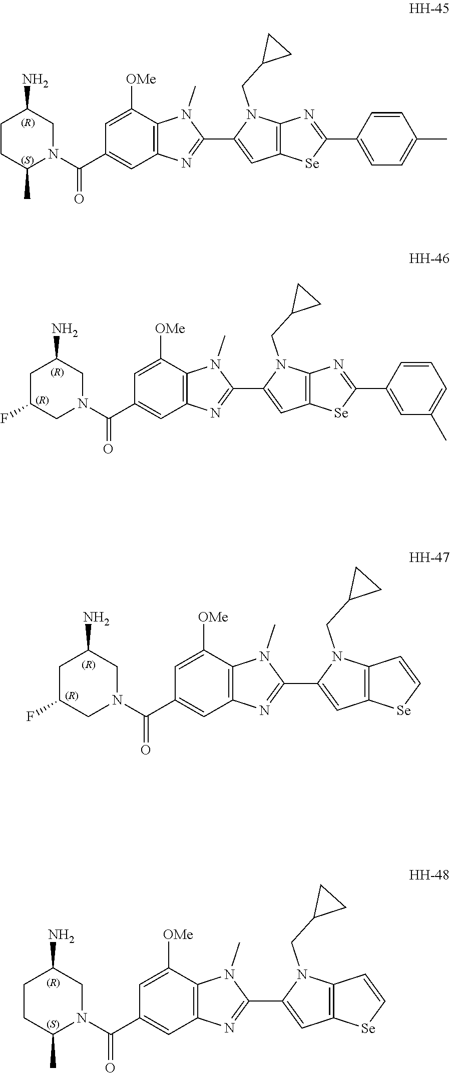

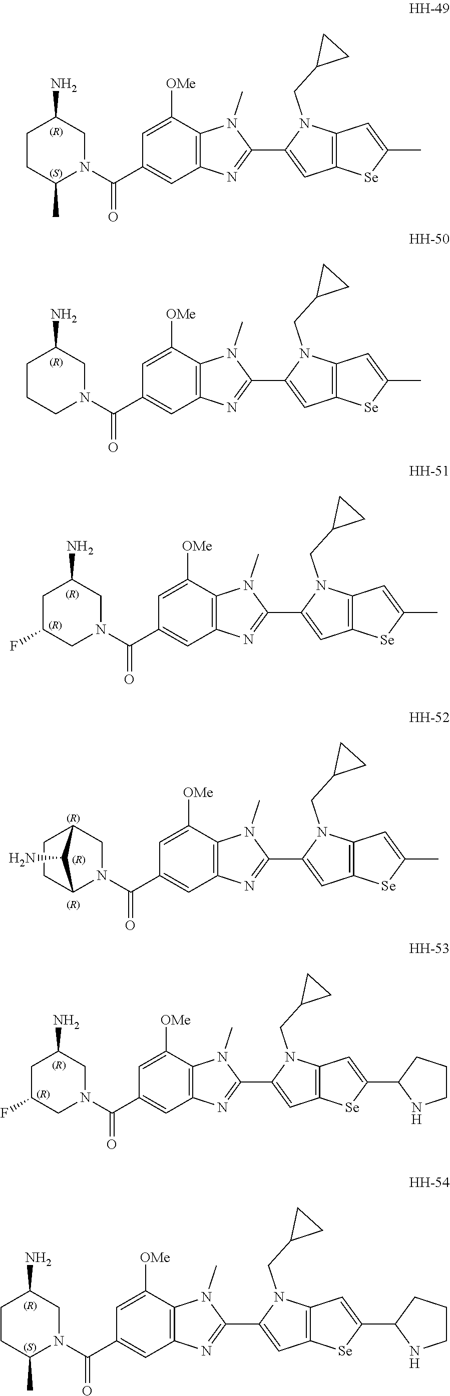

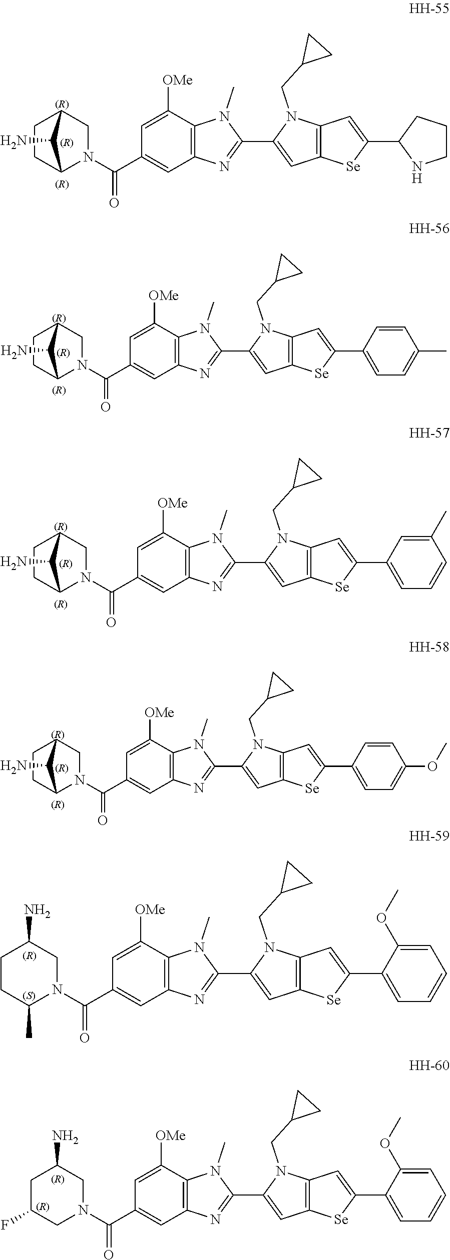

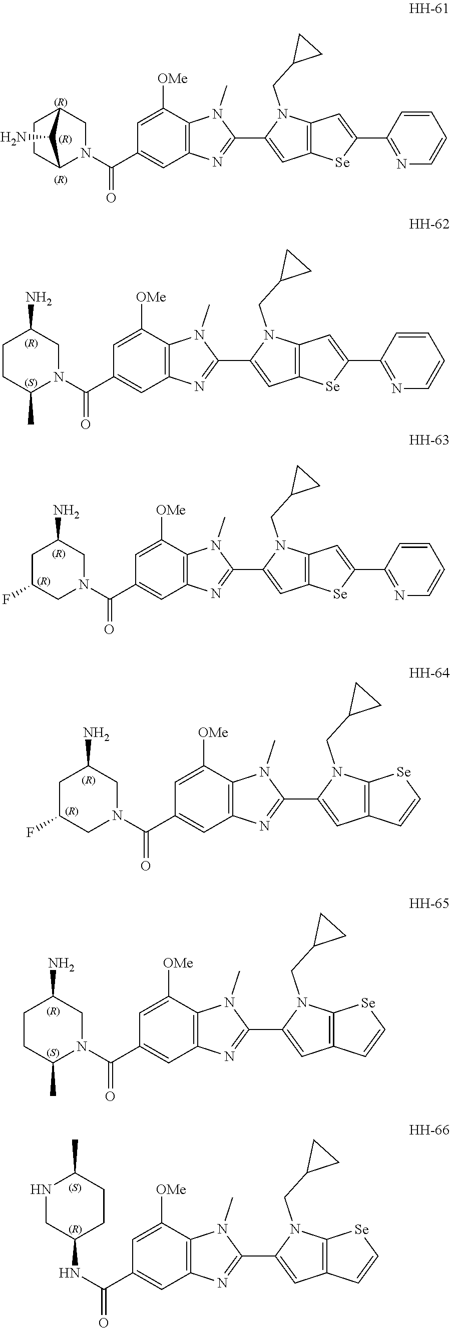

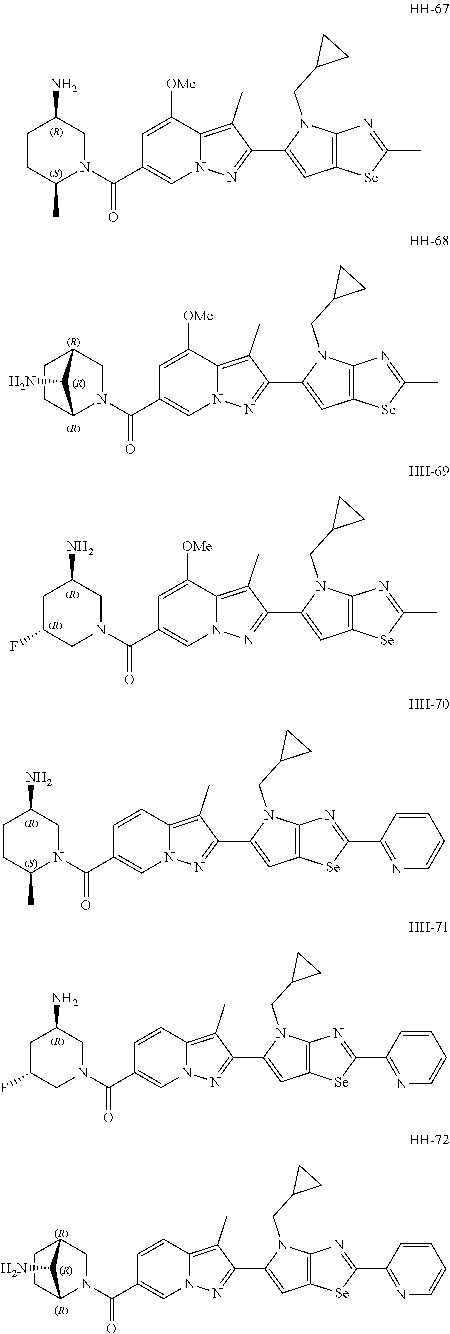

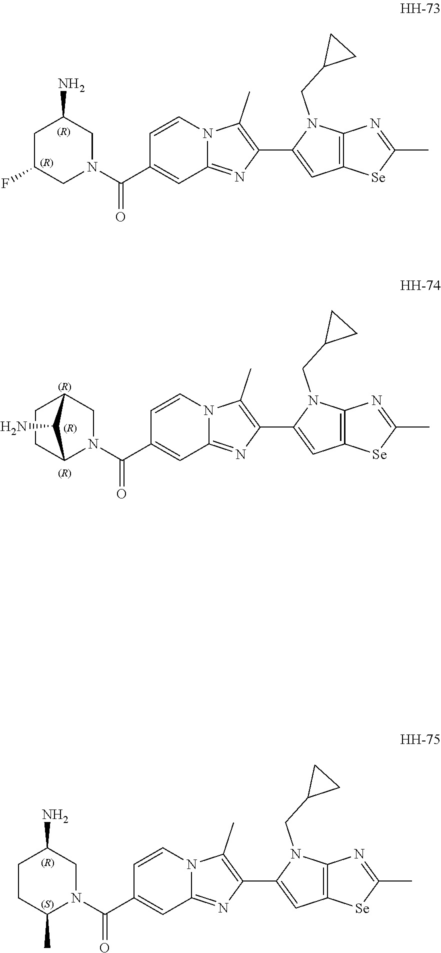

### Biological Assays

The prior art compounds Cpd-118 and Cpd-27, as well as compounds of the present disclosure were assayed as inhibitors of PAD4 using the assay protocol describe below.

Citrullination was detected via ammonia release based on published methodology (Nature Chemical Biology, 2015, 11: 189-191).

### Example 76. In-Vitro Measurement for Enzymatic PAD4 Citrullination Inhibition Activity

Citrullination was detected via ammonia release based on published methodology (Nature Chemical Biology, 2015, 11: 189-191).

PAD4 enzyme was diluted to 30 nM in Assay Buffer (100 mM HEPES, 50 mM NaCl, 2 mM DTT, 0.6 mg/ml BSA, pH 8) and added to wells containing various concentrations of compound or DMSO vehicle (0.8% final) in a high-volume black 96-well plate (Greiner). Following a 60-min preincubation at 27° C., the reaction was initiated by the addition of substrate (3 mM BAEE in 100 mM HEPES, 50 mM NaCl, 500 M CaCl₂, 2 mM DTT, pH 8). The reaction was stopped after 90 min by the addition of stop/detection buffer containing 50 mM EDTA, 2.6 mM o-phthalaldehyde and 2.6 mM DTT. Assays were incubated at 27° C. for 120 min before measuring fluorescence (λₑₓ 410/λₑₘ 476) on an Envision plate reader (PerkinElmer). After which IC₅₀ values were calculated using GraphPad Prism 8.0.

The IC₅₀ of the prior art compounds (Cpd-27, Cpd-118) and the corresponding selenium containing compounds measured according to the above protocol are shown as follows for direct comparison.

TABLE 1
Direct comparison of the IC₅₀ for enzymatic PAD4 citrullination inhibition

| Compound name | Chemical structure | IC₅₀ |
|---|---|---|
| Cpd-27 | 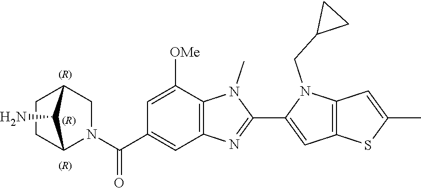 | 272 nM |
| HH-52 | 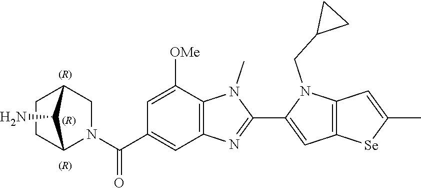 | 25.13 nM |
| Cpd-118 | 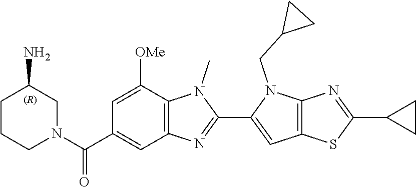 | 66.85 nM |
| HH-4 | 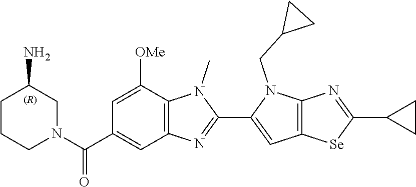 | 22.35 nM |

As can be seen, the PAD4 inhibitory activity of the selenium containing compound has improved 3 folds or more compared with the corresponding sulfur containing compound, and for some specific compounds, the PAD4 inhibitory activity has improved 10 folds or more compared with the corresponding sulfur containing compound.

The IC₅₀ for all the compounds are as shown in Table 2.

TABLE 2
The IC₅₀ of the compounds of the present disclosure

| | IC₅₀ of inhibition enzymatic activity of PAD4 |
|---|---|
| | (Colorimetric assays, Nature Chemical Biology, 2015, |
| Compound Name | 11: 189-191) |
| HH-1 | A |
| HH-2 | A |
| HH-3 | A |
| HH-4 | A |
| HH-5 | A |
| HH-6 | A |
| HH-7 | A |
| HH-8 | A |
| HH-9 | A |
| HH-10 | A |
| HH-11 | C |
| HH-12 | A |
| HH-13 | D |
| HH-14 | C |
| HH-15 | A |
| HH-16 | A |
| HH-17 | A |
| HH-18 | A |
| HH-19 | C |
| HH-20 | C |
| HH-21 | C |
| HH-22 | C |
| HH-23 | C |
| HH-24 | C |
| HH-25 | C |
| HH-26 | C |
| HH-27 | C |
| HH-28 | D |
| HH-29 | C |
| HH-30 | C |
| HH-31 | C |
| HH-32 | C |
| HH-33 | C |
| HH-34 | B |
| HH-35 | B |
| HH-36 | C |
| HH-37 | C |
| HH-38 | C |
| HH-39 | D |
| HH-40 | C |
| HH-41 | C |
| HH-42 | C |
| HH-43 | C |
| HH-44 | C |
| HH-45 | D |
| HH-46 | D |
| HH-47 | B |
| HH-48 | C |
| HH-49 | A |
| HH-50 | B |
| HH-51 | A |
| HH-52 | A |
| HH-53 | B |
| HH-54 | B |
| HH-55 | C |
| HH-56 | C |
| HH-57 | C |
| HH-58 | C |
| HH-59 | A |
| HH-60 | C |
| HH-61 | C |
| HH-62 | B |
| HH-63 | C |
| HH-64 | B |
| HH-65 | B |
| HH-66 | C |
| HH-67 | C |
| HH-68 | B |
| HH-69 | B |
| HH-70 | C |
| HH-71 | C |
| HH-72 | C |
| HH-73 | B |
| HH-74 | B |
| HH-75 | A |

A: ≤ 50 nM; B: 50-100 nM; C: 100-500 nM; D: >500 nM

### Example 77. Combination Therapy

In addition, we studied the pharmacodynamics of selenium-containing PADs inhibitor. Pharmacologic inhibition of PAD4 improves antitumor effect of cell therapy (CAR-T cell, CAR-NIK cell etc.) and/or immune checkpoint inhibitors. In mouse tumor models, targeted inhibition of PAD4 using low dose of the selenium containing heterocycle inhibitor (~1 mpk) with cell therapy and/or immune checkpoint inhibitor significantly reduced both primary tumor growth (tumor growth inhibition percent >90%) and lung metastases, and substantially enhanced the effect of cell therapy, immune checkpoint inhibitors.

### Abbreviations and Specialist Terms

n Number
PBS Phosphate buffer saline
FA Formic acid
THF Tetrahydrofuran
TEA Triethanolamine
ACN Acetonitrile
TFA Trifluoroacetic acid
DMF N,N-Dimethylformamide
DCE Dichloroethane
DCM Dichloromethane
DIEA N,N-Diisopropylethylamine
HATU 2-(7-Azabenzotriazol-1-yl)-N,N,N′,N′-tetramethyluronium hexafluorophosphate
T₃P Propylphosphonic anhydride
DMSO Dimethyl sulfoxide
DCE Dichloroethane
PTSA p-Toluenesulfonamide
NIS N-Iodosuccinimide
DIBAL-H Diisobutyl aluminium hydride
Pd(dppf)Cl₂ Dichloro[1,1′-bis(diphenylphosphino)ferrocene]palladium(II)
THF Tetrahydrofuran
HPLC High Performance Liquid Chromatography
LCMS Liquid Chromatography Mass Spectrometry

What is claimed is:

1. A compound of Formula (I), a pharmaceutically acceptable salt thereof, a stereoisomer thereof, or a solvate thereof, 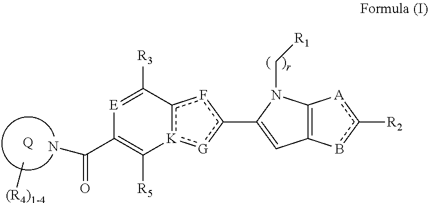
wherein,
one of A and B is Se, and the other one is N or CH;
E is N or CR₆;
K is C or N;
F and G are independently selected from the group consisting of N, NR₇, and CR₇; R₇ is selected from the group consisting of hydrogen and C₁-C₈ alkyl;
Ring Q is 4- to 15-membered heterocyclyl optionally substituted with 1-4 R₄;
the dashed lines denote an optionally existing double bond, wherein a five-membered ring containing A and B is a first heteroaryl ring, and a five-membered ring containing F, G, and H is a second heteroaryl ring;
r, at each occurrence, is independently selected from 0, 1, 2, 3, and 4;
R₁ and R₂ are independently selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl, and C₅-C₁₀ heteroaryl; R₁ and R₂ are optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy, and C₁-C₈ haloalkyl;
R₃, R₅, and R₆ are independently selected from the group consisting of H, F, Cl, Br, C₁-C₄ alkyl, and C₁-C₄ alkoxy;
R₄ is selected from the group consisting of H, F, Cl, Br, —CN, —NH₂, —OH, —NO₂, C₁-C₄ alkyl, —(CH₂)ₚNRₐRₐ, and —(CH₂)ₚOR_{b}, optionally two R₄ groups are taken together to form a carbocyclyl or heterocyclyl;
Rₐ and R_{b}, at each occurrence, is independently selected from H and C₁-C₄ alkyl;
p, at each occurrence, is independently selected from 0, 1, and 2.

2. The compound according to claim 1, the pharmaceutically acceptable salt thereof, the stereoisomer thereof, or the solvate thereof, wherein the compound has Formula (IIa) or (IIb): 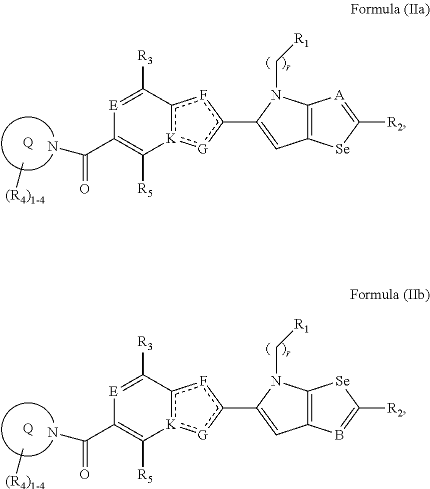
wherein when the compound has the Formula (IIa), A is N or CH; when the compound has the Formula (IIb), B is N or CH;
Ring Q is 4- to 12-membered heterocyclyl, preferably 4- to 7-membered monoheterocyclyl, 7- to 12-membered spiro heterocyclyl, or 6- to 10-membered bridged heterocyclyl; which-Ring Q is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, and —(CH₂)ₚOR_{b};
E, F, G, K, R₁, R₂, R₃, R₄, R₅, R₆, R₇, Rₐ, R_{b}, R_{c}, r, and p have the meanings as defined in claim 1.

3. The compound according to claim 1, the pharmaceutically acceptable salt thereof, the stereoisomer thereof, or the solvate thereof, wherein the compound has Formula (IIIa) or (IIIb): 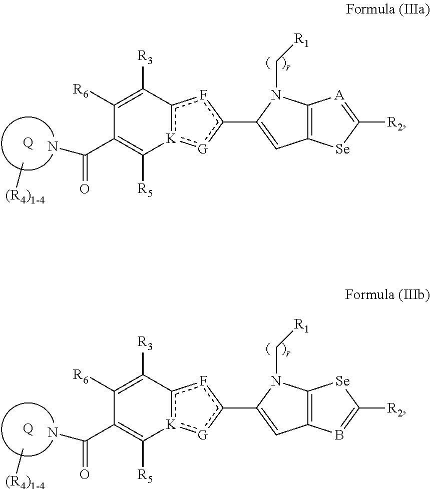
wherein when the compound has the Formula (IIIa), A is N or CH; when the compound has the Formula (IIIb), B is N or CH;
Ring Q is 4- to 12-membered heterocyclyl, preferably 4- to 7-membered monoheterocyclyl, 7- to 12-membered spiro heterocyclyl, or 6- to 10-membered bridged heterocyclyl; Ring Q is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, and —(CH₂)ₚOR_{b};
E, F, G, K, R₁, R₂, R₃, R₄, R₅, R₆, R₇, Rₐ, R_{b}, R_{c}, r, and p have the meanings as defined in claim 1.

4. The compound according to claim 1, the pharmaceutically acceptable salt thereof, the stereoisomer thereof, or the solvate thereof, wherein
B is Se;
A is N or CH;
a bicyclic ring containing E, F, G, and K is selected from the group consisting of the following structures: 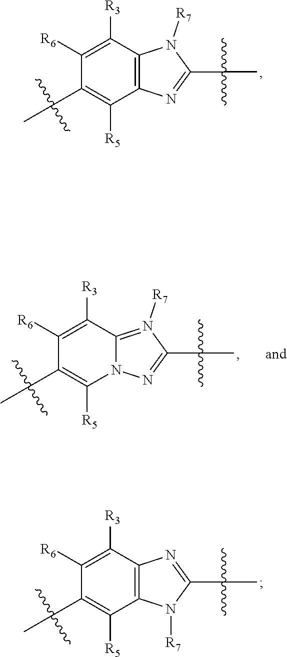
R₁, R₂, R₃, R₄, R₅, R₆, R₇, Rₐ, R_{b}, R_{c}, r, and p have the meanings as defined in claim 1.

5. The compound according to claim 1, the pharmaceutically acceptable salt thereof, the stereoisomer thereof, or the solvate thereof,
wherein Ring Q is selected from the group consisting of: 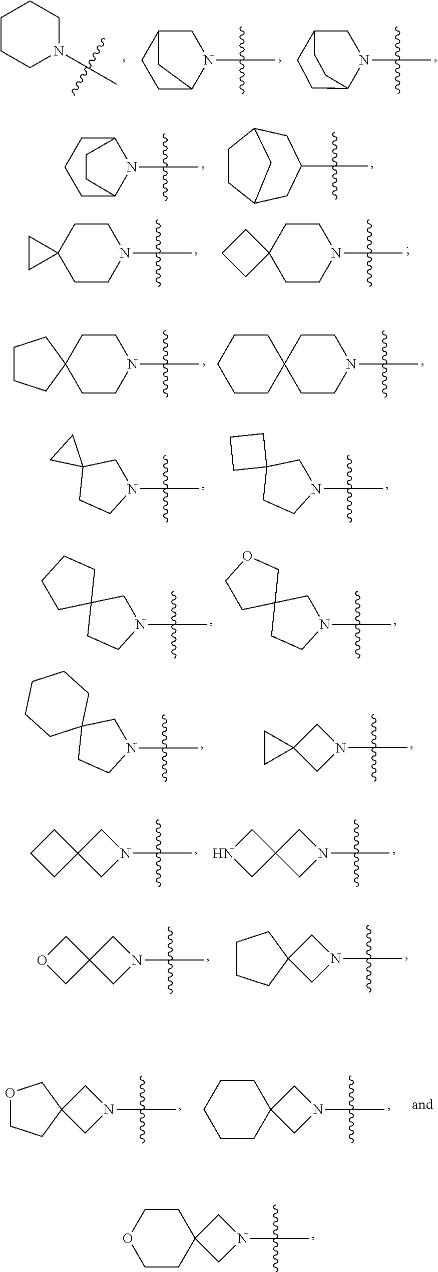
Ring Q is optionally substituted with 1-4 R₄, wherein R₄ is selected from the group consisting of H, F, Cl, Br, —CN, —NH₂, —OH, —NO₂, C₁-C₄ alkyl, —(CH₂)ₚNRₐRₐ, and —(CH₂)ₚOR_{b};
Rₐ, R_{b}, and p have the meanings as defined in claim 1.

6. The compound according to claim 1, the pharmaceutically acceptable salt thereof, the stereoisomer thereof, or the solvate thereof, wherein the compound has Formula (IVa) or (IVb): 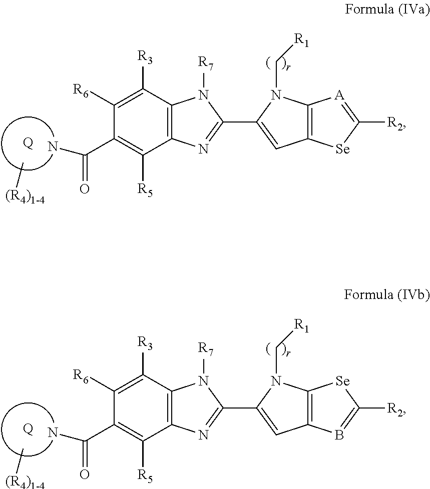
wherein when the compound has the Formula (IVa), A is N or CH; when the compound has the Formula (IVb), B is N or CH;
Ring Q with R₄ substitution is selected from the group consisting of 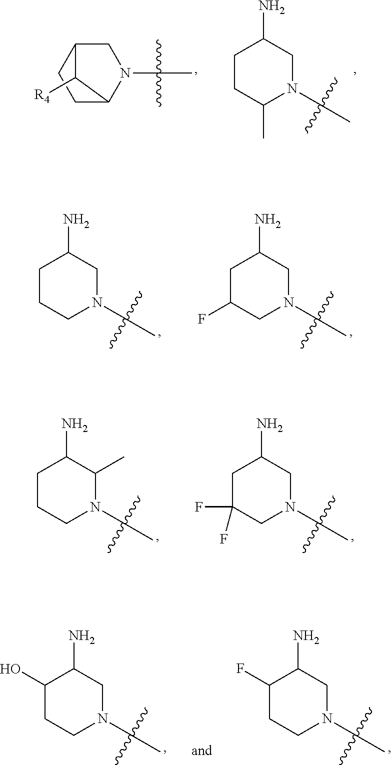
wherein R₄ in 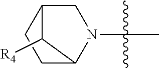
is selected from the group consisting of H, F, Cl, Br, —CN, —NH₂, —OH, —NO₂, C₁-C₄ alkyl, —(CH₂)ₚNRₐRₐ, and —(CH₂)ₚOR_{b};
R₁, R₂, R₃, R₄, R₅, R₆, R₇, Rₐ, R_{b}, R_{c}, r, and p have the meanings as defined in claim 1.

7. The compound according to claim 1, the pharmaceutically acceptable salt thereof, the stereoisomer thereof, or the solvate thereof, wherein the compound has Formula (IVa) or (IVb): 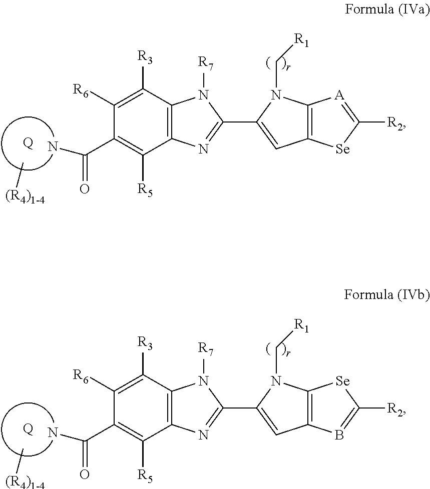
wherein when the compound has the Formula (IVa), A is N or CH; when the compound has the Formula (IVb), B is N or CH;
Ring Q with R₄ substitution is selected from the group consisting of 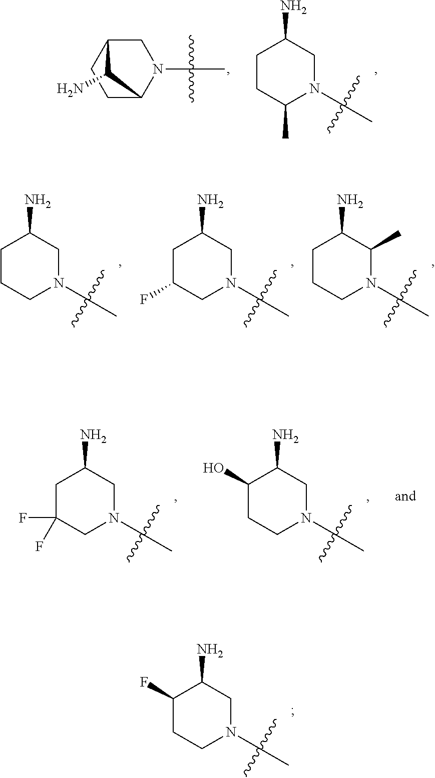
R₁ is selected from the group consisting of H, C₁-C₈ alkyl substituted with 0-5 R_{c}, C₃-C₆ cycloalkyl substituted with 0-5 R_{c}, C₆-C₁₀ aryl substituted with 0-5 R_{c}, and C₅-C₁₀ heteroaryl substituted with 0-4 R_{c};
R₂ is selected from the group consisting of H, C₁-C₈ alkyl substituted with 0-5 R_{c}, C₃-C₆ cycloalkyl substituted with 0-5 R_{c}, C₅-C₉ heterocycloalkyl substituted with 0-5 R_{c}, C₆-C₁₀ aryl substituted with 0-5 R_{c}, and C₅-C₁₀ heteroaryl substituted with 0-4 R_{c};
R₃ is selected from the group consisting of H, F, Cl, Br, C₁-C₄ alkyl, and C₁-C₄ alkoxy, and is preferably H, F, or C₁-C₃ alkoxy;
R₅ and R₆ are independently selected from the group consisting of H, F, Cl, Br, C₁-C₄ alkyl, and C₁-C₄ alkoxy, and are preferably H, F, or C₁-C₃ alkyl;
R₇ is selected from the group consisting of hydrogen and C₁-C₈ alkyl, and is preferably H or C₁-C₃ alkyl;
R_{c} at each occurrence is independently selected from the group consisting of F, Cl, Br, —CN, —NO₂, —OH, —NH₂, C₁-C₃ alkyl, C₁-C₃ alkoxy, and C₁-C₃ haloalkyl;
r is 1 or 2.

8. The compound according to claim 1, the pharmaceutically acceptable salt thereof, the stereoisomer thereof, or the solvate thereof, wherein
R₁ is selected from cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, pyridinyl substituted with 0-2 R_{c};
R₂ is selected from H, F, Cl, Br, methyl, ethyl, cyclopropyl substituted with 0-2 R_{c}, phenyl substituted with 0-2 R_{c}, pyrrolidinyl substituted with 0-2 R_{c}, pyridinyl substituted with 0-2 R_{c}, piperidinyl substituted with 0-2 R_{c};
R_{c} at each occurrence is independently selected from the group consisting of F, C₁-C₃ alkyl, C₁-C₃ alkoxy, and C₁-C₃ haloalkyl, and is preferably methyl, —OMe, or —CF₃;
r is 1.

9. The compound according to claim 1, the pharmaceutically acceptable salt thereof the stereoisomer thereof, or the solvate thereof, wherein the compound is selected from the group consisting of:

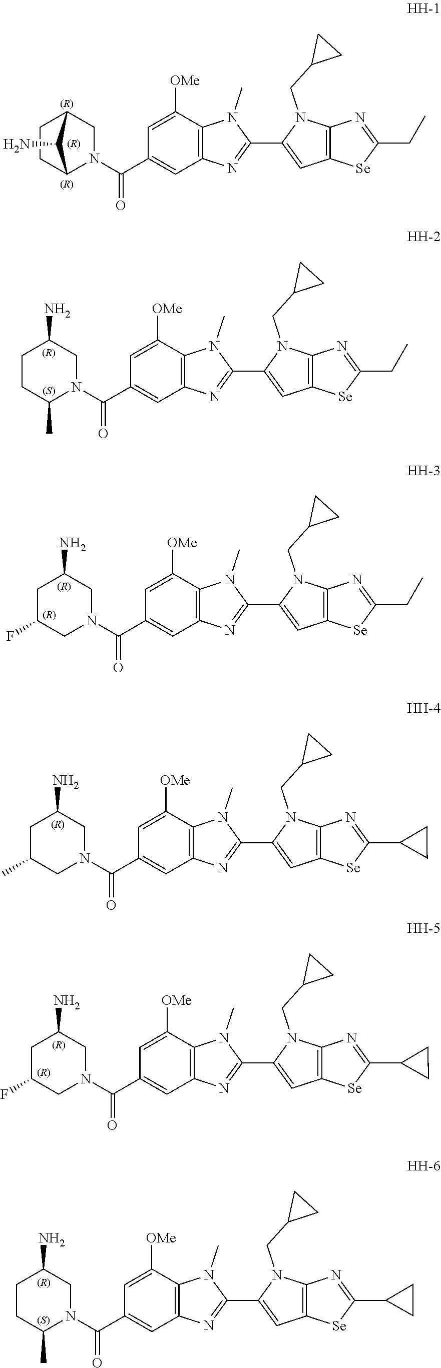

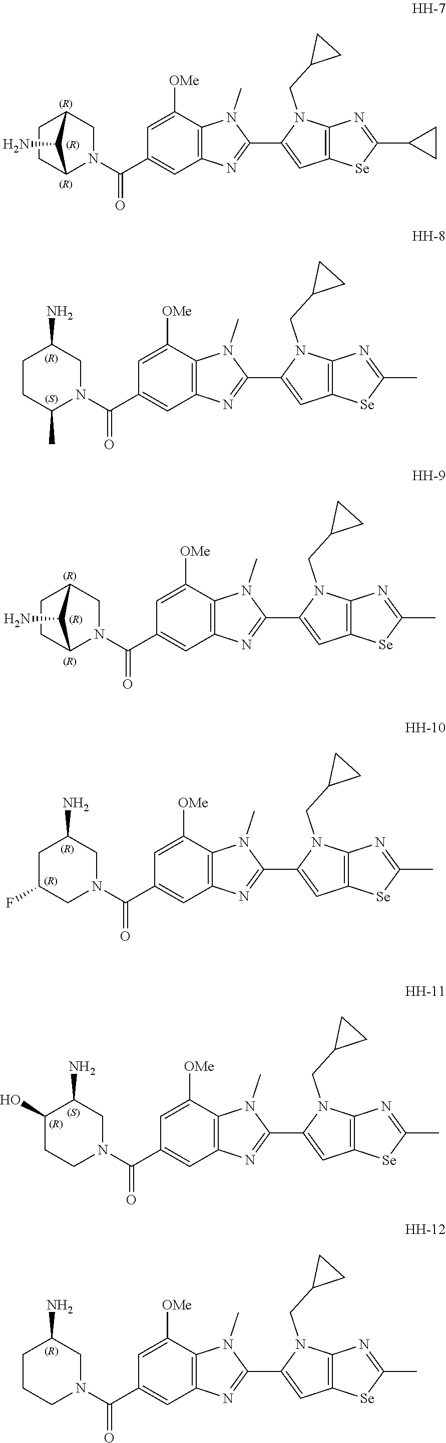

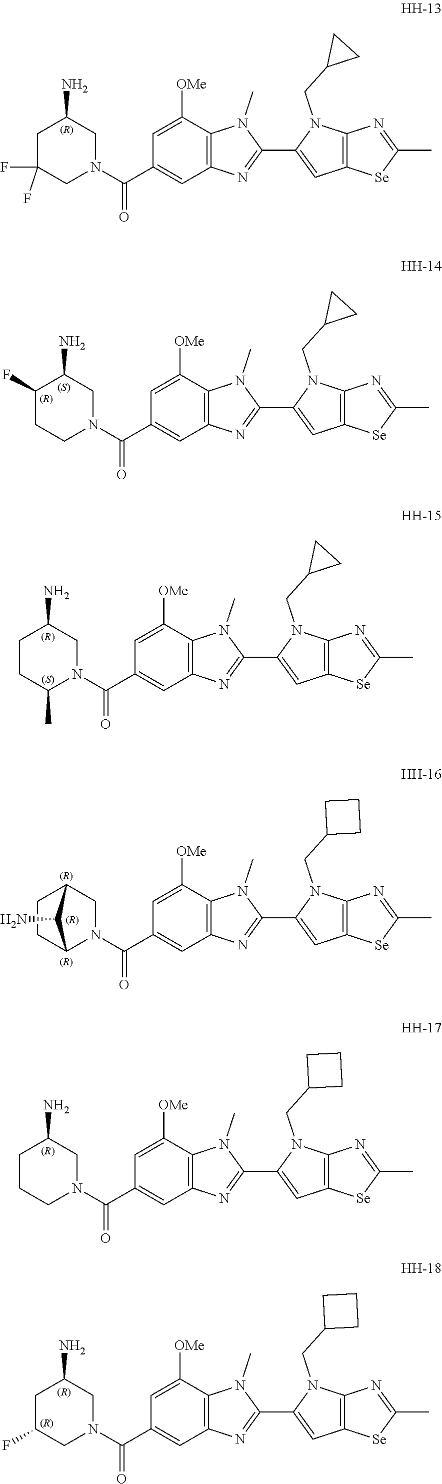

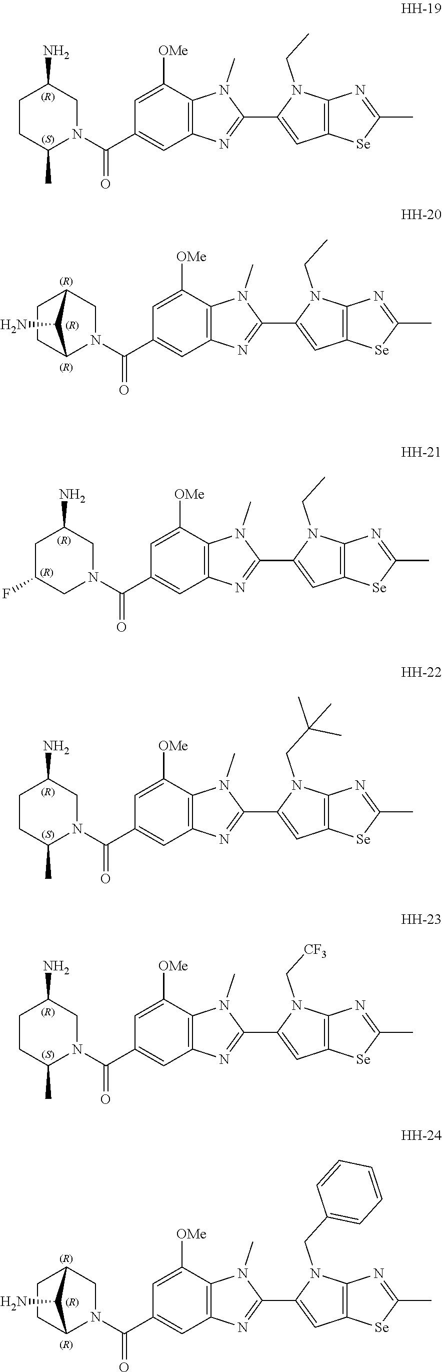

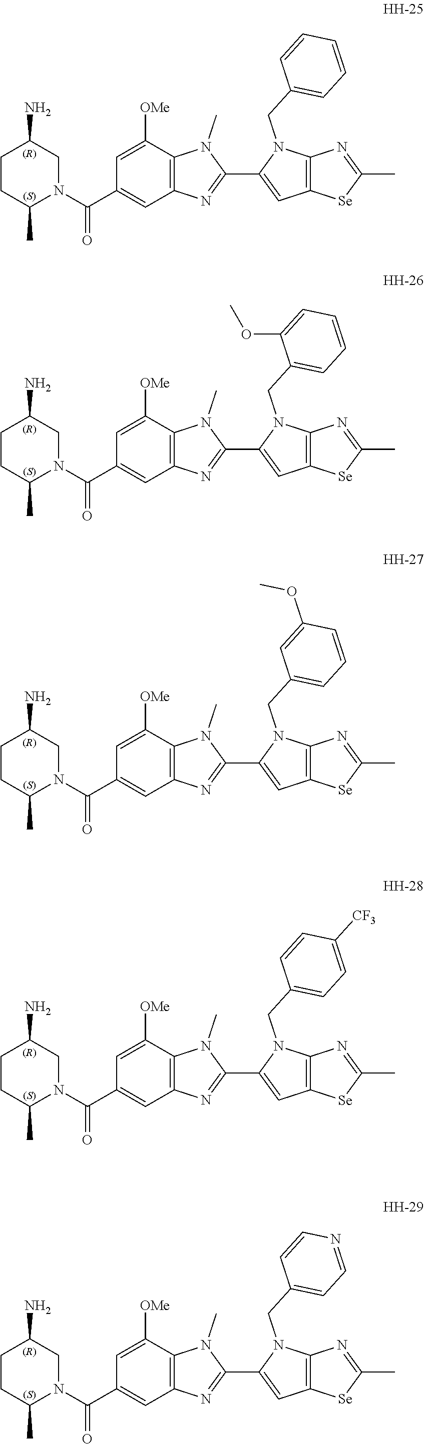

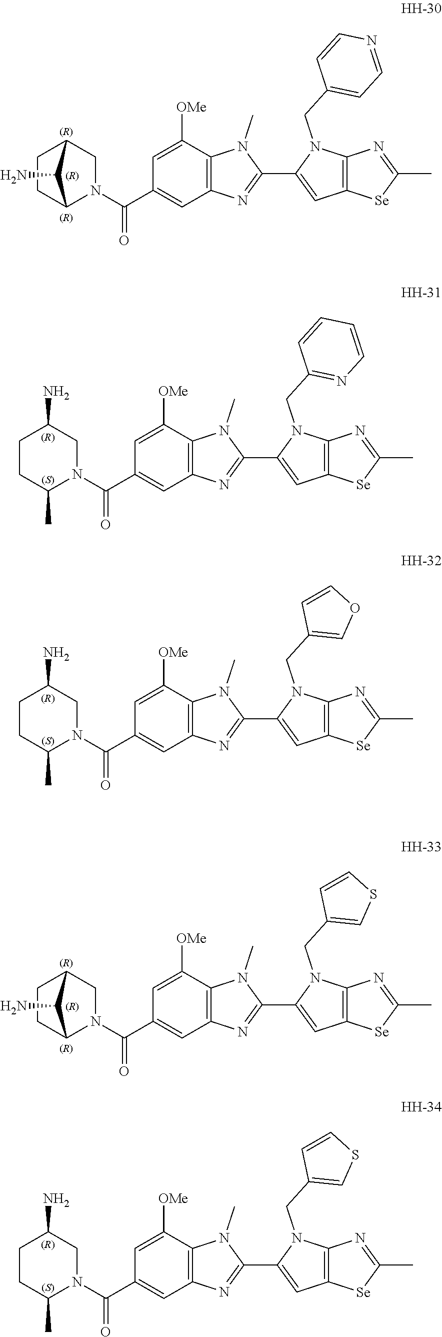

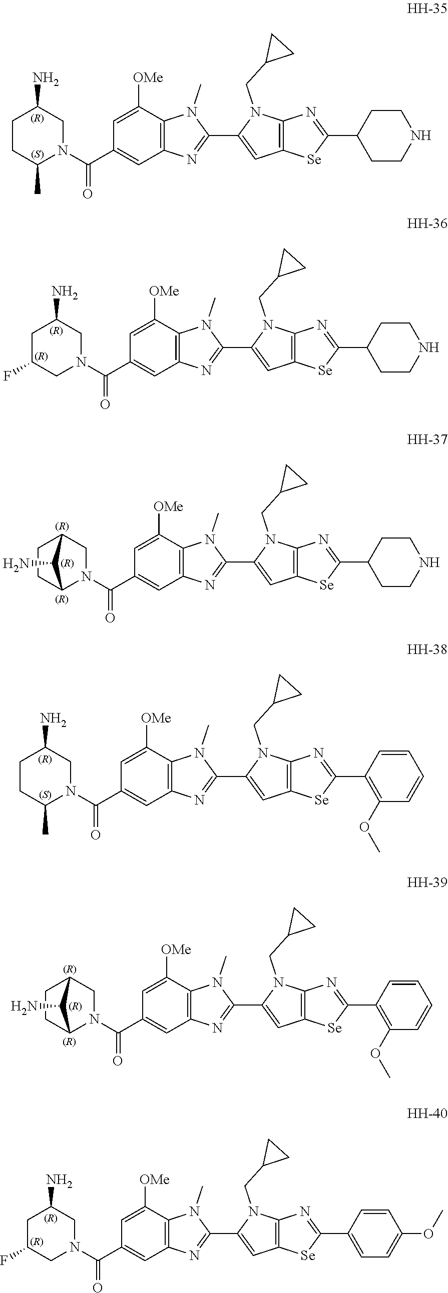

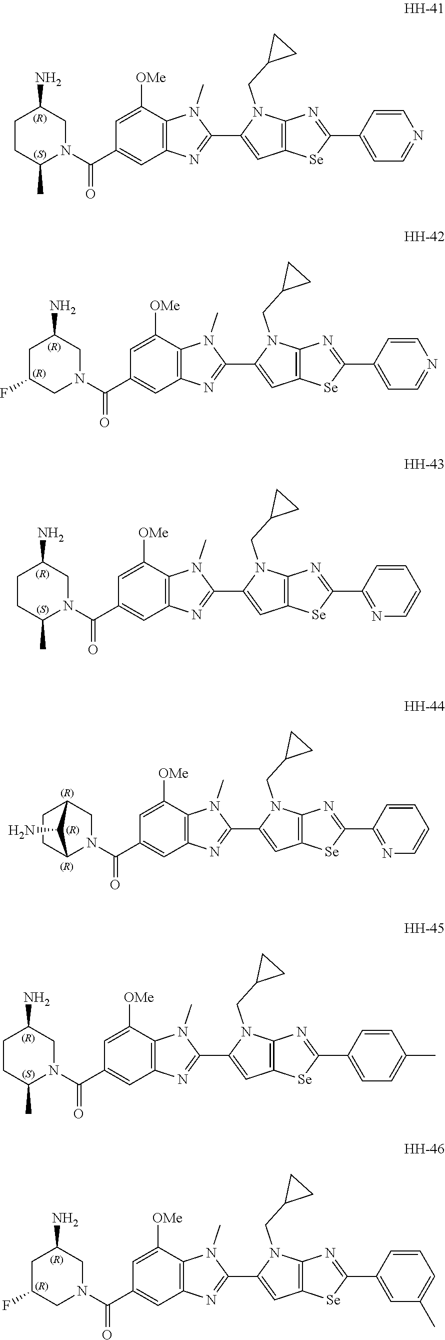

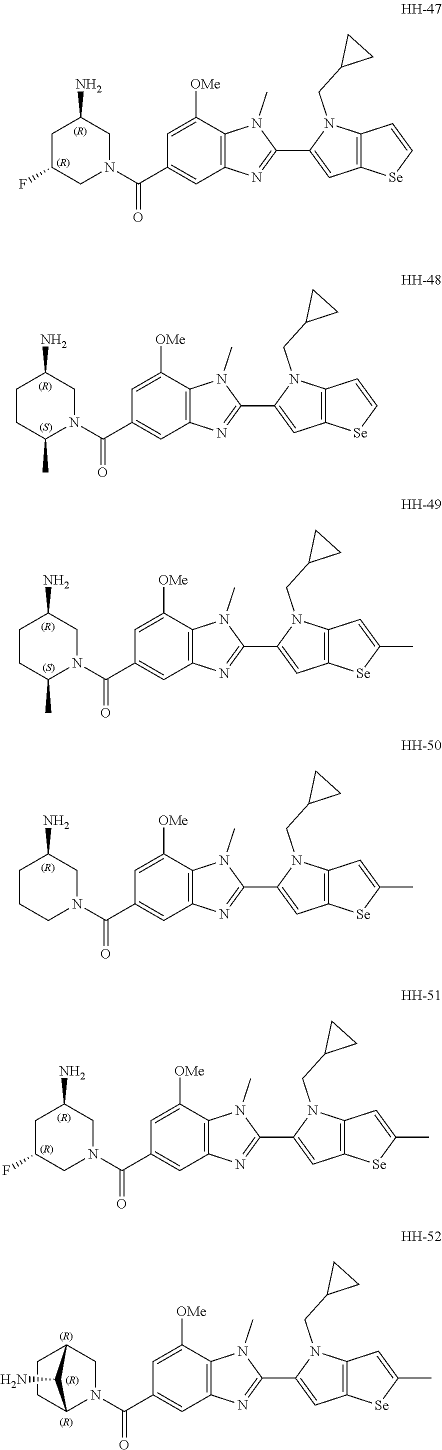

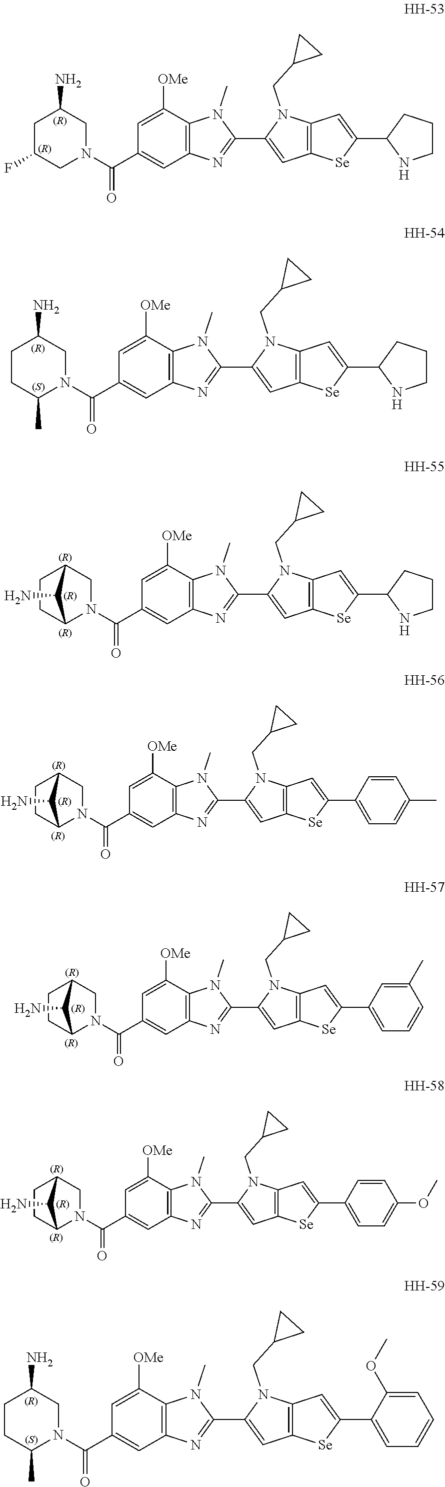

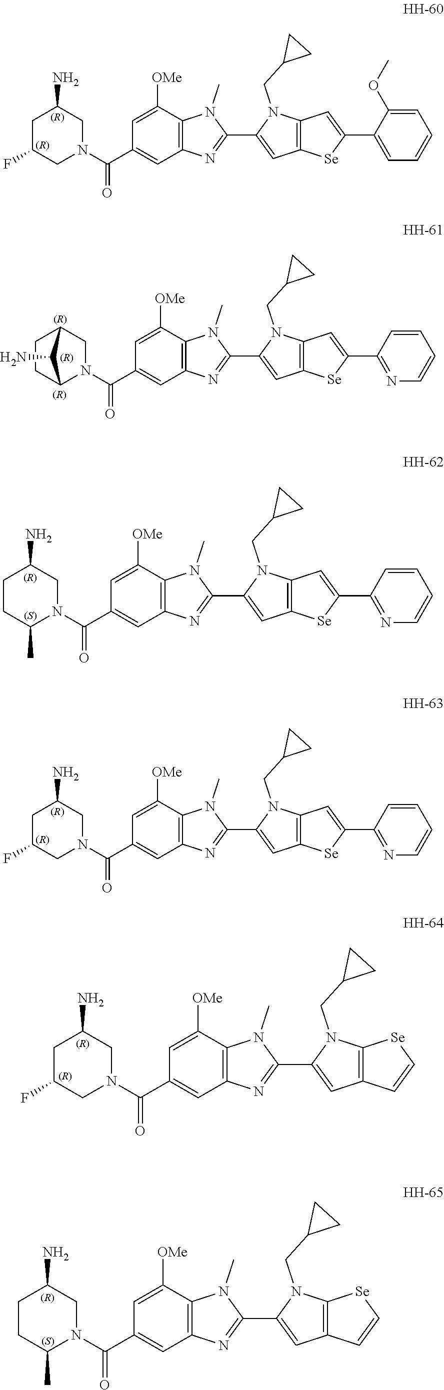

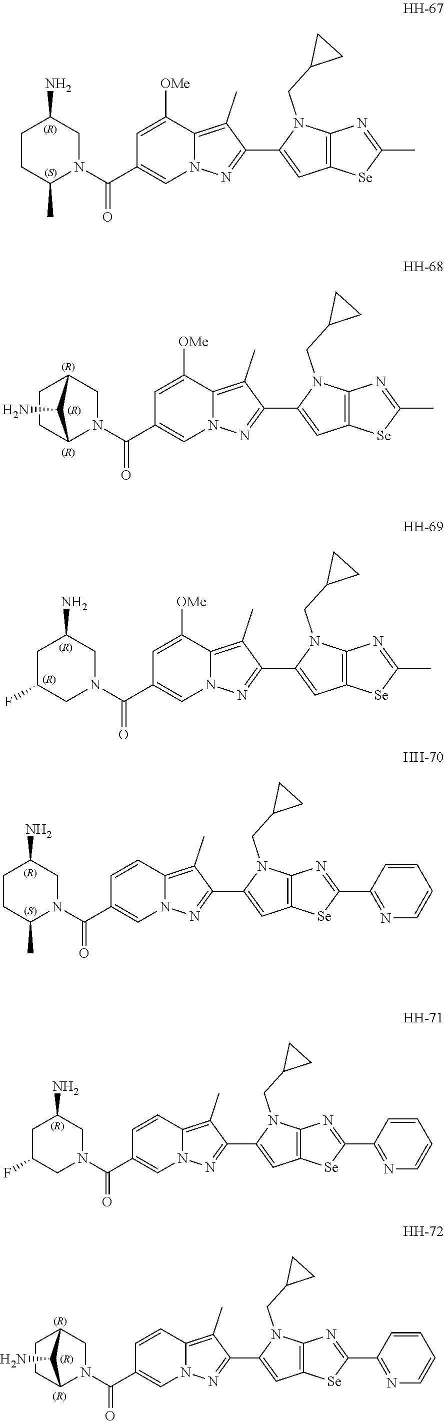

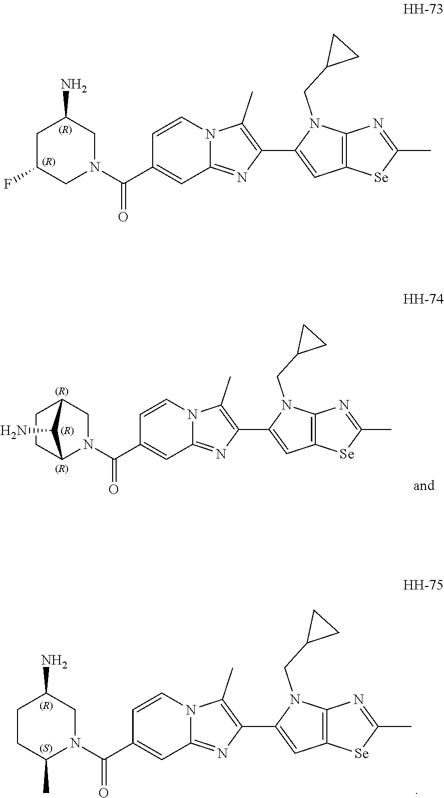

10. A compound of formula (V), a pharmaceutically acceptable salt thereof, a stereoisomer thereof, or a solvate thereof, 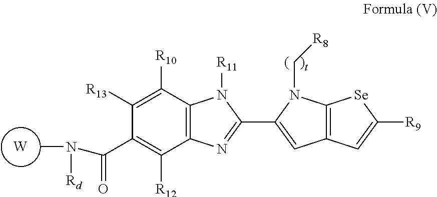
wherein,
Ring W is 4- to 15-membered heterocyclyl, preferably 4- to 7-membered monoheterocyclyl, more preferably 5- to 7-membered monoheterocyclyl, and Ring W is optionally substituted with 1-4 R₁₄;
t, at each occurrence, is independently selected from 0, 1, 2, 3, and 4;
R₈ and R₉ are independently selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl, and C₅-C₁₀ heteroaryl; R₈ and R₉ are optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy, and C₁-C₈ haloalkyl;
R₁₀, R₁₂ and R₁₃ are independently selected from the group consisting of H, F, Cl, Br, C₁-C₄ alkyl, and C₁-C₄ alkoxy;
R₁₁ is selected from the group consisting of hydrogen and C₁-C₈ alkyl;
R₁₄ is selected from the group consisting of H, F, Cl, Br, —CN, —NH₂, —OH, —NO₂, and C₁-C₄ alkyl;
R_{d} is independently selected from H and C₁-C₄ alkyl.

11. The compound according to claim 10, the pharmaceutically acceptable salt thereof, the stereoisomer thereof, or the solvate thereof, wherein Ring W is 6-membered monoheterocyclyl, preferably piperidinyl, and Ring W is optionally substituted with 1 or 2 R₁₄.

12. The compound according to claim 10, the pharmaceutically acceptable salt thereof, the stereoisomer thereof, or the solvate thereof, wherein
t is 1 or 2;
R₈ is selected from the group consisting of H, C₁-C₅ alkyl substituted with 0-5 R_{c}, C₃-C₆ cycloalkyl substituted with 0-5 Re, C₆-C₁₀ aryl substituted with 0-5 R_{c}, and C₅-C₁₀ heteroaryl substituted with 0-4 R_{c};
R₉ is selected from the group consisting of H, C₁-C₈ alkyl substituted with 0-5 R_{c}, C₃-C₆ cycloalkyl substituted with 0-5 R_{c}, C₅-C₉ heterocycloalkyl substituted with 0-5 R_{c}, C₆-C₁₀ aryl substituted with 0-5 R_{c}, and C₅-C₁₀ heteroaryl substituted with 0-4 R_{c};
R₁₀ is selected from the group consisting of H, F, Cl, Br, C₁-C₄ alkyl, and C₁-C₄ alkoxy, and is preferably H, F, or C₁-C₃ alkoxy;
R₁₂ and R₁₃ are independently selected from the group consisting of H, F, Cl, Br, C₁-C₄ alkyl, and C₁-C₄ alkoxy, and are preferably H, F, or C₁-C₃ alkyl;
R₁₁ is selected from the group consisting of hydrogen and C₁-C₈ alkyl, and is preferably H or C₁-C₃ alkyl; and
R_{c} at each occurrence is independently selected from the group consisting of F, Cl, Br, —CN, —NO₂, —OH, —NH₂, C₁-C₃ alkyl, C₁-C₃ alkoxy, and C₁-C₃ haloalkyl.

13. The compound according to claim 10, the pharmaceutically acceptable salt thereof the stereoisomer thereof, or the solvate thereof,
wherein
R₈ is selected from cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, pyridinyl substituted with 0-2 R_{c};
R₉ is selected from H, F, Cl, Br, methyl, ethyl, cyclopropyl substituted with 0-2 R_{c}, phenyl substituted with 0-2 R_{c}, pyrrolidinyl substituted with 0-2 R_{c}, pyridinyl substituted with 0-2 R_{c}, piperidinyl substituted with 0-2 R_{c};
R_{c} at each occurrence is independently selected from the group consisting of F, C₁-C₃ alkyl, C₁-C₃ alkoxy, and C₁-C₃ haloalkyl, and is preferably methyl, —OMe, or —CF₃;
R_{d} is H; and
t is 1.

14. (canceled)

15. A pharmaceutical composition comprising the compound according to claim 1, the pharmaceutically acceptable salt thereof, the stereoisomer thereof, or the solvate thereof, and one or more pharmaceutically acceptable carriers.

16. A method of treating a PAD4-mediated or NET-mediated disease, disorder, or condition in a subject, comprising administering to the subject a therapeutically effective amount of the compound according to claim 1, the pharmaceutically acceptable salt thereof, the stereoisomer thereof, or the solvate thereof, wherein the PAD4-mediated or NET-mediated disease, disorder, or condition is selected from the group consisting of cancers, rheumatoid arthritis, multiple sclerosis, vasculitis, systemic lupus erythematosus, ulcerative colitis, cystic fibrosis, asthma, cutaneous lupus erythematosis, psoriasis, Alzheimer's disease, ischemia-reperfusion injury, and immune responses induced during transplant rejection.

17. The method according to claim 16, wherein the PAD4-mediated or NET-mediated disease, disorder, or condition is selected from the group consisting of lung cancer, liver cancer, blood cancer, esophageal cancer, breast cancer, and colon cancer.

18. A combination therapy, comprising administering to a subject a first therapeutic agent and one or more additional therapeutics, wherein the first therapeutic agent comprises the compound according to claim 1, the pharmaceutically acceptable salt thereof, the stereoisomer thereof, or the solvate thereof, the one or more additional therapeutics is selected from the group consisting of radiotherapy, chemotherapy, cell therapy, and immune checkpoint inhibitor, wherein the cell therapy comprises CAR-T, CAR-NK, CAR-NKT, CAR-M, CAR-Treg, CAR-γδT, TIL, and TCR-T, the immune checkpoint inhibitor comprises PD-1 inhibitor, PD-L1 inhibitor, CTLA-4 inhibitor, B7-H3 inhibitor, LAG3 inhibitor, TIM3 inhibitor, TIGIT inhibitor, anti-PDL1/TGFβ bispecific antibody, anti-EpCAM-CD3 bispecific antibody, and/or CD40 agonists.

19. The combination therapy according to claim 18, wherein the first therapeutic agent and the one or more additional therapeutics are administered together, simultaneously, sequentially, or alternately; and
the first therapeutic agent and the one or more additional therapeutics are administrated in a lower dose in comparison with a dose of the first therapeutic agent or the one or more additional therapeutics when administered alone.

20. (canceled)

21. A method for preparing selenazole compound S-4, comprising the following steps: 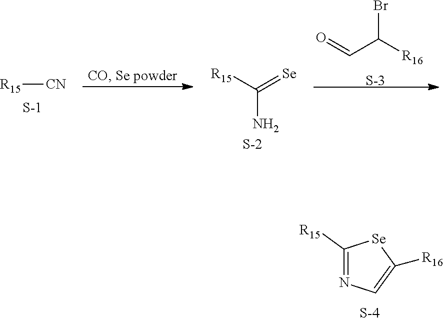
reacting a nitrile compound S-1 with CO and Se powder to yield a compound S-2, which is further reacted with a compound S-3 to yield the selenazole compound S-4, wherein,
R₁₅ is selected from the group consisting of H, F, Cl, Br, C₁-C₈ alkyl, C₂-C₈ alkenyl, C₂-C₈ alkynyl, C₁-C₈ alkoxy, C₁-C₈ haloalkyl, C₁-C₈ alkylamino, C₃-C₈ cycloalkyl, C₃-C₉ heterocyclyl, C₆-C₁₀ aryl, and C₅-C₁₀ heteroaryl; R₁₅ is optionally substituted with 0-5 R_{c}, R_{c} at each occurrence is independently selected from the group consisting of F, Cl, Br, —CN, —NO₂, —OH, —NH₂, C₁-C₈ alkyl, C₁-C₈ alkoxy, and C₁-C₈ haloalkyl; and is preferably selected from the group consisting of H, C₁-C₅ alkyl substituted with 0-5 R_{c}, C₃-C₆ cycloalkyl substituted with 0-5 R_{c}, C₆-C₁₀ aryl substituted with 0-5 R_{c}, and C₅-C₁₀ heteroaryl substituted with 0-4 R_{c}; and is more preferably selected from the group consisting of cyclopropyl substituted with 0-2 R_{c}, cyclobutyl substituted with 0-2 R_{c}, methyl, tert-butyl, phenyl substituted with 0-2 R_{c}, furanyl substituted with 0-2 R_{c}, thienyl substituted with 0-2 R_{c}, and pyridinyl substituted with 0-2 R_{c};
R_{c} is at each occurrence is independently selected from the group consisting of F, C₁-C₃ alkyl, C₁-C₃ alkoxy, and C₁-C₃ haloalkyl, and is preferably methyl, —OMe, or —CF₃;
R₁₆ is CHO.

22. (canceled)

23. A pharmaceutical composition comprising the compound according to claim 10, the pharmaceutically acceptable salt thereof, the stereoisomer thereof, or the solvate thereof, and one or more pharmaceutically acceptable carriers.

24. A method of treating a PAD4-mediated or NET-mediated disease, disorder, or condition in a subject, the method comprising administering to the subject a therapeutically effective amount of the compound according to claim 10, the pharmaceutically acceptable salt thereof, the stereoisomer thereof, or the solvate thereof, wherein the PAD4-mediated or NET-mediated disease, disorder, or condition is selected from the group consisting of cancers, rheumatoid arthritis, multiple sclerosis, vasculitis, systemic lupus erythematosus, ulcerative colitis, cystic fibrosis, asthma, cutaneous lupus erythematosis, psoriasis, Alzheimer's disease, ischemia-reperfusion injury, and immune responses induced during transplant rejection.

25. A combination therapy, comprising administering to a subject a first therapeutic agent and one or more additional therapeutics, wherein the first therapeutic agent comprises the compound according to claim 10, the pharmaceutically acceptable salt thereof, the stereoisomer thereof, or the solvate thereof, the one or more additional therapeutics is selected from the group consisting of radiotherapy, chemotherapy, cell therapy, and immune checkpoint inhibitor, wherein the cell therapy comprises CAR-T, CAR-NK, CAR-NKT, CAR-M, CAR-Treg, CAR-γδT, TIL, and TCR-T, the immune checkpoint inhibitor comprises PD-1 inhibitor, PD-L1 inhibitor, CTLA-4 inhibitor, B7-H3 inhibitor, LAG3 inhibitor, TIM3 inhibitor, TIGIT inhibitor, anti-PDL1/TGFβ bispecific antibody, anti-EpCAM-CD3 bispecific antibody, and/or CD40 agonists.
